(19) United States
(12) Patent Application Publication
(10) Pub. No.: US 2025/0043366 A1
(43) Pub. Date: Feb. 6, 2025

(54) TILED ASSAYS USING CRISPR-CAS BASED DETECTION

(71) Applicants: THE BROAD INSTITUTE, INC., CAMBRIDGE, MA (US); THE GENERAL HOSPITAL CORPORATION, BOSTON, MA (US); MASSACHUSETTS INSTITUTE OF TECHNOLOGY, CAMBRIDGE, MA (US)

(72) Inventors: Gowtham THAKKU, CAMBRIDGE, MA (US); Bwarenaba KAUTU, CAMBRIDGE, MA (US); James GOMEZ, CAMBRIDGE, MA (US); Roby BHATTACHARYYA, CAMBRIDGE, MA (US); Deborah HUNG, CAMBRIDGE, MA (US); Sharon WONG, CAMBRIDGE, MA (US)

(21) Appl. No.: 17/413,802

(22) PCT Filed: Dec. 13, 2019
(86) PCT No.: PCT/US2019/066401

§ 371 Date: Jun. 14, 2021

Related U.S. Application Data
(60) Provisional application No. 62/779,416, filed on Dec. 13, 2018.

(51) Int. Cl. C12Q 1/689; C12Q 1/6818; C12Q 1/682

(52) U.S. Cl. CPC C12Q 1/689; C12Q 1/6818; C12Q 1/682; C12Q 2600/106; C12Q 2600/16

(57) ABSTRACT

Disclosed herein are methods and systems utilizing CRISPR effector systems for assays and diagnostics. Embodiments herein provide tile probes in systems and methods for detection of multiple targets across a given genome or group of genomes, including in circulating nucleic acid samples.

### CROSS-REFERENCE TO RELATED APPLICATIONS

This application is a national stage application of International Application No. PCT/US2019/066401, filed Dec. 13, 2019, which claims the benefit of U.S. Provisional Application No. 62/779,416, filed Dec. 13, 2018. The entire contents of the above-identified applications are hereby fully incorporated herein by reference.

### REFERENCE TO AN ELECTRONIC SEQUENCE LISTING

The content of the Electronic Sequence Listing (BROD-3910US_ST25.txt); Size is 8396 bytes and was created on Dec. 7, 2021) is incorporated herein by reference in its entirety.

### TECHNICAL FIELD

The subject matter disclosed herein is generally directed to diagnostics related to the use of CRISPR effector systems.

### BACKGROUND

Despite gains in recent decades, infectious diseases continue to present a significant healthcare burden, resulting in over 20 million annual deaths worldwide. For many infections, cell cultures from patient samples remain the diagnostic gold standard. There are limitations to this approach, however: infection sites may be difficult to access; pathogen load in accessible sites (blood, urine, saliva) may be low; cultures can take days to weeks to grow. These challenges can lead to delays in identifying disease status and optimal treatment strategies.

Nucleic acid tests (NATs) have been developed that can rapidly identify the presence of pathogens. Typical workflows for these tests comprise of isolation of pathogens from clinical samples, extraction of nucleic acids, followed by amplification of a genomic target that is specific to the species or strain of pathogen being detected. Two key challenges limit the sensitivity and specificity of these tests, however: (1) they detect nucleic acids extracted from intact bacterial cells that may be in low abundance; (2) they target only a single (or handful of) short nucleic acid sequence(s) in genomes that can be millions of base pairs long, since more targets can lead to primer cross-reactivity and noise.

Studies have found a measurable amount of circulating cell-free nucleic acids (cfDNA) in blood. Whereas most of this cfDNA is of host origin, a small fraction has been shown to align with microbial genomes. The cfDNA landscape is also believed to vary with disease state, with a higher abundance of fragments in individuals with active infection. Second, novel nucleic acid sensing technologies based on CRISPR-Cas have been identified. Combined with initial target amplification, these methods can detect a single nucleic acid target at attomolar concentrations (e.g. SHERLOCK, DETECTR). While detection assays have advanced, there remains a need to identifying disease status and optimal treatment strategies.

### SUMMARY

In certain example embodiments, a nucleic acid detection system is provided, comprising one or more sets of proximity dependent probes, each set comprising two or more proximity dependent probes, each proximity dependent probe comprising a guide polynucleotide recognition sequence and a target binding sequence; one or more CRISPR-Cas proteins, a guide polynucleotide for each set of proximity dependent probes, each guide polynucleotide comprising a guide sequence capable of hybridizing to the guide polynucleotide recognition sequence of the one or more ligation dependent probe sets, and designed to form a complex with the one or more CRISPR-Cas proteins; and an oligonucleotide based detection construct comprising a non-target sequence, wherein the CRISPR-Cas protein exhibits collateral activity and cleaves the non-target sequence once activated by the target.

In embodiments, the proximity dependent probes are linked by ligation, splinted ligation, hybridization, or proximity extension. Proximity dependent probes can comprise one or more of a forward primer binding site, a reverse primer binding site, and a reverse polymerase binding site. In some instances, the proximity dependent probes further comprise an origin-specific barcode, a set-specific barcode, and/or a unique molecular identifier (UMI). The proximity dependent probes are, in embodiments, molecular inversion probes (MIPs), padlock probes, or split-ligation probes. The proximity dependent probes can comprise a gap region that, upon binding to a target sequence and gap filling, comprises a gap-filled sequence, in some instances, the gap-filed sequence comprises the guide polynucleotide recognition sequence. The gap-filled sequence can comprise modified nucleotides comprising a capture moiety. Systems can further comprise a capture agent that binds the capture moiety of the modified nucleotides comprising a capture moiety. In embodiments, the modified nucleotides are biotinylated nucleotides and the capture agent is streptavidin or a streptavidin coated surface.

In embodiments, the proximity dependent probe is a molecular inversion probe (MIP), wherein the MIP can comprise a first target binding sequence and a second target binding sequence linked by a linking region, the linking region comprising one or more of a forward primer binding sequence, a reverse primer binding sequence, a RNA polymerase binding sequence, a guide polynucleotide binding sequence, and a barcode. In certain embodiments the first target binding sequence and the second target binding sequence hybridize on the target sequence directly adjacent to one another. In certain embodiments, the first and second target binding sequence hybridize on the target sequence such that there is at least a single nucleotide gap region between the first and second target binding sequence. In embodiments, filling the gap region between the first and second targeting binding sequence generates the guide polynucleotide recognition sequence.

In certain example embodiments, a detection system for detecting the presence of one or more genus or species of an organism in a sample is provided, comprising a set of two or more molecular inversion probes (MIPs), each MIP comprising a guide RNA recognition sequence area and a target of interest sequence; and one or more CRISP detection systems comprising one or more guide RNAs, each guide RNA comprising a sequence designed to identify the presence of a species, genus, or to distinguish a category of organism. In some embodiments, the guide RNA recognition sequence is in the MIP backbone in proximity to the target sequence of interest, is integrated into the target sequence of interest in the MIP, or the MIP is configured that upon hybridization comprises a gap region that when filled, comprises the guide RNA recognition sequence. The MIPs of the systems and methods disclosed herein can comprise one or more of forward and reverse primers for amplification optionally including a T7 handle of RNA transcription, an inter-primer element for MIP linearization, and a barcode.

In embodiments, the tiled guide polynucleotide set comprises 2 to 50 guides per target sequence; in certain embodiments, the proximity probe set is tiled and comprises 2 to 50 guide per target sequence. The tiled guide polynucleotide set can comprise guide polynucleotides that cover at least 10% of a target sequence, or the tiled proximity probe set is tiled and comprises 2 to 50 guides per target sequence. In certain instances, the tiled set of proximity probes or guide polynucleotides are spaced evenly across a genome.

In some embodiments, the target of interest sequence can comprise an antibiotic resistance gene, a repetitive genetic element, a conserved genomic region across one or more genus or species, or a species-specific genomic region.

In embodiments, the MIP comprises a gap region that, upon hybridization to a target sequence and gap-filling, comprises a gap-filled sequence, the MIP is in certain embodiments designed so that the gap-filled sequence comprises the guide RNA recognition sequence. In embodiments, the gap filled region comprises biotinylated nucleotides. In some embodiments, the detection system can further comprise a capture system comprising streptavidin, which can be a streptavidin capture bead. In embodiments, the gap-filled sequence is at least 1 nucleotide in length. In other embodiments, the MIP is configured so that the 5′ and 3′ ends of the MIP are placed immediately adjacent to each other upon hybridization to the target sequence and that no gap region is provided.

In certain example embodiments, cell free nucleic acid detection system for detecting the presence of one or more pathogens and/or discriminating between one or more pathogen strains in a sample, is provided comprising: one or more CRISPR detection systems each comprising an effector protein and one or more guide RNAs tiled to corresponding cell free nucleic acid target sequences unique to one or more pathogen strains or one or more pathogens, a masking construct, and optionally an optical barcode. In some embodiments, the cell free nucleic acid detection system comprises two or more CRISPR systems, wherein the CRISPR system is a Cas13 system, as Cas12 system, or a combination thereof, optionally wherein the Cas 12 is Cpf1 or c2c1, the Cas 13 is Cas 13a, Cas 13b, or Cas 13c. In some embodiments, the CRISPR systems are RNA-targeting effector proteins, DNA-targeting effector proteins, or a combination thereof.

Systems can include in some embodiments RNA-targeting effector proteins that comprise one or more HEPN domains, optionally wherein the one or more HEPN domains comprises a RxxxxH motif sequence. In certain embodiments, the RxxxH motif comprises a R{N/H/K}X1X2X3H (SEQ ID NO:1) sequence, optionally wherein X1 is R, S, D, E, Q, N, G, or Y, and X2 is independently I, S, T, V, or L, and X3 is independently L, F, N, Y, V, I, S, D, E, or A.

Systems according to some embodiments comprise one or more guide RNAs that are about 28 nucleotides in length and have a mismatch of one or less to the corresponding target sequence. In some embodiments, two or more guide RNAs corresponding to target sequences in two or more pathogens, or two or more strains of a pathogen are provided. Systems can also comprise nucleic acid amplification reagents, which, in some embodiments are Polymerase Chain Reaction (PCR) reagents, Recombinase Polymerase Amplification (RPA) reagents, Rolling Circle Amplification (RCA) reagents, or Multiple Displacement Amplification (MDA) reagents. In some embodiments, the amplification reagents are PCR reagents and further comprise Molecular Inversion Probes (MIPs) corresponding to evenly spaced regions of a genome of the one or more pathogens.

In embodiments, the system comprises one or more oligonucleotide-based constructs, in certain embodiments, the system comprises two or more oligonucleotide constructs. In particular embodiments, each construct generates a different detectable signal that can be utilized to detect different species, genus, or other categories of organisms. The RNA-based masking construct of the system can, in some embodiments, suppress generation of a detectable positive signal. In some embodiments, the RNA-based masking construct suppresses generation of a detectable positive signal by masking the detectable positive signal, or generating a detectable negative signal instead; the RNA-based masking construct comprises a silencing RNA that suppresses generation of a gene product encoded by a reporting construct, wherein the gene product generates the detectable positive signal when expressed; and/or the RNA-based masking construct is a ribozyme that generates the negative detectable signal, and wherein the positive detectable signal is generated when the ribozyme is deactivated. In one embodiment, the detectable ligand is a fluorophore and the masking component is a quencher molecule. In one embodiment when the RNA-based masking construct is a ribozyme, the ribozyme converts a substrate to a first color and wherein the substrate converts to a second color when the ribozyme is deactivated.

In an embodiment, the RNA-based masking agent is an RNA aptamer and/or comprises an RNA-tethered inhibitor, which is some instances, sequesters an enzyme, wherein the enzyme generates a detectable signal upon release from the aptamer or RNA tethered inhibitor by acting upon a substrate. In some embodiments, the aptamer is an inhibitory aptamer that inhibits an enzyme and prevents the enzyme from catalyzing generation of a detectable signal from a substrate or wherein the RNA-tethered inhibitor inhibits an enzyme and prevents the enzyme from catalyzing generation of a detectable signal from a substrate. In some instances, when the RNA-tethered inhibits an enzyme, the enzyme is thrombin, protein C, neutrophil elastase, subtilisin, horseradish peroxidase, beta-galactosidase, or calf alkaline phosphatase; in some embodiments, the enzyme is thrombin and the substrate is para-nitroanilide covalently linked to a peptide substrate for thrombin, or 7-amino-4-methylcoumarin covalently linked to a peptide substrate for thrombin. In an aspect, the aptamer sequesters a pair of agents that when released from the aptamers combine to generate a detectable signal.

The RNA-based masking construct can comprise, in some embodiments, an RNA oligonucleotide to which a detectable ligand and a masking component are attached. In some embodiments the RNA-based masking construct comprises a nanoparticle held in aggregate by bridge molecules, wherein at least a portion of the bridge molecules comprises RNA, and wherein the solution undergoes a color shift when the nanoparticle is disbursed in solution. In some instances, the nanoparticle is a colloidal metal, which is, in some instances, colloidal gold. In further embodiments, the RNA-based masking construct comprising a quantum dot linked to one or more quencher molecules by a linking molecule, wherein at least a portion of the linking molecule comprises RNA. In other embodiments, the RNA-based masking construct comprises RNA in complex with an intercalating agent, wherein the intercalating agent changes absorbance upon cleavage of the RNA. In specific embodiments, the intercalating agent is pyronine-Y or methylene blue.

In some embodiments, the system comprises two or more CRISPR systems, and RNA-based masking constructs. Each CRISPR system comprises an effector protein and one or more guide molecules designed to bind to one or more corresponding target molecules of one or more pathogens of interest. In an embodiment, each RNA-based masking construct comprises a cutting motif sequence that is preferentially cut by one of the CRISPR effector proteins after the CRISPR effector protein is activated.

A multiplexed method of detecting the presence of one or more pathogens and/or discriminating between one or more strains in a sample, comprising: conducting target specific or non-specific preamplification on a cell free nucleic acid from a sample; generating a first set of droplets, each droplet in the first set of droplets comprising at least one target molecule from the sample and an optical barcode; generating a second set of droplets, each droplet in the second set of droplets comprising one or more detection CRISPR systems comprising an effector protein and one or more guide RNAs tiled to corresponding target sequences unique to the one or more strains or one or more pathogens, an RNA-based masking construct and an optical barcode; combining the first set and second set of droplets into a pool of droplets and flowing the pool of droplets onto a microfluidic device comprising an array of microwells and at least one flow channel beneath the microwells, the microwells sized to capture at least two droplets; detecting the optical barcodes of the droplets captured in each microwell; merging the droplets captured in each microwell to form merged droplets in each microwell, at least a subset of the merged droplets comprising a detection CRISPR system and a target sequence; initiating the detection reaction by incubating at about 37° C.; and measuring a detectable signal of each merged droplet at one or more time periods.

The initial amplification, or preamplification, can be target specific, optionally selected from PCR, RPA, or RCA, or can comprise preferential amplification of microbial DNA by exploiting methylation sites and size selecting microbial cfDNA, or non-specific, optionally selected from adapter-ligation, degenerate PCR and MDA.

In some embodiments, the amplification is target-specific and probes comprise proximity dependent probes, such as molecular inversion probes. In particular embodiments, molecular inversion probe amplification comprises hybridizing probes to a target of interest; circularizing the hybridized probes; digesting non-hybridized linear probes; and adding a primer pair and amplifying the circularized probes.

In some embodiments, the sample is plasma, blood, or urine. In some embodiments, the sample is from a subject with an active infection. In some embodiments, samples can be from a healthy subject. Extraction of cfDNA from the sample may be performed prior to the step of preamplification. Heating the sample can be performed prior to the step of preamplification.

In some embodiments, guide RNAs for the currently disclosed subject matter can be selected by defining ‘in’ group comprising the genomes of interest and an ‘out’ group comprising genomes not of interest; selecting a reference genome in the ‘in’ group and generating a list of all possible genomic targets of 28 nucleotides; identifying matching sequences with all other genomes in the ‘in’ and ‘out’ groups using a sequence alignment tool; generating a candidate list of possible genomic targets comprising sequences that match with all genomes in the ‘in group’ and do not match with any of the genomes in the ‘out group,’ thereby identifying probes for target sequences. In certain instances, selection of the guide RNAs is further based on one or more of sequence orthogonality, melting temperature and/or genomic distribution, in some embodiments the guide RNAs have a mismatch tolerance of one nucleotide.

In certain embodiments, imaging the droplets is performed at intermittent intervals or continuously to measure fluorescence kinetics and/or quantitation.

Methods of detecting host response to infection are provided and include performing the methods of detecting cfDNA in a host sample obtained at a first time, performing the method on a host sample obtained at a second time, and detecting the presence of one or more pathogens at the first time and the second time. In some embodiments, the host is treated with an antibiotic subsequent to the first time and prior to the second time. In some embodiments, methods can comprise detecting antibiotic resistance and identifying genetic markers associated with antibiotic resistance.

In certain embodiments, preamplification step is performed in the first set of droplets after generating the first set of droplets.

Methods of detecting target cell free nucleic acids in a sample can comprise distributing a sample or set of samples into one or more individual discrete volumes, the individual discrete volumes comprising a CRISPR system as disclosed herein, incubating the sample or set of samples under conditions to allow binding of the one or more guide RNAs to one or more target molecules; activating the CRISPR effector protein via binding of the one or more guide RNAs to the one or more target sequences, wherein activating the CRISPR effector protein results in modification of the RNA-based masking construct such that a detectable positive signal is generated; and detecting the one or more detectable positive signal, wherein detection of the one or more detectable positive signal indicates a presence of one or more target molecules in the sample. In some embodiments, the one or more guide RNAs correspond to one or more target molecules defined by evenly spaced regions of a genome of the one or more pathogens.

Microfluidic devices are provided comprising a sample loading region, and one or more flow channels, each channel comprising a detector region comprising a detection construct and one or more nucleic acid detection systems, and at least a first and second capture region, the first capture region comprising a first binding agent and the second capture region comprising a second binding agent. The region of the microfluidic flow device may comprise a node. In an aspect, each flow channel is arranged radially from a center node, or may be arranged in parallel. The center node may comprise transcription reagents.

In embodiments, the microfluidic devices may comprise one or more thermally differentiated zones disposed between the loading region and the center node. The detection construct may comprise a first molecule on a first end and a second molecule on a second end. In an aspect, the microfluidic device may comprise one or more nucleic acid detection systems that each comprise a Cas protein, and a species-specific guide RNA. One or more amplification reagents may be provided with the microfluidic device, optionally in the sample loading region. In an aspect, the one or more amplification reagents are selected from nucleic acid sequence-based amplification (NASBA), recombinase polymerase amplification (RPA), loop-mediated isothermal amplification (LAMP), strand displacement amplification (SDA), helicase-dependent amplification (HDA), nicking enzyme amplification reaction (NEAR), PCR, multiple displacement amplification (MDA), rolling circle amplification (RCA), ligase chain reaction (LCR), or ramification amplification method (RAM). In an aspect, the microfluidic device may comprise a Cas system wherein the guide RNA is designed to target an amplicon of the sample. In an aspect, the microfluidic device of comprises a molecular inversion probe (MIP) or proximity dependent probe and ligation reagents in the sample loading region. In an aspect, the microfluidic device can comprise species-specific guide RNA designed to target a species-specific binding sequence on the MIP.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1 Depiction of experimental design for exemplary embodiment.

FIG. 2A provides an overview of application of exemplary embodiment; FIG. 2B describes features of several current state of the art detection tests, and the goal of an ideal test that the currently disclosed embodiments satisfy, including providing detection of a broad number of pathogens in a rapid test utilizing a non-invasive sample source that is sensitive and specific; FIG. 2C Steps involved in molecular inversion probe (MIP) based amplification (adapted and modified from Nilsson et al.)

FIG. 3A depicts a genome-wide tiled assay according to an exemplary embodiment FIG. 3B provides an overview of a clinical assay according to an exemplary embodiment.

FIG. 4 provides an overview of molecular inversion probe (MIP) workflow.

FIG. 5 depicts MIP amplification combined with CRISPR/Cas13.

FIG. 6 shows an approach to pooled MIPs for genomic-wide tiling.

FIG. 7 includes an overview of droplet SHERLOCK for multiplexed pathogen detection utilizing the currently disclosed molecular inversion probe approaches.

FIG. 8 Methodology for identifying pathogen genomic target sites; k-mers generated from reference strain is aligned with all genomes in the “in” and “out” groups using Bowtie (SEQ ID NO:2-4).

FIG. 9A-9B Distribution of 28-mer targets across the Staphylococcus aureus genome (Newman strain); FIG. 9A targets conserved across the “in” group; FIG. 9B further filtering to exclude targets present in “out” group.

FIG. 10 charts MIP-based hybridization of S. aureus gDNA (2 ng/reaction) and amplification with qPCR; 10 tiled probes show higher extent of circularization.

FIG. 11A-11D Comparison of SHERLOCK assay in droplets with same assay on plates, error bars indicate standard error (11B and 11D are adapted from Myhrvold et al.) FIGS. 11A and 11C show initial target concentration and SHERLOCK assay results in droplets, FIGS. 11B and 11D show initial target concentration and SHERLOCK assay results when performed in plates.

FIG. 12 bootstrapping analysis to estimate number of replicates needed to confidently discriminate between signals (ancestral crRNA in FIG. 11C).

FIG. 13A Schematic overview of steps involved in current NATs, SHERLOCK, and tiled assay; FIG. 13B spectrum of possible amplification strategies that can be used for tiled assay.

FIG. 14 Workflow for combinatorial droplet loading and imaging for droplet SHERLOCK (modified from Kulesa et al.).

FIG. 15 depicts an exemplary proposed assay compared to traditional nucleic acid amplification technology and SHERLOCK.

FIG. 16 provides an exemplary workflow of the proposed assay, including use of biopsies or bodily fluids for detection.

FIG. 17 includes possible initial target amplification strategies for use in methods and systems described herein.

FIG. 18 provides an exemplary overview of an approach utilizing molecular inversion probe amplification followed by CRISPR detection as disclosed herein.

FIG. 19 details exemplary molecular inversion probed design, including user-defined sequence elements. Exemplary target sequences can include species-specific genomic regions for species-specific ID, conserved genomic regions for lower resolution (e.g. genus-specific) ID, known antibiotic resistance genes, repetitive genomic elements. Optional Gap-filled sequence examples can function as CRISPR-Cas recognition sequence, can improve specificity of ligation (if all gaps in annealed MIPs are a single common nucleotide, the gap filling in the presence of only that nucleotide can eliminate most nonspecific gap-fill/ligation events, can include biotinylated nucleotide during gap filling for subsequent streptavidin-mediated capture. The gap-filled length has a minimum length of 1 nucleotide. Alternative approaches can omit gap filling by placing the 5′ and 3′ ends of the MIP immediately adjacent to each other upon hybridization to target sequence.

FIG. 20 depicts one exemplary system and method showing design of molecular inversion probe and crRNA designed to identify the presence of a specific species, genus, or Gram-negative or Gram-positive bacteria. In one example, the MIP structure with crRNA target sequence is integrated into the MIP backbone. The incorporation of crRNA target sequence is conserved across multiple MIPs to provide diagnostic specificity at the level illustrated.

FIG. 21 depicts another exemplary system and method showing design of molecular inversion probe and crRNA designed to identify generic MIP structure with crRNA target sequence integrated into the gene of interest targeting region.

FIG. 22 depicts the freeze-dried chip concept that allows loading of sample when ready for use onto the chip. Graphs provide results comparing a sample using a freeze dried chip and a non-freeze dried chip.

FIG. 23 includes use of a freeze-dried chip, optionally freeze drying one component of the system. As provided in the chart, bottom left, there is a 20-fold reduction in signal with freeze-drying of detection mix. Freeze drying just crRNA, on the other hand, gives only 3-fold reduction in signal, at half the reaction volume.

FIG. 24 includes an approach to loading using degassing, adapted from Cira et al., Lab on a Chip, 2012 (https://pubs.rsc.org/en/content/articlelanding/2012/lc/c2lc20887c #!divAbstract), an approach that can allow loading of samples into wells without dropletization, doing so with minimal wastage. This approach may aid in making the assay more portable, which includes the ultimate aims of allowing storage of detection mixes at room temperature, no need for sample droplet generation, and readout without expensive microscope

FIG. 25 is a schematic of an approach for portable imaging that can be used with the methods and systems provided herein. Briefly, a light source is passed through a microarray chip and a camera, for example a DSLR or cell phone, or other imaging source is utilized for capturing an image. Band pass filters are used on one or both the light source and the camera and can allow for imaging at 490 nm and/or 520 nm.

FIG. 26 depicts approaches for multiplexed detection of microbial nucleic acids using CRISPR-Cas systems.

FIG. 27 is a schematic of an exemplary process.

FIG. 28A-28C provide three exemplary manifestations of MIP based detection. FIG. 28A shows a first manifestation of MIP based detection comprising target recognition with MIP ligation; rolling circle transcription from circular MIP; guide RNA/Cas binding to the transcript with subsequent collateral cleavage of reporter. FIG. 28B shows a second manifestation of MIP based detection comprising target recognition with MIP ligation; primer-based amplification of circular MI{S, followed by transcription; guide RNA/Cas binding to the transcript with subsequent collateral cleavage of reporter. FIG. 28C shows a third manifestation of MIP based detection comprising target recognition with MIP ligation; rolling circle amplification of circular MIP; T7 transcription followed by guide RNA/Cas binding to the transcript with subsequent collateral cleavage of reporter.

FIG. 29A-29G provides overview of target selection and molecular inversion probe (MIP) design. FIG. 29A depicts MIP functional elements; FIG. 29B shows MIP structure design for direct in vitro transcription; FIG. 29C shows amplification-competent MIP structures; FIG. 29D includes target selection and binding arms FIG. 29E depicts optional inclusion of primer binding sites; FIG. 29F depicts gRNA binding site design; and FIG. 29G Heatmap of SHERLOCK signal showing strength of interactions between eight de novo designed gRNAs (y-axis) and their cognate and non-cognate in vitro transcribed targets (x-axis).

FIG. 30A-30B is a schematic of Step 2 of a exemplary process, hybridization and ligation of MIPs.

FIG. 31 includes details of evaluated amplification strategies.

FIG. 32 charts multiplexed detection of microbial nucleic acids improves assay sensitivity. Boxes indicate minimum/maximum of 2 replicates, center line indicated mean.

FIG. 33 is a schematic of SHERLOCK later flow assay showing flow of a fluid sample across a substrate.

FIG. 34A-34B includes FIG. 34A an exemplary microfluidic set up of species identification using TopA primers, PCR amplification and CRISPR detection using the systems and guides designed as described herein; and FIG. 34B depicts a sample containing Psa run across an exemplary microfluidic device comprising TopA primers, PCR amplification and CRISPR with node showing positive signal for presence of PsA at node comprising detection construct.

FIG. 35A-35B includes FIG. 35A an exemplary microfluidic set up of species identification using MIPs, RPA and CRISPR detection using the systems and guides designed as described herein; and FIG. 35B depicts a sample containing Psa run across an exemplary microfluidic device comprising MIPs, RPA and CRISPR reagents with node showing positive signal for presence of Psa at node comprising detection construct.

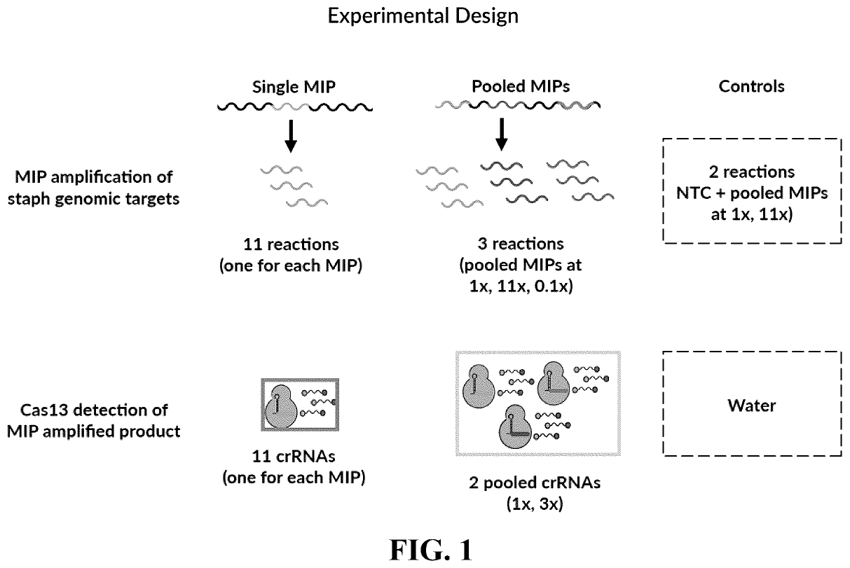

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboraotry Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### OVERVIEW

Embodiments disclosed herein provide a diagnostic platform that can rapidly and accurately identify the presence of bacterial pathogens and discriminate between strains in clinical samples. Advantageously, the diagnostic platform is rapid, sensitive and can be adapted to any microbe through a bioinformatics pipeline that identifies multiple targets across the entire genome. Methods are also provided for detection of pathogen nucleic acids in blood or urine utilizing circulating cell-free nucleic acids and find use in detection of multiple pathogens, identifying markers of antibiotic resistance in pathogens, as well as monitoring host response to infection.

A Clustered Regularly Interspaced Short Palindromic Repeats (CRISPR) and CRISPR associated enzymes (Cas) are together the hallmark of bacterial defense systems. CRISPR-Cas systems have been shown to be reprogrammable for a variety of genome editing applications, and more recently, for CRISPR-based diagnostics. In particular, CRISPR-Cas system that utilize RNA targeting effectors, which may also be referred to as SHERLOCK (Specific High-sensitivity Enzymatic Reporter unLOCKing), has been shown to detect a single genomic target at attomolar concentrations. As described herein, CRISPR detection systems, such as SHERLOCK, are used with tiled genomic probes to provide a robust CRISPR-based diagnostic. The combination of preamplification of nucleic acids in the sample with the CRISPR detection system allows for the detection of even small amounts of pathogenic cfDNA present in samples. The specificity of the preamplification step can be tuned to amplify unique targets, to amplify microbial DNA preferentially, or to amplify non-specifically. Because CRISPR-Cas introduces a second layer of specificity, amplification need not be exclusive to the targets of interest, and non-specific amplification of background may be permissible, with less restrictive initial amplification further increase the system's sensitivity in detecting bacterial DNA. Similarly, the specificity of the guide RNA used in SHERLOCK can be adjusted by the design parameters placed on the genomic targets of interest as described herein.

Using such systems, nucleic acid targets have been detected down to concentrations of 50 fM54. Combined with initial amplification of target, targets have been shown to be detectable at attomolar concentrations, or even lower. Systems utilizing different Cas enzymes can target ssRNA or ds DNA.

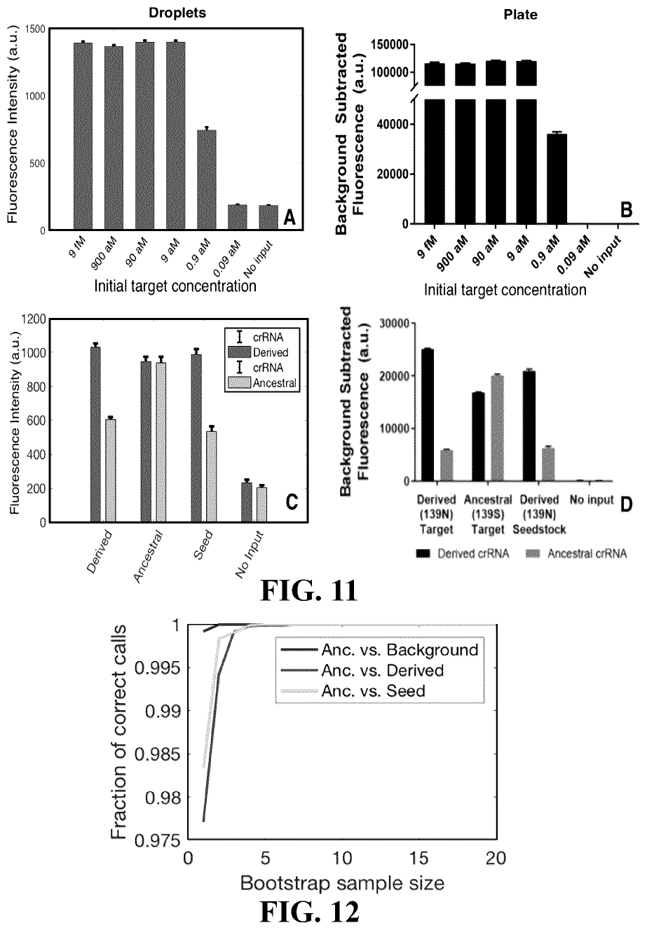

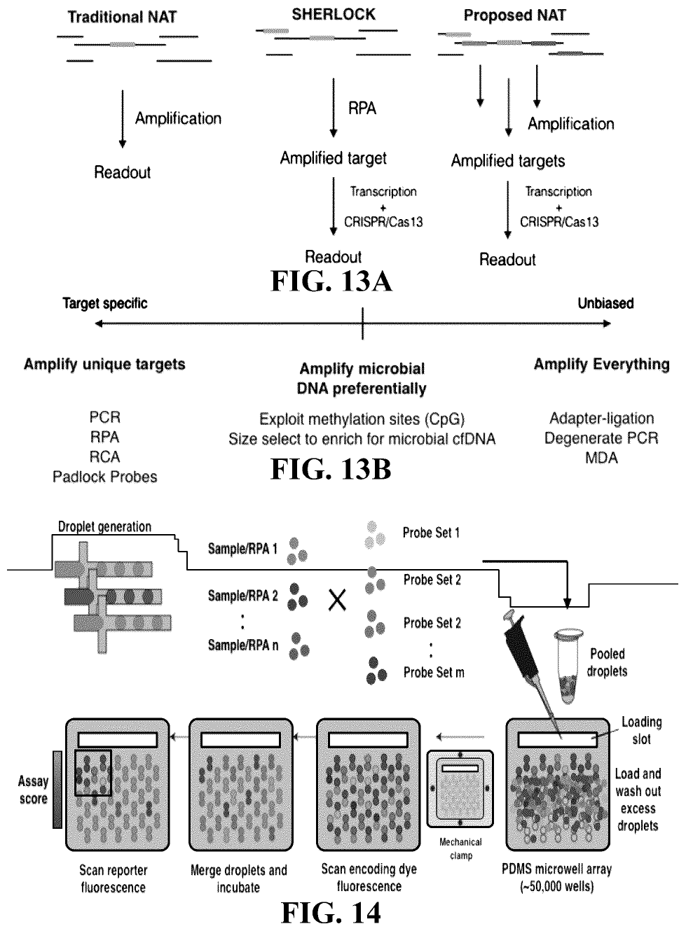

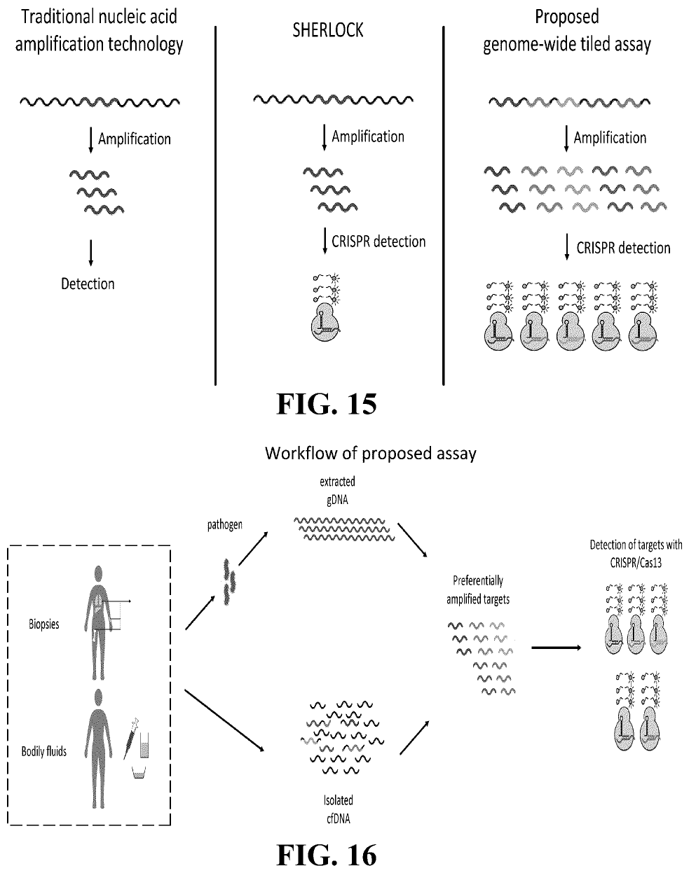

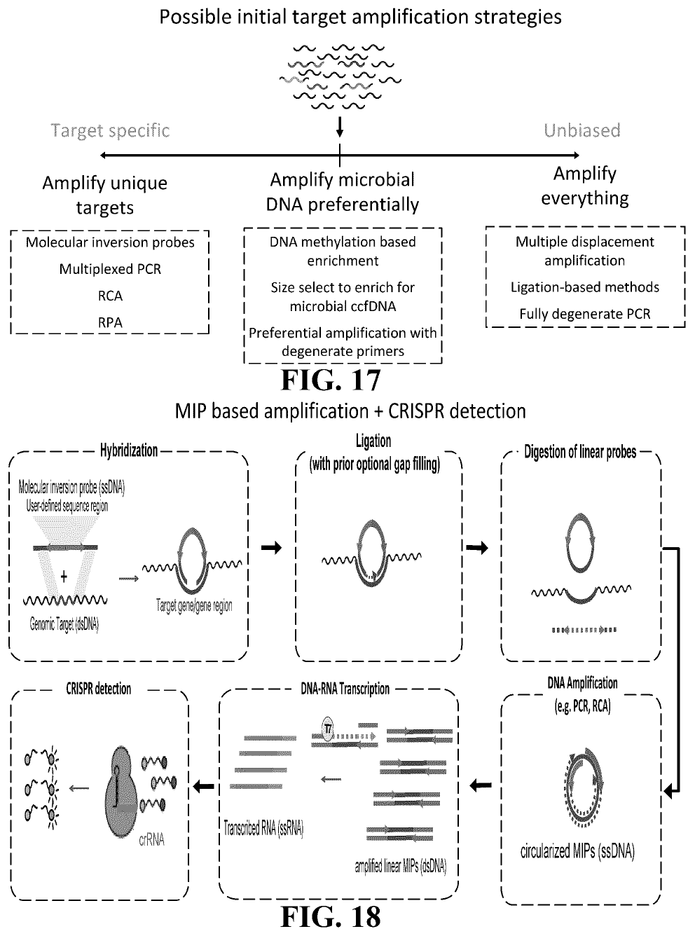

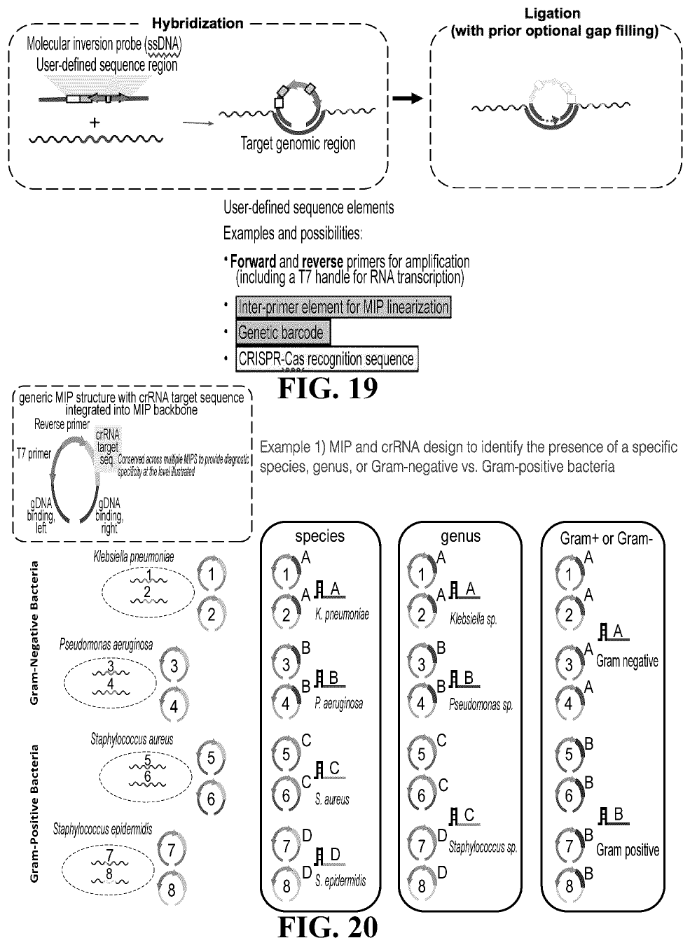

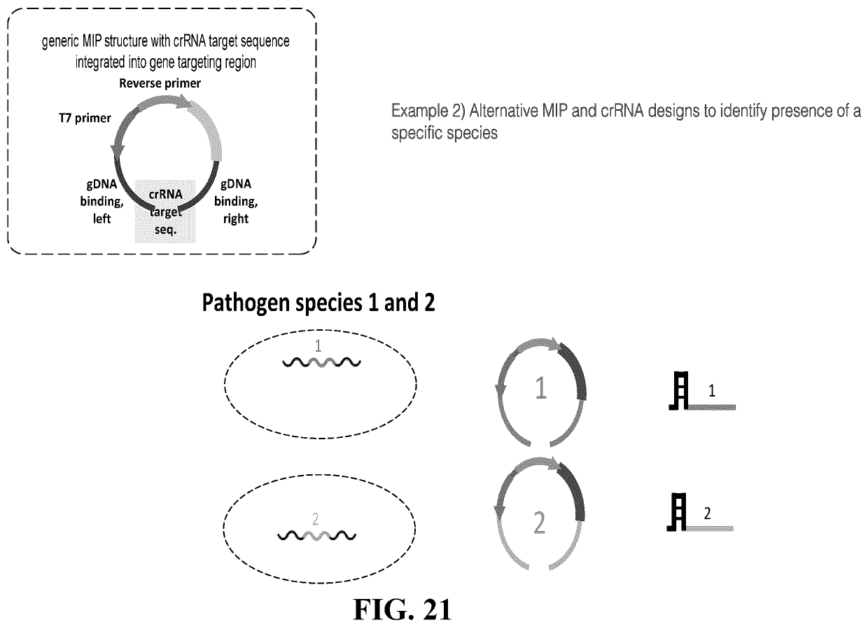

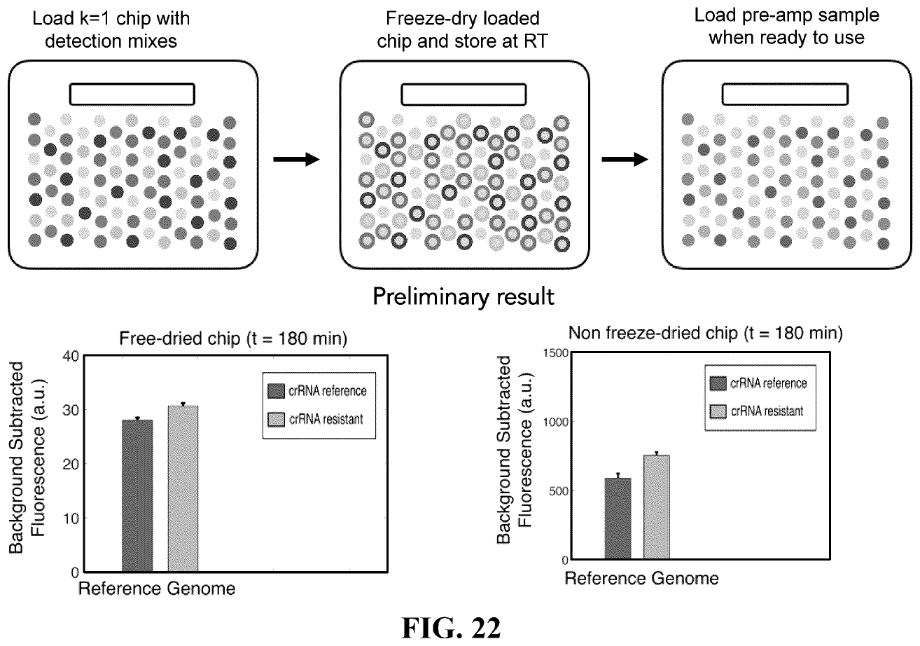

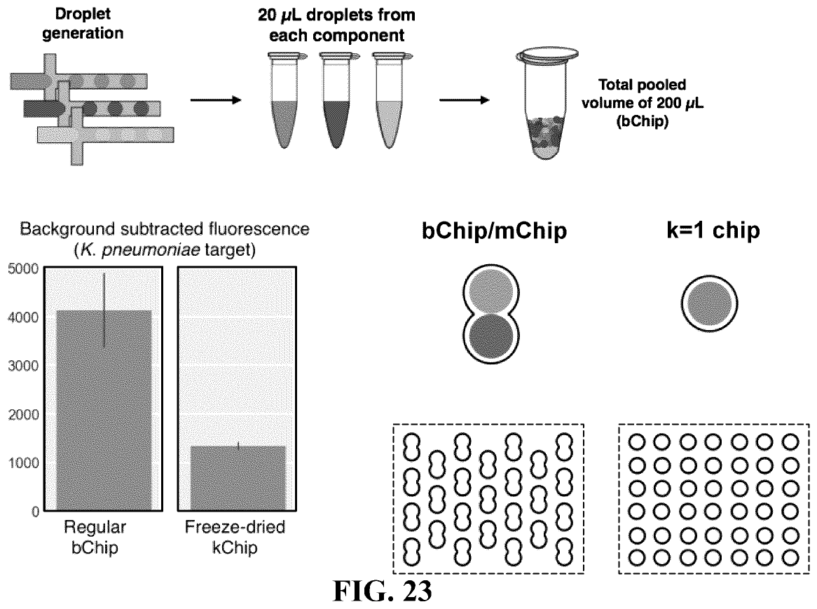

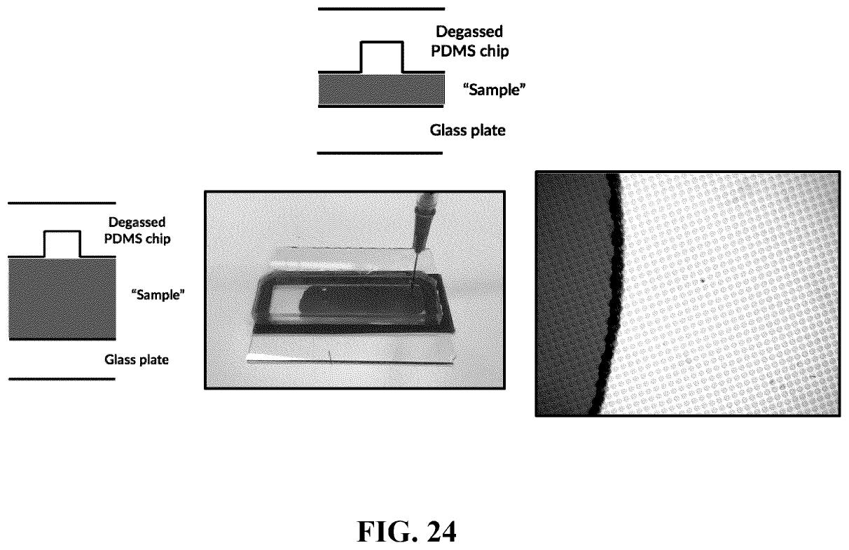

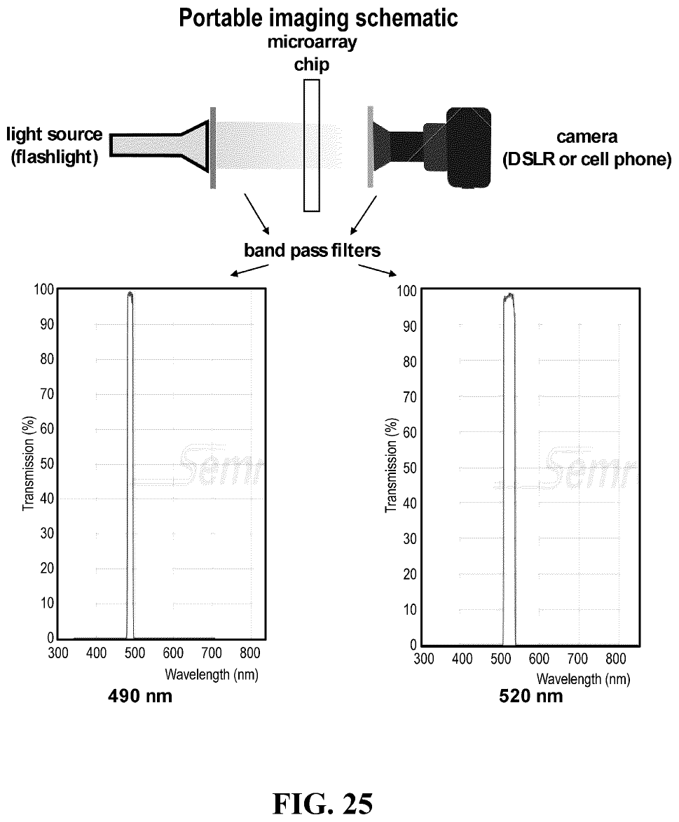

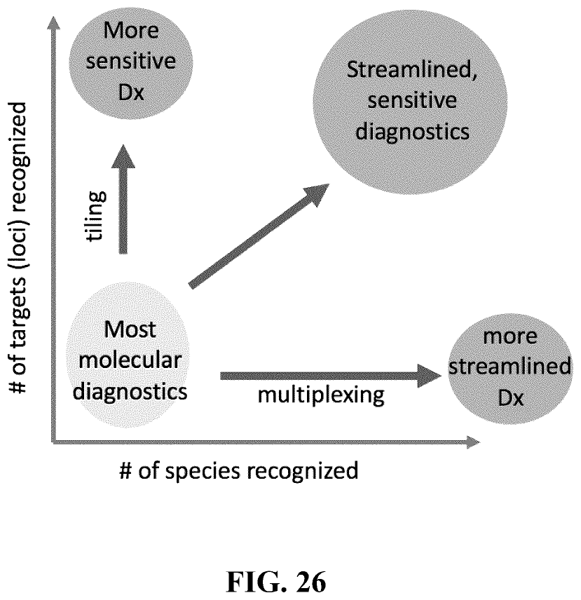

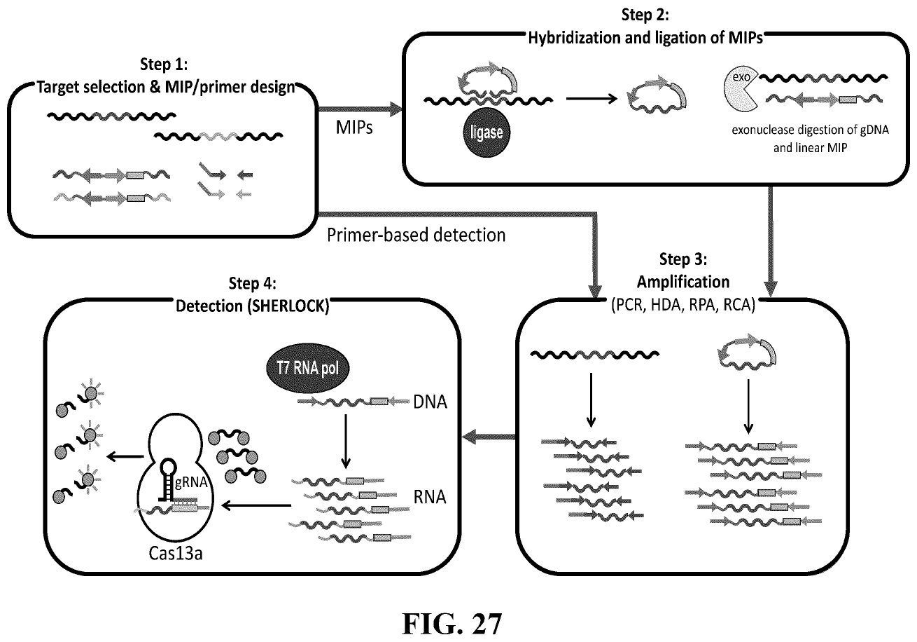

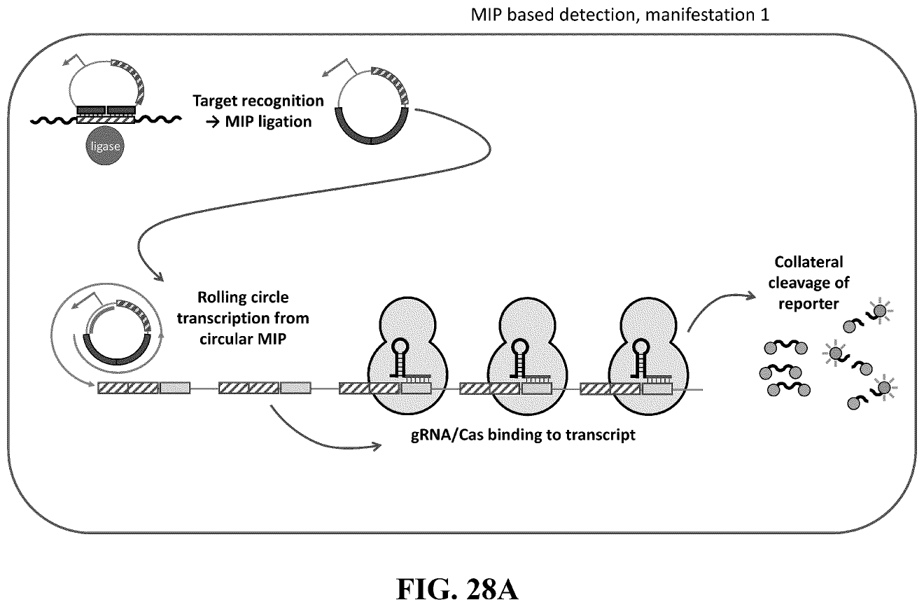

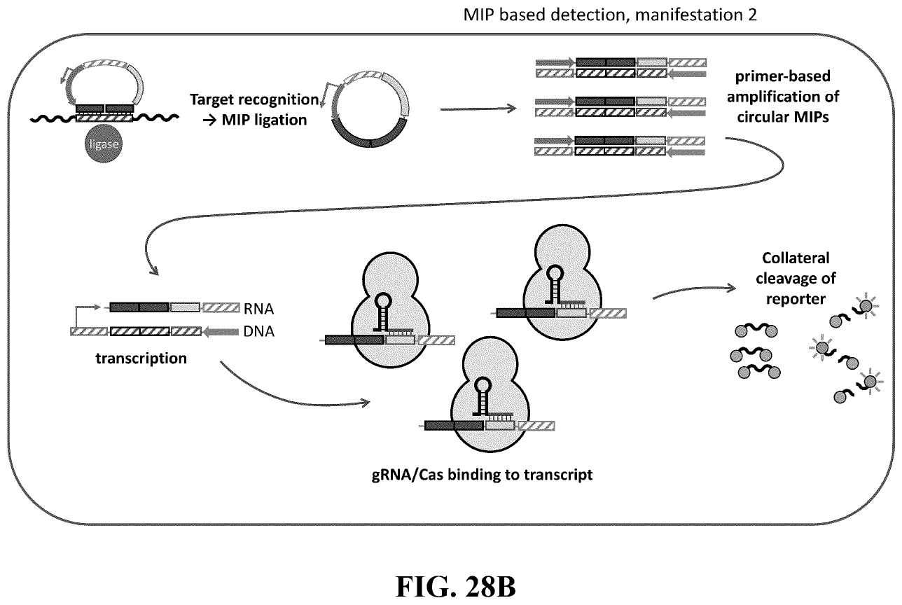

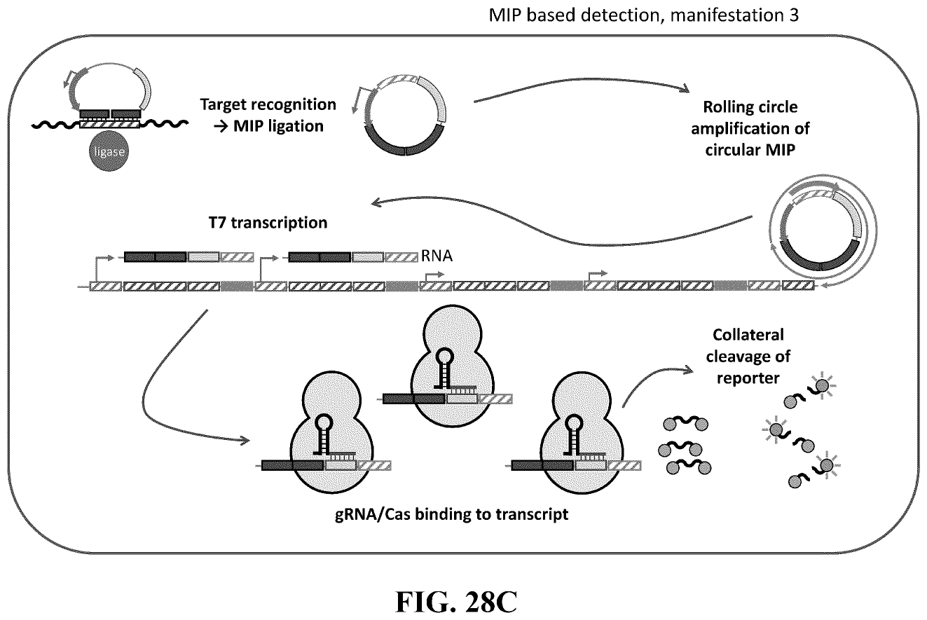

The systems and methods disclosed can be massively multiplexed by performing detection in droplets. As described in U.S. Provisional Application, (Attorney Docket Nos. BROD-3830P, BI-10400), and herein, a SHERLOCK detection can be performed in droplets with a similar limit of detection when running the assay in nanoliter droplets when compared to larger volume reactions on plates for detection of SNPs in Zika viral genomes. (Compare FIG. 11A, 11C with FIG. 11B, 11D).

Cas13s non-specific RNase activity can be leveraged to cleave reporters upon target recognition, allowing for the design of sensitive and specific diagnostics using Cas13, including single nucleotide variants, detection based on rRNA sequences, screening for drug resistance, monitoring microbe outbreaks, genetic perturbations, and screening of environmental samples, as described, for example, in PCT/US18/054472 filed Oct. 22, 2018 at [0183]-[0327], incorporated herein by reference. Reference is made to WO 2017/219027, WO2018/107129, US20180298445, US 2018-0274017, US 2018-0305773, WO 2018/170340, U.S. application Ser. No. 15/922,837, filed Mar. 15, 2018 entitled “Devices for CRISPR Effector System Based Diagnostics”, PCT/US18/50091, filed Sep. 7, 2018 “Multi-Effector CRISPR Based Diagnostic Systems”, PCT/US18/66940 filed Dec. 20, 2018 entitled “CRISPR Effector System Based Multiplex Diagnostics”, PCT/US18/054472 filed Oct. 4, 2018 entitled “CRISPR Effector System Based Diagnostic”, U.S. Provisional 62/740,728 filed Oct. 3, 2018 entitled “CRISPR Effector System Based Diagnostics for Hemorrhagic Fever Detection”, U.S. Provisional 62/690,278 filed Jun. 26, 2018 and U.S. Provisional 62/767,059 filed Nov. 14, 2018 both entitled “CRISPR Double Nickase Based Amplification, Compositions, Systems and Methods”, U.S. Provisional 62/690,160 filed Jun. 26, 2018 and U.S. Pat. No. 62,767,077 filed Nov. 14, 2018, both entitled “CRISPR/CAS and Transposase Based Amplification Compositions, Systems, And Methods”, U.S. Provisional 62/690,257 filed Jun. 26, 2018 and 62/767,052 filed Nov. 14, 2018 both entitled “CRISPR Effector System Based Amplification Methods, Systems, And Diagnostics”, U.S. Provisional 62/767,076 filed Nov. 14, 2018 entitled “Multiplexing Highly Evolving Viral Variants With SHERLOCK” and 62/767,070 filed Nov. 14, 2018 entitled “Droplet SHERLOCK.” Reference is further made to WO2017/127807, WO2017/184786, WO 2017/184768, WO 2017/189308, WO 2018/035388, WO 2018/170333, WO 2018/191388, WO 2018/213708, WO 2019/005866, PCT/US18/67328 filed Dec. 21, 2018 entitled “Novel CRISPR Enzymes and Systems”, PCT/US18/67225 filed Dec. 21, 2018 entitled “Novel CRISPR Enzymes and Systems” and PCT/US18/67307 filed Dec. 21, 2018 entitled “Novel CRISPR Enzymes and Systems”, U.S. 62/712,809 filed Jul. 31, 2018 entitled “Novel CRISPR Enzymes and Systems”, U.S. 62/744,080 filed Oct. 10, 2018 entitled “Novel Cas12b Enzymes and Systems” and U.S. 62/751,196 filed Oct. 26 2018 entitled “Novel Cas12b Enzymes and Systems”, U.S. 715,640 filed August 7, 2-18 entitled “Novel CRISPR Enzymes and Systems”, WO 2016/205711, U.S. Pat. No. 9,790,490, WO 2016/205749, WO 2016/205764, WO 2017/070605, WO 2017/106657, and WO 2016/149661, WO2018/035387, WO2018/194963, Cox D B T, et al., RNA editing with CRISPR-Cas13, Science. 2017 Nov. 24; 358(6366):1019-1027; Gootenberg J S, et al., Multiplexed and portable nucleic acid detection platform with Cas13, Cas12a, and Csm6., Science. 2018-4-27; 360(6387):439-444; Gootenberg J S, et al., Nucleic acid detection with CRISPR-Cas13a/C2c2., Science. 2017 Apr. 28; 356(6336):438-442; Abudayyeh 00, et al., RNA targeting with CRISPR-Cas13, Nature. 2017-10-12; 550(7675):280-284; Smargon A A, et al., Cas13b Is a Type VI-B CRISPR-Associated RNA-Guided RNase Differentially Regulated by Accessory Proteins Csx27 and Csx28. Mol Cell. 2017-2-16; 65(4):618-630.e7; Abudayyeh 00, et al., C2c2 is a single-component programmable RNA-guided RNA-targeting CRISPR effector, Science. 2016 Aug. 5; 353(6299):aaf5573; Yang L, et al., Engineering and optimising deaminase fusions for genome editing. Nat Commun. 2016-11-2; 7:13330, Myrvhold et al., Field deployable viral diagnostics using CRISPR-Cas13, Science 2018 360, 444-448, Shmakov et al. “Diversity and evolution of class 2 CRISPR-Cas systems,” Nat Rev Microbiol. 2017 15(3):169-182, each of which is incorporated herein by reference in its entirety.

Cell free Nucleic Acid Detection System

Cell free nucleic acid detection systems are provided herein. The systems can be used to detect the presence of one or more pathogens and/or discriminating between one or more pathogen strains in a sample. The cell free nucleic acid detection systems can include one or more CRISPR detection systems, each system comprising an effector protein and one or more guide RNAs tiled to corresponding cell free nucleic acid target sequences unique to one or more pathogen strains or one or more pathogens, a masking construct, and optionally an optical barcode. In some embodiments, the cell free nucleic acid sequences comprise two or more CRISPR systems.

The systems described herein may comprise one or more sets of polynucleotides. A set of polynucleotides comprises two or more polynucleotides. In embodiments, the set of polynucleotides comprises 2 to 1000, 2 to 200, or about 2 to 50 polynucleotides. The polynucleotide set may comprise proximity dependent probes, or guide RNAs.

### CRISPR Detection System

CRISPR Cas based systems that allow for detection down to femtomolar sensitivity can be combined with initial amplification of the target to allow for detectable attomolar concentrations, possibly lower. SHERLOCK and DETECTR employ preamplification systems with Cas enzymes, for example Cas13a or Cas 12a that target ssRNA and ds DNA respectively. Although in some embodiments, RPA may be used as an amplification method to initially amplify targets, any alternative amplification strategy can be adapted for use with the CRISPR detections systems disclosed herein.

### Crispr Effector Proteins

In general, a CRISPR-Cas or CRISPR system as used herein and in documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). When the CRISPR protein is a C2c2 protein, a tracrRNA is not required. C2c2 has been described in Abudayyeh et al. (2016) “C2c2 is a single-component programmable RNA-guided RNA-targeting CRISPR effector”; Science; DOI: 10.1126/science.aaf5573; and Shmakov et al. (2015) “Discovery and Functional Characterization of Diverse Class 2 CRISPR-Cas Systems”, Molecular Cell, DOI: dx.doi.org/10.1016/j.molcel.2015.10.008; which are incorporated herein in their entirety by reference. Cas13b has been described in Smargon et al. (2017) “Cas13b Is a Type VI-B CRISPR-Associated RNA-Guided RNases Differentially Regulated by Accessory Proteins Csx27 and Csx28,” Molecular Cell. 65, 1-13; dx.doi.org/10.1016/j.molcel.2016.12.023., which is incorporated herein in its entirety by reference. CRISPR effector proteins described in International Application No. PCT/US2017/065477, Tables 1-6, pages 40-52, can be used in the presently disclosed methods, systems and devices, and are specifically incorporated herein by reference.

In certain embodiments, a protospacer adjacent motif (PAM) or PAM-like motif directs binding of the effector protein complex as disclosed herein to the target locus of interest. In some embodiments, the PAM may be a 5′ PAM (i.e., located upstream of the 5′ end of the protospacer). In other embodiments, the PAM may be a 3′ PAM (i.e., located downstream of the 5′ end of the protospacer). The term “PAM” may be used interchangeably with the term “PFS” or “protospacer flanking site” or “protospacer flanking sequence”.

In a preferred embodiment, the CRISPR effector protein may recognize a 3′ PAM. In certain embodiments, the CRISPR effector protein may recognize a 3′ PAM which is 5′H, wherein H is A, C or U. In certain embodiments, the effector protein may be Leptotrichia shahii C2c2p, more preferably Leptotrichia shahii DSM 19757 C2c2, and the 3′ PAM is a 5′ H.

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to a RNA polynucleotide being or comprising the target sequence. In other words, the target RNA may be a RNA polynucleotide or a part of a RNA polynucleotide to which a part of the gRNA, i.e. the guide sequence, is designed to have complementarity and to which the effector function mediated by the complex comprising CRISPR effector protein and a gRNA is to be directed. In some embodiments, a target sequence is located in the nucleus or cytoplasm of a cell.

The nucleic acid molecule encoding a CRISPR effector protein, in particular C2c2, is advantageously codon optimized CRISPR effector protein. An example of a codon optimized sequence is, in this instance, a sequence optimized for expression in eukaryotes, e.g., humans (i.e. being optimized for expression in humans), or for another eukaryote, animal or mammal as herein discussed; see, e.g., SaCas9 human codon optimized sequence in WO 2014/093622 (PCT/US2013/074667). Whilst this is preferred, it will be appreciated that other examples are possible and codon optimization for a host species other than human, or for codon optimization for specific organs is known. In some embodiments, an enzyme coding sequence encoding a CRISPR effector protein is a codon optimized for expression in particular cells, such as eukaryotic cells. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g. 1, 2, 3, 4, 5, 10, 15, 20, 25, 50, or more, or all codons) in a sequence encoding a Cas correspond to the most frequently used codon for a particular amino acid.

In certain embodiments, more than one CRISPR system effector protein is provided. The CRISPR system effector proteins can be provided with orthogonal base preferences, for example, as provided in U.S. Provisional Application 62/741,501 filed Oct. 4, 2018 at [0217]-[0302], and Example 9, [0662]-[0666].

Example RNA-targeting effector proteins include Cas13b and C2c2 (now known as Cas13a). It will be understood that the term “C2c2” herein is used interchangeably with “Cas13a”. In another example embodiment, the RNA-targeting effector protein is C2c2, which in some embodiments is within 20 kb of a Cas1 gene. In some embodiments, the C2c2 effector protein is from an organism of a genus selected from the group consisting of: Leptotrichia, Listeria, Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, Campylobacter, and Lachnospira.

In some embodiments, the C2c2 or Cas13b effector protein is from an organism selected from the group consisting of: Leptotrichia shahii; Leptotrichia wadei (Lw2); Listeria seeligeri; Lachnospiraceae bacterium MA2020; Lachnospiraceae bacterium NK4A179; [Clostridium]aminophilum DSM 10710; Carnobacterium gallinarum DSM 4847; Carnobacterium gallinarum DSM 4847 (second CRISPR Loci); Paludibacter propionicigenes WB4; Listeria weihenstephanensis FSL R9-0317; Listeriaceae bacterium FSL M6-0635; Leptotrichia wadei F0279; Rhodobacter capsulatus SB 1003; Rhodobacter capsulatus R121; Rhodobacter capsulatus DE442; Leptotrichia buccalis C-1013-b; Herbinix hemicellulosilytica; [Eubacterium] rectale; Eubacteriaceae bacterium CHKCI004; Blautia sp. Marseille-P2398; Leptotrichia sp. oral taxon 879 str. F0557; Lachnospiraceae bacterium NK4A144; Chloroflexus aggregans; Demequina aurantiaca; Thalassospira sp. TSL5-1; Pseudobutyrivibrio sp. OR37; Butyrivibrio sp. YAB3001; Blautia sp. Marseille-P2398; Leptotrichia sp. Marseille-P3007; Bacteroides ihuae; Porphyromonadaceae bacterium KH3CP3RA; Listeria riparia; and Insolitispirillum peregrinum. In one embodiment, the C2c2 effector protein is a L. wadei F0279 or L. wadei F0279 (Lw2) C2C2 effector protein.

In certain embodiments, the methods as described herein may comprise providing a Cas transgenic cell, in particular a C2c2 transgenic cell, in which one or more nucleic acids encoding one or more guide RNAs are provided or introduced operably connected in the cell with a regulatory element comprising a promoter of one or more gene of interest. As used herein, the term “Cas transgenic cell” refers to a cell, such as a eukaryotic cell, in which a Cas gene has been genomically integrated. The nature, type, or origin of the cell are not particularly limiting according to the present invention. Also the way the Cas transgene is introduced in the cell may vary and can be any method as is known in the art. In certain embodiments, the Cas transgenic cell is obtained by introducing the Cas transgene in an isolated cell. In certain other embodiments, the Cas transgenic cell is obtained by isolating cells from a Cas transgenic organism. By means of example, and without limitation, the Cas transgenic cell as referred to herein may be derived from a Cas transgenic eukaryote, such as a Cas knock-in eukaryote. Reference is made to WO 2014/093622 (PCT/US13/74667), incorporated herein by reference. Methods of US Patent Publication Nos. 20120017290 and 20110265198 assigned to Sangamo BioSciences, Inc. directed to targeting the Rosa locus may be modified to utilize the CRISPR Cas system of the present invention. Methods of US Patent Publication No. 20130236946 assigned to Cellectis directed to targeting the Rosa locus may also be modified to utilize the CRISPR Cas system of the present invention. By means of further example reference is made to Platt et. al. (Cell; 159(2):440-455 (2014)), describing a Cas9 knock-in mouse, which is incorporated herein by reference. The Cas transgene can further comprise a Lox-Stop-polyA-Lox (LSL) cassette thereby rendering Cas expression inducible by Cre recombinase. Alternatively, the Cas transgenic cell may be obtained by introducing the Cas transgene in an isolated cell. Delivery systems for transgenes are well known in the art. By means of example, the Cas transgene may be delivered in for instance eukaryotic cell by means of vector (e.g., AAV, adenovirus, lentivirus) and/or particle and/or nanoparticle delivery, as also described herein elsewhere.

It will be understood by the skilled person that the cell, such as the Cas transgenic cell, as referred to herein may comprise further genomic alterations besides having an integrated Cas gene or the mutations arising from the sequence specific action of Cas when complexed with RNA capable of guiding Cas to a target locus.

In certain aspects the invention involves vectors, e.g. for delivering or introducing in a cell Cas and/or RNA capable of guiding Cas to a target locus (i.e. guide RNA), but also for propagating these components (e.g. in prokaryotic cells). A used herein, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). With regards to recombination and cloning methods, mention is made of U.S. patent application Ser. No. 10/815,730, published Sep. 2, 2004 as US 2004-0171156 A1, the contents of which are herein incorporated by reference in their entirety. Thus, the embodiments disclosed herein may also comprise transgenic cells comprising the CRISPR effector system. In certain example embodiments, the transgenic cell may function as an individual discrete volume. In other words, samples comprising a masking construct may be delivered to a cell, for example in a suitable delivery vesicle and if the target is present in the delivery vesicle the CRISPR effector is activated and a detectable signal generated.

The vector(s) can include the regulatory element(s), e.g., promoter(s). The vector(s) can comprise Cas encoding sequences, and/or a single, but possibly also can comprise at least 3 or 8 or 16 or 32 or 48 or 50 guide RNA(s) (e.g., sgRNAs) encoding sequences, such as 1-2, 1-3, 1-4 1-5, 3-6, 3-7, 3-8, 3-9, 3-10, 3-8, 3-16, 3-30, 3-32, 3-48, 3-50 RNA(s) (e.g., sgRNAs). In a single vector there can be a promoter for each RNA (e.g., sgRNA), advantageously when there are up to about 16 RNA(s); and, when a single vector provides for more than 16 RNA(s), one or more promoter(s) can drive expression of more than one of the RNA(s), e.g., when there are 32 RNA(s), each promoter can drive expression of two RNA(s), and when there are 48 RNA(s), each promoter can drive expression of three RNA(s). By simple arithmetic and well-established cloning protocols and the teachings in this disclosure one skilled in the art can readily practice the invention as to the RNA(s) for a suitable exemplary vector such as AAV, and a suitable promoter such as the U6 promoter. For example, the packaging limit of AAV is ˜4.7 kb. The length of a single U6-gRNA (plus restriction sites for cloning) is 361 bp. Therefore, the skilled person can readily fit about 12-16, e.g., 13 U6-gRNA cassettes in a single vector. This can be assembled by any suitable means, such as a golden gate strategy used for TALE assembly (genome-engineering.org/taleffectors/). The skilled person can also use a tandem guide strategy to increase the number of U6-gRNAs by approximately 1.5 times, e.g., to increase from 12-16, e.g., 13 to approximately 18-24, e.g., about 19 U6-gRNAs. Therefore, one skilled in the art can readily reach approximately 18-24, e.g., about 19 promoter-RNAs, e.g., U6-gRNAs in a single vector, e.g., an AAV vector. A further means for increasing the number of promoters and RNAs in a vector is to use a single promoter (e.g., U6) to express an array of RNAs separated by cleavable sequences. And an even further means for increasing the number of promoter-RNAs in a vector, is to express an array of promoter-RNAs separated by cleavable sequences in the intron of a coding sequence or gene; and, in this instance it is advantageous to use a polymerase II promoter, which can have increased expression and enable the transcription of long RNA in a tissue specific manner. (see, e.g., nar.oxfordjoumals.org/content/34/7/e53.short and nature.com/mt/joumal/v16/n9/abs/mt2008144a.html). In an advantageous embodiment, AAV may package U6 tandem gRNA targeting up to about 50 genes. Accordingly, from the knowledge in the art and the teachings in this disclosure the skilled person can readily make and use vector(s), e.g., a single vector, expressing multiple RNAs or guides under the control or operatively or functionally linked to one or more promoters-especially as to the numbers of RNAs or guides discussed herein, without any undue experimentation.

The guide RNA(s) encoding sequences and/or Cas encoding sequences, can be functionally or operatively linked to regulatory element(s) and hence the regulatory element(s) drive expression. The promoter(s) can be constitutive promoter(s) and/or conditional promoter(s) and/or inducible promoter(s) and/or tissue specific promoter(s). The promoter can be selected from the group consisting of RNA polymerases, pol I, pol II, pol III, T7, U6, H1, retroviral Rous sarcoma virus (RSV) LTR promoter, the cytomegalovirus (CMV) promoter, the SV40 promoter, the dihydrofolate reductase promoter, the β-actin promoter, the phosphoglycerol kinase (PGK) promoter, and the EF1α promoter. An advantageous promoter is the promoter U6.

In some embodiments, one or more elements of a nucleic acid-targeting system is derived from a particular organism comprising an endogenous CRISPR RNA-targeting system. In certain example embodiments, the effector protein CRISPR RNA-targeting system comprises at least one HEPN domain, including but not limited to the HEPN domains described herein, HEPN domains known in the art, and domains recognized to be HEPN domains by comparison to consensus sequence motifs. Several such domains are provided herein. In one non-limiting example, a consensus sequence can be derived from the sequences of C2c2 or Cas13b orthologs provided herein. In certain example embodiments, the effector protein comprises a single HEPN domain. In certain other example embodiments, the effector protein comprises two HEPN domains.

In one example embodiment, the effector protein comprises one or more HEPN domains comprising a RxxxxH motif sequence. The RxxxxH motif sequence can be, without limitation, from a HEPN domain described herein or a HEPN domain known in the art. RxxxxH motif sequences further include motif sequences created by combining portions of two or more HEPN domains. As noted, consensus sequences can be derived from the sequences of the orthologs disclosed in PCT/US2017/038154 entitled “Novel Type VI CRISPR Orthologs and Systems,” at, for example, pages 256-264 and 285-336, U.S. Provisional Patent Application 62/432,240 entitled “Novel CRISPR Enzymes and Systems,” U.S. Provisional Patent Application 62/471,710 entitled “Novel Type VI CRISPR Orthologs and Systems” filed on Mar. 15, 2017, and U.S. Provisional Patent Application 62/484,786 entitled “Novel Type VI CRISPR Orthologs and Systems,” filed on Apr. 12, 2017.

In an embodiment of the invention, a HEPN domain comprises at least one RxxxxH motif comprising the sequence of R{N/H/K}X1X2X3H (SEQ ID NO:1). In an embodiment of the invention, a HEPN domain comprises a RxxxxH motif comprising the sequence of R{N/H}X1X2X3H (SEQ ID NO:5). In an embodiment of the invention, a HEPN domain comprises the sequence of R{N/K}X1X2X3H (SEQ ID NO:6). In certain embodiments, X1 is R, S, D, E, Q, N, G, Y, or H. In certain embodiments, X2 is I, S, T, V, or L. In certain embodiments, X3 is L, F, N, Y, V, I, S, D, E, or A.

Additional effectors for use according to the invention can be identified by their proximity to cas1 genes, for example, though not limited to, within the region 20 kb from the start of the cas1 gene and 20 kb from the end of the cas1 gene. In certain embodiments, the effector protein comprises at least one HEPN domain and at least 500 amino acids, and wherein the C2c2 effector protein is naturally present in a prokaryotic genome within 20 kb upstream or downstream of a Cas gene or a CRISPR array. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. In certain example embodiments, the C2c2 effector protein is naturally present in a prokaryotic genome within 20 kb upstream or downstream of a Cas 1 gene. The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of Homologous proteins may but need not be structurally related, or are only partially structurally related. An “orthologue” of a protein as used herein is a protein of a different species which performs the same or a similar function as the protein it is an orthologue of Orthologous proteins may but need not be structurally related, or are only partially structurally related.

In particular embodiments, the Type VI RNA-targeting Cas enzyme is C2c2. In other example embodiments, the Type VI RNA-targeting Cas enzyme is Cas 13b. In particular embodiments, the homologue or orthologue of a Type VI protein such as C2c2 as referred to herein has a sequence homology or identity of at least 30%, or at least 40%, or at least 50%, or at least 60%, or at least 70%, or at least 80%, more preferably at least 85%, even more preferably at least 90%, such as for instance at least 95% with a Type VI protein such as C2c2 (e.g., based on the wild-type sequence of any of Leptotrichia shahii C2c2, Lachnospiraceae bacterium MA2020 C2c2, Lachnospiraceae bacterium NK4A179 C2c2, Clostridium aminophilum (DSM 10710) C2c2, Carnobacterium gallinarum (DSM 4847) C2c2, Paludibacter propionicigenes (WB4) C2c2, Listeria weihenstephanensis (FSL R9-0317) C2c2, Listeriaceae bacterium (FSL M6-0635) C2c2, Listeria newyorkensis (FSL M6-0635) C2c2, Leptotrichia wadei (F0279) C2c2, Rhodobacter capsulatus (SB 1003) C2c2, Rhodobacter capsulatus (R121) C2c2, Rhodobacter capsulatus (DE442) C2c2, Leptotrichia wadei (Lw2) C2c2, or Listeria seeligeri C2c2). In further embodiments, the homologue or orthologue of a Type VI protein such as C2c2 as referred to herein has a sequence identity of at least 30%, or at least 40%, or at least 50%, or at least 60%, or at least 70%, or at least 80%, more preferably at least 85%, even more preferably at least 90%, such as for instance at least 95% with the wild type C2c2 (e.g., based on the wild-type sequence of any of Leptotrichia shahii C2c2, Lachnospiraceae bacterium MA2020 C2c2, Lachnospiraceae bacterium NK4A179 C2c2, Clostridium aminophilum (DSM10710) C2c2, Carnobacterium gallinarum (DSM4847) C2c2, Paludibacter propionicigenes (WB4) C2c2, Listeria weihenstephanensis (FSL R9-0317) C2c2, Listeriaceae bacterium (FSL M6-0635) C2c2, Listeria newyorkensis (FSL M6-0635) C2c2, Leptotrichia wadei (F0279) C2c2, Rhodobacter capsulatus (SB 1003) C2c2, Rhodobacter capsulatus (R121) C2c2, Rhodobacter capsulatus (DE442) C2c2, Leptotrichia wadei (Lw2) C2c2, or Listeria seeligeri C2c2).

In certain other example embodiments, the CRISPR system the effector protein is a C2c2 nuclease. The activity of C2c2 may depend on the presence of two HEPN domains. These have been shown to be RNase domains, i.e. nuclease (in particular an endonuclease) cutting RNA. C2c2 HEPN may also target DNA, or potentially DNA and/or RNA. On the basis that the HEPN domains of C2c2 are at least capable of binding to and, in their wild-type form, cutting RNA, then it is preferred that the C2c2 effector protein has RNase function. Regarding C2c2 CRISPR systems, reference is made to U.S. Provisional 62/351,662 filed on Jun. 17, 2016 and U.S. Provisional 62/376,377 filed on Aug. 17, 2016. Reference is also made to U.S. Provisional 62/351,803 filed on Jun. 17, 2016. Reference is also made to U.S. Provisional entitled “Novel Crispr Enzymes and Systems” filed Dec. 8, 2016 bearing Broad Institute No. 10035.PA4 and Attorney Docket No. 47627.03.2133. Reference is further made to East-Seletsky et al. “Two distinct RNase activities of CRISPR-C2c2 enable guide-RNA processing and RNA detection” Nature doi:10/1038/nature19802 and Abudayyeh et al. “C2c2 is a single-component programmable RNA-guided RNA targeting CRISPR effector” bioRxiv doi:10.1101/054742.

RNase function in CRISPR systems is known, for example mRNA targeting has been reported for certain type III CRISPR-Cas systems (Hale et al., 2014, Genes Dev, vol. 28, 2432-2443; Hale et al., 2009, Cell, vol. 139, 945-956; Peng et al., 2015, Nucleic acids research, vol. 43, 406-417) and provides significant advantages. In the Staphylococcus epidermis type III-A system, transcription across targets results in cleavage of the target DNA and its transcripts, mediated by independent active sites within the Cas10-Csm ribonucleoprotein effector protein complex (see, Samai et al., 2015, Cell, vol. 151, 1164-1174). A CRISPR-Cas system, composition or method targeting RNA via the present effector proteins is thus provided.

In an embodiment, the Cas protein may be a C2c2 ortholog of an organism of a genus which includes but is not limited to Leptotrichia, Listeria, Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, Campylobacter, and Lachnospira. Species of organism of such a genus can be as otherwise herein discussed.

In certain example embodiments, the C2c2 effector proteins of the invention include, without limitation, the following 21 ortholog species (including multiple CRISPR loci: Leptotrichia shahii; Leptotrichia wadei (Lw2); Listeria seeligeri; Lachnospiraceae bacterium MA2020; Lachnospiraceae bacterium NK4A179; [Clostridium]aminophilum DSM 10710; Carnobacterium gallinarum DSM 4847; Carnobacterium gallinarum DSM 4847 (second CRISPR Loci); Paludibacter propionicigenes WB4; Listeria weihenstephanensis FSL R9-0317; Listeriaceae bacterium FSL M6-0635; Leptotrichia wadei F0279; Rhodobacter capsulatus SB 1003; Rhodobacter capsulatus R121; Rhodobacter capsulatus DE442; Leptotrichia buccalis C-1013-b; Herbinix hemicellulosilytica; [Eubacterium] rectale; Eubacteriaceae bacterium CHKCIO04; Blautia sp. Marseille-P2398; and Leptotrichia sp. oral taxon 879 str. F0557. Twelve (12) further non-limiting examples are: Lachnospiraceae bacterium NK4A144; Chloroflexus aggregans; Demequina aurantiaca; Thalassospira sp. TSL5-1; Pseudobutyrivibrio sp. OR37; Butyrivibrio sp. YAB3001; Blautia sp. Marseille-P2398; Leptotrichia sp. Marseille-P3007; Bacteroides ihuae; Porphyromonadaceae bacterium KH3CP3RA; Listeria riparia; and Insolitispirillum peregrinum.

Some methods of identifying orthologues of CRISPR-Cas system enzymes may involve identifying tracr sequences in genomes of interest. Identification of tracr sequences may relate to the following steps: Search for the direct repeats or tracr mate sequences in a database to identify a CRISPR region comprising a CRISPR enzyme. Search for homologous sequences in the CRISPR region flanking the CRISPR enzyme in both the sense and antisense directions. Look for transcriptional terminators and secondary structures. Identify any sequence that is not a direct repeat or a tracr mate sequence but has more than 50% identity to the direct repeat or tracr mate sequence as a potential tracr sequence. Take the potential tracr sequence and analyze for transcriptional terminator sequences associated therewith.

It will be appreciated that any of the functionalities described herein may be engineered into CRISPR enzymes from other orthologs, including chimeric enzymes comprising fragments from multiple orthologs. Examples of such orthologs are described elsewhere herein. Thus, chimeric enzymes may comprise fragments of CRISPR enzyme orthologs of an organism which includes but is not limited to Leptotrichia, Listeria, Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma and Campylobacter. A chimeric enzyme can comprise a first fragment and a second fragment, and the fragments can be of CRISPR enzyme orthologs of organisms of genera herein mentioned or of species herein mentioned; advantageously the fragments are from CRISPR enzyme orthologs of different species.

In embodiments, the C2c2 protein as referred to herein also encompasses a functional variant of C2c2 or a homologue or an orthologue thereof. A “functional variant” of a protein as used herein refers to a variant of such protein which retains at least partially the activity of that protein. Functional variants may include mutants (which may be insertion, deletion, or replacement mutants), including polymorphs, etc. Also included within functional variants are fusion products of such protein with another, usually unrelated, nucleic acid, protein, polypeptide or peptide. Functional variants may be naturally occurring or may be man-made. Advantageous embodiments can involve engineered or non-naturally occurring Type VI RNA-targeting effector protein.

In an embodiment, nucleic acid molecule(s) encoding the C2c2 or an ortholog or homolog thereof, may be codon-optimized for expression in a eukaryotic cell. A eukaryote can be as herein discussed. Nucleic acid molecule(s) can be engineered or non-naturally occurring.

In an embodiment, the C2c2 or an ortholog or homolog thereof, may comprise one or more mutations (and hence nucleic acid molecule(s) coding for same may have mutation(s). The mutations may be artificially introduced mutations and may include but are not limited to one or more mutations in a catalytic domain. Examples of catalytic domains with reference to a Cas9 enzyme may include but are not limited to RuvC I, RuvC II, RuvC III and HNH domains.

In an embodiment, the C2c2 or an ortholog or homolog thereof, may comprise one or more mutations. The mutations may be artificially introduced mutations and may include but are not limited to one or more mutations in a catalytic domain. Examples of catalytic domains with reference to a Cas enzyme may include but are not limited to HEPN domains.

In an embodiment, the C2c2 or an ortholog or homolog thereof, may be used as a generic nucleic acid binding protein with fusion to or being operably linked to a functional domain. Exemplary functional domains may include but are not limited to translational initiator, translational activator, translational repressor, nucleases, in particular ribonucleases, a spliceosome, beads, a light inducible/controllable domain or a chemically inducible/controllable domain.

In certain example embodiments, the C2c2 effector protein may be from an organism selected from the group consisting of, Leptotrichia, Listeria, Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, and Campylobacter.

In certain embodiments, the effector protein may be a Listeria sp. C2c2p, preferably Listeria seeligeria C2c2p, more preferably Listeria seeligeria serovar 1/2b str. SLCC3954 C2c2p and the crRNA sequence may be 44 to 47 nucleotides in length, with a 5′ 29-nt direct repeat (DR) and a 15-nt to 18-nt spacer.

In certain embodiments, the effector protein may be a Leptotrichia sp. C2c2p, preferably Leptotrichia shahii C2c2p, more preferably Leptotrichia shahii DSM 19757 C2c2p and the crRNA sequence may be 42 to 58 nucleotides in length, with a 5′ direct repeat of at least 24 nt, such as a 5′ 24-28-nt direct repeat (DR) and a spacer of at least 14 nt, such as a 14-nt to 28-nt spacer, or a spacer of at least 18 nt, such as 19, 20, 21, 22, or more nt, such as 18-28, 19-28, 20-28, 21-28, or 22-28 nt.

In certain example embodiments, the effector protein may be a Leptotrichia sp., Leptotrichia wadei F0279, or a Listeria sp., preferably Listeria newyorkensis FSL M6-0635.

In certain example embodiments, the C2c2 effector proteins of the invention include, without limitation, the following 21 ortholog species (including multiple CRISPR loci: Leptotrichia shahii; Leptotrichia wadei (Lw2); Listeria seeligeri; Lachnospiraceae bacterium MA2020; Lachnospiraceae bacterium NK4A179; [Clostridium]aminophilum DSM 10710; Carnobacterium gallinarum DSM 4847; Carnobacterium gallinarum DSM 4847 (second CRISPR Loci); Paludibacter propionicigenes WB4; Listeria weihenstephanensis FSL R9-0317; Listeriaceae bacterium FSL M6-0635; Leptotrichia wadei F0279; Rhodobacter capsulatus SB 1003; Rhodobacter capsulatus R121; Rhodobacter capsulatus DE442; Leptotrichia buccalis C-1013-b; Herbinix hemicellulosilytica; [Eubacterium] rectale; Eubacteriaceae bacterium CHKCI004; Blautia sp. Marseille-P2398; and Leptotrichia sp. oral taxon 879 str. F0557. Twelve (12) further non-limiting examples are: Lachnospiraceae bacterium NK4A144; Chloroflexus aggregans; Demequina aurantiaca; Thalassospira sp. TSL5-1; Pseudobutyrivibrio sp. OR37; Butyrivibrio sp. YAB3001; Blautia sp. Marseille-P2398; Leptotrichia sp. Marseille-P3007; Bacteroides ihuae; Porphyromonadaceae bacterium KH3CP3RA; Listeria riparia; and Insolitispirillum peregrinum.

In certain embodiments, the C2c2 protein according to the invention is or is derived from one of the orthologues or is a chimeric protein of two or more of the orthologues as described in this application, or is a mutant or variant of one of the orthologues (or a chimeric mutant or variant), including dead C2c2, split C2c2, destabilized C2c2, etc. as defined herein elsewhere, with or without fusion with a heterologous/functional domain.

In certain example embodiments, the RNA-targeting effector protein is a Type VI-B effector protein, such as Cas13b and Group 29 or Group 30 proteins. In certain example embodiments, the RNA-targeting effector protein comprises one or more HEPN domains. In certain example embodiments, the RNA-targeting effector protein comprises a C-terminal HEPN domain, a N-terminal HEPN domain, or both. Regarding example Type VI-B effector proteins that may be used in the context of this invention, reference is made to U.S. application Ser. No. 15/331,792 entitled “Novel CRISPR Enzymes and Systems” and filed Oct. 21, 2016, International Patent Application No. PCT/US2016/058302 entitled “Novel CRISPR Enzymes and Systems”, and filed Oct. 21, 2016, and Smargon et al. “Cas13b is a Type VI-B CRISPR-associated RNA-Guided RNase differentially regulated by accessory proteins Csx27 and Csx28” Molecular Cell, 65, 1-13 (2017); dx.doi.org/10.1016/j.molcel.2016.12.023, and U.S. Provisional Application No. to be assigned, entitled “Novel Cas13b Orthologues CRISPR Enzymes and System” filed Mar. 15, 2017.

In some aspects, the system further comprises an enrichment CRISPR system, wherein the enrichment CRISPR system is designed to bind the corresponding target molecules prior to detection by the detection CRISPR system. The enrichment CRISPR system, in some embodiments, is designed to bind the corresponding target molecules prior to detection by the detection CRISPR system. The enrichment CRISPR system in a specific embodiment comprises a catalytically inactive CRISPR effector protein, which, in some instances is a catalytically inactive C2c2. The enrichment CRISPR effector protein can, in some embodiments, further comprise a tag, wherein the tag is used to pull down the enrichment CRISPR effector system, or to bind the enrichment CRISPR system to a solid substrate.

### Guide Sequences

As used herein, the term “crRNA” or “guide RNA” or “single guide RNA” or “sgRNA” or “one or more nucleic acid components” of a Type V or Type VI CRISPR-Cas locus effector protein comprises any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible, and will occur to those skilled in the art. A guide sequence, and hence a nucleic acid-targeting guide may be selected to target any target nucleic acid sequence. The target sequence may be DNA. The target sequence may be any RNA sequence. In some embodiments, the target sequence may be a sequence within a RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within a RNA molecule selected from the group consisting of mRNA, pre-mRNA, and rRNA. In some preferred embodiments, the target sequence may be a sequence within a RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin. In an embodiment of the invention, the transcript or transcribed polynucleotide sequence has at least two or more hairpins. In preferred embodiments, the transcript has two, three, four or five hairpins. In a further embodiment of the invention, the transcript has at most five hairpins. In a hairpin structure the portion of the sequence 5′ of the final “N” and upstream of the loop corresponds to the tracr mate sequence, and the portion of the sequence 3′ of the loop corresponds to the tracr sequence.

In general, degree of complementarity is with reference to the optimal alignment of the sca sequence and tracr sequence, along the length of the shorter of the two sequences. Optimal alignment may be determined by any suitable alignment algorithm, and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In general, the CRISPR-Cas, CRISPR-Cas9 or CRISPR system may be as used in the foregoing documents, such as WO 2014/093622 (PCT/US2013/074667) and refers collectively to transcripts and other elements involved in the expression of or directing the activity of CRISPR-associated (“Cas”) genes, including sequences encoding a Cas gene, in particular a Cas9 gene in the case of CRISPR-Cas9, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. The section of the guide sequence through which complementarity to the target sequence is important for cleavage activity is referred to herein as the seed sequence. A target sequence may comprise any polynucleotide, such as DNA or RNA polynucleotides. In some embodiments, a target sequence is located in the nucleus or cytoplasm of a cell, and may include nucleic acids in or from mitochondrial, organelles, vesicles, liposomes or particles present within the cell. In some embodiments, especially for non-nuclear uses, NLSs are not preferred. In some embodiments, a CRISPR system comprises one or more nuclear exports signals (NESs). In some embodiments, a CRISPR system comprises one or more NLSs and one or more NESs. In some embodiments, direct repeats may be identified in silico by searching for repetitive motifs that fulfill any or all of the following criteria: 1. found in a 2 Kb window of genomic sequence flanking the type II CRISPR locus; 2. span from 20 to 50 bp; and 3. interspaced by 20 to 50 bp. In some embodiments, 2 of these criteria may be used, for instance 1 and 2, 2 and 3, or 1 and 3. In some embodiments, all 3 criteria may be used.

In embodiments of the invention the terms guide sequence and guide RNA, i.e. RNA capable of guiding Cas to a target genomic locus, are used interchangeably as in foregoing cited documents such as WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g.the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. Preferably the guide sequence is 10 30 nucleotides long. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

In some embodiments of CRISPR-Cas systems, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and advantageously tracr RNA is 30 or 50 nucleotides in length. However, an aspect of the invention is to reduce off-target interactions, e.g., reduce the guide interacting with a target sequence having low complementarity. Indeed, in the examples, it is shown that the invention involves mutations that result in the CRISPR-Cas system being able to distinguish between target and off-target sequences that have greater than 80% to about 95% complementarity, e.g., 83%-84% or 88-89% or 94-95% complementarity (for instance, distinguishing between a target having 18 nucleotides from an off-target of 18 nucleotides having 1, 2 or 3 mismatches). Accordingly, in the context of the present invention the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In particularly preferred embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e. an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

The methods according to the invention as described herein comprehend inducing one or more mutations in a eukaryotic cell (in vitro, i.e. in an isolated eukaryotic cell) as herein discussed comprising delivering to cell a vector as herein discussed. The mutation(s) can include the introduction, deletion, or substitution of one or more nucleotides at each target sequence of cell(s) via the guide(s) RNA(s) or sgRNA(s). The mutations can include the introduction, deletion, or substitution of 1-75 nucleotides at each target sequence of said cell(s) via the guide(s) RNA(s) or sgRNA(s). The mutations can include the introduction, deletion, or substitution of 1, 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s) or sgRNA(s). The mutations can include the introduction, deletion, or substitution of 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s) or sgRNA(s). The mutations include the introduction, deletion, or substitution of 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s) or sgRNA(s). The mutations can include the introduction, deletion, or substitution of 20, 21, 22, 23, 24, 25, 26, 27, 28, 29, 30, 35, 40, 45, 50, or 75 nucleotides at each target sequence of said cell(s) via the guide(s) RNA(s) or sgRNA(s). The mutations can include the introduction, deletion, or substitution of 40, 45, 50, 75, 100, 200, 300, 400 or 500 nucleotides at each target sequence of said cell(s) via the guide(s) RNA(s) or sgRNA(s).

For minimization of toxicity and off-target effect, it may be important to control the concentration of Cas mRNA and guide RNA delivered. Optimal concentrations of Cas mRNA and guide RNA can be determined by testing different concentrations in a cellular or non-human eukaryote animal model and using deep sequencing the analyze the extent of modification at potential off-target genomic loci. Alternatively, to minimize the level of toxicity and off-target effect, Cas nickase mRNA (for example S. pyogenes Cas9 with the D10A mutation) can be delivered with a pair of guide RNAs targeting a site of interest. Guide sequences and strategies to minimize toxicity and off-target effects can be as in WO 2014/093622 (PCT/US2013/074667); or, via mutation as herein.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence.

### Guide Modifications

In certain embodiments, guides of the invention comprise non-naturally occurring nucleic acids and/or non-naturally occurring nucleotides and/or nucleotide analogs, and/or chemically modifications. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the invention, a guide nucleic acid comprises ribonucleotides and non-ribonucleotides. In one such embodiment, a guide comprises one or more ribonucleotides and one or more deoxyribonucleotides. In an embodiment of the invention, the guide comprises one or more non-naturally occurring nucleotide or nucleotide analog such as a nucleotide with phosphorothioate linkage, boranophosphate linkage, a locked nucleic acid (LNA) nucleotides comprising a methylene bridge between the 2′ and 4′ carbons of the ribose ring, peptide nucleic acids (PNA), or bridged nucleic acids (BNA). Other examples of modified nucleotides include 2′-O-methyl analogs, 2′-deoxy analogs, 2-thiouridine analogs, N6-methyladenosine analogs, or 2′-fluoro analogs. Further examples of modified nucleotides include linkage of chemical moieties at the 2′ position, including but not limited to peptides, nuclear localization sequence (NLS), peptide nucleic acid (PNA), polyethylene glycol (PEG), triethylene glycol, or tetraethyleneglycol (TEG). Further examples of modified bases include, but are not limited to, 2-aminopurine, 5-bromo-uridine, pseudouridine (Ψ), N¹-methylpseudouridine (me¹Ψ), 5-methoxyuridine(5moU), inosine, 7-methylguanosine. Examples of guide RNA chemical modifications include, without limitation, incorporation of 2′-O-methyl (M), 2′-O-methyl-3′-phosphorothioate (MS), phosphorothioate (PS), S-constrained ethyl(cEt), 2′-O-methyl-3′-thioPACE (MSP), or 2′-O-methyl-3′-phosphonoacetate (MP) at one or more terminal nucleotides. Such chemically modified guides can comprise increased stability and increased activity as compared to unmodified guides, though on-target vs. off-target specificity is not predictable. (See, Hendel, 2015, Nat Biotechnol. 33(9):985-9, doi: 10.1038/nbt.3290, published online 29-6-2015; Ragdarm et al., 0215, PNAS, E7110-E7111; Allerson et al., J. Med. Chem. 2005, 48:901-904; Bramsen et al., Front. Genet., 2012, 3:154; Deng et al., PNAS, 2015, 112:11870-11875; Sharma et al., MedChemComm., 2014, 5:1454-1471; Hendel et al., Nat. Biotechnol. (2015) 33(9): 985-989; Li et al., Nature Biomedical Engineering, 2017, 1, 0066 DOI:10.1038/s41551-017-0066; Ryan et al., Nucleic Acids Res. (2018) 46(2): 792-803). In some embodiments, the 5′ and/or 3′ end of a guide RNA is modified by a variety of functional moieties including fluorescent dyes, polyethylene glycol, cholesterol, proteins, or detection tags. (See Kelly et al., 2016, J. Biotech. 233:74-83). In certain embodiments, a guide comprises ribonucleotides in a region that binds to a target DNA and one or more deoxyribonucletides and/or nucleotide analogs in a region that binds to Cas9, Cpf1, or C2c1. In an embodiment of the invention, deoxyribonucleotides and/or nucleotide analogs are incorporated in engineered guide structures, such as, without limitation, 5′ and/or 3′ end, stem-loop regions, and the seed region. In certain embodiments, the modification is not in the 5′-handle of the stem-loop regions. Chemical modification in the 5′-handle of the stem-loop region of a guide may abolish its function (see Li, et al., Nature Biomedical Engineering, 2017, 1:0066). In certain embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or 75 nucleotides of a guide is chemically modified. In some embodiments, 3-5 nucleotides at either the 3′ or the 5′ end of a guide is chemically modified. In some embodiments, only minor modifications are introduced in the seed region, such as 2′-F modifications. In some embodiments, 2′-F modification is introduced at the 3′ end of a guide. In certain embodiments, three to five nucleotides at the 5′ and/or the 3′ end of the guide are chemically modified with 2′-O-methyl (M), 2′-O-methyl-3′-phosphorothioate (MS), S-constrained ethyl(cEt), 2′-O-methyl-3′-thioPACE (MSP), or 2′-O-methyl-3′-phosphonoacetate (MP). Such modification can enhance genome editing efficiency (see Hendel et al., Nat. Biotechnol. (2015) 33(9): 985-989; Ryan et al., Nucleic Acids Res. (2018) 46(2): 792-803). In certain embodiments, all of the phosphodiester bonds of a guide are substituted with phosphorothioates (PS) for enhancing levels of gene disruption. In certain embodiments, more than five nucleotides at the 5′ and/or the 3′ end of the guide are chemically modified with 2′-O-Me, 2′-F or S-constrained ethyl(cEt). Such chemically modified guide can mediate enhanced levels of gene disruption (see Ragdarm et al., 0215, PNAS, E7110-E7111). In an embodiment of the invention, a guide is modified to comprise a chemical moiety at its 3′ and/or 5′ end. Such moieties include, but are not limited to amine, azide, alkyne, thio, dibenzocyclooctyne (DBCO), Rhodamine, peptides, nuclear localization sequence (NLS), peptide nucleic acid (PNA), polyethylene glycol (PEG), triethylene glycol, or tetraethyleneglycol (TEG). In certain embodiment, the chemical moiety is conjugated to the guide by a linker, such as an alkyl chain. In certain embodiments, the chemical moiety of the modified guide can be used to attach the guide to another molecule, such as DNA, RNA, protein, or nanoparticles. Such chemically modified guide can be used to identify or enrich cells generically edited by a CRISPR system (see Lee et al., eLife, 2017, 6:e25312, DOI:10.7554). In some embodiments, 3 nucleotides at each of the 3′ and 5′ ends are chemically modified. In a specific embodiment, the modifications comprise 2′-O-methyl or phosphorothioate analogs. In a specific embodiment, 12 nucleotides in the tetraloop and 16 nucleotides in the stem-loop region are replaced with 2′-O-methyl analogs. Such chemical modifications improve in vivo editing and stability (see Finn et al., Cell Reports (2018), 22: 2227-2235). In some embodiments, more than 60 or 70 nucleotides of the guide are chemically modified. In some embodiments, this modification comprises replacement of nucleotides with 2′-O-methyl or 2′-fluoro nucleotide analogs or phosphorothioate (PS) modification of phosphodiester bonds. In some embodiments, the chemical modification comprises 2′-O-methyl or 2′-fluoro modification of guide nucleotides extending outside of the nuclease protein when the CRISPR complex is formed or PS modification of 20 to 30 or more nucleotides of the 3′-terminus of the guide. In a particular embodiment, the chemical modification further comprises 2′-O-methyl analogs at the 5′ end of the guide or 2′-fluoro analogs in the seed and tail regions. Such chemical modifications improve stability to nuclease degradation and maintain or enhance genome-editing activity or efficiency, but modification of all nucleotides may abolish the function of the guide (see Yin et al., Nat. Biotech. (2018), 35(12): 1179-1187). Such chemical modifications may be guided by knowledge of the structure of the CRISPR complex, including knowledge of the limited number of nuclease and RNA 2′-OH interactions (see Yin et al., Nat. Biotech. (2018), 35(12): 1179-1187). In some embodiments, one or more guide RNA nucleotides may be replaced with DNA nucleotides. In some embodiments, up to 2, 4, 6, 8, 10, or 12 RNA nucleotides of the 5′-end tail/seed guide region are replaced with DNA nucleotides. In certain embodiments, the majority of guide RNA nucleotides at the 3′ end are replaced with DNA nucleotides. In particular embodiments, 16 guide RNA nucleotides at the 3′ end are replaced with DNA nucleotides. In particular embodiments, 8 guide RNA nucleotides of the 5′-end tail/seed region and 16 RNA nucleotides at the 3′ end are replaced with DNA nucleotides. In particular embodiments, guide RNA nucleotides that extend outside of the nuclease protein when the CRISPR complex is formed are replaced with DNA nucleotides. Such replacement of multiple RNA nucleotides with DNA nucleotides leads to decreased off-target activity but similar on-target activity compared to an unmodified guide; however, replacement of all RNA nucleotides at the 3′ end may abolish the function of the guide (see Yin et al., Nat. Chem. Biol. (2018) 14, 311-316). Such modifications may be guided by knowledge of the structure of the CRISPR complex, including knowledge of the limited number of nuclease and RNA 2′-OH interactions (see Yin et al., Nat. Chem. Biol. (2018) 14, 311-316).

In one aspect of the invention, the guide comprises a modified crRNA for Cpf1, having a 5′-handle and a guide segment further comprising a seed region and a 3′-terminus. In some embodiments, the modified guide can be used with a Cpf1 of any one of Acidaminococcus sp. BV3L6 Cpf1 (AsCpf1); Francisella tularensis subsp. Novicida U112 Cpf1 (FnCpf1); L. bacterium MC2017 Cpf1 (Lb3Cpf1); Butyrivibrio proteoclasticus Cpf1 (BpCpf1); Parcubacteria bacterium GWC2011_GWC2_44_17 Cpf1 (PbCpf1); Peregrinibacteria bacterium GW2011_GWA_33_10 Cpf1 (PeCpf1); Leptospira inadai Cpf1 (LiCpf1); Smithella sp. SC_K08D17 Cpf1 (SsCpf1); L. bacterium MA2020 Cpf1 (Lb2Cpf1); Porphyromonas crevioricanis Cpf1 (PcCpf1); Porphyromonas macacae Cpf1 (PmCpf1); Candidatus Methanoplasma termitum Cpf1 (CMtCpf1); Eubacterium eligens Cpf1 (EeCpf1); Moraxella bovoculi 237 Cpf1 (MbCpf1); Prevotella disiens Cpf1 (PdCpf1); or L. bacterium ND2006 Cpf1 (LbCpf1).

In some embodiments, the modification to the guide is a chemical modification, an insertion, a deletion or a split. In some embodiments, the chemical modification includes, but is not limited to, incorporation of 2′-O-methyl (M) analogs, 2′-deoxy analogs, 2-thiouridine analogs, N6-methyladenosine analogs, 2′-fluoro analogs, 2-aminopurine, 5-bromo-uridine, pseudouridine (Ψ), N¹-methylpseudouridine (me¹Ψ), 5-methoxyuridine(5moU), inosine, 7-methylguanosine, 2′-O-methyl-3′-phosphorothioate (MS), S-constrained ethyl(cEt), phosphorothioate (PS), 2′-O-methyl-3′-thioPACE (MSP), or 2′-O-methyl-3′-phosphonoacetate (MP). In some embodiments, the guide comprises one or more of phosphorothioate modifications. In certain embodiments, at least 1, 2, 3, 4, 5, 6, 7, 8, 9, 10, 11, 12, 13, 14, 15, 16, 17, 18, 19, 20, or 25 nucleotides of the guide are chemically modified. In some embodiments, all nucleotides are chemically modified. In certain embodiments, one or more nucleotides in the seed region are chemically modified. In certain embodiments, one or more nucleotides in the 3′-terminus are chemically modified. In certain embodiments, none of the nucleotides in the 5′-handle is chemically modified. In some embodiments, the chemical modification in the seed region is a minor modification, such as incorporation of a 2′-fluoro analog. In a specific embodiment, one nucleotide of the seed region is replaced with a 2′-fluoro analog. In some embodiments, 5 or 10 nucleotides in the 3′-terminus are chemically modified. Such chemical modifications at the 3′-terminus of the Cpf1 CrRNA improve gene cutting efficiency (see Li, et al., Nature Biomedical Engineering, 2017, 1:0066). In a specific embodiment, 5 nucleotides in the 3′-terminus are replaced with 2′-fluoro analogues. In a specific embodiment, 10 nucleotides in the 3′-terminus are replaced with 2′-fluoro analogues. In a specific embodiment, 5 nucleotides in the 3′-terminus are replaced with 2′-O-methyl (M) analogs. In some embodiments, 3 nucleotides at each of the 3′ and 5′ ends are chemically modified. In a specific embodiment, the modifications comprise 2′-O-methyl or phosphorothioate analogs. In a specific embodiment, 12 nucleotides in the tetraloop and 16 nucleotides in the stem-loop region are replaced with 2′-O-methyl analogs. Such chemical modifications improve in vivo editing and stability (see Finn et al., Cell Reports (2018), 22: 2227-2235).

In some embodiments, the loop of the 5′-handle of the guide is modified. In some embodiments, the loop of the 5′-handle of the guide is modified to have a deletion, an insertion, a split, or chemical modifications. In certain embodiments, the loop comprises 3, 4, or 5 nucleotides. In certain embodiments, the loop comprises the sequence of UCUU, UUUU, UAUU, or UGUU. In some embodiments, the guide molecule forms a stemloop with a separate non-covalently linked sequence, which can be DNA or RNA.

### Synthetically Linked Guide

In one aspect, the guide comprises a tracr sequence and a tracr mate sequence that are chemically linked or conjugated via a non-phosphodiester bond. In one aspect, the guide comprises a tracr sequence and a tracr mate sequence that are chemically linked or conjugated via anon-nucleotide loop. In some embodiments, the tracr and tracr mate sequences are joined via a non-phosphodiester covalent linker. Examples of the covalent linker include but are not limited to a chemical moiety selected from the group consisting of carbamates, ethers, esters, amides, imines, amidines, aminotrizines, hydrozone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In some embodiments, the tracr and tracr mate sequences are first synthesized using the standard phosphoramidite synthetic protocol (Herdewijn, P., ed., Methods in Molecular Biology Col 288, Oligonucleotide Synthesis: Methods and Applications, Humana Press, New Jersey (2012)). In some embodiments, the tracr or tracr mate sequences can be functionalized to contain an appropriate functional group for ligation using the standard protocol known in the art (Hermanson, G. T., Bioconjugate Techniques, Academic Press (2013)). Examples of functional groups include, but are not limited to, hydroxyl, amine, carboxylic acid, carboxylic acid halide, carboxylic acid active ester, aldehyde, carbonyl, chlorocarbonyl, imidazolylcarbonyl, hydrozide, semicarbazide, thio semicarbazide, thiol, maleimide, haloalkyl, sufonyl, ally, propargyl, diene, alkyne, and azide. Once the tracr and the tracr mate sequences are functionalized, a covalent chemical bond or linkage can be formed between the two oligonucleotides. Examples of chemical bonds include, but are not limited to, those based on carbamates, ethers, esters, amides, imines, amidines, aminotrizines, hydrozone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In some embodiments, the tracr and tracr mate sequences can be chemically synthesized. In some embodiments, the chemical synthesis uses automated, solid-phase oligonucleotide synthesis machines with 2′-acetoxyethyl orthoester (2′-ACE) (Scaringe et al., J. Am. Chem. Soc. (1998) 120: 11820-11821; Scaringe, Methods Enzymol. (2000) 317: 3-18) or 2′-thionocarbamate (2′-TC) chemistry (Dellinger et al., J. Am. Chem. Soc. (2011) 133: 11540-11546; Hendel et al., Nat. Biotechnol. (2015) 33:985-989).

In some embodiments, the tracr and tracr mate sequences can be covalently linked using various bioconjugation reactions, loops, bridges, and non-nucleotide links via modifications of sugar, intemucleotide phosphodiester bonds, purine and pyrimidine residues. Sletten et al., Angew. Chem. Int. Ed. (2009) 48:6974-6998; Manoharan, M. Curr. Opin. Chem. Biol. (2004) 8: 570-9; Behlke et al., Oligonucleotides (2008) 18: 305-19; Watts, et al., Drug. Discov. Today (2008) 13: 842-55; Shukla, et al., ChemMedChem (2010) 5: 328-49.

In some embodiments, the tracr and tracr mate sequences can be covalently linked using click chemistry. In some embodiments, the tracr and tracr mate sequences can be covalently linked using a triazole linker. In some embodiments, the tracr and tracr mate sequences can be covalently linked using Huisgen 1,3-dipolar cycloaddition reaction involving an alkyne and azide to yield a highly stable triazole linker (He et al., ChemBioChem (2015) 17: 1809-1812; WO 2016/186745). In some embodiments, the tracr and tracr mate sequences are covalently linked by ligating a 5′-hexyne tracrRNA and a 3′-azide crRNA. In some embodiments, either or both of the 5′-hexyne tracrRNA and a 3′-azide crRNA can be protected with 2′-acetoxyethl orthoester (2′-ACE) group, which can be subsequently removed using Dharmacon protocol (Scaringe et al., J. Am. Chem. Soc. (1998) 120: 11820-11821; Scaringe, Methods Enzymol. (2000) 317: 3-18).

In some embodiments, the tracr and tracr mate sequences can be covalently linked via a linker (e.g., a non-nucleotide loop) that comprises a moiety such as spacers, attachments, bioconjugates, chromophores, reporter groups, dye labeled RNAs, and non-naturally occurring nucleotide analogues. More specifically, suitable spacers for purposes of this invention include, but are not limited to, polyethers (e.g., polyethylene glycols, polyalcohols, polypropylene glycol or mixtures of efhylene and propylene glycols), polyamines group (e.g., spennine, spermidine and polymeric derivatives thereof), polyesters (e.g., poly(ethyl acrylate)), polyphosphodiesters, alkylenes, and combinations thereof. Suitable attachments include any moiety that can be added to the linker to add additional properties to the linker, such as but not limited to, fluorescent labels. Suitable bioconjugates include, but are not limited to, peptides, glycosides, lipids, cholesterol, phospholipids, diacyl glycerols and dialkyl glycerols, fatty acids, hydrocarbons, enzyme substrates, steroids, biotin, digoxigenin, carbohydrates, polysaccharides. Suitable chromophores, reporter groups, and dye-labeled RNAs include, but are not limited to, fluorescent dyes such as fluorescein and rhodamine, chemiluminescent, electrochemiluminescent, and bioluminescent marker compounds. The design of example linkers conjugating two RNA components are also described in WO 2004/015075.

The linker (e.g., a non-nucleotide loop) can be of any length. In some embodiments, the linker has a length equivalent to about 0-16 nucleotides. In some embodiments, the linker has a length equivalent to about 0-8 nucleotides. In some embodiments, the linker has a length equivalent to about 0-4 nucleotides. In some embodiments, the linker has a length equivalent to about 2 nucleotides. Example linker design is also described in WO2011/008730.

A typical Type II Cas9 sgRNA comprises (in 5′ to 3′ direction): a guide sequence, a poly U tract, a first complimentary stretch (the “repeat”), a loop (tetraloop), a second complimentary stretch (the “anti-repeat” being complimentary to the repeat), a stem, and further stem loops and stems and a poly A (often poly U in RNA) tail (terminator). In preferred embodiments, certain aspects of guide architecture are retained, certain aspect of guide architecture cam be modified, for example by addition, subtraction, or substitution of features, whereas certain other aspects of guide architecture are maintained. Preferred locations for engineered sgRNA modifications, including but not limited to insertions, deletions, and substitutions include guide termini and regions of the sgRNA that are exposed when complexed with CRISPR protein and/or target, for example the tetraloop and/or loop2.

In certain embodiments, guides of the invention comprise specific binding sites (e.g. aptamers) for adapter proteins, which may comprise one or more functional domains (e.g. via fusion protein). When such a guide forms a CRISPR complex (i.e. CRISPR enzyme binding to guide and target) the adapter proteins bind and, the functional domain associated with the adapter protein is positioned in a spatial orientation which is advantageous for the attributed function to be effective. For example, if the functional domain is a transcription activator (e.g. VP64 or p65), the transcription activator is placed in a spatial orientation which allows it to affect the transcription of the target. Likewise, a transcription repressor will be advantageously positioned to affect the transcription of the target and a nuclease (e.g. Fok1) will be advantageously positioned to cleave or partially cleave the target.

In certain example embodiments, a single guide sequences specific to a single target is placed in separate volumes. Each volume may then receive a different sample or aliquot of the same sample. In certain example embodiments, multiple guide sequences each to separate target may be placed in a single well such that multiple targets may be screened in a different well. In order to detect multiple guide RNAs in a single volume, in certain example embodiments, multiple effector proteins with different specificities may be used. For example, different orthologs with different sequence specificities may be used. For example, one orthologue may preferentially cut A, while others preferentially cut C, G, U/T. Accordingly, masking constructs completely comprising, or comprised of a substantial portion, of a single nucleotide may be generated, each with a different fluorophore that can be detected at differing wavelengths. In this way up to four different targets may be screened in a single individual discrete volume. In certain example embodiments, different orthologues from a same class of CRISPR effector protein may be used, such as two Cas13a orthologues, two Cas13b orthologues, or two Cas13c orthologues. The nucleotide preferences of various Cas13 proteins is shown in FIG. 67. In certain other example embodiments, different orthologues with different nucleotide editing preferences may be used such as a Cas13a and Cas13b orthologs, or a Cas13a and a Cas13c orthologs, or a Cas13b orthologs and a Cas13c orthologs etc. In certain example embodiments, a Cas13 protein with a polyU preference and a Cas13 protein with a polyA preference are used. In certain example embodiments, the Cas13 protein with a polyU preference is a Prevotella intermedia Cas13b. and the Cas13 protein with a polyA preference is a Prevotella sp. MA2106 Cas13b protein (PsmCas13b). In certain example embodiments, the Cas13 protein with a polyU preference is a Leptotrichia wadei Cas13a (LwaCas13a) protein and the Cas13 protein with a poly A preference is a Prevotella sp. MA2106 Cas13b protein. In certain example embodiments, the Cas13 protein with a polyU preference is Capnocytophaga canimorsus Cas13b protein (CcaCas13b).

In addition to single base editing preferences. Additional detection constructs can be designed based on other motif cutting preferences of Cas13 and Cas12 ortholgs. For example, Cas13 or Cas12 orthologs may preferentially cut a dinucleotide sequence, a trinucleotide sequence or more complex motifs comprising 4, 5, 6, 7, 8, 9, or 10 nuleotide motifs. Thus, the upper bound for multiplex assays using the embodiments disclosed herein is primarily limited by the number of distinguishable detectable labels and the detection channels needed to detect them. In certain example embodiments, 2, 3, 4, 5, 6, 7, 8, 9, 10, 11, 12, 13, 14, 15, 16, 17, 18, 19, 20, 21, 22, 23, 24, 25, 25, 27, 28, 29, or 30 different targets are detected. Existing molecular diagnostics generally detect a small number of genomic targets from a single species of pathogen, but approaches disclosed herein such as tiling provide an enhanced opportunity to capture genomic targets when bacterial gDNA is scarce or highly fragmented as with cfDNA. The same principles can be used to expand the number of target, for example the number of pathogens targeted.

The skilled person will understand that modifications to the guide which allow for binding of the adapter+functional domain but not proper positioning of the adapter+functional domain (e.g. due to steric hindrance within the three dimensional structure of the CRISPR complex) are modifications which are not intended. The one or more modified guide may be modified at the tetra loop, the stem loop 1, stem loop 2, or stem loop 3, as described herein, preferably at either the tetra loop or stem loop 2, and most preferably at both the tetra loop and stem loop 2.

The repeat:anti repeat duplex will be apparent from the secondary structure of the sgRNA. It may be typically a first complimentary stretch after (in 5′ to 3′ direction) the poly U tract and before the tetraloop; and a second complimentary stretch after (in 5′ to 3′ direction) the tetraloop and before the poly A tract. The first complimentary stretch (the “repeat”) is complimentary to the second complimentary stretch (the “anti-repeat”). As such, they Watson-Crick base pair to form a duplex of dsRNA when folded back on one another. As such, the anti-repeat sequence is the complimentary sequence of the repeat and in terms to A-U or C-G base pairing, but also in terms of the fact that the anti-repeat is in the reverse orientation due to the tetraloop.

In an embodiment of the invention, modification of guide architecture comprises replacing bases in stemloop 2. For example, in some embodiments, “actt” (“acuu” in RNA) and “aagt” (“aagu” in RNA) bases in stemloop2 are replaced with “cgcc” and “gcgg”. In some embodiments, “actt” and “aagt” bases in stemloop2 are replaced with complimentary GC-rich regions of 4 nucleotides. In some embodiments, the complimentary GC-rich regions of 4 nucleotides are “cgcc” and “gcgg” (both in 5′ to 3′ direction). In some embodiments, the complimentary GC-rich regions of 4 nucleotides are “gcgg” and “cgcc” (both in 5′ to 3′ direction). Other combination of C and G in the complimentary GC-rich regions of 4 nucleotides will be apparent including CCCC and GGGG.

In one aspect, the stemloop 2, e.g., “ACTTgtttAAGT” (SEQ ID NO:7) can be replaced by any “XXXXgtttYYYY” (SEQ ID NO:8), e.g., where XXXX and YYYY represent any complementary sets of nucleotides that together will base pair to each other to create a stem.

In one aspect, the stem comprises at least about 4 bp comprising complementary X and Y sequences, although stems of more, e.g., 5, 6, 7, 8, 9, 10, 11 or 12 or fewer, e.g., 3, 2, base pairs are also contemplated. Thus, for example X2-12 and Y2-12 (wherein X and Y represent any complementary set of nucleotides) may be contemplated. In one aspect, the stem made of the X and Y nucleotides, together with the “gttt,” will form a complete hairpin in the overall secondary structure; and, this may be advantageous and the amount of base pairs can be any amount that forms a complete hairpin. In one aspect, any complementary X:Y basepairing sequence (e.g., as to length) is tolerated, so long as the secondary structure of the entire sgRNA is preserved. In one aspect, the stem can be a form of X:Y basepairing that does not disrupt the secondary structure of the whole sgRNA in that it has a DR:tracr duplex, and 3 stemloops. In one aspect, the “gttt” tetraloop that connects ACTT and AAGT (or any alternative stem made of X:Y basepairs) can be any sequence of the same length (e.g., 4 basepair) or longer that does not interrupt the overall secondary structure of the sgRNA. In one aspect, the stemloop can be something that further lengthens stemloop2, e.g. can be MS2 aptamer.In one aspect, the stemloop3 “GGCACCGagtCGGTGC” (SEQ ID NO:9) can likewise take on a “XXXXXXXagtYYYYYYY” (SEQ ID NO:10) form, e.g., wherein X7 and Y7 represent any complementary sets of nucleotides that together will base pair to each other to create a stem. In one aspect, the stem comprises about 7 bp comprising complementary X and Y sequences, although stems of more or fewer basepairs are also contemplated. In one aspect, the stem made of the X and Y nucleotides, together with the “agt”, will form a complete hairpin in the overall secondary structure. In one aspect, any complementary X:Y basepairing sequence is tolerated, so long as the secondary structure of the entire sgRNA is preserved. In one aspect, the stem can be a form of X:Y basepairing that doesn't disrupt the secondary structure of the whole sgRNA in that it has a DR:tracr duplex, and 3 stemloops. In one aspect, the “agt” sequence of the stemloop 3 can be extended or be replaced by an aptamer, e.g., a MS2 aptamer or sequence that otherwise generally preserves the architecture of stemloop3. In one aspect for alternative Stemloops 2 and/or 3, each X and Y pair can refer to any basepair. In one aspect, non-Watson Crick basepairing is contemplated, where such pairing otherwise generally preserves the architecture of the stemloop at that position.

In one aspect, the DR:tracrRNA duplex can be replaced with the form: gYYYYag(N)NNNNxxxxNNNN(AAN)uuRRRRu (SEQ ID NO:11) (using standard IUPAC nomenclature for nucleotides), wherein (N) and (AAN) represent part of the bulge in the duplex, and “xxxx” represents a linker sequence. NNNN on the direct repeat can be anything so long as it basepairs with the corresponding NNNN portion of the tracrRNA. In one aspect, the DR:tracrRNA duplex can be connected by a linker of any length (xxxx . . . ), any base composition, as long as it does not alter the overall structure.

In one aspect, the sgRNA structural requirement is to have a duplex and 3 stemloops. In most aspects, the actual sequence requirement for many of the particular base requirements are lax, in that the architecture of the DR:tracrRNA duplex should be preserved, but the sequence that creates the architecture, i.e., the stems, loops, bulges, etc., may be altered.

### Aptamers

One guide with a first aptamer/RNA-binding protein pair can be linked or fused to an activator, whilst a second guide with a second aptamer/RNA-binding protein pair can be linked or fused to a repressor. The guides are for different targets (loci), so this allows one gene to be activated and one repressed. For example, the following schematic shows such an approach:
Guide 1—MS2 aptamer-MS2 RNA-binding protein-VP64 activator; and
Guide 2 —PP7 aptamer-PP7 RNA-binding protein-SID4× repressor.

The present invention also relates to orthogonal PP7/MS2 gene targeting. In this example, sgRNA targeting different loci are modified with distinct RNA loops in order to recruit MS2-VP64 or PP7-SID4X, which activate and repress their target loci, respectively. PP7 is the RNA-binding coat protein of the bacteriophage Pseudomonas. Like MS2, it binds a specific RNA sequence and secondary structure. The PP7 RNA-recognition motif is distinct from that of MS2. Consequently, PP7 and MS2 can be multiplexed to mediate distinct effects at different genomic loci simultaneously. For example, an sgRNA targeting locus A can be modified with MS2 loops, recruiting MS2-VP64 activators, while another sgRNA targeting locus B can be modified with PP7 loops, recruiting PP7-SID4X repressor domains. In the same cell, dCas9 can thus mediate orthogonal, locus-specific modifications. This principle can be extended to incorporate other orthogonal RNA-binding proteins such as Q-beta.

An alternative option for orthogonal repression includes incorporating non-coding RNA loops with transactive repressive function into the guide (either at similar positions to the MS2/PP7 loops integrated into the guide or at the 3′ terminus of the guide). For instance, guides were designed with non-coding (but known to be repressive) RNA loops (e.g. using the Alu repressor (in RNA) that interferes with RNA polymerase II in mammalian cells). The Alu RNA sequence was located: in place of the MS2 RNA sequences as used herein (e.g. at tetraloop and/or stem loop 2); and/or at 3′ terminus of the guide. This gives possible combinations of MS2, PP7 or Alu at the tetraloop and/or stemloop 2 positions, as well as, optionally, addition of Alu at the 3′ end of the guide (with or without a linker).

The use of two different aptamers (distinct RNA) allows an activator-adaptor protein fusion and a repressor-adaptor protein fusion to be used, with different guides, to activate expression of one gene, whilst repressing another. They, along with their different guides can be administered together, or substantially together, in a multiplexed approach. A large number of such modified guides can be used all at the same time, for example 10 or 20 or 30 and so forth, whilst only one (or at least a minimal number) of Cas9s to be delivered, as a comparatively small number of Cas9s can be used with a large number of modified guides. The adaptor protein may be associated (preferably linked or fused to) one or more activators or one or more repressors. For example, the adaptor protein may be associated with a first activator and a second activator. The first and second activators may be the same, but they are preferably different activators. For example, one might be VP64, whilst the other might be p65, although these are just examples and other transcriptional activators are envisaged. Three or more or even four or more activators (or repressors) may be used, but package size may limit the number being higher than 5 different functional domains. Linkers are preferably used, over a direct fusion to the adaptor protein, where two or more functional domains are associated with the adaptor protein. Suitable linkers might include the GlySer linker.

It is also envisaged that the enzyme-guide complex as a whole may be associated with two or more functional domains. For example, there may be two or more functional domains associated with the enzyme, or there may be two or more functional domains associated with the guide (via one or more adaptor proteins), or there may be one or more functional domains associated with the enzyme and one or more functional domains associated with the guide (via one or more adaptor proteins).

The fusion between the adaptor protein and the activator or repressor may include a linker. For example, GlySer linkers GGGS can be used. They can be used in repeats of 3 ((GGGGS)₃ (SEQ ID NO:12)) or 6 (SEQ ID NO:13), 9 (SEQ ID NO:14) or even 12 (SEQ ID NO:15) or more, to provide suitable lengths, as required. Linkers can be used between the RNA-binding protein and the functional domain (activator or repressor), or between the CRISPR Enzyme (Cas9) and the functional domain (activator or repressor). The linkers the user to engineer appropriate amounts of “mechanical flexibility”.

### Dead Guides: Guide RNAs Comprising a Dead Guide Sequence May be Used in the Present Invention

In one aspect, the invention provides guide sequences which are modified in a manner which allows for formation of the CRISPR complex and successful binding to the target, while at the same time, not allowing for successful nuclease activity (i.e. without nuclease activity/without indel activity). For matters of explanation such modified guide sequences are referred to as “dead guides” or “dead guide sequences”. These dead guides or dead guide sequences can be thought of as catalytically inactive or conformationally inactive with regard to nuclease activity. Nuclease activity may be measured using surveyor analysis or deep sequencing as commonly used in the art, preferably surveyor analysis. Similarly, dead guide sequences may not sufficiently engage in productive base pairing with respect to the ability to promote catalytic activity or to distinguish on-target and off-target binding activity. Briefly, the surveyor assay involves purifying and amplifying a CRISPR target site for a gene and forming heteroduplexes with primers amplifying the CRISPR target site. After re-anneal, the products are treated with SURVEYOR nuclease and SURVEYOR enhancer S (Transgenomics) following the manufacturer's recommended protocols, analyzed on gels, and quantified based upon relative band intensities.

Hence, in a related aspect, the invention provides a non-naturally occurring or engineered composition Cas9 CRISPR-Cas system comprising a functional Cas9 as described herein, and guide RNA (gRNA) wherein the gRNA comprises a dead guide sequence whereby the gRNA is capable of hybridizing to a target sequence such that the Cas9 CRISPR-Cas system is directed to a genomic locus of interest in a cell without detectable indel activity resultant from nuclease activity of a non-mutant Cas9 enzyme of the system as detected by a SURVEYOR assay. For shorthand purposes, a gRNA comprising a dead guide sequence whereby the gRNA is capable of hybridizing to a target sequence such that the Cas9 CRISPR-Cas system is directed to a genomic locus of interest in a cell without detectable indel activity resultant from nuclease activity of a non-mutant Cas9 enzyme of the system as detected by a SURVEYOR assay is herein termed a “dead gRNA”. It is to be understood that any of the gRNAs according to the invention as described herein elsewhere may be used as dead gRNAs/gRNAs comprising a dead guide sequence as described herein below. Any of the methods, products, compositions and uses as described herein elsewhere is equally applicable with the dead gRNAs/gRNAs comprising a dead guide sequence as further detailed below. By means of further guidance, the following particular aspects and embodiments are provided.

The ability of a dead guide sequence to direct sequence-specific binding of a CRISPR complex to a target sequence may be assessed by any suitable assay. For example, the components of a CRISPR system sufficient to form a CRISPR complex, including the dead guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the dead guide sequence to be tested and a control guide sequence different from the test dead guide sequence, and comparing binding or rate of cleavage at the target sequence between the test and control guide sequence reactions. Other assays are possible, and will occur to those skilled in the art. A dead guide sequence may be selected to target any target sequence. In some embodiments, the target sequence is a sequence within a genome of a cell.

As explained further herein, several structural parameters allow for a proper framework to arrive at such dead guides. Dead guide sequences are shorter than respective guide sequences which result in active Cas9-specific indel formation. Dead guides are 5%, 10%, 20%, 30%, 40%, 50%, shorter than respective guides directed to the same Cas9 leading to active Cas9-specific indel formation.

As explained below and known in the art, one aspect of gRNA—Cas9 specificity is the direct repeat sequence, which is to be appropriately linked to such guides. In particular, this implies that the direct repeat sequences are designed dependent on the origin of the Cas9. Thus, structural data available for validated dead guide sequences may be used for designing Cas9 specific equivalents. Structural similarity between, e.g., the orthologous nuclease domains RuvC of two or more Cas9 effector proteins may be used to transfer design equivalent dead guides. Thus, the dead guide herein may be appropriately modified in length and sequence to reflect such Cas9 specific equivalents, allowing for formation of the CRISPR complex and successful binding to the target, while at the same time, not allowing for successful nuclease activity.

The use of dead guides in the context herein as well as the state of the art provides a surprising and unexpected platform for network biology and/or systems biology in both in vitro, ex vivo, and in vivo applications, allowing for multiplex gene targeting, and in particular bidirectional multiplex gene targeting. Prior to the use of dead guides, addressing multiple targets, for example for activation, repression and/or silencing of gene activity, has been challenging and in some cases not possible. With the use of dead guides, multiple targets, and thus multiple activities, may be addressed, for example, in the same cell, in the same animal, or in the same patient. Such multiplexing may occur at the same time or staggered for a desired timeframe.

For example, the dead guides now allow for the first time to use gRNA as a means for gene targeting, without the consequence of nuclease activity, while at the same time providing directed means for activation or repression. Guide RNA comprising a dead guide may be modified to further include elements in a manner which allow for activation or repression of gene activity, in particular protein adaptors (e.g. aptamers) as described herein elsewhere allowing for functional placement of gene effectors (e.g. activators or repressors of gene activity). One example is the incorporation of aptamers, as explained herein and in the state of the art. By engineering the gRNA comprising a dead guide to incorporate protein-interacting aptamers (Konermann et al., “Genome-scale transcription activation by an engineered CRISPR-Cas9 complex,” doi:10.1038/nature14136, incorporated herein by reference), one may assemble a synthetic transcription activation complex consisting of multiple distinct effector domains. Such may be modeled after natural transcription activation processes. For example, an aptamer, which selectively binds an effector (e.g. an activator or repressor; dimerized MS2 bacteriophage coat proteins as fusion proteins with an activator or repressor), or a protein which itself binds an effector (e.g. activator or repressor) may be appended to a dead gRNA tetraloop and/or a stem-loop 2. In the case of MS2, the fusion protein MS2-VP64 binds to the tetraloop and/or stem-loop 2 and in turn mediates transcriptional up-regulation, for example for Neurog2. Other transcriptional activators are, for example, VP64. P65, HSF1, and MyoD1. By mere example of this concept, replacement of the MS2 stem-loops with PP7-interacting stem-loops may be used to recruit repressive elements.

Thus, one aspect is a gRNA of the invention which comprises a dead guide, wherein the gRNA further comprises modifications which provide for gene activation or repression, as described herein. The dead gRNA may comprise one or more aptamers. The aptamers may be specific to gene effectors, gene activators or gene repressors. Alternatively, the aptamers may be specific to a protein which in turn is specific to and recruits/binds a specific gene effector, gene activator or gene repressor. If there are multiple sites for activator or repressor recruitment, it is preferred that the sites are specific to either activators or repressors. If there are multiple sites for activator or repressor binding, the sites may be specific to the same activators or same repressors. The sites may also be specific to different activators or different repressors. The gene effectors, gene activators, gene repressors may be present in the form of fusion proteins.

In an embodiment, the dead gRNA as described herein or the Cas9 CRISPR-Cas complex as described herein includes a non-naturally occurring or engineered composition comprising two or more adaptor proteins, wherein each protein is associated with one or more functional domains and wherein the adaptor protein binds to the distinct RNA sequence(s) inserted into the at least one loop of the dead gRNA.

Hence, an aspect provides a non-naturally occurring or engineered composition comprising a guide RNA (gRNA) comprising a dead guide sequence capable of hybridizing to a target sequence in a genomic locus of interest in a cell, wherein the dead guide sequence is as defined herein, a Cas9 comprising at least one or more nuclear localization sequences, wherein the Cas9 optionally comprises at least one mutation wherein at least one loop of the dead gRNA is modified by the insertion of distinct RNA sequence(s) that bind to one or more adaptor proteins, and wherein the adaptor protein is associated with one or more functional domains; or, wherein the dead gRNA is modified to have at least one non-coding functional loop, and wherein the composition comprises two or more adaptor proteins, wherein the each protein is associated with one or more functional domains.

In certain embodiments, the adaptor protein is a fusion protein comprising the functional domain, the fusion protein optionally comprising a linker between the adaptor protein and the functional domain, the linker optionally including a GlySer linker.

In certain embodiments, the at least one loop of the dead gRNA is not modified by the insertion of distinct RNA sequence(s) that bind to the two or more adaptor proteins.

In certain embodiments, the one or more functional domains associated with the adaptor protein is a transcriptional activation domain.

In certain embodiments, the one or more functional domains associated with the adaptor protein is a transcriptional activation domain comprising VP64, p65, MyoD1, HSF1, RTA or SET7/9.

In certain embodiments, the one or more functional domains associated with the adaptor protein is a transcriptional repressor domain.

In certain embodiments, the transcriptional repressor domain is a KRAB domain.

In certain embodiments, the transcriptional repressor domain is a NuE domain, NcoR domain, SID domain or a SID4X domain.

In certain embodiments, at least one of the one or more functional domains associated with the adaptor protein have one or more activities comprising methylase activity, demethylase activity, transcription activation activity, transcription repression activity, transcription release factor activity, histone modification activity, DNA integration activity RNA cleavage activity, DNA cleavage activity or nucleic acid binding activity.

In certain embodiments, the DNA cleavage activity is due to a Fok1 nuclease.

In certain embodiments, the dead gRNA is modified so that, after dead gRNA binds the adaptor protein and further binds to the Cas9 and target, the functional domain is in a spatial orientation allowing for the functional domain to function in its attributed function.

In certain embodiments, the at least one loop of the dead gRNA is tetra loop and/or loop2. In certain embodiments, the tetra loop and loop 2 of the dead gRNA are modified by the insertion of the distinct RNA sequence(s).

In certain embodiments, the insertion of distinct RNA sequence(s) that bind to one or more adaptor proteins is an aptamer sequence. In certain embodiments, the aptamer sequence is two or more aptamer sequences specific to the same adaptor protein. In certain embodiments, the aptamer sequence is two or more aptamer sequences specific to different adaptor protein.

In certain embodiments, the adaptor protein comprises MS2, PP7, QP, F2, GA, fr, JP501, M12, R17, BZ13, JP34, JP500, KU1, M11, MX1, TW18, VK, SP, FI, ID2, NL95, TW19, AP205, ϕCb5, ϕCb8r, ϕCb12r, ϕCb23r, 7s, PRR1.

In certain embodiments, the cell is a eukaryotic cell. In certain embodiments, the eukaryotic cell is a mammalian cell, optionally a mouse cell. In certain embodiments, the mammalian cell is a human cell.

In certain embodiments, a first adaptor protein is associated with a p65 domain and a second adaptor protein is associated with a HSF1 domain.

In certain embodiments, the composition comprises a Cas9 CRISPR-Cas complex having at least three functional domains, at least one of which is associated with the Cas9 and at least two of which are associated with dead gRNA.

In certain embodiments, the composition further comprises a second gRNA, wherein the second gRNA is a live gRNA capable of hybridizing to a second target sequence such that a second Cas9 CRISPR-Cas system is directed to a second genomic locus of interest in a cell with detectable indel activity at the second genomic locus resultant from nuclease activity of the Cas9 enzyme of the system.

In certain embodiments, the composition further comprises a plurality of dead gRNAs and/or a plurality of live gRNAs.

One aspect of the invention is to take advantage of the modularity and customizability of the gRNA scaffold to establish a series of gRNA scaffolds with different binding sites (in particular aptamers) for recruiting distinct types of effectors in an orthogonal manner. Again, for matters of example and illustration of the broader concept, replacement of the MS2 stem-loops with PP7-interacting stem-loops may be used to bind/recruit repressive elements, enabling multiplexed bidirectional transcriptional control. Thus, in general, gRNA comprising a dead guide may be employed to provide for multiplex transcriptional control and preferred bidirectional transcriptional control. This transcriptional control is most preferred of genes. For example, one or more gRNA comprising dead guide(s) may be employed in targeting the activation of one or more target genes. At the same time, one or more gRNA comprising dead guide(s) may be employed in targeting the repression of one or more target genes. Such a sequence may be applied in a variety of different combinations, for example the target genes are first repressed and then at an appropriate period other targets are activated, or select genes are repressed at the same time as select genes are activated, followed by further activation and/or repression. As a result, multiple components of one or more biological systems may advantageously be addressed together.

In an aspect, the invention provides nucleic acid molecule(s) encoding dead gRNA or the Cas9 CRISPR-Cas complex or the composition as described herein.

In an aspect, the invention provides a vector system comprising: a nucleic acid molecule encoding dead guide RNA as defined herein. In certain embodiments, the vector system further comprises a nucleic acid molecule(s) encoding Cas9. In certain embodiments, the vector system further comprises a nucleic acid molecule(s) encoding (live) gRNA. In certain embodiments, the nucleic acid molecule or the vector further comprises regulatory element(s) operable in a eukaryotic cell operably linked to the nucleic acid molecule encoding the guide sequence (gRNA) and/or the nucleic acid molecule encoding Cas9 and/or the optional nuclear localization sequence(s).

In another aspect, structural analysis may also be used to study interactions between the dead guide and the active Cas9 nuclease that enable DNA binding, but no DNA cutting. In this way amino acids important for nuclease activity of Cas9 are determined. Modification of such amino acids allows for improved Cas9 enzymes used for gene editing.

A further aspect is combining the use of dead guides as explained herein with other applications of CRISPR, as explained herein as well as known in the art. For example, gRNA comprising dead guide(s) for targeted multiplex gene activation or repression or targeted multiplex bidirectional gene activation/repression may be combined with gRNA comprising guides which maintain nuclease activity, as explained herein. Such gRNA comprising guides which maintain nuclease activity may or may not further include modifications which allow for repression of gene activity (e.g. aptamers). Such gRNA comprising guides which maintain nuclease activity may or may not further include modifications which allow for activation of gene activity (e.g. aptamers). In such a manner, a further means for multiplex gene control is introduced (e.g. multiplex gene targeted activation without nuclease activity/without indel activity may be provided at the same time or in combination with gene targeted repression with nuclease activity).

For example, 1) using one or more gRNA (e.g. 1-50, 1-40, 1-30, 1-20, preferably 1-10, more preferably 1-5) comprising dead guide(s) targeted to one or more genes and further modified with appropriate aptamers for the recruitment of gene activators; 2) may be combined with one or more gRNA (e.g. 1-50, 1-40, 1-30, 1-20, preferably 1-10, more preferably 1-5) comprising dead guide(s) targeted to one or more genes and further modified with appropriate aptamers for the recruitment of gene repressors. 1) and/or 2) may then be combined with 3) one or more gRNA (e.g. 1-50, 1-40, 1-30, 1-20, preferably 1-10, more preferably 1-5) targeted to one or more genes. This combination can then be carried out in turn with 1)+2)+3) with 4) one or more gRNA (e.g. 1-50, 1-40, 1-30, 1-20, preferably 1-10, more preferably 1-5) targeted to one or more genes and further modified with appropriate aptamers for the recruitment of gene activators. This combination can then be carried in turn with 1)+2)+3)+4) with 5) one or more gRNA (e.g. 1-50, 1-40, 1-30, 1-20, preferably 1-10, more preferably 1-5) targeted to one or more genes and further modified with appropriate aptamers for the recruitment of gene repressors. As a result various uses and combinations are included in the invention. For example, combination 1)+2); combination 1)+3); combination 2)+3); combination 1)+2)+3); combination 1)+2)+3)+4); combination 1)+3)+4); combination 2)+3)+4); combination 1)+2)+4); combination 1)+2)+3)+4)+5); combination 1)+3)+4)+5); combination 2)+3)+4)+5); combination 1)+2)+4)+5); combination 1)+2)+3)+5); combination 1)+3)+5); combination 2)+3)+5); combination 1)+2)+5).

In an aspect, the invention provides an algorithm for designing, evaluating, or selecting a dead guide RNA targeting sequence (dead guide sequence) for guiding a Cas9 CRISPR-Cas system to a target gene locus. In particular, it has been determined that dead guide RNA specificity relates to and can be optimized by varying i) GC content and ii) targeting sequence length. In an aspect, the invention provides an algorithm for designing or evaluating a dead guide RNA targeting sequence that minimizes off-target binding or interaction of the dead guide RNA. In an embodiment of the invention, the algorithm for selecting a dead guide RNA targeting sequence for directing a CRISPR system to a gene locus in an organism comprises a) locating one or more CRISPR motifs in the gene locus, analyzing the 20 nt sequence downstream of each CRISPR motif by i) determining the GC content of the sequence; and ii) determining whether there are off-target matches of the 15 downstream nucleotides nearest to the CRISPR motif in the genome of the organism, and c) selecting the 15 nucleotide sequence for use in a dead guide RNA if the GC content of the sequence is 70% or less and no off-target matches are identified. In an embodiment, the sequence is selected for a targeting sequence if the GC content is 60% or less. In certain embodiments, the sequence is selected for a targeting sequence if the GC content is 55% or less, 50% or less, 45% or less, 40% or less, 35% or less or 30% or less. In an embodiment, two or more sequences of the gene locus are analyzed and the sequence having the lowest GC content, or the next lowest GC content, or the next lowest GC content is selected. In an embodiment, the sequence is selected for a targeting sequence if no off-target matches are identified in the genome of the organism. In an embodiment, the targeting sequence is selected if no off-target matches are identified in regulatory sequences of the genome.

In an aspect, the invention provides a method of selecting a dead guide RNA targeting sequence for directing a functionalized CRISPR system to a gene locus in an organism, which comprises: a) locating one or more CRISPR motifs in the gene locus; b) analyzing the 20 nt sequence downstream of each CRISPR motif by: i) determining the GC content of the sequence; and ii) determining whether there are off-target matches of the first 15 nt of the sequence in the genome of the organism; c) selecting the sequence for use in a guide RNA if the GC content of the sequence is 70% or less and no off-target matches are identified. In an embodiment, the sequence is selected if the GC content is 50% or less. In an embodiment, the sequence is selected if the GC content is 40% or less. In an embodiment, the sequence is selected if the GC content is 30% or less. In an embodiment, two or more sequences are analyzed and the sequence having the lowest GC content is selected. In an embodiment, off-target matches are determined in regulatory sequences of the organism. In an embodiment, the gene locus is a regulatory region. An aspect provides a dead guide RNA comprising the targeting sequence selected according to the aforementioned methods.

In an aspect, the invention provides a dead guide RNA for targeting a functionalized CRISPR system to a gene locus in an organism. In an embodiment of the invention, the dead guide RNA comprises a targeting sequence wherein the CG content of the target sequence is 70% or less, and the first 15 nt of the targeting sequence does not match an off-target sequence downstream from a CRISPR motif in the regulatory sequence of another gene locus in the organism. In certain embodiments, the GC content of the targeting sequence 60% or less, 55% or less, 50% or less, 45% or less, 40% or less, 35% or less or 30% or less. In certain embodiments, the GC content of the targeting sequence is from 70% to 60% or from 60% to 50% or from 50% to 40% or from 40% to 30%. In an embodiment, the targeting sequence has the lowest CG content among potential targeting sequences of the locus.

In an embodiment of the invention, the first 15 nt of the dead guide match the target sequence. In another embodiment, first 14 nt of the dead guide match the target sequence. In another embodiment, the first 13 nt of the dead guide match the target sequence. In another embodiment first 12 nt of the dead guide match the target sequence. In another embodiment, first 11 nt of the dead guide match the target sequence. In another embodiment, the first 10 nt of the dead guide match the target sequence. In an embodiment of the invention the first 15 nt of the dead guide does not match an off-target sequence downstream from a CRISPR motif in the regulatory region of another gene locus. In other embodiments, the first 14 nt, or the first 13 nt of the dead guide, or the first 12 nt of the guide, or the first 11 nt of the dead guide, or the first 10 nt of the dead guide, does not match an off-target sequence downstream from a CRISPR motif in the regulatory region of another gene locus. In other embodiments, the first 15 nt, or 14 nt, or 13 nt, or 12 nt, or 11 nt of the dead guide do not match an off-target sequence downstream from a CRISPR motif in the genome.

In certain embodiments, the dead guide RNA includes additional nucleotides at the 3′-end that do not match the target sequence. Thus, a dead guide RNA that includes the first 15 nt, or 14 nt, or 13 nt, or 12 nt, or 11 nt downstream of a CRISPR motif can be extended in length at the 3′ end to 12 nt, 13 nt, 14 nt, 15 nt, 16 nt, 17 nt, 18 nt, 19 nt, 20 nt, or longer.

The invention provides a method for directing a Cas9 CRISPR-Cas system, including but not limited to a dead Cas9 (dCas9) or functionalized Cas9 system (which may comprise a functionalized Cas9 or functionalized guide) to a gene locus. In an aspect, the invention provides a method for selecting a dead guide RNA targeting sequence and directing a functionalized CRISPR system to a gene locus in an organism. In an aspect, the invention provides a method for selecting a dead guide RNA targeting sequence and effecting gene regulation of a target gene locus by a functionalized Cas9 CRISPR-Cas system. In certain embodiments, the method is used to effect target gene regulation while minimizing off-target effects. In an aspect, the invention provides a method for selecting two or more dead guide RNA targeting sequences and effecting gene regulation of two or more target gene loci by a functionalized Cas9 CRISPR-Cas system. In certain embodiments, the method is used to effect regulation of two or more target gene loci while minimizing off-target effects.

In an aspect, the invention provides a method of selecting a dead guide RNA targeting sequence for directing a functionalized Cas9 to a gene locus in an organism, which comprises: a) locating one or more CRISPR motifs in the gene locus; b) analyzing the sequence downstream of each CRISPR motif by: i) selecting 10 to 15 nt adjacent to the CRISPR motif, ii) determining the GC content of the sequence; and c) selecting the 10 to 15 nt sequence as a targeting sequence for use in a guide RNA if the GC content of the sequence is 40% or more. In an embodiment, the sequence is selected if the GC content is 50% or more. In an embodiment, the sequence is selected if the GC content is 60% or more. In an embodiment, the sequence is selected if the GC content is 70% or more. In an embodiment, two or more sequences are analyzed and the sequence having the highest GC content is selected. In an embodiment, the method further comprises adding nucleotides to the 3′ end of the selected sequence which do not match the sequence downstream of the CRISPR motif. An aspect provides a dead guide RNA comprising the targeting sequence selected according to the aforementioned methods.

In an aspect, the invention provides a dead guide RNA for directing a functionalized CRISPR system to a gene locus in an organism wherein the targeting sequence of the dead guide RNA consists of 10 to 15 nucleotides adjacent to the CRISPR motif of the gene locus, wherein the CG content of the target sequence is 50% or more. In certain embodiments, the dead guide RNA further comprises nucleotides added to the 3′ end of the targeting sequence which do not match the sequence downstream of the CRISPR motif of the gene locus.

In an aspect, the invention provides for a single effector to be directed to one or more, or two or more gene loci. In certain embodiments, the effector is associated with a Cas9, and one or more, or two or more selected dead guide RNAs are used to direct the Cas9-associated effector to one or more, or two or more selected target gene loci. In certain embodiments, the effector is associated with one or more, or two or more selected dead guide RNAs, each selected dead guide RNA, when complexed with a Cas9 enzyme, causing its associated effector to localize to the dead guide RNA target. One non-limiting example of such CRISPR systems modulates activity of one or more, or two or more gene loci subject to regulation by the same transcription factor.

In an aspect, the invention provides for two or more effectors to be directed to one or more gene loci. In certain embodiments, two or more dead guide RNAs are employed, each of the two or more effectors being associated with a selected dead guide RNA, with each of the two or more effectors being localized to the selected target of its dead guide RNA. One non-limiting example of such CRISPR systems modulates activity of one or more, or two or more gene loci subject to regulation by different transcription factors. Thus, in one non-limiting embodiment, two or more transcription factors are localized to different regulatory sequences of a single gene. In another non-limiting embodiment, two or more transcription factors are localized to different regulatory sequences of different genes. In certain embodiments, one transcription factor is an activator. In certain embodiments, one transcription factor is an inhibitor. In certain embodiments, one transcription factor is an activator and another transcription factor is an inhibitor. In certain embodiments, gene loci expressing different components of the same regulatory pathway are regulated. In certain embodiments, gene loci expressing components of different regulatory pathways are regulated.

In an aspect, the invention also provides a method and algorithm for designing and selecting dead guide RNAs that are specific for target DNA cleavage or target binding and gene regulation mediated by an active Cas9 CRISPR-Cas system. In certain embodiments, the Cas9 CRISPR-Cas system provides orthogonal gene control using an active Cas9 which cleaves target DNA at one gene locus while at the same time binds to and promotes regulation of another gene locus.

In an aspect, the invention provides an method of selecting a dead guide RNA targeting sequence for directing a functionalized Cas9 to a gene locus in an organism, without cleavage, which comprises a) locating one or more CRISPR motifs in the gene locus; b) analyzing the sequence downstream of each CRISPR motif by i) selecting 10 to 15 nt adjacent to the CRISPR motif, ii) determining the GC content of the sequence, and c) selecting the 10 to 15 nt sequence as a targeting sequence for use in a dead guide RNA if the GC content of the sequence is 30% more, 40% or more. In certain embodiments, the GC content of the targeting sequence is 35% or more, 40% or more, 45% or more, 50% or more, 55% or more, 60% or more, 65% or more, or 70% or more. In certain embodiments, the GC content of the targeting sequence is from 30% to 40% or from 40% to 50% or from 50% to 60% or from 60% to 70%. In an embodiment of the invention, two or more sequences in a gene locus are analyzed and the sequence having the highest GC content is selected.

In an embodiment of the invention, the portion of the targeting sequence in which GC content is evaluated is 10 to 15 contiguous nucleotides of the 15 target nucleotides nearest to the PAM. In an embodiment of the invention, the portion of the guide in which GC content is considered is the 10 to 11 nucleotides or 11 to 12 nucleotides or 12 to 13 nucleotides or 13, or 14, or 15 contiguous nucleotides of the 15 nucleotides nearest to the PAM.

In an aspect, the invention further provides an algorithm for identifying dead guide RNAs which promote CRISPR system gene locus cleavage while avoiding functional activation or inhibition. It is observed that increased GC content in dead guide RNAs of 16 to 20 nucleotides coincides with increased DNA cleavage and reduced functional activation.

It is also demonstrated herein that efficiency of functionalized Cas9 can be increased by addition of nucleotides to the 3′ end of a guide RNA which do not match a target sequence downstream of the CRISPR motif. For example, of dead guide RNA 11 to 15 nt in length, shorter guides may be less likely to promote target cleavage, but are also less efficient at promoting CRISPR system binding and functional control. In certain embodiments, addition of nucleotides that don't match the target sequence to the 3′ end of the dead guide RNA increase activation efficiency while not increasing undesired target cleavage. In an aspect, the invention also provides a method and algorithm for identifying improved dead guide RNAs that effectively promote CRISPRP system function in DNA binding and gene regulation while not promoting DNA cleavage. Thus, in certain embodiments, the invention provides a dead guide RNA that includes the first 15 nt, or 14 nt, or 13 nt, or 12 nt, or 11 nt downstream of a CRISPR motif and is extended in length at the 3′ end by nucleotides that mismatch the target to 12 nt, 13 nt, 14 nt, 15 nt, 16 nt, 17 nt, 18 nt, 19 nt, 20 nt, or longer.

In an aspect, the invention provides a method for effecting selective orthogonal gene control. As will be appreciated from the disclosure herein, dead guide selection according to the invention, taking into account guide length and GC content, provides effective and selective transcription control by a functional Cas9 CRISPR-Cas system, for example to regulate transcription of a gene locus by activation or inhibition and minimize off-target effects. Accordingly, by providing effective regulation of individual target loci, the invention also provides effective orthogonal regulation of two or more target loci.

In certain embodiments, orthogonal gene control is by activation or inhibition of two or more target loci. In certain embodiments, orthogonal gene control is by activation or inhibition of one or more target locus and cleavage of one or more target locus.

In one aspect, the invention provides a cell comprising a non-naturally occurring Cas9 CRISPR-Cas system comprising one or more dead guide RNAs disclosed or made according to a method or algorithm described herein wherein the expression of one or more gene products has been altered. In an embodiment of the invention, the expression in the cell of two or more gene products has been altered. The invention also provides a cell line from such a cell.

In one aspect, the invention provides a multicellular organism comprising one or more cells comprising a non-naturally occurring Cas9 CRISPR-Cas system comprising one or more dead guide RNAs disclosed or made according to a method or algorithm described herein. In one aspect, the invention provides a product from a cell, cell line, or multicellular organism comprising a non-naturally occurring Cas9 CRISPR-Cas system comprising one or more dead guide RNAs disclosed or made according to a method or algorithm described herein.

A further aspect of this invention is the use of gRNA comprising dead guide(s) as described herein, optionally in combination with gRNA comprising guide(s) as described herein or in the state of the art, in combination with systems e.g. cells, transgenic animals, transgenic mice, inducible transgenic animals, inducible transgenic mice) which are engineered for either overexpression of Cas9 or preferably knock in Cas9. As a result a single system (e.g. transgenic animal, cell) can serve as a basis for multiplex gene modifications in systems/network biology. On account of the dead guides, this is now possible in both in vitro, ex vivo, and in vivo.

For example, once the Cas9 is provided for, one or more dead gRNAs may be provided to direct multiplex gene regulation, and preferably multiplex bidirectional gene regulation. The one or more dead gRNAs may be provided in a spatially and temporally appropriate manner if necessary or desired (for example tissue specific induction of Cas9 expression). On account that the transgenic/inducible Cas9 is provided for (e.g. expressed) in the cell, tissue, animal of interest, both gRNAs comprising dead guides or gRNAs comprising guides are equally effective. In the same manner, a further aspect of this invention is the use of gRNA comprising dead guide(s) as described herein, optionally in combination with gRNA comprising guide(s) as described herein or in the state of the art, in combination with systems (e.g. cells, transgenic animals, transgenic mice, inducible transgenic animals, inducible transgenic mice) which are engineered for knockout Cas9 CRISPR-Cas.

As a result, the combination of dead guides as described herein with CRISPR applications described herein and CRISPR applications known in the art results in a highly efficient and accurate means for multiplex screening of systems (e.g. network biology). Such screening allows, for example, identification of specific combinations of gene activities for identifying genes responsible for diseases (e.g. on/off combinations), in particular gene related diseases. A preferred application of such screening is cancer. In the same manner, screening for treatment for such diseases is included in the invention. Cells or animals may be exposed to aberrant conditions resulting in disease or disease like effects. Candidate compositions may be provided and screened for an effect in the desired multiplex environment. For example a patient's cancer cells may be screened for which gene combinations will cause them to die, and then use this information to establish appropriate therapies.

In one aspect, the invention provides a kit comprising one or more of the components described herein. The kit may include dead guides as described herein with or without guides as described herein.

The structural information provided herein allows for interrogation of dead gRNA interaction with the target DNA and the Cas9 permitting engineering or alteration of dead gRNA structure to optimize functionality of the entire Cas9 CRISPR-Cas system. For example, loops of the dead gRNA may be extended, without colliding with the Cas9 protein by the insertion of adaptor proteins that can bind to RNA. These adaptor proteins can further recruit effector proteins or fusions which comprise one or more functional domains.

In some preferred embodiments, the functional domain is a transcriptional activation domain, preferably VP64. In some embodiments, the functional domain is a transcription repression domain, preferably KRAB. In some embodiments, the transcription repression domain is SID, or concatemers of SID (e.g. SID4X). In some embodiments, the functional domain is an epigenetic modifying domain, such that an epigenetic modifying enzyme is provided. In some embodiments, the functional domain is an activation domain, which may be the P65 activation domain.

An aspect of the invention is that the above elements are comprised in a single composition or comprised in individual compositions. These compositions may advantageously be applied to a host to elicit a functional effect on the genomic level.

In general, the dead gRNA are modified in a manner that provides specific binding sites (e.g. aptamers) for adapter proteins comprising one or more functional domains (e.g. via fusion protein) to bind to. The modified dead gRNA are modified such that once the dead gRNA forms a CRISPR complex (i.e. Cas9 binding to dead gRNA and target) the adapter proteins bind and, the functional domain on the adapter protein is positioned in a spatial orientation which is advantageous for the attributed function to be effective. For example, if the functional domain is a transcription activator (e.g. VP64 or p65), the transcription activator is placed in a spatial orientation which allows it to affect the transcription of the target. Likewise, a transcription repressor will be advantageously positioned to affect the transcription of the target and a nuclease (e.g. Fok1) will be advantageously positioned to cleave or partially cleave the target.

The skilled person will understand that modifications to the dead gRNA which allow for binding of the adapter+functional domain but not proper positioning of the adapter+functional domain (e.g. due to steric hindrance within the three dimensional structure of the CRISPR complex) are modifications which are not intended. The one or more modified dead gRNA may be modified at the tetra loop, the stem loop 1, stem loop 2, or stem loop 3, as described herein, preferably at either the tetra loop or stem loop 2, and most preferably at both the tetra loop and stem loop 2.

As explained herein the functional domains may be, for example, one or more domains from the group consisting of methylase activity, demethylase activity, transcription activation activity, transcription repression activity, transcription release factor activity, histone modification activity, RNA cleavage activity, DNA cleavage activity, nucleic acid binding activity, and molecular switches (e.g. light inducible). In some cases it is advantageous that additionally at least one NLS is provided. In some instances, it is advantageous to position the NLS at the N terminus. When more than one functional domain is included, the functional domains may be the same or different.

The dead gRNA may be designed to include multiple binding recognition sites (e.g. aptamers) specific to the same or different adapter protein. The dead gRNA may be designed to bind to the promoter region—1000—+1 nucleic acids upstream of the transcription start site (i.e. TSS), preferably—200 nucleic acids. This positioning improves functional domains which affect gene activation (e.g. transcription activators) or gene inhibition (e.g. transcription repressors). The modified dead gRNA may be one or more modified dead gRNAs targeted to one or more target loci (e.g. at least 1 gRNA, at least 2 gRNA, at least 5 gRNA, at least 10 gRNA, at least 20 gRNA, at least 30 gRNA, at least 50 gRNA) comprised in a composition.

The adaptor protein may be any number of proteins that binds to an aptamer or recognition site introduced into the modified dead gRNA and which allows proper positioning of one or more functional domains, once the dead gRNA has been incorporated into the CRISPR complex, to affect the target with the attributed function. As explained in detail in this application such may be coat proteins, preferably bacteriophage coat proteins. The functional domains associated with such adaptor proteins (e.g. in the form of fusion protein) may include, for example, one or more domains from the group consisting of methylase activity, demethylase activity, transcription activation activity, transcription repression activity, transcription release factor activity, histone modification activity, RNA cleavage activity, DNA cleavage activity, nucleic acid binding activity, and molecular switches (e.g. light inducible). Preferred domains are Fok1, VP64, P65, HSF1, MyoD1. In the event that the functional domain is a transcription activator or transcription repressor it is advantageous that additionally at least an NLS is provided and preferably at the N terminus. When more than one functional domain is included, the functional domains may be the same or different. The adaptor protein may utilize known linkers to attach such functional domains.

Thus, the modified dead gRNA, the (inactivated) Cas9 (with or without functional domains), and the binding protein with one or more functional domains, may each individually be comprised in a composition and administered to a host individually or collectively. Alternatively, these components may be provided in a single composition for administration to a host. Administration to a host may be performed via viral vectors known to the skilled person or described herein for delivery to a host (e.g. lentiviral vector, adenoviral vector, AAV vector). As explained herein, use of different selection markers (e.g. for lentiviral gRNA selection) and concentration of gRNA (e.g. dependent on whether multiple gRNAs are used) may be advantageous for eliciting an improved effect.

On the basis of this concept, several variations are appropriate to elicit a genomic locus event, including DNA cleavage, gene activation, or gene deactivation. Using the provided compositions, the person skilled in the art can advantageously and specifically target single or multiple loci with the same or different functional domains to elicit one or more genomic locus events. The compositions may be applied in a wide variety of methods for screening in libraries in cells and functional modeling in vivo (e.g. gene activation of lincRNA and identification of function; gain-of-function modeling; loss-of-function modeling; the use the compositions of the invention to establish cell lines and transgenic animals for optimization and screening purposes).

The current invention comprehends the use of the compositions of the current invention to establish and utilize conditional or inducible CRISPR transgenic cell/animals, which are not believed prior to the present invention or application. For example, the target cell comprises Cas9 conditionally or inducibly (e.g. in the form of Cre dependent constructs) and/or the adapter protein conditionally or inducibly and, on expression of a vector introduced into the target cell, the vector expresses that which induces or gives rise to the condition of Cas9 expression and/or adaptor expression in the target cell. By applying the teaching and compositions of the current invention with the known method of creating a CRISPR complex, inducible genomic events affected by functional domains are also an aspect of the current invention. One example of this is the creation of a CRISPR knock-in/conditional transgenic animal (e.g. mouse comprising e.g. a Lox-Stop-polyA-Lox(LSL) cassette) and subsequent delivery of one or more compositions providing one or more modified dead gRNA (e.g. —200 nucleotides to TSS of a target gene of interest for gene activation purposes) as described herein (e.g. modified dead gRNA with one or more aptamers recognized by coat proteins, e.g. MS2), one or more adapter proteins as described herein (MS2 binding protein linked to one or more VP64) and means for inducing the conditional animal (e.g. Cre recombinase for rendering Cas9 expression inducible). Alternatively, the adaptor protein may be provided as a conditional or inducible element with a conditional or inducible Cas9 to provide an effective model for screening purposes, which advantageously only requires minimal design and administration of specific dead gRNAs for a broad number of applications.

In another aspect the dead guides are further modified to improve specificity. Protected dead guides may be synthesized, whereby secondary structure is introduced into the 3′ end of the dead guide to improve its specificity. A protected guide RNA (pgRNA) comprises a guide sequence capable of hybridizing to a target sequence in a genomic locus of interest in a cell and a protector strand, wherein the protector strand is optionally complementary to the guide sequence and wherein the guide sequence may in part be hybridizable to the protector strand. The pgRNA optionally includes an extension sequence. The thermodynamics of the pgRNA-target DNA hybridization is determined by the number of bases complementary between the guide RNA and target DNA. By employing ‘thermodynamic protection’, specificity of dead gRNA can be improved by adding a protector sequence. For example, one method adds a complementary protector strand of varying lengths to the 3′ end of the guide sequence within the dead gRNA. As a result, the protector strand is bound to at least a portion of the dead gRNA and provides for a protected gRNA (pgRNA). In turn, the dead gRNA references herein may be easily protected using the described embodiments, resulting in pgRNA. The protector strand can be either a separate RNA transcript or strand or a chimeric version joined to the 3′ end of the dead gRNA guide sequence.

### Tandem Guides and Uses in a Multiplex (Tandem) Targeting Approach

The inventors have shown that CRISPR enzymes as defined herein can employ more than one RNA guide without losing activity. This enables the use of the CRISPR enzymes, systems or complexes as defined herein for targeting multiple DNA targets, genes or gene loci, with a single enzyme, system or complex as defined herein. The guide RNAs may be tandemly arranged, optionally separated by a nucleotide sequence such as a direct repeat as defined herein. The position of the different guide RNAs is the tandem does not influence the activity. It is noted that the terms “CRISPR-Cas system”, “CRISP-Cas complex” “CRISPR complex” and “CRISPR system” are used interchangeably. Also the terms “CRISPR enzyme”, “Cas enzyme”, or “CRISPR-Cas enzyme”, can be used interchangeably. In preferred embodiments, said CRISPR enzyme, CRISP-Cas enzyme or Cas enzyme is Cas9, or any one of the modified or mutated variants thereof described herein elsewhere.

In one aspect, the invention provides a non-naturally occurring or engineered CRISPR enzyme, preferably a class 2 CRISPR enzyme, preferably a Type V or VI CRISPR enzyme as described herein, such as without limitation Cas9 as described herein elsewhere, used for tandem or multiplex targeting. It is to be understood that any of the CRISPR (or CRISPR-Cas or Cas) enzymes, complexes, or systems according to the invention as described herein elsewhere may be used in such an approach. Any of the methods, products, compositions and uses as described herein elsewhere are equally applicable with the multiplex or tandem targeting approach further detailed below. By means of further guidance, the following particular aspects and embodiments are provided.

In one aspect, the invention provides for the use of a Cas9 enzyme, complex or system as defined herein for targeting multiple gene loci. In one embodiment, this can be established by using multiple (tandem or multiplex) guide RNA (gRNA) sequences.

In one aspect, the invention provides methods for using one or more elements of a Cas9 enzyme, complex or system as defined herein for tandem or multiplex targeting, wherein said CRISP system comprises multiple guide RNA sequences. Preferably, said gRNA sequences are separated by a nucleotide sequence, such as a direct repeat as defined herein elsewhere.

The Cas9 enzyme, system or complex as defined herein provides an effective means for modifying multiple target polynucleotides. The Cas9 enzyme, system or complex as defined herein has a wide variety of utility including modifying (e.g., deleting, inserting, translocating, inactivating, activating) one or more target polynucleotides in a multiplicity of cell types. As such the Cas9 enzyme, system or complex as defined herein of the invention has a broad spectrum of applications in, e.g., gene therapy, drug screening, disease diagnosis, and prognosis, including targeting multiple gene loci within a single CRISPR system.

In one aspect, the invention provides a Cas9 enzyme, system or complex as defined herein, i.e. a Cas9 CRISPR-Cas complex having a Cas9 protein having at least one destabilization domain associated therewith, and multiple guide RNAs that target multiple nucleic acid molecules such as DNA molecules, whereby each of said multiple guide RNAs specifically targets its corresponding nucleic acid molecule, e.g., DNA molecule. Each nucleic acid molecule target, e.g., DNA molecule can encode a gene product or encompass a gene locus. Using multiple guide RNAs hence enables the targeting of multiple gene loci or multiple genes. In some embodiments the Cas9 enzyme may cleave the DNA molecule encoding the gene product. In some embodiments expression of the gene product is altered. The Cas9 protein and the guide RNAs do not naturally occur together. The invention comprehends the guide RNAs comprising tandemly arranged guide sequences. The invention further comprehends coding sequences for the Cas9 protein being codon optimized for expression in a eukaryotic cell. In a preferred embodiment the eukaryotic cell is a mammalian cell, a plant cell or a yeast cell and in a more preferred embodiment the mammalian cell is a human cell. Expression of the gene product may be decreased. The Cas9 enzyme may form part of a CRISPR system or complex, which further comprises tandemly arranged guide RNAs (gRNAs) comprising a series of 2, 3, 4, 5, 6, 7, 8, 9, 10, 15, 25, 25, 30, or more than 30 guide sequences, each capable of specifically hybridizing to a target sequence in a genomic locus of interest in a cell. In some embodiments, the functional Cas9 CRISPR system or complex binds to the multiple target sequences. In some embodiments, the functional CRISPR system or complex may edit the multiple target sequences, e.g., the target sequences may comprise a genomic locus, and in some embodiments there may be an alteration of gene expression. In some embodiments, the functional CRISPR system or complex may comprise further functional domains. In some embodiments, the invention provides a method for altering or modifying expression of multiple gene products. The method may comprise introducing into a cell containing said target nucleic acids, e.g., DNA molecules, or containing and expressing target nucleic acid, e.g., DNA molecules; for instance, the target nucleic acids may encode gene products or provide for expression of gene products (e.g., regulatory sequences).

In preferred embodiments the CRISPR enzyme used for multiplex targeting is Cas9, or the CRISPR system or complex comprises Cas9. In some embodiments, the CRISPR enzyme used for multiplex targeting is AsCas9, or the CRISPR system or complex used for multiplex targeting comprises an AsCas9. In some embodiments, the CRISPR enzyme is an LbCas9, or the CRISPR system or complex comprises LbCas9. In some embodiments, the Cas9 enzyme used for multiplex targeting cleaves both strands of DNA to produce a double strand break (DSB). In some embodiments, the CRISPR enzyme used for multiplex targeting is a nickase. In some embodiments, the Cas9 enzyme used for multiplex targeting is a dual nickase. In some embodiments, the Cas9 enzyme used for multiplex targeting is a Cas9 enzyme such as a DD Cas9 enzyme as defined herein elsewhere.

In some general embodiments, the Cas9 enzyme used for multiplex targeting is associated with one or more functional domains. In some more specific embodiments, the CRISPR enzyme used for multiplex targeting is a deadCas9 as defined herein elsewhere.

In an aspect, the present invention provides a means for delivering the Cas9 enzyme, system or complex for use in multiple targeting as defined herein or the polynucleotides defined herein. Non-limiting examples of such delivery means are e.g. particle(s) delivering component(s) of the complex, vector(s) comprising the polynucleotide(s) discussed herein (e.g., encoding the CRISPR enzyme, providing the nucleotides encoding the CRISPR complex). In some embodiments, the vector may be a plasmid or a viral vector such as AAV, or lentivirus. Transient transfection with plasmids, e.g., into HEK cells may be advantageous, especially given the size limitations of AAV and that while Cas9 fits into AAV, one may reach an upper limit with additional guide RNAs.

Also provided is a model that constitutively expresses the Cas9 enzyme, complex or system as used herein for use in multiplex targeting. The organism may be transgenic and may have been transfected with the present vectors or may be the offspring of an organism so transfected. In a further aspect, the present invention provides compositions comprising the CRISPR enzyme, system and complex as defined herein or the polynucleotides or vectors described herein. Also provides are Cas9 CRISPR systems or complexes comprising multiple guide RNAs, preferably in a tandemly arranged format. Said different guide RNAs may be separated by nucleotide sequences such as direct repeats.

Also provided is a method of treating a subject, e.g., a subject in need thereof, comprising inducing gene editing by transforming the subject with the polynucleotide encoding the Cas9 CRISPR system or complex or any of polynucleotides or vectors described herein and administering them to the subject. A suitable repair template may also be provided, for example delivered by a vector comprising said repair template. Also provided is a method of treating a subject, e.g., a subject in need thereof, comprising inducing transcriptional activation or repression of multiple target gene loci by transforming the subject with the polynucleotides or vectors described herein, wherein said polynucleotide or vector encodes or comprises the Cas9 enzyme, complex or system comprising multiple guide RNAs, preferably tandemly arranged. Where any treatment is occurring ex vivo, for example in a cell culture, then it will be appreciated that the term ‘subject’ may be replaced by the phrase “cell or cell culture.”

Compositions comprising Cas9 enzyme, complex or system comprising multiple guide RNAs, preferably tandemly arranged, or the polynucleotide or vector encoding or comprising said Cas9 enzyme, complex or system comprising multiple guide RNAs, preferably tandemly arranged, for use in the methods of treatment as defined herein elsewhere are also provided. A kit of parts may be provided including such compositions. Use of said composition in the manufacture of a medicament for such methods of treatment are also provided. Use of a Cas9 CRISPR system in screening is also provided by the present invention, e.g., gain of function screens. Cells which are artificially forced to overexpress a gene are be able to down regulate the gene over time (re-establishing equilibrium) e.g. by negative feedback loops. By the time the screen starts the unregulated gene might be reduced again. Using an inducible Cas9 activator allows one to induce transcription right before the screen and therefore minimizes the chance of false negative hits. Accordingly, by use of the instant invention in screening, e.g., gain of function screens, the chance of false negative results may be minimized.

In one aspect, the invention provides an engineered, non-naturally occurring CRISPR system comprising a Cas9 protein and multiple guide RNAs that each specifically target a DNA molecule encoding a gene product in a cell, whereby the multiple guide RNAs each target their specific DNA molecule encoding the gene product and the Cas9 protein cleaves the target DNA molecule encoding the gene product, whereby expression of the gene product is altered; and, wherein the CRISPR protein and the guide RNAs do not naturally occur together. The invention comprehends the multiple guide RNAs comprising multiple guide sequences, preferably separated by a nucleotide sequence such as a direct repeat and optionally fused to a tracr sequence. In an embodiment of the invention the CRISPR protein is a type V or VI CRISPR-Cas protein and in a more preferred embodiment the CRISPR protein is a Cas9 protein. The invention further comprehends a Cas9 protein being codon optimized for expression in a eukaryotic cell. In a preferred embodiment the eukaryotic cell is a mammalian cell and in a more preferred embodiment the mammalian cell is a human cell. In a further embodiment of the invention, the expression of the gene product is decreased.

In another aspect, the invention provides an engineered, non-naturally occurring vector system comprising one or more vectors comprising a first regulatory element operably linked to the multiple Cas9 CRISPR system guide RNAs that each specifically target a DNA molecule encoding a gene product and a second regulatory element operably linked coding for a CRISPR protein. Both regulatory elements may be located on the same vector or on different vectors of the system. The multiple guide RNAs target the multiple DNA molecules encoding the multiple gene products in a cell and the CRISPR protein may cleave the multiple DNA molecules encoding the gene products (it may cleave one or both strands or have substantially no nuclease activity), whereby expression of the multiple gene products is altered; and, wherein the CRISPR protein and the multiple guide RNAs do not naturally occur together. In a preferred embodiment the CRISPR protein is Cas9 protein, optionally codon optimized for expression in a eukaryotic cell. In a preferred embodiment the eukaryotic cell is a mammalian cell, a plant cell or a yeast cell and in a more preferred embodiment the mammalian cell is a human cell. In a further embodiment of the invention, the expression of each of the multiple gene products is altered, preferably decreased.

In one aspect, the invention provides a vector system comprising one or more vectors. In some embodiments, the system comprises: (a) a first regulatory element operably linked to a direct repeat sequence and one or more insertion sites for inserting one or more guide sequences up- or downstream (whichever applicable) of the direct repeat sequence, wherein when expressed, the one or more guide sequence(s) direct(s) sequence-specific binding of the CRISPR complex to the one or more target sequence(s) in a eukaryotic cell, wherein the CRISPR complex comprises a Cas9 enzyme complexed with the one or more guide sequence(s) that is hybridized to the one or more target sequence(s); and (b) a second regulatory element operably linked to an enzyme-coding sequence encoding said Cas9 enzyme, preferably comprising at least one nuclear localization sequence and/or at least one NES; wherein components (a) and (b) are located on the same or different vectors of the system. Where applicable, a tracr sequence may also be provided. In some embodiments, component (a) further comprises two or more guide sequences operably linked to the first regulatory element, wherein when expressed, each of the two or more guide sequences direct sequence specific binding of a Cas9 CRISPR complex to a different target sequence in a eukaryotic cell. In some embodiments, the CRISPR complex comprises one or more nuclear localization sequences and/or one or more NES of sufficient strength to drive accumulation of said Cas9 CRISPR complex in a detectable amount in or out of the nucleus of a eukaryotic cell. In some embodiments, the first regulatory element is a polymerase III promoter. In some embodiments, the second regulatory element is a polymerase II promoter. In some embodiments, each of the guide sequences is at least 16, 17, 18, 19, 20, 25 nucleotides, or between 16-30, or between 16-25, or between 16-20 nucleotides in length.

Recombinant expression vectors can comprise the polynucleotides encoding the Cas9 enzyme, system or complex for use in multiple targeting as defined herein in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell).

In some embodiments, a host cell is transiently or non-transiently transfected with one or more vectors comprising the polynucleotides encoding the Cas9 enzyme, system or complex for use in multiple targeting as defined herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art and exemplified herein elsewhere. Cell lines are available from a variety of sources known to those with skill in the art (see, e.g., the American Type Culture Collection (ATCC) (Manassas, Va.)). In some embodiments, a cell transfected with one or more vectors comprising the polynucleotides encoding the Cas9 enzyme, system or complex for use in multiple targeting as defined herein is used to establish a new cell line comprising one or more vector-derived sequences. In some embodiments, a cell transiently transfected with the components of a Cas9 CRISPR system or complex for use in multiple targeting as described herein (such as by transient transfection of one or more vectors, or transfection with RNA), and modified through the activity of a Cas9 CRISPR system or complex, is used to establish a new cell line comprising cells containing the modification but lacking any other exogenous sequence. In some embodiments, cells transiently or non-transiently transfected with one or more vectors comprising the polynucleotides encoding the Cas9 enzyme, system or complex for use in multiple targeting as defined herein, or cell lines derived from such cells are used in assessing one or more test compounds.

The term “regulatory element” is as defined herein elsewhere.

Advantageous vectors include lentiviruses and adeno-associated viruses, and types of such vectors can also be selected for targeting particular types of cells.

In one aspect, the invention provides a eukaryotic host cell comprising (a) a first regulatory element operably linked to a direct repeat sequence and one or more insertion sites for inserting one or more guide RNA sequences up- or downstream (whichever applicable) of the direct repeat sequence, wherein when expressed, the guide sequence(s) direct(s) sequence-specific binding of the Cas9 CRISPR complex to the respective target sequence(s) in a eukaryotic cell, wherein the Cas9 CRISPR complex comprises a Cas9 enzyme complexed with the one or more guide sequence(s) that is hybridized to the respective target sequence(s); and/or (b) a second regulatory element operably linked to an enzyme-coding sequence encoding said Cas9 enzyme comprising preferably at least one nuclear localization sequence and/or NES. In some embodiments, the host cell comprises components (a) and (b). Where applicable, a tracr sequence may also be provided. In some embodiments, component (a), component (b), or components (a) and (b) are stably integrated into a genome of the host eukaryotic cell. In some embodiments, component (a) further comprises two or more guide sequences operably linked to the first regulatory element, and optionally separated by a direct repeat, wherein when expressed, each of the two or more guide sequences direct sequence specific binding of a Cas9 CRISPR complex to a different target sequence in a eukaryotic cell. In some embodiments, the Cas9 enzyme comprises one or more nuclear localization sequences and/or nuclear export sequences or NES of sufficient strength to drive accumulation of said CRISPR enzyme in a detectable amount in and/or out of the nucleus of a eukaryotic cell.

In some embodiments, the Cas9 enzyme is a type V or VI CRISPR system enzyme. In some embodiments, the Cas9 enzyme is a Cas9 enzyme. In some embodiments, the Cas9 enzyme is derived from Francisella tularensis 1, Francisella tularensis subsp. novicida, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens, or Porphyromonas macacae Cas9, and may include further alterations or mutations of the Cas9 as defined herein elsewhere, and can be a chimeric Cas9. In some embodiments, the Cas9 enzyme is codon-optimized for expression in a eukaryotic cell. In some embodiments, the CRISPR enzyme directs cleavage of one or two strands at the location of the target sequence. In some embodiments, the first regulatory element is a polymerase III promoter. In some embodiments, the second regulatory element is a polymerase II promoter. In some embodiments, the one or more guide sequence(s) is (are each) at least 16, 17, 18, 19, 20, 25 nucleotides, or between 16-30, or between 16-25, or between 16-20 nucleotides in length. When multiple guide RNAs are used, they are preferably separated by a direct repeat sequence. In an aspect, the invention provides a non-human eukaryotic organism; preferably a multicellular eukaryotic organism, comprising a eukaryotic host cell according to any of the described embodiments. In other aspects, the invention provides a eukaryotic organism; preferably a multicellular eukaryotic organism, comprising a eukaryotic host cell according to any of the described embodiments. The organism in some embodiments of these aspects may be an animal; for example a mammal. Also, the organism may be an arthropod such as an insect. The organism also may be a plant. Further, the organism may be a fungus.

In one aspect, the invention provides a kit comprising one or more of the components described herein. In some embodiments, the kit comprises a vector system and instructions for using the kit. In some embodiments, the vector system comprises (a) a first regulatory element operably linked to a direct repeat sequence and one or more insertion sites for inserting one or more guide sequences up- or downstream (whichever applicable) of the direct repeat sequence, wherein when expressed, the guide sequence directs sequence-specific binding of a Cas9 CRISPR complex to a target sequence in a eukaryotic cell, wherein the Cas9 CRISPR complex comprises a Cas9 enzyme complexed with the guide sequence that is hybridized to the target sequence; and/or (b) a second regulatory element operably linked to an enzyme-coding sequence encoding said Cas9 enzyme comprising a nuclear localization sequence. Where applicable, a tracr sequence may also be provided. In some embodiments, the kit comprises components (a) and (b) located on the same or different vectors of the system. In some embodiments, component (a) further comprises two or more guide sequences operably linked to the first regulatory element, wherein when expressed, each of the two or more guide sequences direct sequence specific binding of a CRISPR complex to a different target sequence in a eukaryotic cell. In some embodiments, the Cas9 enzyme comprises one or more nuclear localization sequences of sufficient strength to drive accumulation of said CRISPR enzyme in a detectable amount in the nucleus of a eukaryotic cell. In some embodiments, the CRISPR enzyme is a type V or VI CRISPR system enzyme. In some embodiments, the CRISPR enzyme is a Cas9 enzyme. In some embodiments, the Cas9 enzyme is derived from Francisella tularensis 1, Francisella tularensis subsp. novicida, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens, or Porphyromonas macacae Cas9 (e.g., modified to have or be associated with at least one DD), and may include further alteration or mutation of the Cas9, and can be a chimeric Cas9. In some embodiments, the DD-CRISPR enzyme is codon-optimized for expression in a eukaryotic cell. In some embodiments, the DD-CRISPR enzyme directs cleavage of one or two strands at the location of the target sequence. In some embodiments, the DD-CRISPR enzyme lacks or substantially DNA strand cleavage activity (e.g., no more than 5% nuclease activity as compared with a wild type enzyme or enzyme not having the mutation or alteration that decreases nuclease activity). In some embodiments, the first regulatory element is a polymerase III promoter. In some embodiments, the second regulatory element is a polymerase II promoter. In some embodiments, the guide sequence is at least 16, 17, 18, 19, 20, 25 nucleotides, or between 16-30, or between 16-25, or between 16-20 nucleotides in length.

In one aspect, the invention provides a method of modifying multiple target polynucleotides in a host cell such as a eukaryotic cell. In some embodiments, the method comprises allowing a Cas9CRISPR complex to bind to multiple target polynucleotides, e.g., to effect cleavage of said multiple target polynucleotides, thereby modifying multiple target polynucleotides, wherein the Cas9CRISPR complex comprises a Cas9 enzyme complexed with multiple guide sequences each of the being hybridized to a specific target sequence within said target polynucleotide, wherein said multiple guide sequences are linked to a direct repeat sequence. Where applicable, a tracr sequence may also be provided (e.g. to provide a single guide RNA, sgRNA). In some embodiments, said cleavage comprises cleaving one or two strands at the location of each of the target sequence by said Cas9 enzyme. In some embodiments, said cleavage results in decreased transcription of the multiple target genes. In some embodiments, the method further comprises repairing one or more of said cleaved target polynucleotide by homologous recombination with an exogenous template polynucleotide, wherein said repair results in a mutation comprising an insertion, deletion, or substitution of one or more nucleotides of one or more of said target polynucleotides. In some embodiments, said mutation results in one or more amino acid changes in a protein expressed from a gene comprising one or more of the target sequence(s). In some embodiments, the method further comprises delivering one or more vectors to said eukaryotic cell, wherein the one or more vectors drive expression of one or more of: the Cas9 enzyme and the multiple guide RNA sequence linked to a direct repeat sequence. Where applicable, a tracr sequence may also be provided. In some embodiments, said vectors are delivered to the eukaryotic cell in a subject. In some embodiments, said modifying takes place in said eukaryotic cell in a cell culture. In some embodiments, the method further comprises isolating said eukaryotic cell from a subject prior to said modifying. In some embodiments, the method further comprises returning said eukaryotic cell and/or cells derived therefrom to said subject.

In one aspect, the invention provides a method of modifying expression of multiple polynucleotides in a eukaryotic cell. In some embodiments, the method comprises allowing a Cas9 CRISPR complex to bind to multiple polynucleotides such that said binding results in increased or decreased expression of said polynucleotides; wherein the Cas9 CRISPR complex comprises a Cas9 enzyme complexed with multiple guide sequences each specifically hybridized to its own target sequence within said polynucleotide, wherein said guide sequences are linked to a direct repeat sequence. Where applicable, a tracr sequence may also be provided. In some embodiments, the method further comprises delivering one or more vectors to said eukaryotic cells, wherein the one or more vectors drive expression of one or more of: the Cas9 enzyme and the multiple guide sequences linked to the direct repeat sequences. Where applicable, a tracr sequence may also be provided.

In one aspect, the invention provides a recombinant polynucleotide comprising multiple guide RNA sequences up- or downstream (whichever applicable) of a direct repeat sequence, wherein each of the guide sequences when expressed directs sequence-specific binding of a Cas9CRISPR complex to its corresponding target sequence present in a eukaryotic cell. In some embodiments, the target sequence is a viral sequence present in a eukaryotic cell. Where applicable, a tracr sequence may also be provided. In some embodiments, the target sequence is a proto-oncogene or an oncogene.

Aspects of the invention encompass a non-naturally occurring or engineered composition that may comprise a guide RNA (gRNA) comprising a guide sequence capable of hybridizing to a target sequence in a genomic locus of interest in a cell and a Cas9 enzyme as defined herein that may comprise at least one or more nuclear localization sequences.

An aspect of the invention encompasses methods of modifying a genomic locus of interest to change gene expression in a cell by introducing into the cell any of the compositions described herein.

An aspect of the invention is that the above elements are comprised in a single composition or comprised in individual compositions. These compositions may advantageously be applied to a host to elicit a functional effect on the genomic level.

As used herein, the term “guide RNA” or “gRNA” has the leaning as used herein elsewhere and comprises any polynucleotide sequence having sufficient complementarity with a target nucleic acid sequence to hybridize with the target nucleic acid sequence and direct sequence-specific binding of a nucleic acid-targeting complex to the target nucleic acid sequence. Each gRNA may be designed to include multiple binding recognition sites (e.g., aptamers) specific to the same or different adapter protein. Each gRNA may be designed to bind to the promoter region—1000—+1 nucleic acids upstream of the transcription start site (i.e. TSS), preferably—200 nucleic acids. This positioning improves functional domains which affect gene activation (e.g., transcription activators) or gene inhibition (e.g., transcription repressors). The modified gRNA may be one or more modified gRNAs targeted to one or more target loci (e.g., at least 1 gRNA, at least 2 gRNA, at least 5 gRNA, at least 10 gRNA, at least 20 gRNA, at least 30 g RNA, at least 50 gRNA) comprised in a composition. Said multiple gRNA sequences can be tandemly arranged and are preferably separated by a direct repeat.

Thus, gRNA, the CRISPR enzyme as defined herein may each individually be comprised in a composition and administered to a host individually or collectively. Alternatively, these components may be provided in a single composition for administration to a host. Administration to a host may be performed via viral vectors known to the skilled person or described herein for delivery to a host (e.g., lentiviral vector, adenoviral vector, AAV vector). As explained herein, use of different selection markers (e.g., for lentiviral sgRNA selection) and concentration of gRNA (e.g., dependent on whether multiple gRNAs are used) may be advantageous for eliciting an improved effect. On the basis of this concept, several variations are appropriate to elicit a genomic locus event, including DNA cleavage, gene activation, or gene deactivation. Using the provided compositions, the person skilled in the art can advantageously and specifically target single or multiple loci with the same or different functional domains to elicit one or more genomic locus events. The compositions may be applied in a wide variety of methods for screening in libraries in cells and functional modeling in vivo (e.g., gene activation of lincRNA and identification of function; gain-of-function modeling; loss-of-function modeling; the use the compositions of the invention to establish cell lines and transgenic animals for optimization and screening purposes).

The current invention comprehends the use of the compositions of the current invention to establish and utilize conditional or inducible CRISPR transgenic cell/animals; see, e.g., Platt et al., Cell (2014), 159(2): 440-455, or PCT patent publications cited herein, such as WO 2014/093622 (PCT/US2013/074667). For example, cells or animals such as non-human animals, e.g., vertebrates or mammals, such as rodents, e.g., mice, rats, or other laboratory or field animals, e.g., cats, dogs, sheep, etc., may be ‘knock-in’ whereby the animal conditionally or inducibly expresses Cas9 akin to Platt et al. The target cell or animal thus comprises the CRISPR enzyme (e.g., Cas9) conditionally or inducibly (e.g., in the form of Cre dependent constructs), on expression of a vector introduced into the target cell, the vector expresses that which induces or gives rise to the condition of the CRISPR enzyme (e.g., Cas9) expression in the target cell. By applying the teaching and compositions as defined herein with the known method of creating a CRISPR complex, inducible genomic events are also an aspect of the current invention. Examples of such inducible events have been described herein elsewhere.

In some embodiments, phenotypic alteration is preferably the result of genome modification when a genetic disease is targeted, especially in methods of therapy and preferably where a repair template is provided to correct or alter the phenotype.

In some embodiments diseases that may be targeted include those concerned with disease-causing splice defects.

In some embodiments, cellular targets include Hemopoietic Stem/Progenitor Cells (CD34+); Human T cells; and Eye (retinal cells)—for example photoreceptor precursor cells.

In some embodiments Gene targets include: Human Beta Globin—HBB (for treating Sickle Cell Anemia, including by stimulating gene-conversion (using closely related HBD gene as an endogenous template)); CD3 (T-Cells); and CEP920—retina (eye).

In some embodiments disease targets also include: cancer; Sickle Cell Anemia (based on a point mutation); HBV, HIV; Beta-Thalassemia; and ophthalmic or ocular disease—for example Leber Congenital Amaurosis (LCA)—causing Splice Defect.

In some embodiments delivery methods include: Cationic Lipid Mediated “direct” delivery of Enzyme-Guide complex (RiboNucleoProtein) and electroporation of plasmid DNA.

Methods, products and uses described herein may be used for non-therapeutic purposes. Furthermore, any of the methods described herein may be applied in vitro and ex vivo.

In an aspect, provided is a non-naturally occurring or engineered composition comprising:
I. two or more CRISPR-Cas system polynucleotide sequences comprising
(a) a first guide sequence capable of hybridizing to a first target sequence in a polynucleotide locus,
(b) a second guide sequence capable of hybridizing to a second target sequence in a polynucleotide locus,
(c) a direct repeat sequence, and
II. a Cas9 enzyme or a second polynucleotide sequence encoding it,
wherein when transcribed, the first and the second guide sequences direct sequence-specific binding of a first and a second Cas9 CRISPR complex to the first and second target sequences respectively,
wherein the first CRISPR complex comprises the Cas9 enzyme complexed with the first guide sequence that is hybridizable to the first target sequence,
wherein the second CRISPR complex comprises the Cas9 enzyme complexed with the second guide sequence that is hybridizable to the second target sequence, and
   wherein the first guide sequence directs cleavage of one strand of the DNA duplex near the first target sequence and the second guide sequence directs cleavage of the other strand near the second target sequence inducing a double strand break, thereby modifying the organism or the non-human or non-animal organism. Similarly, compositions comprising more than two guide RNAs can be envisaged e.g. each specific for one target, and arranged tandemly in the composition or CRISPR system or complex as described herein.

In another embodiment, the Cas9 is delivered into the cell as a protein. In another and particularly preferred embodiment, the Cas9 is delivered into the cell as a protein or as a nucleotide sequence encoding it. Delivery to the cell as a protein may include delivery of a Ribonucleoprotein (RNP) complex, where the protein is complexed with the multiple guides.

In an aspect, host cells and cell lines modified by or comprising the compositions, systems or modified enzymes of present invention are provided, including stem cells, and progeny thereof.

In an aspect, methods of cellular therapy are provided, where, for example, a single cell or a population of cells is sampled or cultured, wherein that cell or cells is or has been modified ex vivo as described herein, and is then re-introduced (sampled cells) or introduced (cultured cells) into the organism. Stem cells, whether embryonic or induce pluripotent or totipotent stem cells, are also particularly preferred in this regard. But, of course, in vivo embodiments are also envisaged.

Inventive methods can further comprise delivery of templates, such as repair templates, which may be dsODN or ssODN, see below. Delivery of templates may be via the cotemporaneous or separate from delivery of any or all the CRISPR enzyme or guide RNAs and via the same delivery mechanism or different. In some embodiments, it is preferred that the template is delivered together with the guide RNAs and, preferably, also the CRISPR enzyme. An example may be an AAV vector where the CRISPR enzyme is AsCas9 or LbCas9.

Inventive methods can further comprise: (a) delivering to the cell a double-stranded oligodeoxynucleotide (dsODN) comprising overhangs complimentary to the overhangs created by said double strand break, wherein said dsODN is integrated into the locus of interest; or —(b) delivering to the cell a single-stranded oligodeoxynucleotide (ssODN), wherein said ssODN acts as a template for homology directed repair of said double strand break. Inventive methods can be for the prevention or treatment of disease in an individual, optionally wherein said disease is caused by a defect in said locus of interest. Inventive methods can be conducted in vivo in the individual or ex vivo on a cell taken from the individual, optionally wherein said cell is returned to the individual.

The invention also comprehends products obtained from using CRISPR enzyme or Cas enzyme or Cas9 enzyme or CRISPR-CRISPR enzyme or CRISPR-Cas system or CRISPR-Cas9 system for use in tandem or multiple targeting as defined herein.

### Escorted Guides for the Cas9 CRISPR-Cas System According to the Invention

In one aspect the invention provides escorted Cas9 CRISPR-Cas systems or complexes, especially such a system involving an escorted Cas9 CRISPR-Cas system guide. By “escorted” is meant that the Cas9 CRISPR-Cas system or complex or guide is delivered to a selected time or place within a cell, so that activity of the Cas9 CRISPR-Cas system or complex or guide is spatially or temporally controlled. For example, the activity and destination of the Cas9 CRISPR-Cas system or complex or guide may be controlled by an escort RNA aptamer sequence that has binding affinity for an aptamer ligand, such as a cell surface protein or other localized cellular component. Alternatively, the escort aptamer may for example be responsive to an aptamer effector on or in the cell, such as a transient effector, such as an external energy source that is applied to the cell at a particular time.

The escorted Cas9 CRISPR-Cas systems or complexes have a gRNA with a functional structure designed to improve gRNA structure, architecture, stability, genetic expression, or any combination thereof. Such a structure can include an aptamer.

Aptamers are biomolecules that can be designed or selected to bind tightly to other ligands, for example using a technique called systematic evolution of ligands by exponential enrichment (SELEX; Tuerk C, Gold L: “Systematic evolution of ligands by exponential enrichment: RNA ligands to bacteriophage T4 DNA polymerase.” Science 1990, 249:505-510). Nucleic acid aptamers can for example be selected from pools of random-sequence oligonucleotides, with high binding affinities and specificities for a wide range of biomedically relevant targets, suggesting a wide range of therapeutic utilities for aptamers (Keefe, Anthony D., Supriya Pai, and Andrew Ellington. “Aptamers as therapeutics.” Nature Reviews Drug Discovery 9.7 (2010): 537-550). These characteristics also suggest a wide range of uses for aptamers as drug delivery vehicles (Levy-Nissenbaum, Etgar, et al. “Nanotechnology and aptamers: applications in drug delivery.” Trends in biotechnology 26.8 (2008): 442-449; and, Hicke B J, Stephens A W. “Escort aptamers: a delivery service for diagnosis and therapy.” J Clin Invest 2000, 106:923-928.). Aptamers may also be constructed that function as molecular switches, responding to a que by changing properties, such as RNA aptamers that bind fluorophores to mimic the activity of green fluorescent protein (Paige, Jeremy S., Karen Y. Wu, and Samie R. Jaffrey. “RNA mimics of green fluorescent protein.” Science 333.6042 (2011): 642-646). It has also been suggested that aptamers may be used as components of targeted siRNA therapeutic delivery systems, for example targeting cell surface proteins (Zhou, Jiehua, and John J. Rossi. “Aptamer-targeted cell-specific RNA interference.” Silence 1.1 (2010): 4).

Accordingly, provided herein is a gRNA modified, e.g., by one or more aptamer(s) designed to improve gRNA delivery, including delivery across the cellular membrane, to intracellular compartments, or into the nucleus. Such a structure can include, either in addition to the one or more aptamer(s) or without such one or more aptamer(s), moiety(ies) so as to render the guide deliverable, inducible or responsive to a selected effector. The invention accordingly comprehends an gRNA that responds to normal or pathological physiological conditions, including without limitation pH, hypoxia, O₂ concentration, temperature, protein concentration, enzymatic concentration, lipid structure, light exposure, mechanical disruption (e.g. ultrasound waves), magnetic fields, electric fields, or electromagnetic radiation.

An aspect of the invention provides non-naturally occurring or engineered composition comprising an escorted guide RNA (egRNA) comprising:
an RNA guide sequence capable of hybridizing to a target sequence in a genomic locus of interest in a cell; and,
an escort RNA aptamer sequence, wherein the escort aptamer has binding affinity for an aptamer ligand on or in the cell, or the escort aptamer is responsive to a localized aptamer effector on or in the cell, wherein the presence of the aptamer ligand or effector on or in the cell is spatially or temporally restricted.

The escort aptamer may for example change conformation in response to an interaction with the aptamer ligand or effector in the cell.

The escort aptamer may have specific binding affinity for the aptamer ligand.

The aptamer ligand may be localized in a location or compartment of the cell, for example on or in a membrane of the cell. Binding of the escort aptamer to the aptamer ligand may accordingly direct the egRNA to a location of interest in the cell, such as the interior of the cell by way of binding to an aptamer ligand that is a cell surface ligand. In this way, a variety of spatially restricted locations within the cell may be targeted, such as the cell nucleus or mitochondria.

Once intended alterations have been introduced, such as by editing intended copies of a gene in the genome of a cell, continued CRISPR/Cas9 expression in that cell is no longer necessary. Indeed, sustained expression would be undesirable in certain casein case of off-target effects at unintended genomic sites, etc. Thus time-limited expression would be useful. Inducible expression offers one approach, but in addition Applicants have engineered a Self-Inactivating Cas9 CRISPR-Cas system that relies on the use of a non-coding guide target sequence within the CRISPR vector itself. Thus, after expression begins, the CRISPR system will lead to its own destruction, but before destruction is complete it will have time to edit the genomic copies of the target gene (which, with a normal point mutation in a diploid cell, requires at most two edits). Simply, the self-inactivating Cas9 CRISPR-Cas system includes additional RNA (i.e., guide RNA) that targets the coding sequence for the CRISPR enzyme itself or that targets one or more non-coding guide target sequences complementary to unique sequences present in one or more of the following: (a) within the promoter driving expression of the non-coding RNA elements, (b) within the promoter driving expression of the Cas9 gene, (c) within 100 bp of the ATG translational start codon in the Cas9 coding sequence, (d) within the inverted terminal repeat (iTR) of a viral delivery vector, e.g., in an AAV genome.

The egRNA may include an RNA aptamer linking sequence, operably linking the escort RNA sequence to the RNA guide sequence.

In embodiments, the egRNA may include one or more photolabile bonds or non-naturally occurring residues.

In one aspect, the escort RNA aptamer sequence may be complementary to a target miRNA, which may or may not be present within a cell, so that only when the target miRNA is present is there binding of the escort RNA aptamer sequence to the target miRNA which results in cleavage of the egRNA by an RNA-induced silencing complex (RISC) within the cell.

In embodiments, the escort RNA aptamer sequence may for example be from 10 to 200 nucleotides in length, and the egRNA may include more than one escort RNA aptamer sequence.

It is to be understood that any of the RNA guide sequences as described herein elsewhere can be used in the egRNA described herein. In certain embodiments of the invention, the guide RNA or mature crRNA comprises, consists essentially of, or consists of a direct repeat sequence and a guide sequence or spacer sequence. In certain embodiments, the guide RNA or mature crRNA comprises, consists essentially of, or consists of a direct repeat sequence linked to a guide sequence or spacer sequence. In certain embodiments the guide RNA or mature crRNA comprises 19 nts of partial direct repeat followed by 23-25 nt of guide sequence or spacer sequence. In certain embodiments, the effector protein is a FnCas9 effector protein and requires at least 16 nt of guide sequence to achieve detectable DNA cleavage and a minimum of 17 nt of guide sequence to achieve efficient DNA cleavage in vitro. In certain embodiments, the direct repeat sequence is located upstream (i.e., 5′) from the guide sequence or spacer sequence. In a preferred embodiment the seed sequence (i.e. the sequence essential critical for recognition and/or hybridization to the sequence at the target locus) of the FnCas9 guide RNA is approximately within the first 5 nt on the 5′ end of the guide sequence or spacer sequence.

The egRNA may be included in a non-naturally occurring or engineered Cas9 CRISPR-Cas complex composition, together with a Cas9 which may include at least one mutation, for example a mutation so that the Cas9 has no more than 5% of the nuclease activity of a Cas9 not having the at least one mutation, for example having a diminished nuclease activity of at least 97%, or 100% as compared with the Cas9 not having the at least one mutation. The Cas9 may also include one or more nuclear localization sequences. Mutated Cas9 enzymes having modulated activity such as diminished nuclease activity are described herein elsewhere.

The engineered Cas9 CRISPR-Cas composition may be provided in a cell, such as a eukaryotic cell, a mammalian cell, or a human cell.

In embodiments, the compositions described herein comprise a Cas9 CRISPR-Cas complex having at least three functional domains, at least one of which is associated with Cas9 and at least two of which are associated with egRNA.

The compositions described herein may be used to introduce a genomic locus event in a host cell, such as an eukaryotic cell, in particular a mammalian cell, or a non-human eukaryote, in particular a non-human mammal such as a mouse, in vivo. The genomic locus event may comprise affecting gene activation, gene inhibition, or cleavage in a locus. The compositions described herein may also be used to modify a genomic locus of interest to change gene expression in a cell. Methods of introducing a genomic locus event in a host cell using the Cas9 enzyme provided herein are described herein in detail elsewhere. Delivery of the composition may for example be by way of delivery of a nucleic acid molecule(s) coding for the composition, which nucleic acid molecule(s) is operatively linked to regulatory sequence(s), and expression of the nucleic acid molecule(s) in vivo, for example by way of a lentivirus, an adenovirus, or an AAV.

The present invention provides compositions and methods by which gRNA-mediated gene editing activity can be adapted. The invention provides gRNA secondary structures that improve cutting efficiency by increasing gRNA and/or increasing the amount of RNA delivered into the cell. The gRNA may include light labile or inducible nucleotides.

To increase the effectiveness of gRNA, for example gRNA delivered with viral or non-viral technologies, Applicants added secondary structures into the gRNA that enhance its stability and improve gene editing. Separately, to overcome the lack of effective delivery, Applicants modified gRNAs with cell penetrating RNA aptamers; the aptamers bind to cell surface receptors and promote the entry of gRNAs into cells. Notably, the cell-penetrating aptamers can be designed to target specific cell receptors, in order to mediate cell-specific delivery. Applicants also have created guides that are inducible.

Light responsiveness of an inducible system may be achieved via the activation and binding of cryptochrome-2 and CIB1. Blue light stimulation induces an activating conformational change in cryptochrome-2, resulting in recruitment of its binding partner CIB1. This binding is fast and reversible, achieving saturation in <15 sec following pulsed stimulation and returning to baseline <15 min after the end of stimulation. These rapid binding kinetics result in a system temporally bound only by the speed of transcription/translation and transcript/protein degradation, rather than uptake and clearance of inducing agents. Crytochrome-2 activation is also highly sensitive, allowing for the use of low light intensity stimulation and mitigating the risks of phototoxicity. Further, in a context such as the intact mammalian brain, variable light intensity may be used to control the size of a stimulated region, allowing for greater precision than vector delivery alone may offer.

The invention contemplates energy sources such as electromagnetic radiation, sound energy or thermal energy to induce the guide. Advantageously, the electromagnetic radiation is a component of visible light. In a preferred embodiment, the light is a blue light with a wavelength of about 450 to about 495 nm. In an especially preferred embodiment, the wavelength is about 488 nm. In another preferred embodiment, the light stimulation is via pulses. The light power may range from about 0-9 mW/cm². In a preferred embodiment, a stimulation paradigm of as low as 0.25 sec every 15 sec should result in maximal activation.

Cells involved in the practice of the present invention may be a prokaryotic cell or a eukaryotic cell, advantageously an animal cell a plant cell or a yeast cell, more advantageously a mammalian cell.

The chemical or energy sensitive guide may undergo a conformational change upon induction by the binding of a chemical source or by the energy allowing it act as a guide and have the Cas9 CRISPR-Cas system or complex function. The invention can involve applying the chemical source or energy so as to have the guide function and the Cas9 CRISPR-Cas system or complex function; and optionally further determining that the expression of the genomic locus is altered.

There are several different designs of this chemical inducible system: 1. ABI-PYL based system inducible by Abscisic Acid (ABA) (see, e.g., http://stke.sciencemag.org/cgi/content/abstract/sigtrans;4/164/rs2), 2. FKBP-FRB based system inducible by rapamycin (or related chemicals based on rapamycin) (see, e.g., http://www.nature.com/nmeth/joumal/v2/n6/full/nmeth763.html), 3. GID1-GAI based system inducible by Gibberellin (GA) (see, e.g., http://www.nature.com/nchembio/joumal/v8/n5/full/nchembio.922.html).

Another system contemplated by the present invention is a chemical inducible system based on change in sub-cellular localization. Applicants also developed a system in which the polypeptide include a DNA binding domain comprising at least five or more Transcription activator-like effector (TALE) monomers and at least one or more half-monomers specifically ordered to target the genomic locus of interest linked to at least one or more effector domains are further linker to a chemical or energy sensitive protein. This protein will lead to a change in the sub-cellular localization of the entire polypeptide (i.e. transportation of the entire polypeptide from cytoplasm into the nucleus of the cells) upon the binding of a chemical or energy transfer to the chemical or energy sensitive protein. This transportation of the entire polypeptide from one sub-cellular compartments or organelles, in which its activity is sequestered due to lack of substrate for the effector domain, into another one in which the substrate is present would allow the entire polypeptide to come in contact with its desired substrate (i.e. genomic DNA in the mammalian nucleus) and result in activation or repression of target gene expression.

This type of system could also be used to induce the cleavage of a genomic locus of interest in a cell when the effector domain is a nuclease.

A chemical inducible system can be an estrogen receptor (ER) based system inducible by 4-hydroxytamoxifen (4OHT) (see, e.g., http://www.pnas.org/content/104/3/1027.abstract). A mutated ligand-binding domain of the estrogen receptor called ERT2 translocates into the nucleus of cells upon binding of 4-hydroxytamoxifen. In further embodiments of the invention any naturally occurring or engineered derivative of any nuclear receptor, thyroid hormone receptor, retinoic acid receptor, estrogen receptor, estrogen-related receptor, glucocorticoid receptor, progesterone receptor, androgen receptor may be used in inducible systems analogous to the ER based inducible system.

Another inducible system is based on the design using Transient receptor potential (TRP) ion channel based system inducible by energy, heat or radio-wave (see, e.g., http://www.sciencemag.org/content/336/6081/604). These TRP family proteins respond to different stimuli, including light and heat. When this protein is activated by light or heat, the ion channel will open and allow the entering of ions such as calcium into the plasma membrane. This influx of ions will bind to intracellular ion interacting partners linked to a polypeptide including the guide and the other components of the Cas9 CRISPR-Cas complex or system, and the binding will induce the change of sub-cellular localization of the polypeptide, leading to the entire polypeptide entering the nucleus of cells. Once inside the nucleus, the guide protein and the other components of the Cas9 CRISPR-Cas complex will be active and modulating target gene expression in cells.

This type of system could also be used to induce the cleavage of a genomic locus of interest in a cell; and, in this regard, it is noted that the Cas9 enzyme is a nuclease. The light could be generated with a laser or other forms of energy sources. The heat could be generated by raise of temperature results from an energy source, or from nano-particles that release heat after absorbing energy from an energy source delivered in the form of radio-wave.

While light activation may be an advantageous embodiment, sometimes it may be disadvantageous especially for in vivo applications in which the light may not penetrate the skin or other organs. In this instance, other methods of energy activation are contemplated, in particular, electric field energy and/or ultrasound which have a similar effect.

Electric field energy is preferably administered substantially as described in the art, using one or more electric pulses of from about 1 Volt/cm to about 10 kVolts/cm under in vivo conditions. Instead of or in addition to the pulses, the electric field may be delivered in a continuous manner. The electric pulse may be applied for between 1 μs and 500 milliseconds, preferably between 1 μs and 100 milliseconds. The electric field may be applied continuously or in a pulsed manner for 5 about minutes.

As used herein, ‘electric field energy’ is the electrical energy to which a cell is exposed. Preferably the electric field has a strength of from about 1 Volt/cm to about 10 kVolts/cm or more under in vivo conditions (see WO97/49450).

As used herein, the term “electric field” includes one or more pulses at variable capacitance and voltage and including exponential and/or square wave and/or modulated wave and/or modulated square wave forms. References to electric fields and electricity should be taken to include reference the presence of an electric potential difference in the environment of a cell. Such an environment may be set up by way of static electricity, alternating current (AC), direct current (DC), etc, as known in the art. The electric field may be uniform, non-uniform or otherwise, and may vary in strength and/or direction in a time dependent manner.

Single or multiple applications of electric field, as well as single or multiple applications of ultrasound are also possible, in any order and in any combination. The ultrasound and/or the electric field may be delivered as single or multiple continuous applications, or as pulses (pulsatile delivery).

Electroporation has been used in both in vitro and in vivo procedures to introduce foreign material into living cells. With in vitro applications, a sample of live cells is first mixed with the agent of interest and placed between electrodes such as parallel plates. Then, the electrodes apply an electrical field to the cell/implant mixture. Examples of systems that perform in vitro electroporation include the Electro Cell Manipulator ECM600 product, and the Electro Square Porator T820, both made by the BTX Division of Genetronics, Inc (see U.S. Pat. No. 5,869,326).

The known electroporation techniques (both in vitro and in vivo) function by applying a brief high voltage pulse to electrodes positioned around the treatment region. The electric field generated between the electrodes causes the cell membranes to temporarily become porous, whereupon molecules of the agent of interest enter the cells. In known electroporation applications, this electric field comprises a single square wave pulse on the order of 1000 V/cm, of about 100.mu.s duration. Such a pulse may be generated, for example, in known applications of the Electro Square Porator T820.

Preferably, the electric field has a strength of from about 1 V/cm to about 10 kV/cm under in vitro conditions. Thus, the electric field may have a strength of 1 V/cm, 2 V/cm, 3 V/cm, 4 V/cm, 5 V/cm, 6 V/cm, 7 V/cm, 8 V/cm, 9 V/cm, 10 V/cm, 20 V/cm, 50 V/cm, 100 V/cm, 200 V/cm, 300 V/cm, 400 V/cm, 500 V/cm, 600 V/cm, 700 V/cm, 800 V/cm, 900 V/cm, 1 kV/cm, 2 kV/cm, 5 kV/cm, 10 kV/cm, 20 kV/cm, 50 kV/cm or more. More preferably from about 0.5 kV/cm to about 4.0 kV/cm under in vitro conditions. Preferably the electric field has a strength of from about 1 V/cm to about 10 kV/cm under in vivo conditions. However, the electric field strengths may be lowered where the number of pulses delivered to the target site are increased. Thus, pulsatile delivery of electric fields at lower field strengths is envisaged.

Preferably the application of the electric field is in the form of multiple pulses such as double pulses of the same strength and capacitance or sequential pulses of varying strength and/or capacitance. As used herein, the term “pulse” includes one or more electric pulses at variable capacitance and voltage and including exponential and/or square wave and/or modulated wave/square wave forms.

Preferably the electric pulse is delivered as a waveform selected from an exponential wave form, a square wave form, a modulated wave form and a modulated square wave form.

A preferred embodiment employs direct current at low voltage. Thus, Applicants disclose the use of an electric field which is applied to the cell, tissue or tissue mass at a field strength of between 1V/cm and 20V/cm, for a period of 100 milliseconds or more, preferably 15 minutes or more.

Ultrasound is advantageously administered at a power level of from about 0.05 W/cm² to about 100 W/cm². Diagnostic or therapeutic ultrasound may be used, or combinations thereof.

As used herein, the term “ultrasound” refers to a form of energy which consists of mechanical vibrations the frequencies of which are so high they are above the range of human hearing. Lower frequency limit of the ultrasonic spectrum may generally be taken as about 20 kHz. Most diagnostic applications of ultrasound employ frequencies in the range 1 and 15 MHz' (From Ultrasonics in Clinical Diagnosis, P. N. T. Wells, ed., 2nd. Edition, Publ. Churchill Livingstone [Edinburgh, London & NY, 1977]).

Ultrasound has been used in both diagnostic and therapeutic applications. When used as a diagnostic tool (“diagnostic ultrasound”), ultrasound is typically used in an energy density range of up to about 100 mW/cm² (FDA recommendation), although energy densities of up to 750 mW/cm² have been used. In physiotherapy, ultrasound is typically used as an energy source in a range up to about 3 to 4 W/cm² (WHO recommendation). In other therapeutic applications, higher intensities of ultrasound may be employed, for example, HIFU at 100 W/cm up to 1 kW/cm² (or even higher) for short periods of time. The term “ultrasound” as used in this specification is intended to encompass diagnostic, therapeutic and focused ultrasound.

Focused ultrasound (FUS) allows thermal energy to be delivered without an invasive probe (see Morocz et al 1998 Journal of Magnetic Resonance Imaging Vol. 8, No. 1, pp. 136-142. Another form of focused ultrasound is high intensity focused ultrasound (HIFU) which is reviewed by Moussatov et al in Ultrasonics (1998) Vol. 36, No. 8, pp. 893-900 and TranHuuHue et al in Acustica (1997) Vol. 83, No. 6, pp. 1103-1106.

Preferably, a combination of diagnostic ultrasound and a therapeutic ultrasound is employed. This combination is not intended to be limiting, however, and the skilled reader will appreciate that any variety of combinations of ultrasound may be used. Additionally, the energy density, frequency of ultrasound, and period of exposure may be varied.

Preferably the exposure to an ultrasound energy source is at a power density of from about 0.05 to about 100 Wcm⁻². Even more preferably, the exposure to an ultrasound energy source is at a power density of from about 1 to about 15 Wcm².

Preferably the exposure to an ultrasound energy source is at a frequency of from about 0.015 to about 10.0 MHz. More preferably the exposure to an ultrasound energy source is at a frequency of from about 0.02 to about 5.0 MHz or about 6.0 MHz. Most preferably, the ultrasound is applied at a frequency of 3 MHz.

Preferably the exposure is for periods of from about 10 milliseconds to about 60 minutes. Preferably the exposure is for periods of from about 1 second to about 5 minutes. More preferably, the ultrasound is applied for about 2 minutes. Depending on the particular target cell to be disrupted, however, the exposure may be for a longer duration, for example, for 15 minutes.

Advantageously, the target tissue is exposed to an ultrasound energy source at an acoustic power density of from about 0.05 Wcm⁻² to about 10 Wcm⁻² with a frequency ranging from about 0.015 to about 10 MHz (see WO 98/52609). However, alternatives are also possible, for example, exposure to an ultrasound energy source at an acoustic power density of above 100 Wcm⁻², but for reduced periods of time, for example, 1000 Wcm⁻² for periods in the millisecond range or less.

Preferably the application of the ultrasound is in the form of multiple pulses; thus, both continuous wave and pulsed wave (pulsatile delivery of ultrasound) may be employed in any combination. For example, continuous wave ultrasound may be applied, followed by pulsed wave ultrasound, or vice versa. This may be repeated any number of times, in any order and combination. The pulsed wave ultrasound may be applied against a background of continuous wave ultrasound, and any number of pulses may be used in any number of groups.

Preferably, the ultrasound may comprise pulsed wave ultrasound. In a highly preferred embodiment, the ultrasound is applied at a power density of 0.7 Wcm⁻² or 1.25 Wcm⁻² as a continuous wave. Higher power densities may be employed if pulsed wave ultrasound is used.

Use of ultrasound is advantageous as, like light, it may be focused accurately on a target. Moreover, ultrasound is advantageous as it may be focused more deeply into tissues unlike light. It is therefore better suited to whole-tissue penetration (such as but not limited to a lobe of the liver) or whole organ (such as but not limited to the entire liver or an entire muscle, such as the heart) therapy. Another important advantage is that ultrasound is a non-invasive stimulus which is used in a wide variety of diagnostic and therapeutic applications. By way of example, ultrasound is well known in medical imaging techniques and, additionally, in orthopedic therapy. Furthermore, instruments suitable for the application of ultrasound to a subject vertebrate are widely available and their use is well known in the art.

The rapid transcriptional response and endogenous targeting of the instant invention make for an ideal system for the study of transcriptional dynamics. For example, the instant invention may be used to study the dynamics of variant production upon induced expression of a target gene. On the other end of the transcription cycle, mRNA degradation studies are often performed in response to a strong extracellular stimulus, causing expression level changes in a plethora of genes. The instant invention may be utilized to reversibly induce transcription of an endogenous target, after which point stimulation may be stopped and the degradation kinetics of the unique target may be tracked.

The temporal precision of the instant invention may provide the power to time genetic regulation in concert with experimental interventions. For example, targets with suspected involvement in long-term potentiation (LTP) may be modulated in organotypic or dissociated neuronal cultures, but only during stimulus to induce LTP, so as to avoid interfering with the normal development of the cells. Similarly, in cellular models exhibiting disease phenotypes, targets suspected to be involved in the effectiveness of a particular therapy may be modulated only during treatment. Conversely, genetic targets may be modulated only during a pathological stimulus. Any number of experiments in which timing of genetic cues to external experimental stimuli is of relevance may potentially benefit from the utility of the instant invention.

The in vivo context offers equally rich opportunities for the instant invention to control gene expression. Photoinducibility provides the potential for spatial precision. Taking advantage of the development of optrode technology, a stimulating fiber optic lead may be placed in a precise brain region. Stimulation region size may then be tuned by light intensity. This may be done in conjunction with the delivery of the Cas9 CRISPR-Cas system or complex of the invention, or, in the case of transgenic Cas9 animals, guide RNA of the invention may be delivered and the optrode technology can allow for the modulation of gene expression in precise brain regions. A transparent Cas9 expressing organism, can have guide RNA of the invention administered to it and then there can be extremely precise laser induced local gene expression changes.

A culture medium for culturing host cells includes a medium commonly used for tissue culture, such as M199-earle base, Eagle MEM (E-MEM), Dulbecco MEM (DMEM), SC-UCM102, UP-SFM (GIBCO BRL), EX-CELL302 (Nichirei), EX-CELL293-S(Nichirei), TFBM-01 (Nichirei), ASF104, among others. Suitable culture media for specific cell types may be found at the American Type Culture Collection (ATCC) or the European Collection of Cell Cultures (ECACC). Culture media may be supplemented with amino acids such as L-glutamine, salts, anti-fungal or anti-bacterial agents such as Fungizone®, penicillin-streptomycin, animal serum, and the like. The cell culture medium may optionally be serum-free.

The invention may also offer valuable temporal precision in vivo. The invention may be used to alter gene expression during a particular stage of development. The invention may be used to time a genetic cue to a particular experimental window. For example, genes implicated in learning may be overexpressed or repressed only during the learning stimulus in a precise region of the intact rodent or primate brain. Further, the invention may be used to induce gene expression changes only during particular stages of disease development. For example, an oncogene may be overexpressed only once a tumor reaches a particular size or metastatic stage. Conversely, proteins suspected in the development of Alzheimer's may be knocked down only at defined time points in the animal's life and within a particular brain region. Although these examples do not exhaustively list the potential applications of the invention, they highlight some of the areas in which the invention may be a powerful technology.

Protected Guides: Enzymes According to the Invention can be Used in Combination with Protected Guide RNAs

In one aspect, an object of the current invention is to further enhance the specificity of Cas9 given individual guide RNAs through thermodynamic tuning of the binding specificity of the guide RNA to target DNA. This is a general approach of introducing mismatches, elongation or truncation of the guide sequence to increase/decrease the number of complimentary bases vs. mismatched bases shared between a genomic target and its potential off-target loci, in order to give thermodynamic advantage to targeted genomic loci over genomic off-targets.

In one aspect, the invention provides for the guide sequence being modified by secondary structure to increase the specificity of the Cas9 CRISPR-Cas system and whereby the secondary structure can protect against exonuclease activity and allow for 3′ additions to the guide sequence.

In one aspect, the invention provides for hybridizing a “protector RNA” to a guide sequence, wherein the “protector RNA” is an RNA strand complementary to the 5′ end of the guide RNA (gRNA), to thereby generate a partially double-stranded gRNA. In an embodiment of the invention, protecting the mismatched bases with a perfectly complementary protector sequence decreases the likelihood of target DNA binding to the mismatched base pairs at the 3′ end. In embodiments of the invention, additional sequences comprising an extended length may also be present.

Guide RNA (gRNA) extensions matching the genomic target provide gRNA protection and enhance specificity. Extension of the gRNA with matching sequence distal to the end of the spacer seed for individual genomic targets is envisaged to provide enhanced specificity. Matching gRNA extensions that enhance specificity have been observed in cells without truncation. Prediction of gRNA structure accompanying these stable length extensions has shown that stable forms arise from protective states, where the extension forms a closed loop with the gRNA seed due to complimentary sequences in the spacer extension and the spacer seed. These results demonstrate that the protected guide concept also includes sequences matching the genomic target sequence distal of the 20mer spacer-binding region. Thermodynamic prediction can be used to predict completely matching or partially matching guide extensions that result in protected gRNA states. This extends the concept of protected gRNAs to interaction between X and Z, where X will generally be of length 17-20 nt and Z is of length 1-30 nt. Thermodynamic prediction can be used to determine the optimal extension state for Z, potentially introducing small numbers of mismatches in Z to promote the formation of protected conformations between X and Z. Throughout the present application, the terms “X” and seed length (SL) are used interchangeably with the term exposed length (EpL) which denotes the number of nucleotides available for target DNA to bind; the terms “Y” and protector length (PL) are used interchangeably to represent the length of the protector; and the terms “Z”, “E”, “E′” and “EL” are used interchangeably to correspond to the term extended length (ExL) which represents the number of nucleotides by which the target sequence is extended.

An extension sequence which corresponds to the extended length (ExL) may optionally be attached directly to the guide sequence at the 3′ end of the protected guide sequence. The extension sequence may be 2 to 12 nucleotides in length. Preferably ExL may be denoted as 0, 2, 4, 6, 8, 10 or 12 nucleotides in length. In a preferred embodiment the ExL is denoted as 0 or 4 nucleotides in length. In a more preferred embodiment, the ExL is 4 nucleotides in length. The extension sequence may or may not be complementary to the target sequence.

An extension sequence may further optionally be attached directly to the guide sequence at the 5′ end of the protected guide sequence as well as to the 3′ end of a protecting sequence. As a result, the extension sequence serves as a linking sequence between the protected sequence and the protecting sequence. Without wishing to be bound by theory, such a link may position the protecting sequence near the protected sequence for improved binding of the protecting sequence to the protected sequence. It will be understood that the above-described relationship of seed, protector, and extension applies where the distal end (i.e., the targeting end) of the guide is the 5′ end, e.g. a guide that functions is a Cas9 system. In an embodiment wherein the distal end of the guide is the 3′ end, the relationship will be the reverse. In such an embodiment, the invention provides for hybridizing a “protector RNA” to a guide sequence, wherein the “protector RNA” is an RNA strand complementary to the 3′ end of the guide RNA (gRNA), to thereby generate a partially double-stranded gRNA.

Addition of gRNA mismatches to the distal end of the gRNA can demonstrate enhanced specificity. The introduction of unprotected distal mismatches in Y or extension of the gRNA with distal mismatches (Z) can demonstrate enhanced specificity. This concept as mentioned is tied to X, Y, and Z components used in protected gRNAs. The unprotected mismatch concept may be further generalized to the concepts of X, Y, and Z described for protected guide RNAs.

### Cas9.

In one aspect, the invention provides for enhanced Cas9 specificity wherein the double stranded 3′ end of the protected guide RNA (pgRNA) allows for two possible outcomes: (1) the guide RNA-protector RNA to guide RNA-target DNA strand exchange will occur and the guide will fully bind the target, or (2) the guide RNA will fail to fully bind the target and because Cas9 target cleavage is a multiple step kinetic reaction that requires guide RNA:target DNA binding to activate Cas9-catalyzed DSBs, wherein Cas9 cleavage does not occur if the guide RNA does not properly bind. According to particular embodiments, the protected guide RNA improves specificity of target binding as compared to a naturally occurring CRISPR-Cas system. According to particular embodiments, the protected modified guide RNA improves stability as compared to a naturally occurring CRISPR-Cas. According to particular embodiments, the protector sequence has a length between 3 and 120 nucleotides and comprises 3 or more contiguous nucleotides complementary to another sequence of guide or protector. According to particular embodiments, the protector sequence forms a hairpin. According to particular embodiments, the guide RNA further comprises a protected sequence and an exposed sequence. According to particular embodiments, the exposed sequence is 1 to 19 nucleotides. More particularly, the exposed sequence is at least 75%, at least 90% or about 100% complementary to the target sequence. According to particular embodiments, the guide sequence is at least 90% or about 100% complementary to the protector strand. According to particular embodiments, the guide sequence is at least 75%, at least 90% or about 100% complementary to the target sequence. According to particular embodiments, the guide RNA further comprises an extension sequence. More particularly, when the distal end of the guide is the 3′ end, the extension sequence is operably linked to the 3′ end of the protected guide sequence, and optionally directly linked to the 3′ end of the protected guide sequence. According to particular embodiments, the extension sequence is 1-12 nucleotides. According to particular embodiments, the extension sequence is operably linked to the guide sequence at the 3′ end of the protected guide sequence and the 5′ end of the protector strand and optionally directly linked to the 3′ end of the protected guide sequence and the 5′ end of the protector strand, wherein the extension sequence is a linking sequence between the protected sequence and the protector strand. According to particular embodiments, the extension sequence is 100% not complementary to the protector strand, optionally at least 95%, at least 90%, at least 80%, at least 70%, at least 60%, or at least 50% not complementary to the protector strand. According to particular embodiments, the guide sequence further comprises mismatches appended to the end of the guide sequence, wherein the mismatches thermodynamically optimize specificity.

According to the invention, in certain embodiments, guide modifications that impede strand invasion will be desireable. For example, to minimize off-target actifity, in certain embodiments, it will be desireable to design or modify a guide to impede strand invasiom at off-target sites. In certain such embodiments, it may be acceptable or useful to design or modify a guide at the expense of on-target binding efficiency. In certain embodiments, guide-target mismatches at the target site may be tolerated that substantially reduce off-target activity.

In certain embodiments of the invention, it is desirable to adjust the binding characteristics of the protected guide to minimize off-target CRISPR activity. Accordingly, thermodynamic prediction algorithms are used to predict strengths of binding on target and off target. Alternatively, or in addition, selection methods are used to reduce or minimize off-target effects, by absolute measures or relative to on-target effects.

Design options include, without limitation, i) adjusting the length of protector strand that binds to the protected strand, ii) adjusting the length of the portion of the protected strand that is exposed, iii) extending the protected strand with a stem-loop located external (distal) to the protected strand (i.e. designed so that the stem loop is external to the protected strand at the distal end), iv) extending the protected strand by addition of a protector strand to form a stem-loop with all or part of the protected strand, v) adjusting binding of the protector strand to the protected strand by designing in one or more base mismatches and/or one or more non-canonical base pairings, vi) adjusting the location of the stem formed by hybridization of the protector strand to the protected strand, and vii) addition of a non-structured protector to the end of the protected strand.

In one aspect, the invention provides an engineered, non-naturally occurring CRISPR-Cas system comprising a Cas9 protein and a protected guide RNA that targets a DNA molecule encoding a gene product in a cell, whereby the protected guide RNA targets the DNA molecule encoding the gene product and the Cas9 protein cleaves the DNA molecule encoding the gene product, whereby expression of the gene product is altered; and, wherein the Cas9 protein and the protected guide RNA do not naturally occur together. The invention comprehends the protected guide RNA comprising a guide sequence fused to a direct repeat sequence. The invention further comprehends the CRISPR protein being codon optimized for expression in a eukaryotic cell. In a preferred embodiment the eukaryotic cell is a mammalian cell, a plant cell or a yeast cell and in a more preferred embodiment the mammalian cell is a human cell. In a further embodiment of the invention, the expression of the gene product is decreased. In some embodiments the CRISPR protein is Cas9. In some embodiments the CRISPR protein is Cas12a. In some embodiments, the Cas12a protein is Acidaminococcus sp. BV3L6, Lachnospiraceae bacterium or Francisella Novicida Cas12a, and may include mutated Cas12a derived from these organisms. The protein may be a further Cas9 or Cas12a homolog or ortholog. In some embodiments, the nucleotide sequence encoding the Csa9 or Cas12a protein is codon-optimized for expression in a eukaryotic cell. In some embodiments, the Cas9 or Cas12a protein directs cleavage of one or two strands at the location of the target sequence. In some embodiments, the first regulatory element is a polymerase III promoter. In some embodiments, the second regulatory element is a polymerase II promoter. In general, and throughout this specification, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell).

Advantageous vectors include lentiviruses and adeno-associated viruses, and types of such vectors can also be selected for targeting particular types of cells.

In one aspect, the invention provides a eukaryotic host cell comprising (a) a first regulatory element operably linked to a direct repeat sequence and one or more insertion sites for inserting one or more guide sequences downstream of the direct repeat sequence, wherein when expressed, the guide sequence directs sequence-specific binding of a CRISPR complex to a target sequence in a eukaryotic cell, wherein the CRISPR complex comprises a CRISPR enzyme complexed with the guide RNA comprising the guide sequence that is hybridized to the target sequence and/or (b) a second regulatory element operably linked to an enzyme-coding sequence encoding said Cas9 enzyme comprising a nuclear localization sequence. In some embodiments, the host cell comprises components (a) and (b). In some embodiments, component (a), component (b), or components (a) and (b) are stably integrated into a genome of the host eukaryotic cell. In some embodiments, component (a) further comprises two or more guide sequences operably linked to the first regulatory element, wherein when expressed, each of the two or more guide sequences direct sequence specific binding of a CRISPR complex to a different target sequence in a eukaryotic cell. In some embodiments, the Cas9 enzyme directs cleavage of one or two strands at the location of the target sequence. In some embodiments, the Cas9 enzyme lacks DNA strand cleavage activity. In some embodiments, the first regulatory element is a polymerase III promoter. In some embodiments, the second regulatory element is a polymerase II promoter.

In an aspect, the invention provides a non-human eukaryotic organism; preferably a multicellular eukaryotic organism, comprising a eukaryotic host cell according to any of the described embodiments. In other aspects, the invention provides a eukaryotic organism; preferably a multicellular eukaryotic organism, comprising a eukaryotic host cell according to any of the described embodiments. The organism in some embodiments of these aspects may be an animal; for example a mammal. Also, the organism may be an arthropod such as an insect. The organism also may be a plant or a yeast. Further, the organism may be a fungus.

In one aspect, the invention provides a kit comprising one or more of the components described herein above. In some embodiments, the kit comprises a vector system and instructions for using the kit. In some embodiments, the vector system comprises (a) a first regulatory element operably linked to a direct repeat sequence and one or more insertion sites for inserting one or more guide sequences downstream of the direct repeat sequence, wherein when expressed, the guide sequence directs sequence-specific binding of a Cas9 CRISPR complex to a target sequence in a eukaryotic cell, wherein the CRISPR complex comprises a Cas9 enzyme complexed with the protected guide RNA comprising the guide sequence that is hybridized to the target sequence and/or (b) a second regulatory element operably linked to an enzyme-coding sequence encoding said Cas9 enzyme comprising a nuclear localization sequence. In some embodiments, the kit comprises components (a) and (b) located on the same or different vectors of the system. In some embodiments, component (a) further comprises two or more guide sequences operably linked to the first regulatory element, wherein when expressed, each of the two or more guide sequences direct sequence specific binding of a CRISPR complex to a different target sequence in a eukaryotic cell. In some embodiments, the Cas9 enzyme comprises one or more nuclear localization sequences of sufficient strength to drive accumulation of said Cas9 enzyme in a detectable amount in the nucleus of a eukaryotic cell. In some embodiments, the Cas9 enzyme is Acidaminococcus sp. BV3L6, Lachnospiraceae bacterium MA2020 or Francisella tularensis 1 Novicida Cas9, and may include mutated Cas9 derived from these organisms. The enzyme may be a Cas9 homolog or ortholog. In some embodiments, the CRISPR enzyme is codon-optimized for expression in a eukaryotic cell. In some embodiments, the CRISPR enzyme directs cleavage of one or two strands at the location of the target sequence. In some embodiments, the CRISPR enzyme lacks DNA strand cleavage activity. In some embodiments, the first regulatory element is a polymerase III promoter. In some embodiments, the second regulatory element is a polymerase II promoter.

In one aspect, the invention provides a method of modifying a target polynucleotide in a eukaryotic cell. In some embodiments, the method comprises allowing a CRISPR complex to bind to the target polynucleotide to effect cleavage of said target polynucleotide thereby modifying the target polynucleotide, wherein the CRISPR complex comprises a Cas9 enzyme complexed with protected guide RNA comprising a guide sequence hybridized to a target sequence within said target polynucleotide. In some embodiments, said cleavage comprises cleaving one or two strands at the location of the target sequence by said Cas9 enzyme. In some embodiments, said cleavage results in decreased transcription of a target gene. In some embodiments, the method further comprises repairing said cleaved target polynucleotide by non-homologous end joining (NHEJ)-based gene insertion mechanisms, more particularly with an exogenous template polynucleotide, wherein said repair results in a mutation comprising an insertion, deletion, or substitution of one or more nucleotides of said target polynucleotide. In some embodiments, said mutation results in one or more amino acid changes in a protein expressed from a gene comprising the target sequence. In some embodiments, the method further comprises delivering one or more vectors to said eukaryotic cell, wherein the one or more vectors drive expression of one or more of: the Cas9 enzyme, the protected guide RNA comprising the guide sequence linked to direct repeat sequence. In some embodiments, said vectors are delivered to the eukaryotic cell in a subject. In some embodiments, said modifying takes place in said eukaryotic cell in a cell culture. In some embodiments, the method further comprises isolating said eukaryotic cell from a subject prior to said modifying. In some embodiments, the method further comprises returning said eukaryotic cell and/or cells derived therefrom to said subject.

In one aspect, the invention provides a method of modifying expression of a polynucleotide in a eukaryotic cell. In some embodiments, the method comprises allowing a Cas9 CRISPR complex to bind to the polynucleotide such that said binding results in increased or decreased expression of said polynucleotide; wherein the CRISPR complex comprises a Cas9 enzyme complexed with a protected guide RNA comprising a guide sequence hybridized to a target sequence within said polynucleotide. In some embodiments, the method further comprises delivering one or more vectors to said eukaryotic cells, wherein the one or more vectors drive expression of one or more of: the Cas9 enzyme and the protected guide RNA.

In one aspect, the invention provides a method of generating a model eukaryotic cell comprising a mutated disease gene. In some embodiments, a disease gene is any gene associated an increase in the risk of having or developing a disease. In some embodiments, the method comprises (a) introducing one or more vectors into a eukaryotic cell, wherein the one or more vectors drive expression of one or more of: a Cas9 enzyme and a protected guide RNA comprising a guide sequence linked to a direct repeat sequence; and (b) allowing a CRISPR complex to bind to a target polynucleotide to effect cleavage of the target polynucleotide within said disease gene, wherein the CRISPR complex comprises the Cas9 enzyme complexed with the guide RNA comprising the sequence that is hybridized to the target sequence within the target polynucleotide, thereby generating a model eukaryotic cell comprising a mutated disease gene. In some embodiments, said cleavage comprises cleaving one or two strands at the location of the target sequence by said Cas9 enzyme. In some embodiments, said cleavage results in decreased transcription of a target gene. In some embodiments, the method further comprises repairing said cleaved target polynucleotide by non-homologous end joining (NHEJ)-based gene insertion mechanisms with an exogenous template polynucleotide, wherein said repair results in a mutation comprising an insertion, deletion, or substitution of one or more nucleotides of said target polynucleotide. In some embodiments, said mutation results in one or more amino acid changes in a protein expression from a gene comprising the target sequence.

In one aspect, the invention provides a method for developing a biologically active agent that modulates a cell signaling event associated with a disease gene. In some embodiments, a disease gene is any gene associated an increase in the risk of having or developing a disease. In some embodiments, the method comprises (a) contacting a test compound with a model cell of any one of the described embodiments; and (b) detecting a change in a readout that is indicative of a reduction or an augmentation of a cell signaling event associated with said mutation in said disease gene, thereby developing said biologically active agent that modulates said cell signaling event associated with said disease gene.

In one aspect, the invention provides a recombinant polynucleotide comprising a protected guide sequence downstream of a direct repeat sequence, wherein the protected guide sequence when expressed directs sequence-specific binding of a CRISPR complex to a corresponding target sequence present in a eukaryotic cell. In some embodiments, the target sequence is a viral sequence present in a eukaryotic cell. In some embodiments, the target sequence is a proto-oncogene or an oncogene.

In one aspect the invention provides for a method of selecting one or more cell(s) by introducing one or more mutations in a gene in the one or more cell (s), the method comprising: introducing one or more vectors into the cell (s), wherein the one or more vectors drive expression of one or more of: a Cas9 enzyme, a protected guide RNA comprising a guide sequence, and an editing template; wherein the editing template comprises the one or more mutations that abolish Cas9 enzyme cleavage; allowing non-homologous end joining (NHEJ)-based gene insertion mechanisms of the editing template with the target polynucleotide in the cell(s) to be selected; allowing a CRISPR complex to bind to a target polynucleotide to effect cleavage of the target polynucleotide within said gene, wherein the CRISPR complex comprises the Cas9 enzyme complexed with the protected guide RNA comprising a guide sequence that is hybridized to the target sequence within the target polynucleotide, wherein binding of the CRISPR complex to the target polynucleotide induces cell death, thereby allowing one or more cell(s) in which one or more mutations have been introduced to be selected. In a preferred embodiment of the invention the cell to be selected may be a eukaryotic cell. Aspects of the invention allow for selection of specific cells without requiring a selection marker or a two-step process that may include a counter-selection system.

With respect to mutations of the Cas9 enzyme, when the enzyme is not FnCas9, mutations may be as described herein elsewhere; conservative substitution for any of the replacement amino acids is also envisaged. In an aspect the invention provides as to any or each or all embodiments herein-discussed wherein the CRISPR enzyme comprises at least one or more, or at least two or more mutations, wherein the at least one or more mutation or the at least two or more mutations are selected from those described herein elsewhere.

In a further aspect, the invention involves a computer-assisted method for identifying or designing potential compounds to fit within or bind to CRISPR-Cas9 system or a functional portion thereof or vice versa (a computer-assisted method for identifying or designing potential CRISPR-Cas9 systems or a functional portion thereof for binding to desired compounds) or a computer-assisted method for identifying or designing potential CRISPR-Cas9 systems (e.g., with regard to predicting areas of the CRISPR-Cas9 system to be able to be manipulated—for instance, based on crystal structure data or based on data of Cas9 orthologs, or with respect to where a functional group such as an activator or repressor can be attached to the CRISPR-Cas9 system, or as to Cas9 truncations or as to designing nickases), said method comprising:
using a computer system, e.g., a programmed computer comprising a processor, a data storage system, an input device, and an output device, the steps of:
(a) inputting into the programmed computer through said input device data comprising the three-dimensional co-ordinates of a subset of the atoms from or pertaining to the CRISPR-Cas9 crystal structure, e.g., in the CRISPR-Cas9 system binding domain or alternatively or additionally in domains that vary based on variance among Cas9 orthologs or as to Cas9s or as to nickases or as to functional groups, optionally with structural information from CRISPR-Cas9 system complex(es), thereby generating a data set;
(b) comparing, using said processor, said data set to a computer database of structures stored in said computer data storage system, e.g., structures of compounds that bind or putatively bind or that are desired to bind to a CRISPR-Cas9 system or as to Cas9 orthologs (e.g., as Cas9s or as to domains or regions that vary amongst Cas9 orthologs) or as to the CRISPR-Cas9 crystal structure or as to nickases or as to functional groups;
(c) selecting from said database, using computer methods, structure(s)—e.g., CRISPR-Cas9 structures that may bind to desired structures, desired structures that may bind to certain CRISPR-Cas9 structures, portions of the CRISPR-Cas9 system that may be manipulated, e.g., based on data from other portions of the CRISPR-Cas9 crystal structure and/or from Cas9 orthologs, truncated Cas9s, novel nickases or particular functional groups, or positions for attaching functional groups or functional-group-CRISPR-Cas9 systems;
   (d) constructing, using computer methods, a model of the selected structure(s); and
   (e) outputting to said output device the selected structure(s);
   and optionally synthesizing one or more of the selected structure(s);
   and further optionally testing said synthesized selected structure(s) as or in a CRISPR-Cas9 system;
   or, said method comprising: providing the co-ordinates of at least two atoms of the CRISPR-Cas9 crystal structure, e.g., at least two atoms of the herein Crystal Structure Table of the CRISPR-Cas9 crystal structure or co-ordinates of at least a sub-domain of the CRISPR-Cas9 crystal structure (“selected co-ordinates”), providing the structure of a candidate comprising a binding molecule or of portions of the CRISPR-Cas9 system that may be manipulated, e.g., based on data from other portions of the CRISPR-Cas9 crystal structure and/or from Cas9 orthologs, or the structure of functional groups, and fitting the structure of the candidate to the selected co-ordinates, to thereby obtain product data comprising CRISPR-Cas9 structures that may bind to desired structures, desired structures that may bind to certain CRISPR-Cas9 structures, portions of the CRISPR-Cas9 system that may be manipulated, truncated Cas9s, novel nickases, or particular functional groups, or positions for attaching functional groups or functional-group-CRISPR-Cas9 systems, with output thereof; and optionally synthesizing compound(s) from said product data and further optionally comprising testing said synthesized compound(s) as or in a CRISPR-Cas9 system.

The testing can comprise analyzing the CRISPR-Cas9 system resulting from said synthesized selected structure(s), e.g., with respect to binding, or performing a desired function.

The output in the foregoing methods can comprise data transmission, e.g., transmission of information via telecommunication, telephone, video conference, mass communication, e.g., presentation such as a computer presentation (e.g. POWERPOINT), internet, email, documentary communication such as a computer program (e.g. WORD) document and the like. Accordingly, the invention also comprehends computer readable media containing: atomic co-ordinate data according to the herein-referenced Crystal Structure, said data defining the three dimensional structure of CRISPR-Cas9 or at least one sub-domain thereof, or structure factor data for CRISPR-Cas9, said structure factor data being derivable from the atomic co-ordinate data of herein-referenced Crystal Structure. The computer readable media can also contain any data of the foregoing methods. The invention further comprehends methods a computer system for generating or performing rational design as in the foregoing methods containing either: atomic co-ordinate data according to herein-referenced Crystal Structure, said data defining the three dimensional structure of CRISPR-Cas9 or at least one sub-domain thereof, or structure factor data for CRISPR-Cas9, said structure factor data being derivable from the atomic co-ordinate data of herein-referenced Crystal Structure. The invention further comprehends a method of doing business comprising providing to a user the computer system or the media or the three dimensional structure of CRISPR-Cas9 or at least one sub-domain thereof, or structure factor data for CRISPR-Cas9, said structure set forth in and said structure factor data being derivable from the atomic co-ordinate data of herein-referenced Crystal Structure, or the herein computer media or a herein data transmission.

A “binding site” or an “active site” comprises or consists essentially of or consists of a site (such as an atom, a functional group of an amino acid residue or a plurality of such atoms and/or groups) in a binding cavity or region, which may bind to a compound such as a nucleic acid molecule, which is/are involved in binding.

By “fitting”, is meant determining by automatic, or semi-automatic means, interactions between one or more atoms of a candidate molecule and at least one atom of a structure of the invention, and calculating the extent to which such interactions are stable. Interactions include attraction and repulsion, brought about by charge, steric considerations and the like. Various computer-based methods for fitting are described further

By “root mean square (or rms) deviation”, we mean the square root of the arithmetic mean of the squares of the deviations from the mean.

By a “computer system”, is meant the hardware means, software means and data storage means used to analyze atomic coordinate data. The minimum hardware means of the computer-based systems of the present invention typically comprises a central processing unit (CPU), input means, output means and data storage means. Desirably a display or monitor is provided to visualize structure data. The data storage means may be RAM or means for accessing computer readable media of the invention. Examples of such systems are computer and tablet devices running Unix, Windows or Apple operating systems.

By “computer readable media”, is meant any medium or media, which can be read and accessed directly or indirectly by a computer e.g., so that the media is suitable for use in the above-mentioned computer system. Such media include, but are not limited to: magnetic storage media such as floppy discs, hard disc storage medium and magnetic tape; optical storage media such as optical discs or CD-ROM; electrical storage media such as RAM and ROM; thumb drive devices; cloud storage devices and hybrids of these categories such as magnetic/optical storage media.

The invention comprehends the use of the protected guides described herein above in the optimized functional CRISPR-Cas enzyme systems described herein.

In certain embodiments, the CRISPR system as provided herein can make use of a crRNA or analogous polynucleotide comprising a guide sequence, wherein the polynucleotide is an RNA, a DNA or a mixture of RNA and DNA, and/or wherein the polynucleotide comprises one or more nucleotide analogs. The sequence can comprise any structure, including but not limited to a structure of a native crRNA, such as a bulge, a hairpin or a stem loop structure. In certain embodiments, the polynucleotide comprising the guide sequence forms a duplex with a second polynucleotide sequence which can be an RNA or a DNA sequence.

In certain embodiments, use is made of chemically modified guide RNAs. Examples of guide RNA chemical modifications include, without limitation, incorporation of 2′-O-methyl (M), 2′-O-methyl 3′phosphorothioate (MS), or 2′-O-methyl 3′thioPACE (MSP) at one or more terminal nucleotides. Such chemically modified guide RNAs can comprise increased stability and increased activity as compared to unmodified guide RNAs, though on-target vs. off-target specificity is not predictable. (See, Hendel, 2015, Nat Biotechnol. 33(9):985-9, doi: 10.1038/nbt.3290, published online 29-6-2015). Chemically modified guide RNAs further include, without limitation, RNAs with phosphorothioate linkages and locked nucleic acid (LNA) nucleotides comprising a methylene bridge between the 2′ and 4′ carbons of the ribose ring.

In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. Preferably the guide sequence is 10 to 30 nucleotides long. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay. Similarly, cleavage of a target RNA may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

In some embodiments, the modification to the guide is a chemical modification, an insertion, a deletion or a split. In some embodiments, the chemical modification includes, but is not limited to, incorporation of 2′-O-methyl (M) analogs, 2′-deoxy analogs, 2-thiouridine analogs, N6-methyladenosine analogs, 2′-fluoro analogs, 2-aminopurine, 5-bromo-uridine, pseudouridine (Ψ), N¹-methylpseudouridine (me¹Ψ), 5-methoxyuridine(5moU), inosine, 7-methylguanosine, 2′-O-methyl-3′-phosphorothioate (MS), S-constrained ethyl(cEt), phosphorothioate (PS), or 2′-O-methyl-3′-thioPACE (MSP). In some embodiments, the guide comprises one or more of phosphorothioate modifications. In certain embodiments, at least 1, 2, 3, 4, 5, 6, 7, 8, 9, 10, 11, 12, 13, 14, 15, 16, 17, 18, 19, 20, or 25 nucleotides of the guide are chemically modified. In certain embodiments, one or more nucleotides in the seed region are chemically modified. In certain embodiments, one or more nucleotides in the 3′-terminus are chemically modified. In certain embodiments, none of the nucleotides in the 5′-handle is chemically modified. In some embodiments, the chemical modification in the seed region is a minor modification, such as incorporation of a 2′-fluoro analog. In a specific embodiment, one nucleotide of the seed region is replaced with a 2′-fluoro analog. In some embodiments, 5 or 10 nucleotides in the 3′-terminus are chemically modified. Such chemical modifications at the 3′-terminus of the Cpf1 CrRNA improve gene cutting efficiency (see Li, et al., Nature Biomedical Engineering, 2017, 1:0066). In a specific embodiment, 5 nucleotides in the 3′-terminus are replaced with 2′-fluoro analogues. In a specific embodiment, 10 nucleotides in the 3′-terminus are replaced with 2′-fluoro analogues. In a specific embodiment, 5 nucleotides in the 3′-terminus are replaced with 2′—O-methyl (M) analogs.

In some embodiments, the loop of the 5′-handle of the guide is modified. In some embodiments, the loop of the 5′-handle of the guide is modified to have a deletion, an insertion, a split, or chemical modifications. In certain embodiments, the loop comprises 3, 4, or 5 nucleotides. In certain embodiments, the loop comprises the sequence of UCUU, UUUU, UAUU, or UGUU.

A guide sequence, and hence a nucleic acid-targeting guide RNA may be selected to target any target nucleic acid sequence. In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to a RNA polynucleotide being or comprising the target sequence. In other words, the target RNA may be a RNA polynucleotide or a part of a RNA polynucleotide to which a part of the gRNA, i.e. the guide sequence, is designed to have complementarity and to which the effector function mediated by the complex comprising CRISPR effector protein and a gRNA is to be directed. In some embodiments, a target sequence is located in the nucleus or cytoplasm of a cell. The target sequence may be DNA. The target sequence may be any RNA sequence. In some embodiments, the target sequence may be a sequence within a RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nuclear RNA (snoRNA), double stranded RNA (dsRNA), non-coding RNA (ncRNA), long non-coding RNA (lncRNA), and small cytoplasmic RNA (scRNA). In some preferred embodiments, the target sequence may be a sequence within a RNA molecule selected from the group consisting of mRNA, pre-mRNA, and rRNA. In some preferred embodiments, the target sequence may be a sequence within a RNA molecule selected from the group consisting of ncRNA, and lncRNA. In some more preferred embodiments, the target sequence may be a sequence within an mRNA molecule or a pre-mRNA molecule.

In certain embodiments, the spacer length of the guide RNA is less than 28 nucleotides. In certain embodiments, the spacer length of the guide RNA is at least 18 nucleotides and less than 28 nucleotides. In certain embodiments, the spacer length of the guide RNA is between 19 and 28 nucleotides. In certain embodiments, the spacer length of the guide RNA is between 19 and 25 nucleotides. In certain embodiments, the spacer length of the guide RNA is 20 nucleotides. In certain embodiments, the spacer length of the guide RNA is 23 nucleotides. In certain embodiments, the spacer length of the guide RNA is 25 nucleotides.

In certain embodiments, modulations of cleavage efficiency can be exploited by introduction of mismatches, e.g. 1 or more mismatches, such as 1 or 2 mismatches between spacer sequence and target sequence, including the position of the mismatch along the spacer/target. The more central (i.e. not 3′ or 5′) for instance a double mismatch is, the more cleavage efficiency is affected. Accordingly, by choosing mismatch position along the spacer, cleavage efficiency can be modulated. By means of example, if less than 100% cleavage of targets is desired (e.g. in a cell population), 1 or more, such as preferably 2 mismatches between spacer and target sequence may be introduced in the spacer sequences. The more central along the spacer of the mismatch position, the lower the cleavage percentage.

In certain example embodiments, the cleavage efficiency may be exploited to design single guides that can distinguish two or more targets that vary by a single nucleotide, such as a single nucleotide polymorphism (SNP), variation, or (point) mutation. The CRISPR effector may have reduced sensitivity to SNPs (or other single nucleotide variations) and continue to cleave SNP targets with a certain level of efficiency. Thus, for two targets, or a set of targets, a guide RNA may be designed with a nucleotide sequence that is complementary to one of the targets i.e. the on-target SNP. The guide RNA is further designed to have a synthetic mismatch. As used herein a “synthetic mismatch” refers to a non-naturally occurring mismatch that is introduced upstream or downstream of the naturally occurring SNP, such as at most 5 nucleotides upstream or downstream, for instance 4, 3, 2, or 1 nucleotide upstream or downstream, preferably at most 3 nucleotides upstream or downstream, more preferably at most 2 nucleotides upstream or downstream, most preferably 1 nucleotide upstream or downstream (i.e. adjacent the SNP). When the CRISPR effector binds to the on-target SNP, only a single mismatch will be formed with the synthetic mismatch and the CRISPR effector will continue to be activated and a detectable signal produced. When the guide RNA hybridizes to an off-target SNP, two mismatches will be formed, the mismatch from the SNP and the synthetic mismatch, and no detectable signal generated. Thus, the systems disclosed herein may be designed to distinguish SNPs within a population. For, example the systems may be used to distinguish pathogenic strains that differ by a single SNP or detect certain disease specific SNPs, such as but not limited to, disease associated SNPs, such as without limitation cancer associated SNPs.

In certain embodiments, the guide RNA is designed such that the SNP is located on position 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the spacer sequence (starting at the 5′ end). In certain embodiments, the guide RNA is designed such that the SNP is located on position 1, 2, 3, 4, 5, 6, 7, 8, or 9 of the spacer sequence (starting at the 5′ end). In certain embodiments, the guide RNA is designed such that the SNP is located on position 2, 3, 4, 5, 6, or 7 of the spacer sequence (starting at the 5′ end). In certain embodiments, the guide RNA is designed such that the SNP is located on position 3, 4, 5, or 6 of the spacer sequence (starting at the 5′ end). In certain embodiments, the guide RNA is designed such that the SNP is located on position 3 of the spacer sequence (starting at the 5′ end).

In certain embodiments, the guide RNA is designed such that the mismatch (e.g.the synthetic mismatch, i.e. an additional mutation besides a SNP) is located on position 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the spacer sequence (starting at the 5′ end). In certain embodiments, the guide RNA is designed such that the mismatch is located on position 1, 2, 3, 4, 5, 6, 7, 8, or 9 of the spacer sequence (starting at the 5′ end). In certain embodiments, the guide RNA is designed such that the mismatch is located on position 4, 5, 6, or 7 of the spacer sequence (starting at the 5′ end. In certain embodiments, the guide RNA is designed such that the mismatch is located on position 5 of the spacer sequence (starting at the 5′ end).

In certain embodiments, the guide RNA is designed such that the mismatch is located 2 nucleotides upstream of the SNP (i.e. one intervening nucleotide).

In certain embodiments, the guide RNA is designed such that the mismatch is located 2 nucleotides downstream of the SNP (i.e. one intervening nucleotide).

In certain embodiments, the guide RNA is designed such that the mismatch is located on position 5 of the spacer sequence (starting at the 5′ end) and the SNP is located on position 3 of the spacer sequence (starting at the 5′ end).

The embodiments described herein comprehend inducing one or more nucleotide modifications in a eukaryotic cell (in vitro, i.e. in an isolated eukaryotic cell) as herein discussed comprising delivering to cell a vector as herein discussed. The mutation(s) can include the introduction, deletion, or substitution of one or more nucleotides at each target sequence of cell(s) via the guide(s) RNA(s). The mutations can include the introduction, deletion, or substitution of 1-75 nucleotides at each target sequence of said cell(s) via the guide(s) RNA(s). The mutations can include the introduction, deletion, or substitution of 1, 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s). The mutations can include the introduction, deletion, or substitution of 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s). The mutations include the introduction, deletion, or substitution of 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s). The mutations can include the introduction, deletion, or substitution of 20, 21, 22, 23, 24, 25, 26, 27, 28, 29, 30, 35, 40, 45, 50, or 75 nucleotides at each target sequence of said cell(s) via the guide(s) RNA(s). The mutations can include the introduction, deletion, or substitution of 40, 45, 50, 75, 100, 200, 300, 400 or 500 nucleotides at each target sequence of said cell(s) via the guide(s) RNA(s).

Typically, in the context of an endogenous CRISPR system, formation of a CRISPR complex (comprising a guide sequence hybridized to a target sequence and complexed with one or more Cas proteins) results in cleavage in or near (e.g. within 1, 2, 3, 4, 5, 6, 7, 8, 9, 10, 20, 50, or more base pairs from) the target sequence, but may depend on for instance secondary structure, in particular in the case of RNA targets.

In one aspect, the embodiments disclosed herein are directed to a nucleic acid detection system comprising two or more CRISPR systems one or more guide RNAs designed to bind to corresponding target molecules, a masking construct, and optional amplification reagents to amplify target nucleic acid molecules in a sample. In certain example embodiments, the system may further comprise one or more detection aptamers. The one or more detection aptamers may comprise a RNA polymerase site or primer binding site. The one or more detection aptamers specifically bind one or more target polypeptides and are configured such that the RNA polymerase site or primer binding site is exposed only upon binding of the detection aptamer to a target peptide. Exposure of the RNA polymerase site facilitates generation of a trigger RNA oligonucleotide using the aptamer sequence as a template. Accordingly, in such embodiments the one or more guide RNAs are configured to bind to a trigger RNA.

In another aspect, the embodiments disclosed herein are directed to a diagnostic device comprising a plurality of individual discrete volumes. Each individual discrete volume comprises a CRISPR effector protein, one or more guide RNAs designed to bind to a corresponding target molecule, and a masking construct. In certain example embodiments, RNA amplification reagents may be pre-loaded into the individual discrete volumes or be added to the individual discrete volumes concurrently with or subsequent to addition of a sample to each individual discrete volume. The device may be a microfluidic based device, a wearable device, or device comprising a flexible material substrate on which the individual discrete volumes are defined.

In another aspect, the embodiments disclosed herein are directed to a method for detecting target nucleic acids in a sample comprising distributing a sample or set of samples into a set of individual discrete volumes, each individual discrete volume comprising a CRISPR effector protein, one or more guide RNAs designed to bind to one target oligonucleotides, and a masking construct. The set of samples are then maintained under conditions sufficient to allow binding of the one or more guide RNAs to one or more target molecules. Binding of the one or more guide RNAs to a target nucleic acid in turn activates the CRISPR effector protein. Once activated, the CRISPR effector protein then deactivates the masking construct, for example, by cleaving the masking construct such that a detectable positive signal is unmasked, released, or generated. Detection of the positive detectable signal in an individual discrete volume indicates the presence of the target molecules.

In yet another aspect, the embodiments disclosed herein are directed to a method for detecting polypeptides. The method for detecting polypeptides is similar to the method for detecting target nucleic acids described above. However, a peptide detection aptamer is also included. The peptide detection aptamers function as described above and facilitate generation of a trigger oligonucleotide upon binding to a target polypeptide. The guide RNAs are designed to recognize the trigger oligonucleotides thereby activating the CRISPR effector protein. Deactivation of the masking construct by the activated CRISPR effector protein leads to unmasking, release, or generation of a detectable positive signal.

### Masking Construct

As used herein, a “masking construct” refers to a molecule that can be cleaved or otherwise deactivated by an activated CRISPR system effector protein described herein. The term “masking construct” may also be referred to in the alternative as a “detection construct.” In certain example embodiments, the masking construct is a RNA-based masking construct. The RNA-based masking construct comprises a RNA element that is cleavable by a CRISPR effector protein. Cleavage of the RNA element releases agents or produces conformational changes that allow a detectable signal to be produced. Example constructs demonstrating how the RNA element may be used to prevent or mask generation of detectable signal are described below and embodiments of the invention comprise variants of the same. Prior to cleavage, or when the masking construct is in an ‘active’ state, the masking construct blocks the generation or detection of a positive detectable signal. It will be understood that in certain example embodiments a minimal background signal may be produced in the presence of an active RNA masking construct. A positive detectable signal may be any signal that can be detected using optical, fluorescent, chemiluminescent, electrochemical or other detection methods known in the art. The term “positive detectable signal” is used to differentiate from other detectable signals that may be detectable in the presence of the masking construct. For example, in certain embodiments a first signal may be detected when the masking agent is present (i.e. a negative detectable signal), which then converts to a second signal (e.g.the positive detectable signal) upon detection of the target molecules and cleavage or deactivation of the masking agent by the activated CRISPR effector protein.

In certain example embodiments, the masking construct may suppress generation of a gene product. The gene product may be encoded by a reporter construct that is added to the sample. The masking construct may be an interfering RNA involved in a RNA interference pathway, such as a short hairpin RNA (shRNA) or small interfering RNA (siRNA). The masking construct may also comprise microRNA (miRNA). While present, the masking construct suppresses expression of the gene product. The gene product may be a fluorescent protein or other RNA transcript or proteins that would otherwise be detectable by a labeled probe, aptamer, or antibody but for the presence of the masking construct. Upon activation of the effector protein the masking construct is cleaved or otherwise silenced allowing for expression and detection of the gene product as the positive detectable signal.

In certain example embodiments, the masking construct may sequester one or more reagents needed to generate a detectable positive signal such that release of the one or more reagents from the masking construct results in generation of the detectable positive signal. The one or more reagents may combine to produce a colorimetric signal, a chemiluminescent signal, a fluorescent signal, or any other detectable signal and may comprise any reagents known to be suitable for such purposes. In certain example embodiments, the one or more reagents are sequestered by RNA aptamers that bind the one or more reagents. The one or more reagents are released when the effector protein is activated upon detection of a target molecule and the RNA aptamers are degraded.

In certain example embodiments, the masking construct may be immobilized on a solid substrate in an individual discrete volume (defined further below) and sequesters a single reagent. For example, the reagent may be a bead comprising a dye. When sequestered by the immobilized reagent, the individual beads are too diffuse to generate a detectable signal, but upon release from the masking construct are able to generate a detectable signal, for example by aggregation or simple increase in solution concentration. In certain example embodiments, the immobilized masking agent is a RNA-based aptamer that can be cleaved by the activated effector protein upon detection of a target molecule.

In certain other example embodiments, the masking construct binds to an immobilized reagent in solution thereby blocking the ability of the reagent to bind to a separate labeled binding partner that is free in solution. Thus, upon application of a washing step to a sample, the labeled binding partner can be washed out of the sample in the absence of a target molecule. However, if the effector protein is activated, the masking construct is cleaved to a degree sufficient to interfere with the ability of the masking construct to bind the reagent thereby allowing the labeled binding partner to bind to the immobilized reagent. Thus, the labeled binding partner remains after the wash step indicating the presence of the target molecule in the sample. In certain aspects, the masking construct that binds the immobilized reagent is an RNA aptamer. The immobilized reagent may be a protein and the labeled minding partner may be a labeled antibody. Alternatively, the immobilized reagent may be streptavidin and the labeled binding partner may be labeled biotin. The label on the binding partner used in the above embodiments may be any detectable label known in the art. In addition, other known binding partners may be used in accordance with the overall design described herein.

In certain example embodiments, the masking construct may comprise a ribozyme. Ribozymes are RNA molecules having catalytic properties. Ribozymes, both naturally and engineered, comprise or consist of RNA that may be targeted by the effector proteins disclosed herein. The ribozyme may be selected or engineered to catalyze a reaction that either generates a negative detectable signal or prevents generation of a positive control signal. Upon deactivation of the ribozyme by the activated effector protein the reaction generating a negative control signal, or preventing generation of a positive detectable signal, is removed thereby allowing a positive detectable signal to be generated. In one example embodiment, the ribozyme may catalyze a colorimetric reaction causing a solution to appear as a first color. When the ribozyme is deactivated the solution then turns to a second color, the second color being the detectable positive signal. An example of how ribozymes can be used to catalyze a colorimetric reaction are described in Zhao et al. “Signal amplification of glucosamine-6-phosphate based on ribozyme glmS,” Biosens Bioelectron. 2014; 16:337-42, and provide an example of how such a system could be modified to work in the context of the embodiments disclosed herein. Alternatively, ribozymes, when present can generate cleavage products of, for example, RNA transcripts. Thus, detection of a positive detectable signal may comprise detection of non-cleaved RNA transcripts that are only generated in the absence of the ribozyme.

In certain example embodiments, the one or more reagents is a protein, such as an enzyme, capable of facilitating generation of a detectable signal, such as a colorimetric, chemiluminescent, or fluorescent signal, that is inhibited or sequestered such that the protein cannot generate the detectable signal by the binding of one or more RNA aptamers to the protein. Upon activation of the effector proteins disclosed herein, the RNA aptamers are cleaved or degraded to an extent that they no longer inhibit the protein's ability to generate the detectable signal. In certain example embodiments, the aptamer is a thrombin inhibitor aptamer. In certain example embodiments the thrombin inhibitor aptamer has a sequence of GGGAACAAAGCUGAAGUACUUACCC (SEQ ID NO: 16). When this aptamer is cleaved, thrombin will become active and will cleave a peptide colorimetric or fluorescent substrate. In certain example embodiments, the colorimetric substrate is para-nitroanilide (pNA) covalently linked to the peptide substrate for thrombin. Upon cleavage by thrombin, pNA is released and becomes yellow in color and easily visible to the eye. In certain example embodiments, the fluorescent substrate is 7-amino-4-methylcoumarin a blue fluorophore that can be detected using a fluorescence detector. Inhibitory aptamers may also be used for horseradish peroxidase (HRP), beta-galactosidase, or calf alkaline phosphatase (CAP) and within the general principals laid out above.

In certain embodiments, RNAse activity is detected colorimetrically via cleavage of enzyme-inhibiting aptamers. One potential mode of converting RNAse activity into a colorimetric signal is to couple the cleavage of an RNA aptamer with the re-activation of an enzyme that is capable of producing a colorimetric output. In the absence of RNA cleavage, the intact aptamer will bind to the enzyme target and inhibit its activity. The advantage of this readout system is that the enzyme provides an additional amplification step: once liberated from an aptamer via collateral activity (e.g. Cas13a collateral activity), the colorimetric enzyme will continue to produce colorimetric product, leading to a multiplication of signal.

Unlike the DNA endonucleases Cas9 and Cpf1, which cleave only its DNA target, RNA-guided RNases, like Cas13a and Cpf1, remain active after cleaving its RNA or DNA target, leading to “collateral” cleavage of non-targeted RNAs in proximity (Abudayyeh et al., 2016), which may also be termed collateral activity. This crRNA-programmed collateral RNA cleavage activity presents the opportunity to use RNA-guided RNases to detect the presence of a specific RNA by triggering in vivo programmed cell death or in vitro non-specific RNA degradation that can serve as a readout (Abudayyeh et al., 2016; East-Seletsky et al., 2016).

In certain embodiments, an existing aptamer that inhibits an enzyme with a colorimetric readout is used. Several aptamer/enzyme pairs with colorimetric readouts exist, such as thrombin, protein C, neutrophil elastase, and subtilisin. These proteases have colorimetric substrates based upon pNA and are commercially available. In certain embodiments, a novel aptamer targeting a common colorimetric enzyme is used. Common and robust enzymes, such as beta-galactosidase, horseradish peroxidase, or calf intestinal alkaline phosphatase, could be targeted by engineered aptamers designed by selection strategies such as SELEX. Such strategies allow for quick selection of aptamers with nanomolar binding efficiencies and could be used for the development of additional enzyme/aptamer pairs for colorimetric readout.

In certain embodiments, RNAse activity is detected colorimetrically via cleavage of RNA-tethered inhibitors. Many common colorimetric enzymes have competitive, reversible inhibitors: for example, beta-galactosidase can be inhibited by galactose. Many of these inhibitors are weak, but their effect can be increased by increases in local concentration. By linking local concentration of inhibitors to RNAse activity, colorimetric enzyme and inhibitor pairs can be engineered into RNAse sensors. The colorimetric RNAse sensor based upon small-molecule inhibitors involves three components: the colorimetric enzyme, the inhibitor, and a bridging RNA that is covalently linked to both the inhibitor and enzyme, tethering the inhibitor to the enzyme. In the uncleaved configuration, the enzyme is inhibited by the increased local concentration of the small molecule; when the RNA is cleaved (e.g. by Cas13a collateral cleavage), the inhibitor will be released and the colorimetric enzyme will be activated.

In certain embodiments, RNAse activity is detected colorimetrically via formation and/or activation of G-quadruplexes. G quadraplexes in DNA can complex with heme (iron (III)-protoporphyrin IX) to form a DNAzyme with peroxidase activity. When supplied with a peroxidase substrate (e.g. ABTS: (2,2′-Azinobis [3-ethylbenzothiazoline-6-sulfonic acid]-diammonium salt)), the G-quadraplex-heme complex in the presence of hydrogen peroxide causes oxidation of the substrate, which then forms a green color in solution. An example G-quadraplex forming DNA sequence is: GGGTAGGGCGGGTTGGGA (SEQ ID NO: 17). By hybridizing an RNA sequence to this DNA aptamer, formation of the G-quadraplex structure will be limited. Upon RNAse collateral activation (e.g. C2c2-complex collateral activation), the RNA staple will be cleaved allowing the G quadraplex to form and heme to bind. This strategy is particularly appealing because color formation is enzymatic, meaning there is additional amplification beyond RNAse activation.

In certain example embodiments, the masking construct may be immobilized on a solid substrate in an individual discrete volume (defined further below) and sequesters a single reagent. For example, the reagent may be a bead comprising a dye. When sequestered by the immobilized reagent, the individual beads are too diffuse to generate a detectable signal, but upon release from the masking construct are able to generate a detectable signal, for example by aggregation or simple increase in solution concentration. In certain example embodiments, the immobilized masking agent is a RNA-based aptamer that can be cleaved by the activated effector protein upon detection of a target molecule.

In one example embodiment, the masking construct comprises a detection agent that changes color depending on whether the detection agent is aggregated or dispersed in solution. For example, certain nanoparticles, such as colloidal gold, undergo a visible purple to red color shift as they move from aggregates to dispersed particles. Accordingly, in certain example embodiments, such detection agents may be held in aggregate by one or more bridge molecules. At least a portion of the bridge molecule comprises RNA. Upon activation of the effector proteins disclosed herein, the RNA portion of the bridge molecule is cleaved allowing the detection agent to disperse and resulting in the corresponding change in color. See e.g. FIG. 46. In certain example embodiments, the bridge molecule is a RNA molecule. In certain example embodiments, the detection agent is a colloidal metal. The colloidal metal material may include water-insoluble metal particles or metallic compounds dispersed in a liquid, a hydrosol, or a metal sol. The colloidal metal may be selected from the metals in groups IA, IB, IIB and IIIB of the periodic table, as well as the transition metals, especially those of group VIII. Preferred metals include gold, silver, aluminum, ruthenium, zinc, iron, nickel and calcium. Other suitable metals also include the following in all of their various oxidation states: lithium, sodium, magnesium, potassium, scandium, titanium, vanadium, chromium, manganese, cobalt, copper, gallium, strontium, niobium, molybdenum, palladium, indium, tin, tungsten, rhenium, platinum, and gadolinium. The metals are preferably provided in ionic form, derived from an appropriate metal compound, for example the A13+, Ru3+, Zn2+, Fe3+, Ni2+ and Ca2+ ions.

When the RNA bridge is cut by the activated CRISPR effector, the beforementioned color shift is observed. In certain example embodiments the particles are colloidal metals. In certain other example embodiments, the colloidal metal is a colloidal gold. In certain example embodiments, the colloidal nanoparticles are 15 nm gold nanoparticles (AuNPs). Due to the unique surface properties of colloidal gold nanoparticles, maximal absorbance is observed at 520 nm when fully dispersed in solution and appear red in color to the naked eye. Upon aggregation of AuNPs, they exhibit a red-shift in maximal absorbance and appear darker in color, eventually precipitating from solution as a dark purple aggregate. In certain example embodiments the nanoparticles are modified to include DNA linkers extending from the surface of the nanoparticle. Individual particles are linked together by single-stranded RNA (ssRNA) bridges that hybridize on each end of the RNA to at least a portion of the DNA linkers. Thus, the nanoparticles will form a web of linked particles and aggregate, appearing as a dark precipitate. Upon activation of the CRISPR effectors disclosed herein, the ssRNA bridge will be cleaved, releasing the AU NPS from the linked mesh and producing a visible red color. Example DNA linkers and RNA bridge sequences are listed below. Thiol linkers on the end of the DNA linkers may be used for surface conjugation to the AuNPS. Other forms of conjugation may be used. In certain example embodiments, two populations of AuNPs may be generated, one for each DNA linker. This will help facilitate proper binding of the ssRNA bridge with proper orientation. In certain example embodiments, a first DNA linker is conjugated by the 3′ end while a second DNA linker is conjugated by the 5′ end.

| | |
|---|---|
| C2c2 | TTATAACTATTCCTAAAAAAAAAAA/ |
| colorimetric | 3ThioMC3-D/ |
| DNA1 | (SEQ ID NO: 18) |
| C2c2 | /5ThioMC6-D/ |
| colorimetric | AAAAAAAAAACTCCCCTAATAACAAT |
| DNA2 | (SEQ ID NO: 19) |
| C2c2 | GGGUAGGAAUAGUUAUAAUUUCCCUU |
| colorimetric | UCCCAUUGUUAUUAGGGAG (SEQ ID |
| bridge | NO: 20) |

In certain other example embodiments, the masking construct may comprise an RNA oligonucleotide to which are attached a detectable label and a masking agent of that detectable label. Two or more oligonucleotide constructs can be provided that allow for detection of more than one signal. An example of a detectable label/masking agent pair is a fluorophore and a quencher of the fluorophore. Quenching of the fluorophore can occur as a result of the formation of a non-fluorescent complex between the fluorophore and another fluorophore or non-fluorescent molecule. This mechanism is known as ground-state complex formation, static quenching, or contact quenching. Accordingly, the RNA oligonucleotide may be designed so that the fluorophore and quencher are in sufficient proximity for contact quenching to occur. Fluorophores and their cognate quenchers are known in the art and can be selected for this purpose by one having ordinary skill in the art. The particular fluorophore/quencher pair is not critical in the context of this invention, only that selection of the fluorophore/quencher pairs ensures masking of the fluorophore. Upon activation of the effector proteins disclosed herein, the RNA oligonucleotide is cleaved thereby severing the proximity between the fluorophore and quencher needed to maintain the contact quenching effect. Accordingly, detection of the fluorophore may be used to determine the presence of a target molecule in a sample.

In certain other example embodiments, the masking construct may comprise one or more RNA oligonucleotides to which are attached one or more metal nanoparticles, such as gold nanoparticles. In some embodiments, the masking construct comprises a plurality of metal nanoparticles crosslinked by a plurality of RNA oligonucleotides forming a closed loop. In one embodiment, the masking construct comprises three gold nanoparticles crosslinked by three RNA oligonucleotides forming a closed loop. In some embodiments, the cleavage of the RNA oligonucleotides by the CRISPR effector protein leads to a detectable signal produced by the metal nanoparticles.

In certain other example embodiments, the masking construct may comprise one or more RNA oligonucleotides to which are attached one or more quantum dots. In some embodiments, the cleavage of the RNA oligonucleotides by the CRISPR effector protein leads to a detectable signal produced by the quantum dots.

In one example embodiment, the masking construct may comprise a quantum dot. The quantum dot may have multiple linker molecules attached to the surface. At least a portion of the linker molecule comprises RNA. The linker molecule is attached to the quantum dot at one end and to one or more quenchers along the length or at terminal ends of the linker such that the quenchers are maintained in sufficient proximity for quenching of the quantum dot to occur. The linker may be branched. As above, the quantum dot/quencher pair is not critical, only that selection of the quantum dot/quencher pair ensures masking of the fluorophore. Quantum dots and their cognate quenchers are known in the art and can be selected for this purpose by one having ordinary skill in the art Upon activation of the effector proteins disclosed herein, the RNA portion of the linker molecule is cleaved thereby eliminating the proximity between the quantum dot and one or more quenchers needed to maintain the quenching effect. In certain example embodiments the quantum dot is streptavidin conjugated. RNA are attached via biotin linkers and recruit quenching molecules with the sequences /5Biosg/UCUCGUACGUUC/3IAbRQSp/(SEQ ID NO: 21) or /5Biosg/UCUCGUACGUUCUCUCGUACGUUC/3IAbRQSp/(SEQ ID NO: 22), where /5Biosg/ is a biotin tag and/31AbRQSp/ is an Iowa black quencher. Upon cleavage, by the activated effectors disclosed herein the quantum dot will fluoresce visibly.

In a similar fashion, fluorescence energy transfer (FRET) may be used to generate a detectable positive signal. FRET is a non-radiative process by which a photon from an energetically excited fluorophore (i.e. “donor fluorophore”) raises the energy state of an electron in another molecule (i.e. “the acceptor”) to higher vibrational levels of the excited singlet state. The donor fluorophore returns to the ground state without emitting a fluoresce characteristic of that fluorophore. The acceptor can be another fluorophore or non-fluorescent molecule. If the acceptor is a fluorophore, the transferred energy is emitted as fluorescence characteristic of that fluorophore. If the acceptor is a non-fluorescent molecule the absorbed energy is loss as heat. Thus, in the context of the embodiments disclosed herein, the fluorophore/quencher pair is replaced with a donor fluorophore/acceptor pair attached to the oligonucleotide molecule. When intact, the masking construct generates a first signal (negative detectable signal) as detected by the fluorescence or heat emitted from the acceptor. Upon activation of the effector proteins disclosed herein the RNA oligonucleotide is cleaved and FRET is disrupted such that fluorescence of the donor fluorophore is now detected (positive detectable signal).

In certain example embodiments, the masking construct comprises the use of intercalating dyes which change their absorbance in response to cleavage of long RNAs to short nucleotides. Several such dyes exist. For example, pyronine-Y will complex with RNA and form a complex that has an absorbance at 572 nm. Cleavage of the RNA results in loss of absorbance and a color change. Methylene blue may be used in a similar fashion, with changes in absorbance at 688 nm upon RNA cleavage. Accordingly, in certain example embodiments the masking construct comprises a RNA and intercalating dye complex that changes absorbance upon the cleavage of RNA by the effector proteins disclosed herein.

In certain example embodiments, the masking construct may comprise an initiator for an HCR reaction. See e.g. Dirks and Pierce. PNAS 101, 15275-15728 (2004). HCR reactions utilize the potential energy in two hairpin species. When a single-stranded initiator having a portion of complementary to a corresponding region on one of the hairpins is released into the previously stable mixture, it opens a hairpin of one species. This process, in turn, exposes a single-stranded region that opens a hairpin of the other species. This process, in turn, exposes a single stranded region identical to the original initiator. The resulting chain reaction may lead to the formation of a nicked double helix that grows until the hairpin supply is exhausted. Detection of the resulting products may be done on a gel or colorimetrically. Example colorimetric detection methods include, for example, those disclosed in Lu et al. “Ultra-sensitive colorimetric assay system based on the hybridization chain reaction-triggered enzyme cascade amplification ACS Appl Mater Interfaces, 2017, 9(1):167-175, Wang et al. “An enzyme-free colorimetric assay using hybridization chain reaction amplification and split aptamers” Analyst 2015, 150, 7657-7662, and Song et al. “Non covalent fluorescent labeling of hairpin DNA probe coupled with hybridization chain reaction for sensitive DNA detection.” Applied Spectroscopy, 70(4): 686-694 (2016).

In certain example embodiments, the masking construct may comprise a HCR initiator sequence and a cleavable structural element, such as a loop or hairpin, that prevents the initiator from initiating the HCR reaction. Upon cleavage of the structure element by an activated CRISPR effector protein, the initiator is then released to trigger the HCR reaction, detection thereof indicating the presence of one or more targets in the sample. In certain example embodiments, the masking construct comprises a hairpin with a RNA loop. When an activated CRISPR effector protein cuts the RNA loop, the initiator can be released to trigger the HCR reaction.

Utilizing two or more oligonucleotide probes allows for the ability to simultaneously detect multiple sample inputs, also allowing for multiplexed detection panels or for in sample controls. Orthogonal base preferences of the Cas13 enzymes as described herein offer the opportunity to have multiplexed detection systems. Applicant can assay the collateral activity of different Cas13 enzymes in the same reaction via fluorescent homopolymer sensors of different base identities and fluorophore colors, enabling multiple targets to be simultaneously measured.

### Tiled Probes

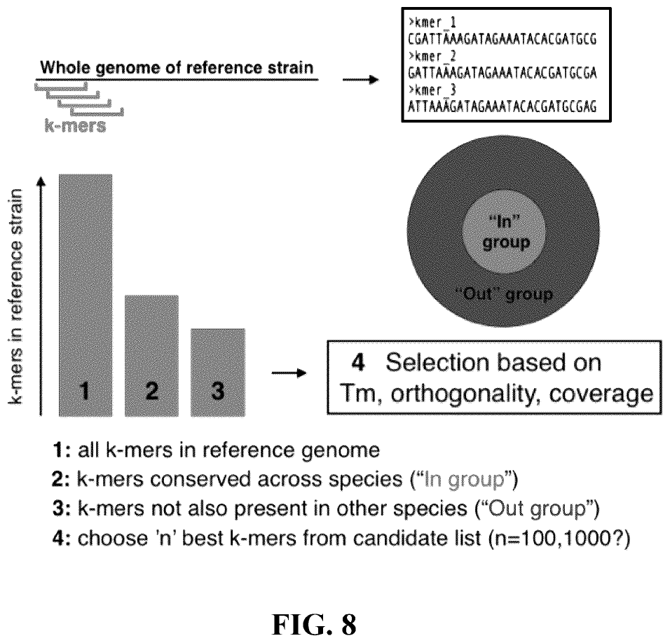

In embodiments, tiled probes are provided. In embodiments, the guide RNAs are tiled. Tiled guide RNAs are tile probes/guide RNAs that span across all or some portion of a genome of interest. In some embodiments, the tiled guide RNAs used in the CRISPR detection systems and methods for cell free nucleic acid detection of pathogens of interest utilize a genomic target sequence unique to the pathogens of interest, genus of interest, or subset species of interest. In some embodiments, bioinformatics tools are used to identify an exhaustive list of genomic targets that are unique to a given strain or species of microbe is conducted. In some preferred embodiments, one or more of the target conservation across the pathogens of interest. An exemplary methodology developed for target identification is described below, and illustrated in FIG. 8.

Sets of tiled probes can be provided. A set of probes includes two or more probes, with a tiled probe within a set designed to hybridize to a different portion of a same target sequence. In instances in which the guide RNA is tiled, the guide molecule is also designed to form a complex with the one or more CRISPR-Cas proteins.

Depending on the particular application, “pathogens of interest” may encompass all strains within a species, or include just a single strain. To accommodate varying levels of resolution, “in” and “out” groups are defined. The “in” group encompasses all genomes of interest. The “out” group comprises of all genomes that are not desired to detect as signal (theoretically all other genomes). Once the “in” and “out” groups are defined, a reference genome within the “in” group. Is chosen. This reference genome is used to generate a list of all possible genomic targets of a pre-defined size. Next, a sequence alignment tool, for example, Bowtie57, is used to identify matching sequences with all other genomes in the “in” and “out” groups. A candidate list of possible genomic targets comprises of those sequences that match with all genomes in the “in” group, and do not match with any of the genomes in the “out” group. Because there is some evidence that suggests that species-specific targets are likely to cover a large fraction of the genome, pooled CRISPR RNA (crRNA) guides for these targets will be computationally selected and can be empirically tested for efficiency using microbial genomic DNA (gDNA) samples that mimic the size profile of cfDNA fragments.

In particular embodiments, the guide RNAs can be selected by one or more of sequence orthogonality, melting temperature and/or genomic distribution. In preferred embodiments, the guide RNAs are 28 nucleotides in length and contain one or no mismatch with the target nucleic acid, e.g. contain a mismatch tolerance of one nucleotide.

Tiled assay performance can be optimized and benchmarked for detecting infection from blood samples. Clinical samples contain a variety of inhibitors that may impede the performance of the assay. In some instances, assays can be optimized utilizing blood samples collected from patients with active infections using protocols to preserve cfDNA, Collection of clinical samples in specialized tubes that stabilize cell membranes in whole blood to minimize cell lysis can aid in stabilizing the sample prior to testing but may introduce agents that hinder molecular analysis of cfDNA. The sensitivity and specificity of the multiplexed assay can be evaluated against alternative NATs, such as real-time PCR and digital droplet PCR.

### Optical Barcodes, Barcodes, and Unique Molecular Identifier (UMI)

Systems as disclosed herein may comprise optical barcodes for one or more target molecules and an optical barcodes associated with the detection CRISPR system. For example, barcodes for one or more target molecules and a sample of interest comprising the target molecule can be merged with CRISPR detection system-containing droplets containing optical barcodes.

The term “barcode” as used herein refers to a short sequence of nucleotides (for example, DNA or RNA) that is used as an identifier for an associated molecule, such as a target molecule and/or target nucleic acid, or as an identifier of the source of an associated molecule, such as a cell-of-origin. A barcode may also refer to any unique, non-naturally occurring, nucleic acid sequence that may be used to identify the originating source of a nucleic acid fragment. Although it is not necessary to understand the mechanism of an invention, it is believed that the barcode sequence provides a high-quality individual read of a barcode associated with a single cell, a viral vector, labeling ligand (e.g., an aptamer), protein, shRNA, sgRNA or cDNA such that multiple species can be sequenced together.

Barcoding may be performed based on any of the compositions or methods disclosed in patent publication WO 2014047561 A1, Compositions and methods for labeling of agents, incorporated herein in its entirety. In certain embodiments barcoding uses an error correcting scheme (T. K. Moon, Error Correction Coding: Mathematical Methods and Algorithms (Wiley, New York, ed. 1, 2005)). Not being bound by a theory, amplified sequences from single cells can be sequenced together and resolved based on the barcode associated with each cell.

Optically encoded particles may be delivered to the discrete volumes randomly resulting in a random combination of optically encoded particles in each well, or a unique combination of optically encoded particles may be specifically assigned to each discrete volume. The observable combination of optically encoded particles may then be used to identify each discrete volume. Optical assessments, such as phenotype, may be made and recorded for each discrete volume. In some instances, the barcode may be an optically detectable barcode that can be visualized with light or fluorescence microscopy. In certain example embodiments, the optical barcode comprises a sub-set of fluorophores or quantum dots of distinguishable colors from a set of defined colors. In some instances, optically encoded particles may be delivered to the discrete volumes randomly resulting in a random combination of optically encoded particles in each well, or a unique combination of optically encoded particles may be specifically assigned to each discrete volume.

In an exemplary embodiment, 3 fluorescent dyes, e.g. Alexa Fluor 555, 594, 647, at different levels, 105 barcodes can be generated. The addition of a fourth dye can be used and can be extended to scale to hundreds of unique barcodes; similarly, five colors can increase the number of unique barcodes that may be achieved by varying the ratios of the colors. By labeling with distinct ratios of dyes, dye ratios can be chosen so that after normalization the dyes are evenly spaced in logarithmic coordinates.

In one embodiment, the assigned or random subset(s) of fluorophores received in each droplet or discrete volume dictates the observable pattern of discrete optically encoded particles in each discrete volume thereby allowing each discrete volume to be independently identified. Each discrete volume is imaged with the appropriate imaging technique to detect the optically encoded particles. For example, if the optically encoded particles are fluorescently labeled each discrete volume is imaged using a fluorescent microscope. In another example, if the optically encoded particles are colorimetrically labeled each discrete volume is imaged using a microscope having one or more filters that match the wave length or absorption spectrum or emission spectrum inherent to each color label. Other detection methods are contemplated that match the optical system used, e.g., those known in the art for detecting quantum dots, dyes, etc. The pattern of observed discrete optically encoded particles for each discrete volume may be recorded for later use.

Optical barcodes can optionally include a unique oligonucleotide sequence, method for generating can be as described in, for example, International Patent Application Publication No. WO/2014/047561 at [050]-[0115]. In one example embodiment, a primer particle identifier is incorporated in the target molecules. Next generation sequencing (NGS) techniques known in the art can be used for sequencing, with clustering by sequence similarity of the one or more target sequences. Alignment by sequence variation will allow for identification of optically encoded particles delivered to a discrete volume based on the particle identifiers incorporated in the aligned sequence information. In one embodiment, the particle identifier of each primer incorporated in the aligned sequence information indicates the pattern of optically encoded particles that is observable in the corresponding discrete volume from which the amplicons are generated. In this way the nucleic acid sequence variation can be correlated back to the originating discrete volume and further matched to the optical assessments, such as phenotype, made of the nucleic acid containing specimens in that discrete volume.

In preferred embodiments, sequencing is performed using unique molecular identifiers (UMI). The term “unique molecular identifiers” (UMI) as used herein refers to a sequencing linker or a subtype of nucleic acid barcode used in a method that uses molecular tags to detect and quantify unique amplified products. A UMI is used to distinguish effects through a single clone from multiple clones. The term “clone” as used herein may refer to a single mRNA or target nucleic acid to be sequenced. The UMI may also be used to determine the number of transcripts that gave rise to an amplified product, or in the case of target barcodes as described herein, the number of binding events. In preferred embodiments, the amplification is by PCR or multiple displacement amplification (MDA).

In certain embodiments, an UMI with a random sequence of between 4 and 20 base pairs is added to a template, which is amplified and sequenced. In preferred embodiments, the UMI is added to the 5′ end of the template. Sequencing allows for high resolution reads, enabling accurate detection of true variants. As used herein, a “true variant” will be present in every amplified product originating from the original clone as identified by aligning all products with a UMI. Each clone amplified will have a different random UMI that will indicate that the amplified product originated from that clone. Background caused by the fidelity of the amplification process can be eliminated because true variants will be present in all amplified products and background representing random error will only be present in single amplification products (See e.g., Islam S. et al., 2014. Nature Methods No:11, 163-166). Not being bound by a theory, the UMI's are designed such that assignment to the original can take place despite up to 4-7 errors during amplification or sequencing. Not being bound by a theory, an UMI may be used to discriminate between true barcode sequences.

Unique molecular identifiers can be used, for example, to normalize samples for variable amplification efficiency. For example, in various embodiments, featuring a solid or semisolid support (for example a hydrogel bead), to which nucleic acid barcodes (for example a plurality of barcodes sharing the same sequence) are attached, each of the barcodes may be further coupled to a unique molecular identifier, such that every barcode on the particular solid or semisolid support receives a distinct unique molecule identifier. A unique molecular identifier can then be, for example, transferred to a target molecule with the associated barcode, such that the target molecule receives not only a nucleic acid barcode, but also an identifier unique among the identifiers originating from that solid or semisolid support.

A nucleic acid barcode or UMI can have a length of at least, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 nucleotides, and can be in single- or double-stranded form. Target molecule and/or target nucleic acids can be labeled with multiple nucleic acid barcodes in combinatorial fashion, such as a nucleic acid barcode concatemer. Typically, a nucleic acid barcode is used to identify a target molecule and/or target nucleic acid as being from a particular discrete volume, having a particular physical property (for example, affinity, length, sequence, etc.), or having been subject to certain treatment conditions. Target molecule and/or target nucleic acid can be associated with multiple nucleic acid barcodes to provide information about all of these features (and more). Each member of a given population of UMIs, on the other hand, is typically associated with (for example, covalently bound to or a component of the same molecule as) individual members of a particular set of identical, specific (for example, discreet volume-, physical property-, or treatment condition-specific) nucleic acid barcodes. Thus, for example, each member of a set of origin-specific nucleic acid barcodes, or other nucleic acid identifier or connector oligonucleotide, having identical or matched barcode sequences, may be associated with (for example, covalently bound to or a component of the same molecule as) a distinct or different UMI.

As disclosed herein, unique nucleic acid identifiers are used to label the target molecules and/or target nucleic acids, for example origin-specific barcodes and the like. The nucleic acid identifiers, nucleic acid barcodes, can include a short sequence of nucleotides that can be used as an identifier for an associated molecule, location, or condition. In certain embodiments, the nucleic acid identifier further includes one or more unique molecular identifiers and/or barcode receiving adapters. A nucleic acid identifier can have a length of about, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 base pairs (bp) or nucleotides (nt). In certain embodiments, a nucleic acid identifier can be constructed in combinatorial fashion by combining randomly selected indices (for example, about 1, 2, 3, 4, 5, 6, 7, 8, 9, or 10 indexes). Each such index is a short sequence of nucleotides (for example, DNA, RNA, or a combination thereof) having a distinct sequence. An index can have a length of about, for example, 4, 5, 6, 7, 8, 9, 10, 11, 12, 13, 14, 15, 16, 17, 18, 19, 20, 21, 22, 23, 24, or 25 bp or nt. Nucleic acid identifiers can be generated, for example, by split-pool synthesis methods, such as those described, for example, in International Patent Publication Nos. WO 2014/047556 and WO 2014/143158, each of which is incorporated by reference herein in its entirety.

One or more nucleic acid identifiers (for example a nucleic acid barcode) can be attached, or “tagged,” to atarget molecule. This attachment can be direct (for example, covalent or noncovalent binding of the nucleic acid identifier to the target molecule) or indirect (for example, via an additional molecule). Such indirect attachments may, for example, include a barcode bound to a specific-binding agent that recognizes a target molecule. In certain embodiments, a barcode is attached to protein G and the target molecule is an antibody or antibody fragment. Attachment of a barcode to target molecules (for example, proteins and other biomolecules) can be performed using standard methods well known in the art. For example, barcodes can be linked via cysteine residues (for example, C-terminal cysteine residues). In other examples, barcodes can be chemically introduced into polypeptides (for example, antibodies) via a variety of functional groups on the polypeptide using appropriate group-specific reagents (see for example www.drmr.com/abcon). In certain embodiments, barcode tagging can occur via a barcode receiving adapter associate with (for example, attached to) a target molecule, as described herein.

Target molecules can be optionally labeled with multiple barcodes in combinatorial fashion (for example, using multiple barcodes bound to one or more specific binding agents that specifically recognizing the target molecule), thus greatly expanding the number of unique identifiers possible within a particular barcode pool. In certain embodiments, barcodes are added to a growing barcode concatemer attached to a target molecule, for example, one at a time. In other embodiments, multiple barcodes are assembled prior to attachment to a target molecule. Compositions and methods for concatemerization of multiple barcodes are described, for example, in International Patent Publication No. WO 2014/047561, which is incorporated herein by reference in its entirety.

In some embodiments, a nucleic acid identifier (for example, a nucleic acid barcode) may be attached to sequences that allow for amplification and sequencing (for example, SBS3 and P5 elements for Illumina sequencing). In certain embodiments, a nucleic acid barcode can further include a hybridization site for a primer (for example, a single-stranded DNA primer) attached to the end of the barcode. For example, an origin-specific barcode may be a nucleic acid including a barcode and a hybridization site for a specific primer. In particular embodiments, a set of origin-specific barcodes includes a unique primer specific barcode made, for example, using a randomized oligo type NNNNNNNNNNNN (SEQ ID NO:23).

A nucleic acid identifier can further include a unique molecular identifier and/or additional barcodes specific to, for example, a common support to which one or more of the nucleic acid identifiers are attached. Thus, a pool of target molecules can be added, for example, to a discrete volume containing multiple solid or semisolid supports (for example, beads) representing distinct treatment conditions (and/or, for example, one or more additional solid or semisolid support can be added to the discreet volume sequentially after introduction of the target molecule pool), such that the precise combination of conditions to which a given target molecule was exposed can be subsequently determined by sequencing the unique molecular identifiers associated with it.

Labeled target molecules and/or target nucleic acids associated origin-specific nucleic acid barcodes (optionally in combination with other nucleic acid barcodes as described herein) can be amplified by methods known in the art, such as polymerase chain reaction (PCR). For example, the nucleic acid barcode can contain universal primer recognition sequences that can be bound by a PCR primer for PCR amplification and subsequent high-throughput sequencing. In certain embodiments, the nucleic acid barcode includes or is linked to sequencing adapters (for example, universal primer recognition sequences) such that the barcode and sequencing adapter elements are both coupled to the target molecule. In particular examples, the sequence of the origin specific barcode is amplified, for example using PCR. In some embodiments, an origin-specific barcode further comprises a sequencing adaptor. In some embodiments, an origin-specific barcode further comprises universal priming sites. A nucleic acid barcode (or a concatemer thereof), a target nucleic acid molecule (for example, a DNA or RNA molecule), a nucleic acid encoding a target peptide or polypeptide, and/or a nucleic acid encoding a specific binding agent may be optionally sequenced by any method known in the art, for example, methods of high-throughput sequencing, also known as next generation sequencing or deep sequencing. A nucleic acid target molecule labeled with a barcode (for example, an origin-specific barcode) can be sequenced with the barcode to produce a single read and/or contig containing the sequence, or portions thereof, of both the target molecule and the barcode. Exemplary next generation sequencing technologies include, for example, Illumina sequencing, Ion Torrent sequencing, 454 sequencing, SOLiD sequencing, and nanopore sequencing amongst others. In some embodiments, the sequence of labeled target molecules is determined by non-sequencing based methods. For example, variable length probes or primers can be used to distinguish barcodes (for example, origin-specific barcodes) labeling distinct target molecules by, for example, the length of the barcodes, the length of target nucleic acids, or the length of nucleic acids encoding target polypeptides. In other instances, barcodes can include sequences identifying, for example, the type of molecule for a particular target molecule (for example, polypeptide, nucleic acid, small molecule, or lipid). For example, in a pool of labeled target molecules containing multiple types of target molecules, polypeptide target molecules can receive one identifying sequence, while target nucleic acid molecules can receive a different identifying sequence. Such identifying sequences can be used to selectively amplify barcodes labeling particular types of target molecules, for example, by using PCR primers specific to identifying sequences specific to particular types of target molecules. For example, barcodes labeling polypeptide target molecules can be selectively amplified from a pool, thereby retrieving only the barcodes from the polypeptide subset of the target molecule pool.

A nucleic acid barcode can be sequenced, for example, after cleavage, to determine the presence, quantity, or other feature of the target molecule. In certain embodiments, a nucleic acid barcode can be further attached to a further nucleic acid barcode. For example, a nucleic acid barcode can be cleaved from a specific-binding agent after the specific-binding agent binds to a target molecule or a tag (for example, an encoded polypeptide identifier element cleaved from a target molecule), and then the nucleic acid barcode can be ligated to an origin-specific barcode. The resultant nucleic acid barcode concatemer can be pooled with other such concatemers and sequenced. The sequencing reads can be used to identify which target molecules were originally present in which discrete volumes.

### Barcodes Reversibly Coupled to Solid Substrate

In some embodiments, the origin-specific barcodes are reversibly coupled to a solid or semisolid substrate. In some embodiments, the origin-specific barcodes further comprise a nucleic acid capture sequence that specifically binds to the target nucleic acids and/or a specific binding agent that specifically binds to the target molecules. In specific embodiments, the origin-specific barcodes include two or more populations of origin-specific barcodes, wherein a first population comprises the nucleic acid capture sequence and a second population comprises the specific binding agent that specifically binds to the target molecules. In some examples, the first population of origin-specific barcodes further comprises a target nucleic acid barcode, wherein the target nucleic acid barcode identifies the population as one that labels nucleic acids. In some examples, the second population of origin-specific barcodes further comprises a target molecule barcode, wherein the target molecule barcode identifies the population as one that labels target molecules.

Barcode with Cleavage Sites

A nucleic acid barcode may be cleavable from a specific binding agent, for example, after the specific binding agent has bound to a target molecule. In some embodiments, the origin-specific barcode further comprises one or more cleavage sites. In some examples, at least one cleavage site is oriented such that cleavage at that site releases the origin-specific barcode from a substrate, such as a bead, for example a hydrogel bead, to which it is coupled. In some examples, at least one cleavage site is oriented such that the cleavage at the site releases the origin-specific barcode from the target molecule specific binding agent. In some examples, a cleavage site is an enzymatic cleavage site, such an endonuclease site present in a specific nucleic acid sequence. In other embodiments, a cleavage site is a peptide cleavage site, such that a particular enzyme can cleave the amino acid sequence. In still other embodiments, a cleavage site is a site of chemical cleavage.

### Barcode Adapters

In some embodiments, the target molecule is attached to an origin-specific barcode receiving adapter, such as a nucleic acid. In some examples, the origin-specific barcode receiving adapter comprises an overhang and the origin-specific barcode comprises a sequence capable of hybridizing to the overhang. A barcode receiving adapter is a molecule configured to accept or receive a nucleic acid barcode, such as an origin-specific nucleic acid barcode. For example, a barcode receiving adapter can include a single-stranded nucleic acid sequence (for example, an overhang) capable of hybridizing to a given barcode (for example, an origin-specific barcode), for example, via a sequence complementary to a portion or the entirety of the nucleic acid barcode. In certain embodiments, this portion of the barcode is a standard sequence held constant between individual barcodes. The hybridization couples the barcode receiving adapter to the barcode. In some embodiments, the barcode receiving adapter may be associated with (for example, attached to) a target molecule. As such, the barcode receiving adapter may serve as the means through which an origin-specific barcode is attached to a target molecule. A barcode receiving adapter can be attached to a target molecule according to methods known in the art. For example, a barcode receiving adapter can be attached to a polypeptide target molecule at a cysteine residue (for example, a C-terminal cysteine residue). A barcode receiving adapter can be used to identify a particular condition related to one or more target molecules, such as a cell of origin or a discreet volume of origin. For example, a target molecule can be a cell surface protein expressed by a cell, which receives a cell-specific barcode receiving adapter. The barcode receiving adapter can be conjugated to one or more barcodes as the cell is exposed to one or more conditions, such that the original cell of origin for the target molecule, as well as each condition to which the cell was exposed, can be subsequently determined by identifying the sequence of the barcode receiving adapter/barcode concatemer.

Barcode with Capture Moiety

In some embodiments, an origin-specific barcode further includes a capture moiety, covalently or non-covalently linked. Thus, in some embodiments the origin-specific barcode, and anything bound or attached thereto, that include a capture moiety are captured with a specific binding agent that specifically binds the capture moiety. In some embodiments, the capture moiety is adsorbed or otherwise captured on a surface. In specific embodiments, a targeting probe is labeled with biotin, for instance by incorporation of biotin-16-UTP during in vitro transcription, allowing later capture by streptavidin. Other means for labeling, capturing, and detecting an origin-specific barcode include: incorporation of aminoallyl-labeled nucleotides, incorporation of sulfhydryl-labeled nucleotides, incorporation of allyl- or azide-containing nucleotides, and many other methods described in Bioconjugate Techniques (2^{nd} Ed), Greg T. Hermanson, Elsevier (2008), which is specifically incorporated herein by reference. In some embodiments, the targeting probes are covalently coupled to a solid support or other capture device prior to contacting the sample, using methods such as incorporation of aminoallyl-labeled nucleotides followed by 1-Ethyl-3-(3-dimethylaminopropyl)carbodiimide (EDC) coupling to a carboxy-activated solid support, or other methods described in Bioconjugate Techniques. In some embodiments, the specific binding agent has been immobilized for example on a solid support, thereby isolating the origin-specific barcode.

### Other Barcoding Embodiments

DNA barcoding is also a taxonomic method that uses a short genetic marker in an organism's DNA to identify it as belonging to a particular species. It differs from molecular phylogeny in that the main goal is not to determine classification but to identify an unknown sample in terms of a known classification. Kress et al., “Use of DNA barcodes to identify flowering plants” Proc. Natl. Acad. Sci. U.S.A. 102(23):8369-8374 (2005). Barcodes are sometimes used in an effort to identify unknown species or assess whether species should be combined or separated. Koch H., “Combining morphology and DNA barcoding resolves the taxonomy of Western Malagasy Liotrigona Moure, 1961” African Invertebrates 51(2): 413-421 (2010); and Seberg et al., “How many loci does it take to DNA barcode a crocus?” PLoS One 4(2):e4598 (2009). Barcoding has been used, for example, for identifying plant leaves even when flowers or fruit are not available, identifying the diet of an animal based on stomach contents or feces, and/or identifying products in commerce (for example, herbal supplements or wood). Soininen et al., “Analysing diet of small herbivores: the efficiency of DNA barcoding coupled with high-throughput pyrosequencing for deciphering the composition of complex plant mixtures” Frontiers in Zoology 6:16 (2009).

It has been suggested that a desirable locus for DNA barcoding should be standardized so that large databases of sequences for that locus can be developed. Most of the taxa of interest have loci that are sequencable without species-specific PCR primers. CBOL Plant Working Group, “A DNA barcode for land plants” PNAS 106(31):12794-12797 (2009). Further, these putative barcode loci are believed short enough to be easily sequenced with current technology. Kress et al., “DNA barcodes: Genes, genomics, and bioinformatics” PNAS 105(8):2761-2762 (2008). Consequently, these loci would provide a large variation between species in combination with a relatively small amount of variation within a species. Lahaye et al., “DNA barcoding the floras of biodiversity hotspots” Proc Natl Acad Sci USA 105(8):2923-2928 (2008).

DNA barcoding is based on a relatively simple concept. For example, most eukaryote cells contain mitochondria, and mitochondrial DNA (mtDNA) has a relatively fast mutation rate, which results in significant variation in mtDNA sequences between species and, in principle, a comparatively small variance within species. A 648-bp region of the mitochondrial cytochrome c oxidase subunit 1 (CO1) gene was proposed as a potential ‘barcode’. As of 2009, databases of CO1 sequences included at least 620,000 specimens from over 58,000 species of animals, larger than databases available for any other gene. Ausubel, J., “A botanical macroscope” Proceedings of the National Academy of Sciences 106(31):12569 (2009).

Software for DNA barcoding requires integration of a field information management system (FIMS), laboratory information management system (LIMS), sequence analysis tools, workflow tracking to connect field data and laboratory data, database submission tools and pipeline automation for scaling up to eco-system scale projects. Geneious Pro can be used for the sequence analysis components, and the two plugins made freely available through the Moorea Biocode Project, the Biocode LIMS and Genbank Submission plugins handle integration with the FIMS, the LIMS, workflow tracking and database submission.

Additionally, other barcoding designs and tools have been described (see e.g., Birrell et al., (2001) Proc. Natl Acad. Sci. USA 98, 12608-12613; Giaever, et al., (2002) Nature 418, 387-391; Winzeler et al., (1999) Science 285, 901-906; and Xu et al., (2009) Proc Natl Acad Sci USA. February 17; 106(7):2289-94).

### Amplification of Target

In certain example embodiments, target RNAs and/or DNAs may be amplified prior to activating the CRISPR effector protein, also referred to as preamplification. Any suitable RNA or DNA amplification technique may be used. In embodiments, preamplifying cell free nucleic acid from a blood or urine sample can be target specific, or non-specific and may be tuned according to, for example, pathogens of interest, and sensitivity and specificity of the guide RNAs. In certain embodiments, the amplification is non-specific, optionally selected from adapter-ligation, degenerate PCR and MDA. In embodiments, the preamplification is target specific, optionally selected from PCR, RPA, or RCA. The probes for preamplification can be designed as disclosed herein for specific or non-specific amplification.

Methods of amplification are provided to allow for sensitive detection of cell free DNA. The most widely used nucleic acid amplification technique is PCR, which employs two target specific primers and a polymerase enzyme, and requires precise cycling of temperatures for denaturation, annealing and extension. Improvements on this basic technique, such as the use of intercalating dyes or hydrolysis probes, have allowed for quantitative readout of signal. Digital droplet PCR (ddPCR), based on performing PCR in water-oil emulsion droplets, has further allowed for precise, absolute quantitation of nucleic acid targets down to single molecule sensitivity.

In some embodiments, the probes are utilized in PCR reactions. In some embodiments, the probes are a set of target specific primers, and may further comprise a polymerase enzyme, intercalating dyes, and/or hydrolysis probes. Quantitative PCR may also be used.

Isothermal amplification can be used in some embodiments. In some instances, isothermal amplification methods may not be as sensitive as PCR, but may be preferred for not requiring a thermocycler. In certain example embodiments, the isothermal amplification may be nucleic-acid sequenced-based amplification (NASBA), recombinase polymerase amplification (RPA), loop-mediated isothermal amplification (LAMP), strand displacement amplification (SDA), helicase-dependent amplification (HDA), or nicking enzyme amplification reaction (NEAR). In certain example embodiments, non-isothermal amplification methods may be used which include, but are not limited to, PCR, multiple displacement amplification (MDA), rolling circle amplification (RCA), ligase chain reaction (LCR), or ramification amplification method (RAM).

In certain embodiments, preferential amplification of microbial DNA is effected by exploiting methylation sites and size selecting microbial cfDNA. In some instances, enrichment can be used. For example, the NEBNext® Microbiome DNA Enrichment Kit available from New England BioLabs facilitates enrichment of microbial DNA from samples containing methylated host DNA (including human), by selective binding and removal of the CpG-methylated host DNA using a magnetic bead based method to selectively bind and remove CpG-methylated host DNA. Magnetic based rods, as in the BioSprint96 from Indical Bioscience can also be used to purify viral nucleic acids and bacterial DNA from samples, including cell-free body fluids. The application of a magnetic field pulls out the CpG-methylated (host) DNA, leaving the non-CpG-methylated (microbial) DNA, with microbial diversity remaining intact after enrichment. In some embodiments size selection can be used with sizes that can be based in part on the different targets of interest. Exemplary size determination and separation methods can include methods as described in Grunt et al., Trans. Cancer Res. (2018) 7:2, Table 2, incorporated herein by reference

In some embodiments, the method may further comprise delivering one or more ligation dependent probes to the cells. A ligation dependent probe (or proximity probe) is a probe that comprises a target binding region configured to bind a target polynucleotide and a primer binding site region. Ligation dependent probes may be used in a set of two or more. Ligation dependent probes may comprise a set of individual ligation dependent probes, with each individual ligation dependent probe configured to hybridize to a specific target nucleic acid sequence on a target polynucleotide. Target sequences on the target polynucleotide are selected to be close enough in distance on the target polynucleotide such that ligation dependent probes hybridized to said target nucleic acid sequences may be subsequently ligated together. Accordingly, in certain embodiments, ligation dependent probe pairs may bind within 1 nucleotides of one another. In some embodiments, the ligation dependent probe pairs may bind within 2 to 500 nucleotides of one another, the gap between which is filled through polymerase extension, or another polynucleotide filler, prior to ligation.

Alternatively, a ligation dependent probe may be a single molecule comprising two or more target binding regions connected by linker sequences. The target binding regions comprise a nucleic acid sequence selected to hybridize to a target region on a target polynucleotide. Linker sequences are selected such that the molecule may adapt a conformation that allows the individual target binding regions to hybridize to adjacent regions on the target polynucleotide. Target sequences on the target polynucleotide are selected to be close enough in distance on the target polynucleotide such that ligation dependent probes hybridized to said target nucleic acid sequences may be subsequently ligated together. Accordingly, in certain embodiments, ligation dependent probe pairs may bind within 1, 2, 3, 4, or 5 nucleotides of one another. In certain example embodiments, the ligation dependent probes comprising two or more target binding regions may be based on molecule inversion probes (MIP), or “padlock probes.” See e.g. Niedzicka et al. Sci Rep. 2016; 6:24501.

In the case of MIPs, padlock probes, and rolling circle probes, constructs for generating labeled target sequences are formed by circularizing a linear version of the probe in a template-driven reaction on a target polynucleotide followed by digestion of non-circularized polynucleotides in the reaction mixture, such as target polynucleotides, unligated probe, probe concatemers, and the like, with an exonuclease, such as exonuclease I.

Ligation dependent probes may be RNA, DNA, or a combination thereof. Ligation dependent probes may vary in length from 10 to 200 nucleotides. To allow for amplification, the ligation dependent probes may further comprise a primer binding site. The same or different primer binding site may be found on each ligation dependent probe. In certain embodiments, a set of ligation dependent probes, each ligation dependent probe comprising target binding region to a different target nucleic acid sequence on the same or different target polynucleotide, but the same primer binding set on each ligation dependent probe.

In one embodiment, the ligation dependent probes are designed to bind one or more target RNA molecules. The ligation dependent probes may be configured to bind to select RNA fragments or RNA exons for the purpose of quantifying the amount of the selected RNA fragment or exon in a sample, or configured to hybridize to a specific RNA sequence variant to detect and identify the presence of said variant in a sample.

Ligation dependent probes are delivered to a sample containing the target molecules of interest. The method of delivery will depend on the sample type. Samples sources may include biological samples of a subject, or environmental samples. These samples may be solids or liquids. The biological samples may include, but are not limited to, animal tissues such as those obtained by biopsy or post mortem, including saliva, blood, semen, plasma, sera, stool, urine, sputum, mucous, lymph, synovial fluid, spinal fluid, cerebrospinal fluid, a swab from skin or a mucosal membrane, or combination thereof. Other biological samples may include plant tissues such as leaves, roots, stems, fruit, and seeds, or sap or other liquids obtained when plant tissues are cut or plant cells are lysed or crushed. Environmental samples may include surfaces or fluids. In an example embodiment, the environmental sample is taken from a solid surface, such as a surface used in the preparation of food or other sensitive compositions and materials.

In specific embodiments, ligation dependent probes may comprise sequences that bind in proximate locations on a target RNA, as well as a first primer handle sequence, a second primer handle sequence, or both. The bound ligation dependent probes may then be linked. The oligonucleotide of the composition and the linked ligation dependent probes may be amplified using barcoded PCR primers. The barcode may be incorporated into each resulting amplicon and the target protein abundance may be quantified and/or target protein localization may be determined based at least in part on sequencing of amplicons as described herein.

Methods for linking the one or more ligation dependent probes include any methods known in the art such as, but not necessarily limited to, ligation, splinted ligation, hybridization, or proximity extension.

In specific embodiments, the protein binding molecule may be an antibody as described herein. In specific embodiments, the cells may be fixed before delivering the ligation dependent probes. In specific embodiments, the amplification reagents may be rolling circle amplification reagents. As described herein, the ligation dependent probes may be molecular inversion probes (MIPs), padlock probes, or split-ligation probes.

In certain preferred embodiments, molecular inversion probes (MIP), referred to herein interchangeably with the term padlock probes, can be used to address limitations of some other primer approaches. MIPs are ligation dependent probes.

Padlock Probe (PP) technology is a multiplex genomic enrichment method allowing for accurate targeted high-throughput sequencing. PP technology has been used to perform highly multiplexed genotyping, digital allele quantification, targeted bisulfite sequencing, and exome sequencing. See Hardenbol, P. et al., (2005) Genome Res. 15: 269-275. Molecular inversion probes have been used for multiplexed capture and amplification of over 10,000 targets in the same reaction.

Ligation dependent probes may be RNA, DNA, or a combination thereof. Ligation dependent probes may vary in length from 10 to 200 nucleotides. To allow for amplification, the ligation dependent probes may further comprise a primer binding site. The same or different primer binding site may be found on each ligation dependent probe. In certain embodiments, a set of ligation dependent probes, each ligation dependent probe comprising target binding region to a different target nucleic acid sequence on the same or different target polynucleotide, but the same primer binding set on each ligation dependent probe.

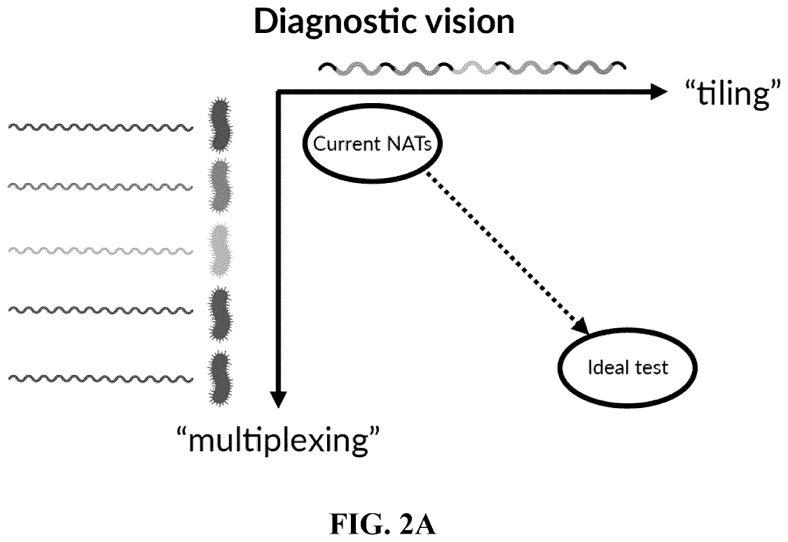

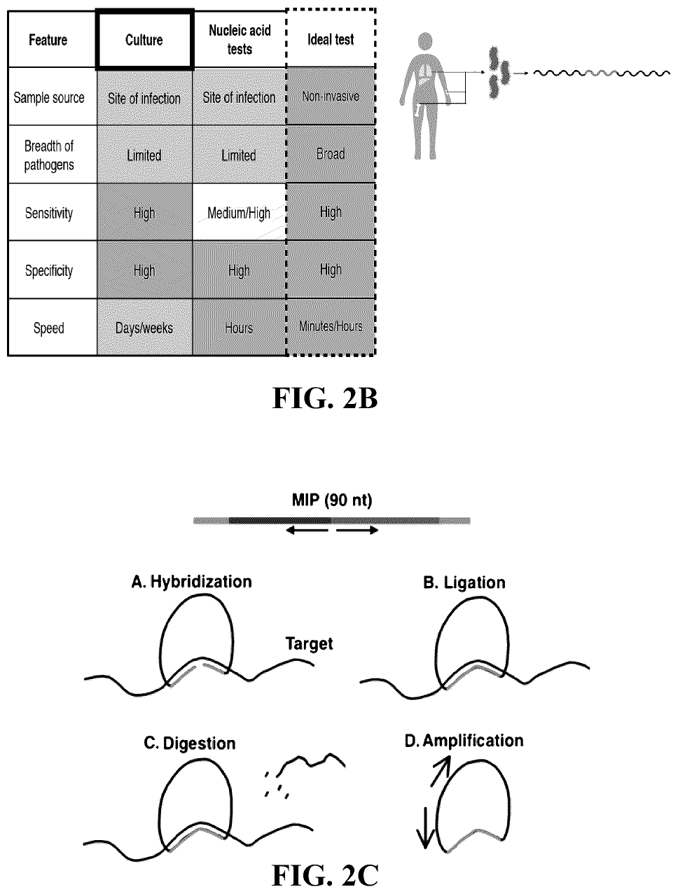

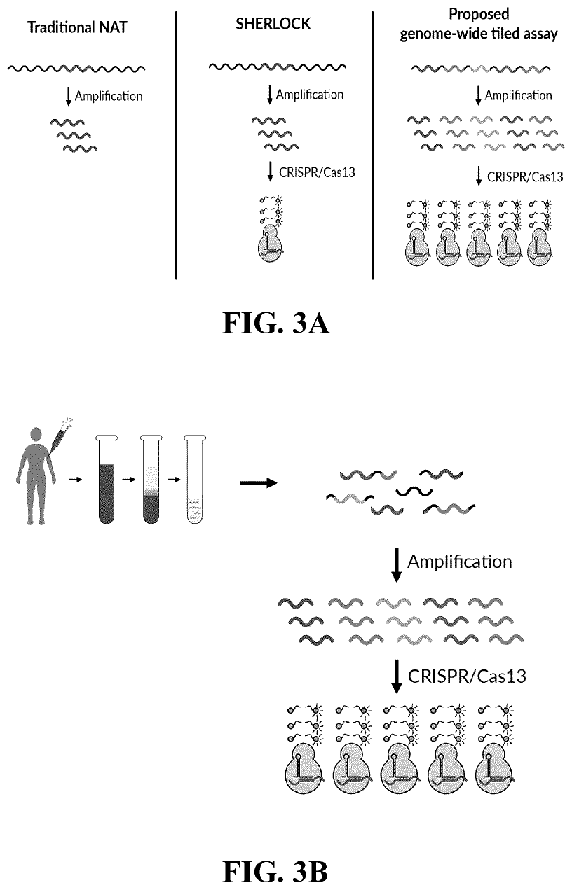

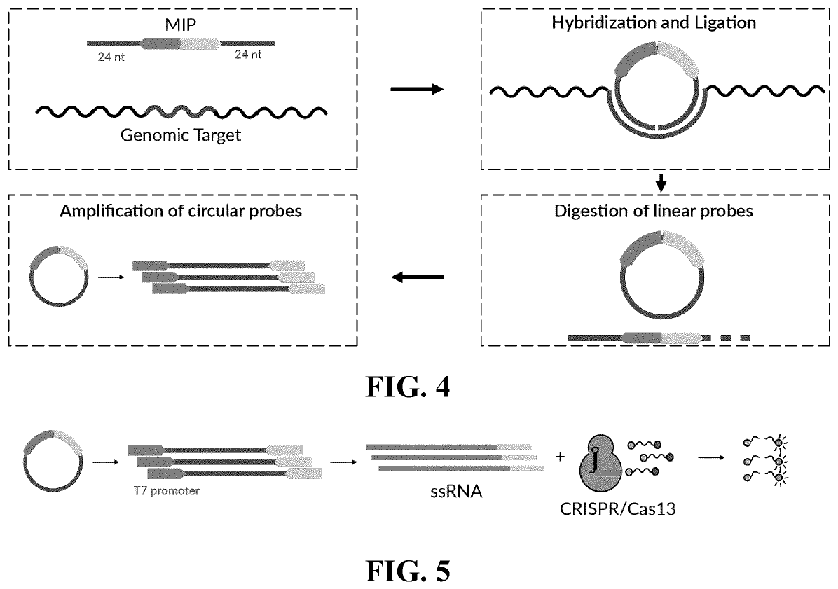

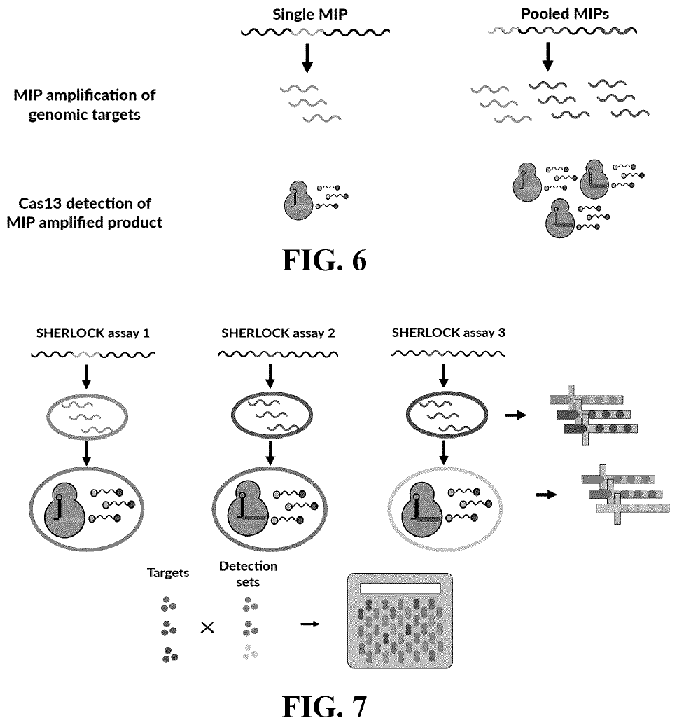

In an embodiment the approach involves the use of long DNA probes with variable target binding sites at the two ends (orange in FIG. 2C), and conserved primer binding sites in between (green and blue in FIG. 2C). The steps involved in a MIP assay are: (1) hybridization of probes to target; (2) ligation (circularization) of hybridized probes; (3) digestion of non-hybridized, linear probes; (4) addition of primer pair and amplification of circularized probes (FIG. 2C). In some embodiments, the MIP probes are about 50 to about 200 nucleotides, in some embodiments, the MIP probes are about 90 nucleotides.

In some embodiments, the probes are evenly spaced across a genome of interest, or across a variable region of a genome of interest. In some embodiments, the MIP probes comprise about 15 to about 30 contiguous nucleotides, preferably about 20 to about 30, or about 22 to 26, or about 24 nucleotides on each end of the probes complementary to a target nucleic acid molecule.

The proximity dependent probes, in some instances, padlock probes, comprise two ends complementary to the target molecule of interest. Each end of the complementary nucleotides can be about 10 to about 100 nucleotides, about 15 to about 50-80 nucleotides, about 20 to 30 nucleotides, in some embodiments, about 24 nucleotides on each end of the probe. In embodiments, the MIPs are about 80 to 200 nucleotides long, in some embodiments, about 80 to 95, or about 90 nucleotides long, with conserved primer binding sites in between the two ends complementary to the target molecule. In particular embodiments, the MIPs can be tiled across a genome of interest, or a portion of a gene of interest. The variable region of the genome of interest, or target sequence of interest is about 5 to about 150 nucleotides in length, about 10 to about 100 nucleotides length, or about 10, 15, 20, 25, 30, 35, 40, 45, 50, 55, 60 65, 70, 75, 80, 85, 90, 95 100, 105, 110, 115, 120 or 125 nucleotides in length. In some embodiments, a guide RNA recognition sequence is provided and integrated within the sequence complementary to the target molecule of interest, or provided in proximity to a sequence complementary to the target molecule of interest. In proximity may be within about 50, 40, 30, about 20, about 10, about 9, 8, 7, 6, 5, 4, 3, 2, 1 or 0 nucleotides of the sequence complementary to the target molecule of interest.

The MIPs and other proximity probes are flexible in configuration and can be designed in several ways for use in the systems and methods disclosed herein. In one embodiment, the MIP structure comprises a guide polynucleotide binding sequence integrated into the target of interest. A specific species can be identified in this manner, and the probe may contain a binding site comprising a genomic target of interest (gDNA binding)—guide polynucleotide binding sequence—genomic target of interest (gDNA binding) in the MIP structure.

In embodiments wherein the proximity dependent probe is a MIP, the MIP can comprise a first binding sequence and a second target binding sequence linked by a linking region, or backbone. The linking region can comprise one or more of a forward primer binding sequence, a reverse primer binding sequence, a RNA polymerase binding sequence, a guide polynucleotide binding sequence, and a barcode. In particular instances, the first target binding sequence and the second target binding sequence hybridize on the target sequence directly adjacent to one another, on other instances, there is at least a single nucleotide gap region between the first and second target binding sequences. A gap region is the

In embodiments, the MIP structure can include a guide polynucleotide binding sequence integrated into the MIP linking region, or can be integrated into the genomic target sequence. In certain embodiments, the guide polynucleotide binding sequence is in proximity to one of the genomic target of interest gDNA binding sites within the MIP, but is not integrated into the target of interest. Inclusion of the crRNA target sequence into the MIP backbone can allow for a first targeting of, for example, a common region of interest amongst a category of organism that may be species specific, genus specific, or across several different organisms, for example, gram-negative or gram-positive bacteria. The guide RNA can then be utilized to further select for species, genus, or other category of interest, as exemplified in FIG. 20.

In an embodiment, the proximity dependent probe is designed to form a gap region upon binding to the target of interest. The gap region is the area between the two target sequence complementary ends of the probe. As described herein, the gap region can be designed to comprise 1 to 100 nucleotides. Gap filling of the proximity probe can be effected prior to ligation and amplification, thereby creating a circularized probe with a gap-filled region. The gap-filled region can be designed to generate the guide polynucleotide recognition sequence. Optionally, the nucleotides used for gap filling can be modified nucleotides comprising a capture moiety, which includes incorporation of aminoallyl-labeled nucleotides, incorporation of sulfhydryl-labeled nucleotides, incorporation of allyl- or azide-containing nucleotides, and many other methods described in Bioconjugate Techniques (2^{nd} Ed), Greg T. Hermanson, Elsevier (2008). Systems can further comprise a capture agent that binds the capture moiety of the modified nucleotides. Exemplary systems can comprise biotinylated nucleotides with a capture agent comprising streptavidin or a streptavidin-functionalized surface. Exemplary surfaces can include particles, beads, and solid surfaces. Capture moieties, capture agents and surfaces can be as described elsewhere herein for barcode capture moieties.

Methods of developing probes are also provided herein, with the probes utilized in the amplification step, or, in some embodiments, as guide RNAs. Tiled probes of the present invention can, in embodiments, be utilized for the guide sequence and/or the proximity dependent probes. In particular embodiments, tiled probes can be designed across variable regions of a genome. In particular embodiments, the probes are evenly spaced across the length of a genome, across the variable region of a genome, or across a portion of a genome. Sets of tiled probes can be used in one reaction, in some embodiments, a larger set of probes yields a stronger signal in detection reactions.

In particular embodiments, the genome of interest is microbial. In some embodiments, the genome of interest is viral or bacterial. In some embodiments, gram negative or gram positive bacteria are the targets of interest; in embodiments, species specific targets of interest are the genome of interest, in other embodiments, all of the species of a genus of interest are targeted. In particular embodiments, the genome of interest is Klebsiella pheuminiae, Pseudomonas aeruginosa, S. aureus, M tuberculosis, Staphylococcus epidemidis or A. fumigatus. In some embodiments, the padlock probes can be designed across variable regions of a genome.

In an exemplary embodiment, one or more tiled MIPs across a genome are detected, and integrating a common recognition sequence that can be detected by a guide RNA. In some instances, detection of the common sequence by the guide RNA for target sequences of interest across a genome that have been selected by the MIPs. This common sequence detected by the guide RNAs can work to increase a final signal detected, that is, a signal common across all probes form a sample. In an exemplary method, MIPs are tiled across a genome of interest, each of the individual MIPs further comprising a common guide RNA recognition sequence. When the common guide RNA is utilized to detect sequences comprising the common sequence, a signal common across all probes from the sample can be generated. Allowing for a simpler diagnostic.

In certain other example embodiments, a recombinase polymerase amplification (RPA) reaction may be used to amplify the target nucleic acids. RPA reactions employ recombinases which are capable of pairing sequence-specific primers with homologous sequence in duplex DNA. If target DNA is present, DNA amplification is initiated and no other sample manipulation such as thermal cycling or chemical melting is required. The entire RPA amplification system is stable as a dried formulation and can be transported safely without refrigeration. RPA reactions may also be carried out at isothermal temperatures with an optimum reaction temperature of 37-42° C. The sequence specific primers are designed to amplify a sequence comprising the target nucleic acid sequence to be detected. In certain example embodiments, a RNA polymerase promoter, such as a T7 promoter, is added to one of the primers. This results in an amplified double-stranded DNA product comprising the target sequence and a RNA polymerase promoter. After, or during, the RPA reaction, a RNA polymerase is added that will produce RNA from the double-stranded DNA templates. The amplified target RNA can then in turn be detected by the CRISPR effector system. In this way target DNA can be detected using the embodiments disclosed herein. RPA reactions can also be used to amplify target RNA. The target RNA is first converted to cDNA using a reverse transcriptase, followed by second strand DNA synthesis, at which point the RPA reaction proceeds as outlined above.

Accordingly, in certain example embodiments the systems disclosed herein may include amplification reagents. Different components or reagents useful for amplification of nucleic acids are described herein. For example, an amplification reagent as described herein may include a buffer, such as a Tris buffer. A Tris buffer may be used at any concentration appropriate for the desired application or use, for example including, but not limited to, a concentration of 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 25 mM, 50 mM, 75 mM, 1 M, or the like. One of skill in the art will be able to determine an appropriate concentration of a buffer such as Tris for use with the present invention.

A salt, such as magnesium chloride (MgCl₂), potassium chloride (KCl), or sodium chloride (NaCl), may be included in an amplification reaction, such as PCR, in order to improve the amplification of nucleic acid fragments. Although the salt concentration will depend on the particular reaction and application, in some embodiments, nucleic acid fragments of a particular size may produce optimum results at particular salt concentrations. Larger products may require altered salt concentrations, typically lower salt, in order to produce desired results, while amplification of smaller products may produce better results at higher salt concentrations. One of skill in the art will understand that the presence and/or concentration of a salt, along with alteration of salt concentrations, may alter the stringency of a biological or chemical reaction, and therefore any salt may be used that provides the appropriate conditions for a reaction of the present invention and as described herein.

Other components of a biological or chemical reaction may include a cell lysis component in order to break open or lyse a cell for analysis of the materials therein. A cell lysis component may include, but is not limited to, a detergent, a salt as described above, such as NaCl, KCl, ammonium sulfate [(NH₄)₂SO₄], or others. Detergents that may be appropriate for the invention may include Triton X-100, sodium dodecyl sulfate (SDS), CHAPS (3-[(3-cholamidopropyl)dimethylammonio]-1-propanesulfonate), ethyl trimethyl ammonium bromide, nonyl phenoxypolyethoxylethanol (NP-40). Concentrations of detergents may depend on the particular application, and may be specific to the reaction in some cases. Amplification reactions may include dNTPs and nucleic acid primers used at any concentration appropriate for the invention, such as including, but not limited to, a concentration of 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, or the like. Likewise, a polymerase useful in accordance with the invention may be any specific or general polymerase known in the art and useful or the invention, including Taq polymerase, Q5 polymerase, or the like.

In some embodiments, amplification reagents as described herein may be appropriate for use in hot-start amplification. Hot start amplification may be beneficial in some embodiments to reduce or eliminate dimerization of adaptor molecules or oligos, or to otherwise prevent unwanted amplification products or artifacts and obtain optimum amplification of the desired product. Many components described herein for use in amplification may also be used in hot-start amplification. In some embodiments, reagents or components appropriate for use with hot-start amplification may be used in place of one or more of the composition components as appropriate. For example, a polymerase or other reagent may be used that exhibits a desired activity at a particular temperature or other reaction condition. In some embodiments, reagents may be used that are designed or optimized for use in hot-start amplification, for example, a polymerase may be activated after transposition or after reaching a particular temperature. Such polymerases may be antibody-based or aptamer-based. Polymerases as described herein are known in the art. Examples of such reagents may include, but are not limited to, hot-start polymerases, hot-start dNTPs, and photo-caged dNTPs. Such reagents are known and available in the art. One of skill in the art will be able to determine the optimum temperatures as appropriate for individual reagents.

Amplification of nucleic acids may be performed using specific thermal cycle machinery or equipment, and may be performed in single reactions or in bulk, such that any desired number of reactions may be performed simultaneously. In some embodiments, amplification may be performed using microfluidic or robotic devices, or may be performed using manual alteration in temperatures to achieve the desired amplification. In some embodiments, optimization may be performed to obtain the optimum reactions conditions for the particular application or materials. One of skill in the art will understand and be able to optimize reaction conditions to obtain sufficient amplification.

In some instances, the nucleic acid amplification reagents comprise recombinase polymerase amplification (RPA) reagents, nucleic acid sequence-based amplification (NASBA) reagents, loop-mediated isothermal amplification (LAMP) reagents, strand displacement amplification (SDA) reagents, helicase-dependent amplification (HDA) reagents, nicking enzyme amplification reaction (NEAR) reagents, PCR or RT-PCR reagents, multiple displacement amplification (MDA) reagents, rolling circle amplification (RCA) reagents, ligase chain reaction (LCR) reagents, ramification amplification method (RAM) reagents, transposase based amplification reagents; or Programmable CRISPR Nicking Amplification (PCNA)reagents.

In certain embodiments, detection of DNA with the methods or systems of the invention requires transcription of the (amplified) DNA into RNA prior to detection.

It will be evident that detection methods of the invention can involve nucleic acid amplification and detection procedures in various combinations. The nucleic acid to be detected can be any naturally occurring or synthetic nucleic acid, including but not limited to DNA and RNA, which may be amplified by any suitable method to provide an intermediate product that can be detected. Detection of the intermediate product can be by any suitable method including but not limited to binding and activation of a CRISPR protein which produces a detectable signal moiety by direct or collateral activity.

In embodiments, methods of increasing hybridization efficiency of a probe, such as an MIP probe and a target nucleic acid sequence in a cell free sample is provided. In some embodiments, method includes heating a sample, such as a cell free sample, comprising at least one target RNA to a temperature of about 80° C. and about 95° C. for a time sufficient to interfere with, for example disrupt, secondary structure of the RNA, wherein the time is short enough, such that the RNA in the sample are not significantly degraded in which the sample includes a chemical denaturant. Although not bound by theory, the heating step in the presence of a chemical denaturant is believed to make regions of RNA more accessible to a probe, by removing interfering proteins and secondary and/or tertiary elements from the target RNA. In exemplary embodiments of the disclosed method, the samples, such as a cell free sample, is contacted with at least one detectable probe, in some embodiments, a padlock probe, that specifically hybridizes to the target RNA in the sample. In example embodiments, the sample is not allowed to cool appreciably before contact with the probe, such that the RNA present in the sample is not allowed to reanneal and form secondary structure that may interfere with probe binding. In particular embodiments, heating the sample to a temperature of between about 80° C. and about 95° C. for a time sufficient to interfere with secondary structure of the RNA increases the detected hybridization between the probe and the target RNA relative to the hybridization between the probe and the target RNA in the absence of the heating step. In some embodiments, hybridization between the probe and the target RNA detects the presence of the target RNA in the sample. Methods of heating can be used as described in U.S. Patent Publication No. 20160304942, incorporated herein in its entirety, and methods of heating at [0075]—[0136].

### Target Molecules

Target molecules, as described herein can include any target nucleic acid sequence, that, in embodiments, the one or more guide RNAs are designed to bind to one or more target molecules that are diagnostic for a disease state. In further embodiments, the disease state is an infection, an organ disease, a blood disease, an immune system disease, a cancer, a brain and nervous system disease, an endocrine disease, a pregnancy or childbirth-related disease, an inherited disease, or an environmentally-acquired disease. In still further embodiments, the disease state is an infection, including a microbial infection.

In one aspect, all possible genomic targets for a given set of pathogens, unique to the pathogens of interest are identified. In some preferred embodiments, the genomic target sequence also includes target conservation across the pathogens of interest.

Depending on the particular application, “pathogens of interest” may encompass all strains within a species, or include just a single strain. To accommodate varying levels of resolution, “in” and “out” groups can be identified. The “in” group encompasses all genomes to detect, while the “out” group comprises of all genomes do not want to detect as signal (theoretically all other genomes). Once the “in” and “out” groups are defined, a reference genome is chosen within the “in” group. This reference genome is used to generate a list of all possible genomic targets of a pre-defined size. Next, a sequence alignment tool (Bowtie) is used to identify matching sequences with all other genomes in the “in” and “out” groups. A candidate list of possible genomic targets comprises of those sequences that match with all genomes in the “in” group, and do not match with any of the genomes in the “out” group.

In further embodiments, the infection is caused by a virus, a bacterium, or a fungus, or the infection is a viral infection as described herein. In specific embodiments, the viral infection is caused by a double-stranded RNA virus, a positive sense RNA virus, a negative sense RNA virus, a retrovirus, or a combination thereof. In certain embodiments, the application can achieve multiplexed strain discrimination. In some embodiments, one or more pathogens are detected. In some embodiments, the target sequences are from strains of the same bacterial species. In some embodiments, the pathogens of interest comprise one or more of Staphylococcus aureus, Aspergillus fumigatus and Mycobacterium tuberculosis. Co-infections, such as HIV/TB co-infections, can be detected.

As described herein, a sample containing target molecules for use with the invention may be a biological sample. Biological samples may be crude samples and/or the one or more target molecules may not be purified or amplified from the sample prior to application of the method. Identification of microbes may be useful and/or needed for any number of applications, and thus any type of sample from any source deemed appropriate by one of skill in the art may be used in accordance with the invention.

In some embodiments, the biological sample may include, but is not necessarily limited to, blood, plasma, serum, urine, stool, sputum, mucous, lymph fluid, synovial fluid, bile, ascites, pleural effusion, seroma, saliva, cerebrospinal fluid, aqueous or vitreous humor, or any bodily secretion, a transudate, an exudate, or fluid obtained from a joint, or a swab of skin or mucosal membrane surface.

In specific embodiments, the sample may be blood, plasma or serum obtained from a human patient. Owing to the increased sensitivity of the embodiments disclosed herein, in certain example embodiments, the assays and methods may be run on crude samples or samples where the target molecules to be detected are not further fractionated or purified from the sample.

### Example Microbes

The embodiment disclosed herein may be used to detect a number of different microbes. The term microbe as used herein includes bacteria, fungus, protozoa, parasites and viruses.

### Bacteria

The following provides an example list of the types of microbes that might be detected using the embodiments disclosed herein. In certain example embodiments, the microbe is a bacterium. Examples of bacteria that can be detected in accordance with the disclosed methods include without limitation any one or more of (or any combination of) Acinetobacter baumanii, Actinobacillus sp., Actinomycetes, Actinomyces sp. (such as Actinomyces israelii and Actinomyces naeslundii), Aeromonas sp. (such as Aeromonas hydrophila, Aeromonas veronii biovar sobria (Aeromonas sobria), and Aeromonas caviae), Anaplasma phagocytophilum, Anaplasma marginale Alcaligenes xylosoxidans, Acinetobacter baumanii, Actinobacillus actinomycetemcomitans, Bacillus sp. (such as Bacillus anthracis, Bacillus cereus, Bacillus subtilis, Bacillus thuringiensis, and Bacillus stearothermophilus), Bacteroides sp. (such as Bacteroides fragilis), Bartonella sp. (such as Bartonella bacilliformis and Bartonella henselae, Bifidobacterium sp., Bordetella sp. (such as Bordetella pertussis, Bordetella parapertussis, and Bordetella bronchiseptica), Borrelia sp. (such as Borrelia recurrentis, and Borrelia burgdorferi), Brucella sp. (such as Brucella abortus, Brucella canis, Brucella melintensis and Brucella suis), Burkholderia sp. (such as Burkholderia pseudomallei and Burkholderia cepacia), Campylobacter sp. (such as Campylobacter jejuni, Campylobacter coli, Campylobacter lari and Campylobacter fetus), Capnocytophaga sp., Cardiobacterium hominis, Chlamydia trachomatis, Chlamydophila pneumoniae, Chlamydophila psittaci, Citrobacter sp. Coxiella burnetii, Corynebacterium sp. (such as, Corynebacterium diphtheriae, Corynebacterium jeikeum and Corynebacterium), Clostridium sp. (such as Clostridium perfringens, Clostridium difficile, Clostridium botulinum and Clostridium tetani), Eikenella corrodens, Enterobacter sp. (such as Enterobacter aerogenes, Enterobacter agglomerans, Enterobacter cloacae and Escherichia coli, including opportunistic Escherichia coli, such as enterotoxigenic E. coli, enteroinvasive E. coli, enteropathogenic E. coli, enterohemorrhagic E. coli, enteroaggregative E. coli and uropathogenic E. coli) Enterococcus sp. (such as Enterococcus faecalis and Enterococcus faecium) Ehrlichia sp. (such as Ehrlichia chafeensia and Ehrlichia canis), Epidermophyton floccosum, Erysipelothrix rhusiopathiae, Eubacterium sp., Francisella tularensis, Fusobacterium nucleatum, Gardnerella vaginalis, Gemella morbillorum, Haemophilus sp. (such as Haemophilus influenzae, Haemophilus ducreyi, Haemophilus aegyptius, Haemophilus parainfluenzae, Haemophilus haemolyticus and Haemophilus parahaemolyticus, Helicobacter sp. (such as Helicobacter pylori, Helicobacter cinaedi and Helicobacter fennelliae), Kingella kingii, Klebsiella sp. (such as Klebsiella pneumoniae, Klebsiella granulomatis and Klebsiella oxytoca), Lactobacillus sp., Listeria monocytogenes, Leptospira interrogans, Legionella pneumophila, Leptospira interrogans, Peptostreptococcus sp., Mannheimia hemolytica, Microsporum canis, Moraxella catarrhalis, Morganella sp., Mobiluncus sp., Micrococcus sp., Mycobacterium sp. (such as Mycobacterium leprae, Mycobacterium tuberculosis, Mycobacterium paratuberculosis, Mycobacterium intracellulare, Mycobacterium avium, Mycobacterium bovis, and Mycobacterium marinum), Mycoplasm sp. (such as Mycoplasma pneumoniae, Mycoplasma hominis, and Mycoplasma genitalium), Nocardia sp. (such as Nocardia asteroides, Nocardia cyriacigeorgica and Nocardia brasiliensis), Neisseria sp. (such as Neisseria gonorrhoeae and Neisseria meningitidis), Pasteurella multocida, Pityrosporum orbiculare (Malassezia furfur), Plesiomonas shigelloides. Prevotella sp., Porphyromonas sp., Prevotella melaninogenica, Proteus sp. (such as Proteus vulgaris and Proteus mirabilis), Providencia sp. (such as Providencia alcalifaciens, Providencia rettgeri and Providencia stuartii), Pseudomonas aeruginosa, Propionibacterium acnes, Rhodococcus equi, Rickettsia sp. (such as Rickettsia rickettsii, Rickettsia akari and Rickettsia prowazekii, Orientia tsutsugamushi (formerly: Rickettsia tsutsugamushi) and Rickettsia typhi), Rhodococcus sp., Serratia marcescens, Stenotrophomonas maltophilia, Salmonella sp. (such as Salmonella enterica, Salmonella typhi, Salmonella paratyphi, Salmonella enteritidis, Salmonella cholerasuis and Salmonella typhimurium), Serratia sp. (such as Serratia marcesans and Serratia liquifaciens), Shigella sp. (such as Shigella dysenteriae, Shigella flexneri, Shigella boydii and Shigella sonnei), Staphylococcus sp. (such as Staphylococcus aureus, Staphylococcus epidermidis, Staphylococcus hemolyticus, Staphylococcus saprophyticus), Streptococcus sp. (such as Streptococcus pneumoniae (for example chloramphenicol-resistant serotype 4 Streptococcus pneumoniae, spectinomycin-resistant serotype 6B Streptococcus pneumoniae, streptomycin-resistant serotype 9V Streptococcus pneumoniae, erythromycin-resistant serotype 14 Streptococcus pneumoniae, optochin-resistant serotype 14 Streptococcus pneumoniae, rifampicin-resistant serotype 18C Streptococcus pneumoniae, tetracycline-resistant serotype 19F Streptococcus pneumoniae, penicillin-resistant serotype 19F Streptococcus pneumoniae, and trimethoprim-resistant serotype 23F Streptococcus pneumoniae, chloramphenicol-resistant serotype 4 Streptococcus pneumoniae, spectinomycin-resistant serotype 6B Streptococcus pneumoniae, streptomycin-resistant serotype 9V Streptococcus pneumoniae, optochin-resistant serotype 14 Streptococcus pneumoniae, rifampicin-resistant serotype 18C Streptococcus pneumoniae, penicillin-resistant serotype 19F Streptococcus pneumoniae, or trimethoprim-resistant serotype 23F Streptococcus pneumoniae), Streptococcus agalactiae, Streptococcus mutans, Streptococcus pyogenes, Group A streptococci, Streptococcus pyogenes, Group B streptococci, Streptococcus agalactiae, Group C streptococci, Streptococcus anginosus, Streptococcus equismilis, Group D streptococci, Streptococcus bovis, Group F streptococci, and Streptococcus anginosus Group G streptococci), Spirillum minus, Streptobacillus moniliformi, Treponema sp. (such as Treponema carateum, Treponema petenue, Treponema pallidum and Treponema endemicum, Trichophyton rubrum, T. mentagrophytes, Tropheryma whippeihi, Ureaplasma urealyticum, Veillonella sp., Vibrio sp. (such as Vibrio cholerae, Vibrio parahemolyticus, Vibrio vulnificus, Vibrio parahaemolyticus, Vibrio vulnificus, Vibrio alginolyticus, Vibrio mimicus, Vibrio hollisae, Vibrio fluvialis, Vibrio metchnikovii, Vibrio damsela and Vibrio furnisii), Yersinia sp. (such as Yersinia enterocolitica, Yersinia pestis, and Yersinia pseudotuberculosis) andXanthomonas maltophilia among others.

### Fungi

In certain example embodiments, the microbe is a fungus or a fungal species. Examples of fungi that can be detected in accordance with the disclosed methods include without limitation any one or more of (or any combination of), Aspergillus, Blastomyces, Candidiasis, Coccidiodomycosis, Cryptococcus neoformans, Cryptococcus gatti, sp. Histoplasma sp. (such as Histoplasma capsulatum), Pneumocystis sp. (such as Pneumocystis jirovecii), Stachybotrys (such as Stachybotrys chartarum), Mucroymcosis, Sporothrix, fungal eye infections ringworm, Exserohilum, Cladosporium.

In certain example embodiments, the fungus is a yeast. Examples of yeast that can be detected in accordance with disclosed methods include without limitation one or more of (or any combination of), Aspergillus species (such as Aspergillus fumigatus, Aspergillus flavus and Aspergillus clavatus), Cryptococcus sp. (such as Cryptococcus neoformans, Cryptococcus gattii, Cryptococcus laurentii and Cryptococcus albidus), a Geotrichum species, a Saccharomyces species, a Hansenula species, a Candida species (such as Candida albicans), a Kluyveromyces species, a Debaryomyces species, a Pichia species, or combination thereof. In certain example embodiments, the fungi is a mold. Example molds include, but are not limited to, a Penicillium species, a Cladosporium species, a Byssochlamys species, or a combination thereof.

### Protozoa

In certain example embodiments, the microbe is a protozoa. Examples of protozoa that can be detected in accordance with the disclosed methods and devices include without limitation any one or more of (or any combination of), Euglenozoa, Heterolobosea, Diplomonadida, Amoebozoa, Blastocystic, and Apicomplexa. Example Euglenoza include, but are not limited to, Trypanosoma cruzi (Chagas disease), T. brucei gambiense, T. brucei rhodesiense, Leishmania braziliensis, L. infantum, L. mexicana, L. major, L. tropica, and L. donovani. Example Heterolobosea include, but are not limited to, Naegleriafowleri. Example Diplomonadids include, but are not limited to, Giardia intestinalis (G. lamblia, G. duodenalis). Example Amoebozoa include, but are not limited to, Acanthamoeba castellanii, Balamuthia madrillaris, Entamoeba histolytica. Example Blastocysts include, but are not limited to, Blastocystic hominis. Example Apicomplexa include, but are not limited to, Babesia microti, Cryptosporidium parvum, Cyclospora cayetanensis, Plasmodium falciparum, P. vivax, P. ovale, P. malariae, and Toxoplasma gondii.

### Parasites

In certain example embodiments, the microbe is a parasite. Examples of parasites that can be detected in accordance with disclosed methods include without limitation one or more of (or any combination of), an Onchocerca species and a Plasmodium species.

### Viruses

In certain example embodiments, the systems, devices, and methods, disclosed herein are directed to detecting viruses in a sample. The embodiments disclosed herein may be used to detect viral infection (e.g. of a subject or plant), or determination of a viral strain, including viral strains that differ by a single nucleotide polymorphism. The virus may be a DNA virus, a RNA virus, or a retrovirus. Non-limiting example of viruses useful with the present invention include, but are not limited to Ebola, measles, SARS, Chikungunya, hepatitis, Marburg, yellow fever, MERS, Dengue, Lassa, influenza, rhabdovirus or HIV. A hepatitis virus may include hepatitis A, hepatitis B, or hepatitis C. An influenza virus may include, for example, influenza A or influenza B. An HIV may include HIV 1 or HIV 2. In certain example embodiments, the viral sequence may be a human respiratory syncytial virus, Sudan ebola virus, Bundibugyo virus, Tai Forest ebola virus, Reston ebola virus, Achimota, Aedes flavivirus, Aguacate virus, Akabane virus, Alethinophid reptarenavirus, Allpahuayo mammarenavirus, Amapari mmarenavirus, Andes virus, Apoi virus, Aravan virus, Aroa virus, Arumwot virus, Atlantic salmon paramyxovirus, Australian bat lyssavirus, Avian bomavirus, Avian metapneumovirus, Avian paramyxoviruses, penguin or Falkland Islandsvirus, BK polyomavirus, Bagaza virus, Banna virus, Bat herpesvirus, Bat sapovirus, Bear Canon mammarenavirus, Beilong virus, Betacoronavirus, Betapapillomavirus 1-6, Bhanja virus, Bokeloh bat lyssavirus, Borna disease virus, Bourbon virus, Bovine hepacivirus, Bovine parainfluenza virus 3, Bovine respiratory syncytial virus, Brazoran virus, Bunyamwera virus, Caliciviridae virus. California encephalitis virus, Candiru virus, Canine distemper virus, Canine pneumovirus, Cedar virus, Cell fusing agent virus, Cetacean morbillivirus, Chandipura virus, Chaoyang virus, Chapare mammarenavirus, Chikungunya virus, Colobus monkey papillomavirus, Colorado tick fever virus, Cowpox virus, Crimean-Congo hemorrhagic fever virus, Culex flavivirus, Cupixi mammarenavirus, Dengue virus, Dobrava-Belgrade virus, Donggang virus, Dugbe virus, Duvenhage virus, Eastern equine encephalitis virus, Entebbe bat virus, Enterovirus A-D, European bat lyssavirus 1-2, Eyach virus, Feline morbillivirus, Fer-de-Lance paramyxovirus, Fitzroy River virus, Flaviviridae virus, Flexal mammarenavirus, GB virus C, Gairo virus, Gemycircularvirus, Goose paramyxovirus SF02, Great Island virus, Guanarito mammarenavirus, Hantaan virus, Hantavirus Z10, Heartland virus, Hendra virus, Hepatitis A/B/C/E, Hepatitis delta virus, Human bocavirus, Human coronavirus, Human endogenous retrovirus K, Human enteric coronavirus, Human genital-associated circular DNA virus-1, Human herpesvirus 1-8, Human immunodeficiency virus 1/2, Human mastadenovirus A-G, Human papillomavirus, Human parainfluenza virus 1-4, Human paraechovirus, Human picornavirus, Human smacovirus, Ikoma lyssavirus, Ilheus virus, Influenza A-C, Ippy mammarenavirus, Irkut virus, J-virus, JC polyomavirus, Japanese encephalitis virus, Junin mammarenavirus, KI polyomavirus, Kadipiro virus, Kamiti River virus, Kedougou virus, Khujand virus, Kokobera virus, Kyasanur forest disease virus, Lagos bat virus, Langat virus, Lassa mammarenavirus, Latino mammarenavirus, Leopards Hill virus, Liao ning virus, Ljungan virus, Lloviu virus, Louping ill virus, Lujo mammarenavirus, Luna mammarenavirus, Lunk virus, Lymphocytic choriomeningitis mammarenavirus, Lyssavirus Ozernoe, MSSI2\.225 virus, Machupo mammarenavirus, Mamastrovirus 1, Manzanilla virus, Mapuera virus, Marburg virus, Mayaro virus, Measles virus, Menangle virus, Mercadeo virus, Merkel cell polyomavirus, Middle East respiratory syndrome coronavirus, Mobala mammarenavirus, Modoc virus, Moijang virus, Mokolo virus, Monkeypox virus, Montana myotis leukoenchalitis virus, Mopeia lassa virus reassortant 29, Mopeia mammarenavirus, Morogoro virus, Mossman virus, Mumps virus, Murine pneumonia virus, Murray Valley encephalitis virus, Nariva virus, Newcastle disease virus, Nipah virus, Norwalk virus, Norway rat hepacivirus, Ntaya virus, O'nyong-nyong virus, Oliveros mammarenavirus, Omsk hemorrhagic fever virus, Oropouche virus, Parainfluenza virus 5, Parana mammarenavirus, Parramatta River virus, Peste-des-petits-ruminants virus, Pichande mammarenavirus, Picornaviridae virus, Pirital mammarenavirus, Piscihepevirus A, Porcine parainfluenza virus 1, porcine rubulavirus, Powassan virus, Primate T-lymphotropic virus 1-2, Primate erythroparvovirus 1, Punta Toro virus, Puumala virus, Quang Binh virus, Rabies virus, Razdan virus, Reptile bornavirus 1, Rhinovirus A-B, Rift Valley fever virus, Rinderpest virus, Rio Bravo virus, Rodent Torque Teno virus, Rodent hepacivirus, Ross River virus, Rotavirus A-I, Royal Farm virus, Rubella virus, Sabia mammarenavirus, Salem virus, Sandfly fever Naples virus, Sandfly fever Sicilian virus, Sapporo virus, Sathuperi virus, Seal anellovirus, Semliki Forest virus, Sendai virus, Seoul virus, Sepik virus, Severe acute respiratory syndrome-related coronavirus, Severe fever with thrombocytopenia syndrome virus, Shamonda virus, Shimoni bat virus, Shuni virus, Simbu virus, Simian torque teno virus, Simian virus 40-41, Sin Nombre virus, Sindbis virus, Small anellovirus, Sosuga virus, Spanish goat encephalitis virus, Spondweni virus, St. Louis encephalitis virus, Sunshine virus, TTV-like mini virus, Tacaribe mammarenavirus, Taila virus, Tamana bat virus, Tamiami mammarenavirus, Tembusu virus, Thogoto virus, Thottapalayam virus, Tick-borne encephalitis virus, Tioman virus, Togaviridae virus, Torque teno canis virus, Torque teno douroucouli virus, Torque teno felis virus, Torque teno midi virus, Torque teno sus virus, Torque teno tamarin virus, Torque teno virus, Torque teno zalophus virus, Tuhoko virus, Tula virus, Tupaia paramyxovirus, Usutu virus, Uukuniemi virus, Vaccinia virus, Variola virus, Venezuelan equine encephalitis virus, Vesicular stomatitis Indiana virus, WU Polyomavirus, Wesselsbron virus, West Caucasian bat virus, West Nile virus, Western equine encephalitis virus, Whitewater Arroyo mammarenavirus, Yellow fever virus, Yokose virus, Yug Bogdanovac virus, Zaire ebolavirus, Zika virus, or Zygosaccharomyces bailii virus Z viral sequence. Examples of RNA viruses that may be detected include one or more of (or any combination of) Coronaviridae virus, a Picornaviridae virus, a Caliciviridae virus, a Flaviviridae virus, a Togaviridae virus, a Bomaviridae, a Filoviridae, a Paramyxoviridae, a Pneumoviridae, a Rhabdoviridae, an Arenaviridae, a Bunyaviridae, an Orthomyxoviridae, or a Deltavirus. In certain example embodiments, the virus is Coronavirus, SARS, Poliovirus, Rhinovirus, Hepatitis A, Norwalk virus, Yellow fever virus, West Nile virus, Hepatitis C virus, Dengue fever virus, Zika virus, Rubella virus, Ross River virus, Sindbis virus, Chikungunya virus, Boma disease virus, Ebola virus, Marburg virus, Measles virus, Mumps virus, Nipah virus, Hendra virus, Newcastle disease virus, Human respiratory syncytial virus, Rabies virus, Lassa virus, Hantavirus, Crimean-Congo hemorrhagic fever virus, Influenza, or Hepatitis D virus.

In certain example embodiments, the virus may be a plant virus selected from the group comprising Tobacco mosaic virus (TMV), Tomato spotted wilt virus (TSWV), Cucumber mosaic virus (CMV), Potato virus Y (PVY), the RT virus Cauliflower mosaic virus (CaMV), Plum pox virus (PPV), Brome mosaic virus (BMV), Potato virus X (PVX), Citrus tristeza virus (CTV), Barley yellow dwarf virus (BYDV), Potato leafroll virus (PLRV), Tomato bushy stunt virus (TBSV), rice tungro spherical virus (RTSV), rice yellow mottle virus (RYMV), rice hoja blanca virus (RHBV), maize rayado fino virus (MRFV), maize dwarf mosaic virus (MDMV), sugarcane mosaic virus (SCMV), Sweet potato feathery mottle virus (SPFMV), sweet potato sunken vein closterovirus (SPSVV), Grapevine fanleaf virus (GFLV), Grapevine virus A (GVA), Grapevine virus B (GVB), Grapevine fleck virus (GFkV), Grapevine leafroll-associated virus-1, -2, and -3, (GLRaV-1, -2, and -3), Arabis mosaic virus (ArMV), or Rupestris stem pitting-associated virus (RSPaV). In a preferred embodiment, the target RNA molecule is part of said pathogen or transcribed from a DNA molecule of said pathogen. For example, the target sequence may be comprised in the genome of an RNA virus. It is further preferred that CRISPR effector protein hydrolyzes said target RNA molecule of said pathogen in said plant if said pathogen infects or has infected said plant. It is thus preferred that the CRISPR system is capable of cleaving the target RNA molecule from the plant pathogen both when the CRISPR system (or parts needed for its completion) is applied therapeutically, i.e. after infection has occurred or prophylactically, i.e. before infection has occurred.

In certain example embodiments, the virus may be a retrovirus. Example retroviruses that may be detected using the embodiments disclosed herein include one or more of or any combination of viruses of the Genus Alpharetrovirus, Betaretrovirus, Gammaretrovirus, Deltaretrovirus, Epsilonretrovirus, Lentivirus, Spumavirus, or the Family Metaviridae, Pseudoviridae, and Retroviridae (including HIV), Hepadnaviridae (including Hepatitis B virus), and Caulimoviridae (including Cauliflower mosaic virus).

In certain example embodiments, the virus is a DNA virus. Example DNA viruses that may be detected using the embodiments disclosed herein include one or more of (or any combination of) viruses from the Family Myoviridae, Podoviridae, Siphoviridae, Alloherpesviridae, Herpesviridae (including human herpes virus, and Varicella Zozter virus), Malocoherpesviridae, Lipothrixviridae, Rudiviridae, Adenoviridae, Ampullaviridae, Ascoviridae, Asfarviridae (including African swine fever virus), Baculoviridae, Cicaudaviridae, Clavaviridae, Corticoviridae, Fuselloviridae, Globuloviridae, Guttaviridae, Hytrosaviridae, Iridoviridae, Maseilleviridae, Mimiviridae, Nudiviridae, Nimaviridae, Pandoraviridae, Papillomaviridae, Phycodnaviridae, Plasmaviridae, Polydnaviruses, Polyomaviridae (including Simian virus 40, JC virus, BK virus), Poxviridae (including Cowpox and smallpox), Sphaerolipoviridae, Tectiviridae, Turriviridae, Dinodnavirus, Salterprovirus, Rhizidovirus, among others. In some embodiments, a method of diagnosing a species-specific bacterial infection in a subject suspected of having a bacterial infection is described as obtaining a sample comprising bacterial ribosomal ribonucleic acid from the subject; contacting the sample with one or more of the probes described, and detecting hybridization between the bacterial ribosomal ribonucleic acid sequence present in the sample and the probe, wherein the detection of hybridization indicates that the subject is infected with Escherichia coli, Klebsiella pneumoniae, Pseudomonas aeruginosa, Staphylococcus aureus, Acinetobacter baumannii, Candida albicans, Enterobacter cloacae, Enterococcus faecalis, Enterococcus faecium, Proteus mirabilis, Staphylococcus agalactiae, or Staphylococcus maltophilia or a combination thereof. The virus may also be a virus or a virus of the genus/family as described in Tables 8 and 9 of International Patent Publication WO2018/170340, incorporated herein by reference.

In certain embodiments, the virus is a drug resistant virus. By means of example, and without limitation, the virus may be a ribavirin resistant virus. Ribavirin is a very effective antiviral that hits a number of RNA viruses. Below are a few important viruses that have evolved ribavirin resistance. Foot and Mouth Disease Virus: doi:10.1128/JVI.03594-13. Polio virus: www.pnas.org/content/100/12/7289.full.pdf Hepatitis C Virus: jvi.asm.org/content/79/4/2346.full. A number of other persistent RNA viruses, such as hepatitis and HIV, have evolved resistance to existing antiviral drugs. Hepatitis B Virus (lamivudine, tenofovir, entecavir): doi:10.1002/hep.22900. Hepatitis C Virus (Telaprevir, BILN2061, ITMN-191, SCH6, Boceprevir, AG-021541, ACH-806): doi:10.1002/hep.22549. HIV has many drug resistant mutations, see hivdb.stanford.edu/for more information. Aside from drug resistance, there are a number of clinically relevant mutations that could be targeted with the CRISPR systems according to the invention as described herein. For instance, persistent versus acute infection in LCMV: doi:10.1073/pnas.1019304108; or increased infectivity of Ebola: doi:10.1016/j.cell.2016.10.014 and doi:10.1016/j.cell.2016.10.013.

In certain embodiments, the target sequences are diagnostic for monitoring drug resistance to treatment against malaria or other infectious diseases. Plasmodium, notably Plasmodia species affecting humans such as Plasmodium falciparum, Plasmodium vivax, Plasmodium ovale, Plasmodium malariae, and Plasmodium knowlesi are exemplary.

Further target sequences include sequences include target molecules/nucleic acid molecules coding for proteins involved in essential biological process for the Plasmodium parasite and notably transporter proteins, such as protein from drug/metabolite transporter family, the ATP-binding cassette (ABC) protein involved in substrate translocation, such as the ABC transporter C subfamily or the Na+/H+ exchanger, membrane glutathione S-transferase; proteins involved in the folate pathway, such as the dihydropteroate synthase, the dihydrofolate reductase activity or the dihydrofolate reductase-thymidylate synthase; and proteins involved in the translocation of protons across the inner mitochondrial membrane and notably the cytochrome b complex. Additional target may also include the gene(s) coding for the heme polymerase.

The invention allows to detect one or more mutation(s) and notably one or more single nucleotide polymorphisms in target nucleic acids/molecules. Accordingly, mutations can be used as drug resistance marker and can be detected according to the invention.

### Microfluidic Device

Microfluidic devices are also provided herein, that can be used for low volume, high-throughout sample processing; the various steps from sample collection to diagnostic read-out can be integrated into a reusable microfluidic chip. In some embodiments, the microfluidic devices are provided as part of the cfDNA detection systems. In some embodiments, the systems couple channel-based microfluidics with micro-well systems for target detection.

A microfluidic device comprising a sample loading region, and one or more flow channels, each channel comprising a detector region comprising a detection construct and one or more nucleic acid detection systems, and at least a first and second capture region, the first capture region comprising a first binding agent and the second capture region comprising a second binding agent.

The microfluidic device may comprise a node at each region of the microfluidic flow device. A node as utilized herein may also be a bubble, or can be referred to herein as an individual discrete volume. The node, bubble, or indiscrete volume may comprise an aperture that allows for accessibility, or may be a closed system that is optionally accessed by a removable lid or cover. The nodes or bubbles may be pre-loaded with one or more of the reagents disclosed herein, including detection constructs. Any of the pre-loaded components may be provided as freeze-dried reagents for corresponding steps of the detection reactions disclosed herein. The microfluidic device, can comprise flow channels arranged in any manner that allows the flow of the sample in the direction of the detection steps disclosed herein. In particular embodiments, the flow channel is arranged radially or in parallel from a center node. In embodiments, the sample input is disposed to run in a flow direction to a center node that radially expands out from the center node. One or more flow channels may be disposed between the sample input and a central node that radially expands out. The center node may be the sample input. In another aspect, the flow channels run parallel to each other for multiplexing detection reactions. In an aspect, the center node comprises transcription reagents. The microfluidic device can comprise one or more thermally differentiated zones disposed between the sample loading region and the center node.

The detection constructs can be as described elsewhere herein, and can be provided separate from the microfluidic device or disposed within the microfluidic device nodes.

The microfluidic device can comprise one or more nucleic acid detection systems wherein each comprise a Cas protein, and one or more species-specific guide RNA, as described elsewhere herein. In particular embodiments, the Cas protein is a Cas 13 protein, as detailed elsewhere herein. In an aspect, the Cas protein is a Cas13a, 13b or 13c protein.

The microfluidic device may further comprise one or more amplification reagents, optionally in the sample loading region. The microfluidic device may comprise amplification reagents, MIPs, primers, guides, and CRISPR systems specific for one or more targets, which, as described herein may include, genus, species, variants, and other desired targets. In embodiments, each flow channel is specific for a desired target. Reagents and other detection system components can be comprised at distinct nodes in a flow channel. The direction of the flow of the fluid sample allows the sequential reaction of the sample along the one or more flow channels.

In embodiments, the guide RNA utilized in the microfluidic device is designed to target an amplicon of the sample. The microfluidic device may comprise molecular inversion probes as described herein and ligation reagents in the sample loading region. In an aspect, species-specific guide RNA designed to target a species-specific binding sequence on the MIP.

The microfluidic device may comprise one or more amplification reagents, which may be selected from nucleic acid sequence-based amplification (NASBA), recombinase polymerase amplification (RPA), loop-mediated isothermal amplification (LAMP), strand displacement amplification (SDA), helicase-dependent amplification (HDA), nicking enzyme amplification reaction (NEAR), PCR, multiple displacement amplification (MDA), rolling circle amplification (RCA), ligase chain reaction (LCR), or ramification amplification method (RAM). In particular embodiments, amplification reagents may comprise primers and may be provided at a sample input region such that the sample, the amplification reagents flow through thermally differentiated zones thereby amplifying one or more sequences in the sample prior to the sample flowing further through the microfluidic device. Subsequent to the amplification, transcription reagents can be optionally included to transcribe the sample. Continuing flow through the microfluidic device, the sample can flow in parallel or radially outward to a variety of flow channels or branches where CRISPR systems, guide sequences, and detection constructs are provided in nodes or bubbles of the flow channel. Reactions may occur at each node of the flow channel, depending on the content of the sample, the presence of targets, the designed probes, and the detection constructs. Readouts of the reaction can be generated in this manner, and can allow for detection of a plurality of targets, for example, pathogens, in each flow channel.

Kits comprising the microfluidic device with the detection systems as provided herein are also provided for detection of one or more pathogens and/or discriminating between one or more diseases.

Utilizing microfluidic devices in exemplary embodiments can include mixing each component with a unique combination of three encoding dyes. Droplets are then generated separately for each component, pooled together, and loaded onto a microwell array with tens of thousands of wells, each designed to hold exactly two droplets. Droplets settle into wells in a stochastic fashion, giving rise to all possible pair-wise combination of components. Fluorescence microscopy is then used to determine droplet identity based on encoding dye signal. The droplets are then merged using a corona generator (which destabilizes droplet surfaces) and a fourth fluorescence channel is used to readout an assay score after incubation, and is amenable to CRISPR-diagnostics, as disclosed herein.

Microfluidic devices comprise an array of microwells with at least one flow channel beneath the microwells. In certain example embodiments, the device is a microfluidic device that generates and/or merges different droplets (i.e. individual discrete volumes). For example, a first set of droplets may be formed containing samples to be screened and a second set of droplets formed containing the elements of the systems described herein. The first and second set of droplets are then merged and then diagnostic methods as described herein are carried out on the merged droplet set.

Microfluidic devices disclosed herein may be silicone-based chips and may be fabricated using a variety of techniques, including, but not limited to, hot embossing, molding of elastomers, injection molding, LIGA, soft lithography, silicon fabrication and related thin film processing techniques. Suitable materials for fabricating the microfluidic devices include, but are not limited to, cyclic olefin copolymer (COC), polycarbonate, poly(dimethylsiloxane) (PDMS), and poly(methylacrylate) (PMMA). In one embodiment, soft lithography in PDMS may be used to prepare the microfluidic devices. For example, a mold may be made using photolithography which defines the location of flow channels, valves, and filters within a substrate. The substrate material is poured into a mold and allowed to set to create a stamp. The stamp is then sealed to a solid support, such as but not limited to, glass. Due to the hydrophobic nature of some polymers, such as PDMS, which absorbs some proteins and may inhibit certain biological processes, a passivating agent may be necessary (Schoffner et al. Nucleic Acids Research, 1996, 24:375-379). Suitable passivating agents are known in the art and include, but are not limited to, silanes, parylene, n-Dodecyl-b-D-matoside (DDM), pluronic, Tween-20, other similar surfactants, polyethylene glycol (PEG), albumin, collagen, and other similar proteins and peptides.

An example of microfluidic device that may be used in the context of the invention is described in Kulesa, et al. PNAS, 115, 6685-6690, incorporated herein by reference.

In certain example embodiments, the device may comprise individual wells, such as microplate wells. The size of the microplate wells may be the size of standard 6, 24, 96, 384, 1536, 3456, or 9600 sized wells. In certain embodiments, the microwells can number at more than 40,0000 or more than 190,000. In certain example embodiments, the elements of the systems described herein may be freeze dried and applied to the surface of the well prior to distribution and use.

Microwell chips can be designed as disclosed in U.S. Provisional Application Attorney Docket No. 52199-505P03US. In one embodiment, the microwell chip can be designed in a format measuring around 6.2×7.2 cm, containing 49200 microwells, or a larger format, measuring 7.4×10 cm, containing 97, 194 microwells. The array of microwells can be shaped, for example, as two circles of a diameter of about 50-300 m, in particular embodiments at 150 μm diameter set at 10% overlap. The array of microwells can be arranged in a hexagonal lattice at 50 μm inter-well spacing. In some instances, the microwells can be arranged in other shapes, spacing and sizes in order to hold a varying number of droplets. The microwell chips are advantageously, in some embodiments, sized for use with standard laboratory equipment, including imaging equipment such as microscopes. In some embodiments, the microwell chips can be configured to be utilized with standard microscopes as well as microscopes that include means for incubation. In instances where imaging and incubation can be coupled, assays can enable fluorescence kinetics and quantitation.

In an exemplary method, compounds can be mixed with a unique ratio of fluorescent dyes (e.g. Alexa Fluor 555, 594, 647). Each mixture of target molecule with a dye mixture can be emulsified into droplets. Similarly, each detection CRISPR system with optical barcode can be emulsified into droplets. In some embodiments, the droplets are approximately 1 nL each. The CRISPR detection system droplets and target molecule droplets can then be combined and applied to the microwell chip. The droplets can be combined by simple mixing or other methods of combination. In one exemplary embodiment, the microwell chip is suspended on a platform such as a hydrophobic glass slide with removable spacers that can be clamped from above and below by clamps or other securing means, which can be, for example, neodymium magnets. The gap between the chip and the glass created by the spacers can be loaded with oil, and the pool of droplets injected into the chip, continuing to flow the droplets by injecting more oil and draining excess droplets. After loading is completed, the chip can be washed with oil, and spacers can be removed to seal microwells against the glass slide and clamp closed. The chip can be imaged, for example with an epifluorescence microscope, droplets merged to mix the compounds in each microwell by applying an AC electric field, for example, supplied by a corona treater, and subsequently treated according to desired protocols. In one embodiment, the microwell can be incubated at 37° C. with measurement of fluorescence using epifluoresecnce microscope. Following manipulation of the droplets, the droplets can be eluted off of the microwell as described herein for additional analyses, processing and/or manipulations.

The devices disclosed may further comprise inlet and outlet ports, or openings, which in turn may be connected to valves, tubes, channels, chambers, and syringes and/or pumps for the introduction and extraction of fluids into and from the device. In certain embodiments, the channel-based microfluidic devices are coupled with micro-well systems, or valve and channel-based microfluidic devices are used. The devices may be connected to fluid flow actuators that allow directional movement of fluids within the microfluidic device. Example actuators include, but are not limited to, syringe pumps, mechanically actuated recirculating pumps, electroosmotic pumps, bulbs, bellows, diaphragms, or bubbles intended to force movement of fluids. In certain example embodiments, the devices are connected to controllers with programmable valves that work together to move fluids through the device. In certain example embodiments, the devices are connected to the controllers discussed in further detail below. The devices may be connected to flow actuators, controllers, and sample loading devices by tubing that terminates in metal pins for insertion into inlet ports on the device.

Digital microfluidics can also be utilized with the methods and systems disclosed herein0. The present invention may be used with a wireless lab-on-chip (LOC) diagnostic sensor system (see e.g., U.S. Pat. No. 9,470,699 “Diagnostic radio frequency identification sensors and applications thereof”). In certain embodiments, the present invention is performed in a LOC controlled by a wireless device (e.g., a cell phone, a personal digital assistant (PDA), a tablet) and results are reported to said device.

Radio frequency identification (RFID) tag systems include an RFID tag that transmits data for reception by an RFID reader (also referred to as an interrogator). In a typical RFID system, individual objects (e.g., store merchandise) are equipped with a relatively small tag that contains a transponder. The transponder has a memory chip that is given a unique electronic product code. The RFID reader emits a signal activating the transponder within the tag through the use of a communication protocol. Accordingly, the RFID reader is capable of reading and writing data to the tag. Additionally, the RFID tag reader processes the data according to the RFID tag system application. Currently, there are passive and active type RFID tags. The passive type RFID tag does not contain an internal power source, but is powered by radio frequency signals received from the RFID reader. Alternatively, the active type RFID tag contains an internal power source that enables the active type RFID tag to possess greater transmission ranges and memory capacity. The use of a passive versus an active tag is dependent upon the particular application.

Lab-on-the chip technology is well described in the scientific literature and consists of multiple microfluidic channels, input or chemical wells. Reactions in wells can be measured using radio frequency identification (RFID) tag technology since conductive leads from RFID electronic chip can be linked directly to each of the test wells. An antenna can be printed or mounted in another layer of the electronic chip or directly on the back of the device. Furthermore, the leads, the antenna and the electronic chip can be embedded into the LOC chip, thereby preventing shorting of the electrodes or electronics. Since LOC allows complex sample separation and analyses, this technology allows LOC tests to be done independently of a complex or expensive reader. Rather a simple wireless device such as a cell phone or a PDA can be used. In one embodiment, the wireless device also controls the separation and control of the microfluidics channels for more complex LOC analyses. In one embodiment, a LED and other electronic measuring or sensing devices are included in the LOC-RFID chip. Not being bound by a theory, this technology is disposable and allows complex tests that require separation and mixing to be performed outside of a laboratory.

In preferred embodiments, the LOC may be a microfluidic device. The LOC may be a passive chip, wherein the chip is powered and controlled through a wireless device. In certain embodiments, the LOC includes a microfluidic channel for holding reagents and a channel for introducing a sample. In certain embodiments, a signal from the wireless device delivers power to the LOC and activates mixing of the sample and assay reagents. Specifically, in the case of the present invention, the system may include a masking agent, CRISPR effector protein, and guide RNAs specific for a target molecule. Upon activation of the LOC, the microfluidic device may mix the sample and assay reagents. Upon mixing, a sensor detects a signal and transmits the results to the wireless device. In certain embodiments, the unmasking agent is a conductive RNA molecule. The conductive RNA molecule may be attached to the conductive material. Conductive molecules can be conductive nanoparticles, conductive proteins, metal particles that are attached to the protein or latex or other beads that are conductive. In certain embodiments, if DNA or RNA is used then the conductive molecules can be attached directly to the matching DNA or RNA strands. The release of the conductive molecules may be detected across a sensor. The assay may be a one step process.

Since the electrical conductivity of the surface area can be measured precisely quantitative results are possible on the disposable wireless RFID electro-assays. Furthermore, the test area can be very small allowing for more tests to be done in a given area and therefore resulting in cost savings. In certain embodiments, separate sensors each associated with a different CRISPR effector protein and guide RNA immobilized to a sensor are used to detect multiple target molecules. Not being bound by a theory, activation of different sensors may be distinguished by the wireless device.

In addition to the conductive methods described herein, other methods may be used that rely on RFID or Bluetooth as the basic low-cost communication and power platform for a disposable RFID assay. For example, optical means may be used to assess the presence and level of a given target molecule. In certain embodiments, an optical sensor detects unmasking of a fluorescent masking agent.

In certain embodiments, the device of the present invention may include handheld portable devices for diagnostic reading of an assay (see e.g., Vashist et al., Commercial Smartphone-Based Devices and Smart Applications for Personalized Healthcare Monitoring and Management, Diagnostics 2014, 4(3), 104-128; mReader from Mobile Assay; and Holomic Rapid Diagnostic Test Reader).

As noted herein, certain embodiments allow detection via colorimetric change which has certain attendant benefits when embodiments are utilized in POC situations and or in resource poor environments where access to more complex detection equipment to readout the signal may be limited. However, portable embodiments disclosed herein may also be coupled with hand-held spectrophotometers that enable detection of signals outside the visible range. An example of a hand-held spectrophotometer device that may be used in combination with the present invention is described in Das et al. “Ultra-portable, wireless smartphone spectrophotometer for rapid, non-destructive testing of fruit ripeness.” Nature Scientific Reports. 2016, 6:32504, DOI: 10.1038/srep32504. Finally, in certain embodiments utilizing quantum dot-based masking constructs, use of a hand-held UV light, or other suitable device, may be successfully used to detect a signal owing to the near complete quantum yield provided by quantum dots.

An “individual discrete volume” is a discrete volume or discrete space, such as a container, receptacle, or other defined volume or space that can be defined by properties that prevent and/or inhibit migration of nucleic acids, CRISPR detection systems, and reagents necessary to carry out the methods disclosed herein, for example a volume or space defined by physical properties such as walls, for example the walls of a well, tube, or a surface of a droplet, which may be impermeable or semipermeable, or as defined by other means such as chemical, diffusion rate limited, electro-magnetic, or light illumination, or any combination thereof. In particularly preferred embodiments, the individual discrete volumes are droplets. By “diffusion rate limited” (for example diffusion defined volumes) is meant spaces that are only accessible to certain molecules or reactions because diffusion constraints effectively defining a space or volume as would be the case for two parallel laminar streams where diffusion will limit the migration of a target molecule from one stream to the other. By “chemical” defined volume or space is meant spaces where only certain target molecules can exist because of their chemical or molecular properties, such as size, where for example gel beads may exclude certain species from entering the beads but not others, such as by surface charge, matrix size or other physical property of the bead that can allow selection of species that may enter the interior of the bead. By “electro-magnetically” defined volume or space is meant spaces where the electro-magnetic properties of the target molecules or their supports such as charge or magnetic properties can be used to define certain regions in a space such as capturing magnetic particles within a magnetic field or directly on magnets. By “optically” defined volume is meant any region of space that may be defined by illuminating it with visible, ultraviolet, infrared, or other wavelengths of light such that only target molecules within the defined space or volume may be labeled. One advantage to the use of non-walled, or semipermeable is that some reagents, such as buffers, chemical activators, or other agents maybe passed in or through the discrete volume, while other material, such as target molecules, maybe maintained in the discrete volume or space. As explained herein, a droplet system allows for the separation of compounds until initiation of a reaction is desired. Typically, a discrete volume will include a fluid medium, (for example, an aqueous solution, an oil, a buffer, and/or a media capable of supporting cell growth) suitable for labeling of the target molecule with the indexable nucleic acid identifier under conditions that permit labeling. Exemplary discrete volumes or spaces useful in the disclosed methods include droplets (for example, microfluidic droplets and/or emulsion droplets), hydrogel beads or other polymer structures (for example poly-ethylene glycol di-acrylate beads or agarose beads), tissue slides (for example, fixed formalin paraffin embedded tissue slides with particular regions, volumes, or spaces defined by chemical, optical, or physical means), microscope slides with regions defined by depositing reagents in ordered arrays or random patterns, tubes (such as, centrifuge tubes, microcentrifuge tubes, test tubes, cuvettes, conical tubes, and the like), bottles (such as glass bottles, plastic bottles, ceramic bottles, Erlenmeyer flasks, scintillation vials and the like), wells (such as wells in a plate), plates, pipettes, or pipette tips among others. In certain example embodiments, the individual discrete volumes are droplets.

### Droplets

The droplets as provided herein are typically water-in-oil microemulsions formed with an oil input channel and an aqueous input channel. The droplets can be formed by a variety of dispersion methods known in the art. In one particular embodiment, a large number of uniform droplets in oil phase can be made by microemulsion. Exemplary methods can include, for example, R-junction geometry where an aqueous phase is sheared by oil and thereby generates droplets; flow-focusing geometry where droplets are produced by shearing the aqueous stream from two directions; or co-flow geometry where an aqueous phase is ejected through a thin capillary, placed coaxially inside a bigger capillary through which oil is pumped.

The use of monodisperse aqueous droplets can be generated by a microfluidic device as a water-in-oil emulsion. In one embodiment, the droplets are carried in a flowing oil phase and stabilized by a surfactant. In one aspect single cells or single organelles or single molecules (proteins, RNA, DNA) are encapsulated into uniform droplets from an aqueous solution/dispersion. In a related aspect, multiple cells or multiple molecules may take the place of single cells or single molecules.

The aqueous droplets of volume ranging from 1 pL to 10 nL work as individual reactors. 104 to 10′ single cells in droplets may be processed and analyzed in a single run. To utilize microdroplets for rapid large-scale chemical screening or complex biological library identification, different species of microdroplets, each containing the specific chemical compounds or biological probes cells or molecular barcodes of interest, have to be generated and combined at the preferred conditions, e.g., mixing ratio, concentration, and order of combination. Each species of droplet is introduced at a confluence point in a main microfluidic channel from separate inlet microfluidic channels. Preferably, droplet volumes are chosen by design such that one species is larger than others and moves at a different speed, usually slower than the other species, in the carrier fluid, as disclosed in U.S. Publication No. US 2007/0195127 and International Publication No. WO 2007/089541, each of which are incorporated herein by reference in their entirety. The channel width and length is selected such that faster species of droplets catch up to the slowest species. Size constraints of the channel prevent the faster moving droplets from passing the slower moving droplets resulting in a train of droplets entering a merge zone. Multi-step chemical reactions, biochemical reactions, or assay detection chemistries often require a fixed reaction time before species of different type are added to a reaction. Multi-step reactions are achieved by repeating the process multiple times with a second, third or more confluence points each with a separate merge point. Highly efficient and precise reactions and analysis of reactions are achieved when the frequencies of droplets from the inlet channels are matched to an optimized ratio and the volumes of the species are matched to provide optimized reaction conditions in the combined droplets. Fluidic droplets may be screened or sorted within a fluidic system of the invention by altering the flow of the liquid containing the droplets. For instance, in one set of embodiments, a fluidic droplet may be steered or sorted by directing the liquid surrounding the fluidic droplet into a first channel, a second channel, etc. In another set of embodiments, pressure within a fluidic system, for example, within different channels or within different portions of a channel, can be controlled to direct the flow of fluidic droplets. For example, a droplet can be directed toward a channel junction including multiple options for further direction of flow (e.g., directed toward a branch, or fork, in a channel defining optional downstream flow channels). Pressure within one or more of the optional downstream flow channels can be controlled to direct the droplet selectively into one of the channels, and changes in pressure can be effected on the order of the time required for successive droplets to reach the junction, such that the downstream flow path of each successive droplet can be independently controlled.

The invention can thus involve forming sample droplets. The droplets are aqueous droplets that are surrounded by an immiscible carrier fluid. Methods of forming such droplets are shown for example in Link et al. (U.S. patent application numbers 2008/0014589, 2008/0003142, and 2010/0137163), Stone et al. (U.S. Pat. No. 7,708,949 and U.S. patent application number 2010/0172803), Anderson et al. (U.S. Pat. No. 7,041,481 and which reissued as RE41,780) and European publication number EP2047910 to Raindance Technologies Inc. The content of each of which is incorporated by reference herein in its entirety. The present invention may relate to systems and methods for manipulating droplets within a high throughput microfluidic system.

From this disclosure and herein cited documents and knowledge in the art, it is within the ambit of the skilled person to develop flow rates, channel lengths, and channel geometries; and establish droplets containing random or specified reagent combinations can be generated on demand and merged with the “reaction chamber” droplets containing the samples/cells/substrates of interest. By incorporating a plurality of unique tags into the additional droplets and joining the tags to a solid support designed to be specific to the primary droplet, the conditions that the primary droplet is exposed to may be encoded and recorded. For example, nucleic acid tags can be sequentially ligated to create a sequence reflecting conditions and order of same. Alternatively, the tags can be added independently appended to solid support. Non-limiting examples of a dynamic labeling system that may be used to bioinformatically record information can be found at US Provisional Patent Application entitled “Compositions and Methods for Unique Labeling of Agents” filed Sep. 21, 2012 and Nov. 29, 2012. In this way, two or more droplets may be exposed to a variety of different conditions, where each time a droplet is exposed to a condition, a nucleic acid encoding the condition is added to the droplet each ligated together or to a unique solid support associated with the droplet such that, even if the droplets with different histories are later combined, the conditions of each of the droplets are remain available through the different nucleic acids. Non-limiting examples of methods to evaluate response to exposure to a plurality of conditions can be found at US Provisional Patent Application filed Sep. 21, 2012, and U.S. patent application Ser. No. 15/303,874 filed Apr. 17, 2015 entitled “Systems and Methods for Droplet Tagging.” Accordingly, in or as to the invention it is envisioned that there can be the dynamic generation of molecular barcodes (e.g., DNA oligonucleotides, fluorophores, etc.) either independent from or in concert with the controlled delivery of various compounds of interest (siRNA, CRISPR guide RNAs, reagents, etc.). For example, unique molecular barcodes can be created in one array of nozzles while individual compounds or combinations of compounds can be generated by another nozzle array. Barcodes/compounds of interest can then be merged with CRISPR detection system-containing droplets. An electronic record in the form of a computer log file is kept to associate the barcode delivered with the downstream reagent(s) delivered. This methodology makes it possible to efficiently screen a large population of samples according to the methods disclosed herein. The device and techniques of the disclosed invention facilitate efforts to perform studies that require data resolution at the single cell (or single molecule) level and in a cost-effective manner. A high throughput and high-resolution delivery of reagents to individual emulsion droplets that may contain samples of target molecules for further evaluation through the use of monodisperse aqueous droplets that are generated one by one in a microfluidic chip as a water-in-oil emulsion.

### Methods

Detection methods can comprise an overall process comprising several steps that can be optimized according to the desired targets. In a first step, target selection and MIP/primer design is selected. When MIPs are utilized, a second step of hybridization and ligation of MIPs is included in the process. When utilizing primer-based detection, such ligation is not required. The next step is amplification, which may be optional, can include PCR, HDA, RPA, or RCA as exemplary amplifications. Detection can include optional T7 RNA polymerase to transcribe when using Cas13 proteins that collaterally cleave RNA.

Regarding target selection and MIP/primer design, there are advantages aned disadvantages to each approach, which may be selected and designed based on some of the following considerations when tailoring detection methods according to the present invention. Advantages of MIPs include MIP backbone provides for specificity of amplification and detection, use of common primers and shared gRNA binding sites greatly enhancing possibilities for multiplexing, can detect small (˜20 to about 100 bp, or about 40 to about 50 bp, or about 46 to about 52 bp, or about 48 bp) gDNA fragments. Some disadvantages using MIPs include that additional step(s) can be required, as well as some contribution to signal from linear (unligated) MIP being possible depending on amplification strategy. Regarding direct amplification, advantages include no ligation step and that direct amplification strategies typically have single-copy sensitivity. Disadvantages to direct amplification include the requirement of slightly larger fragments, e.g. >75 bp, a unique gRNA is required for each target, and that tiling is more challenging due to complex mixtures of primers required.

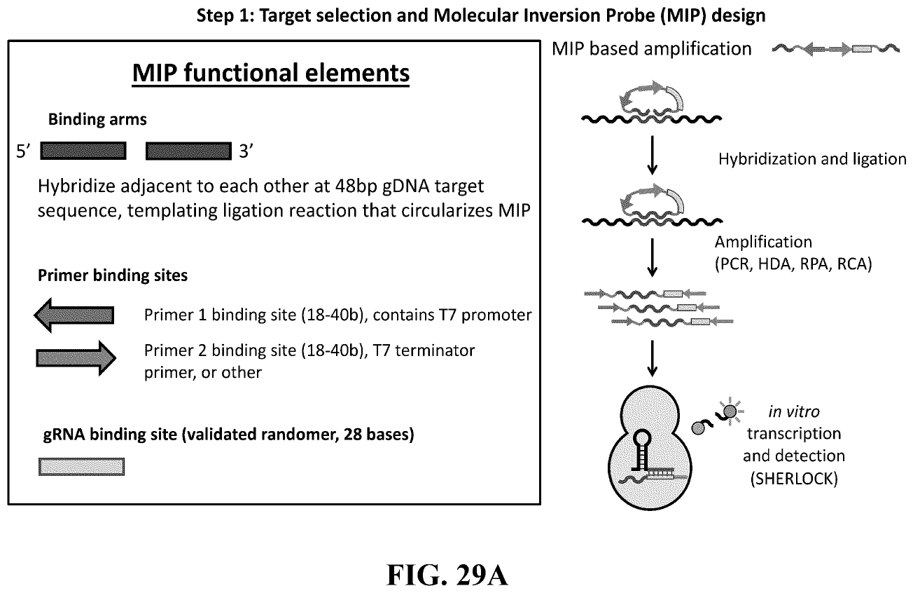

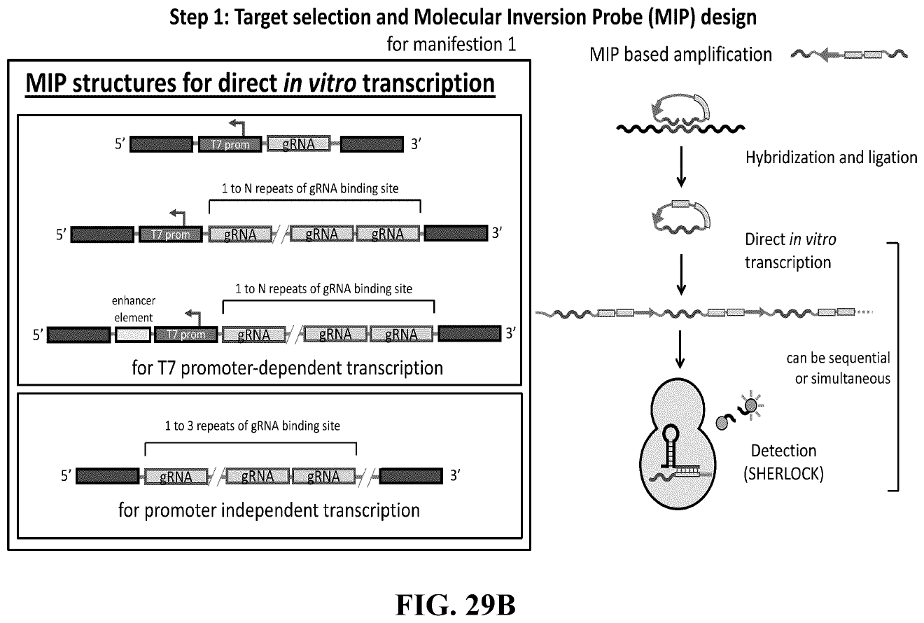

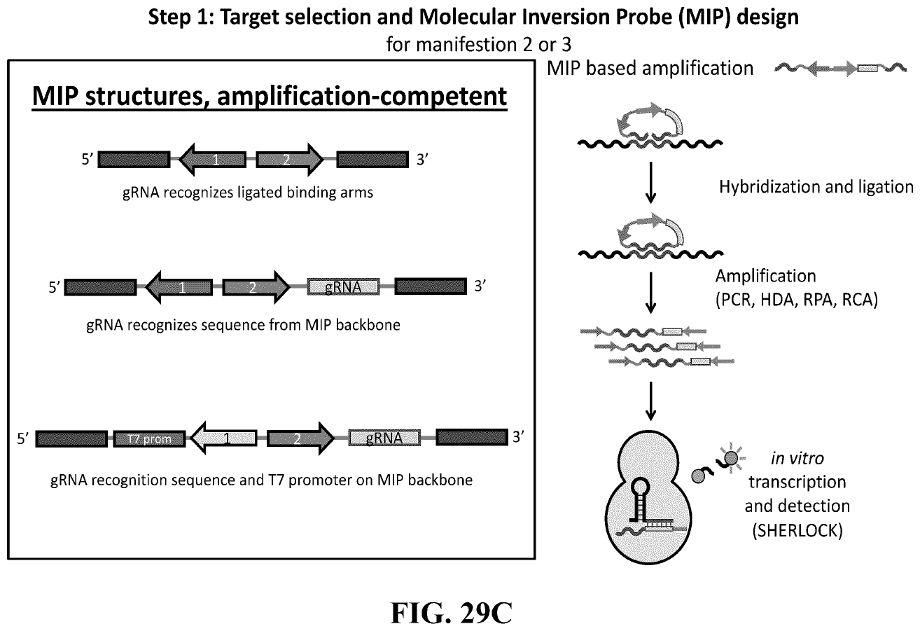

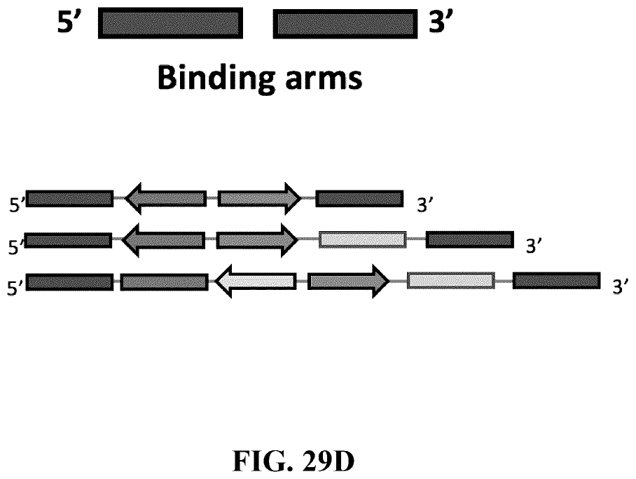

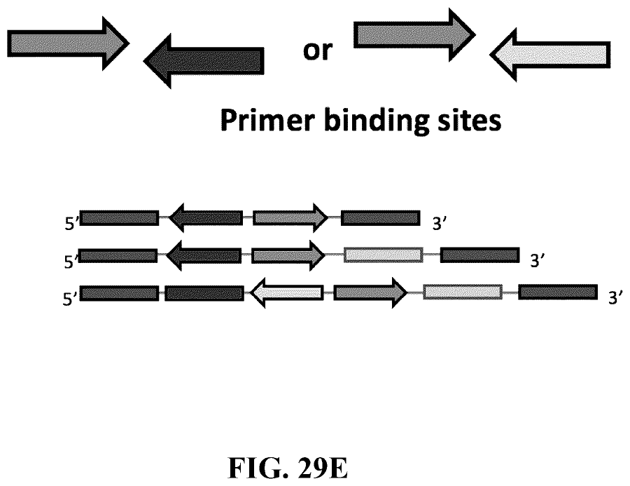

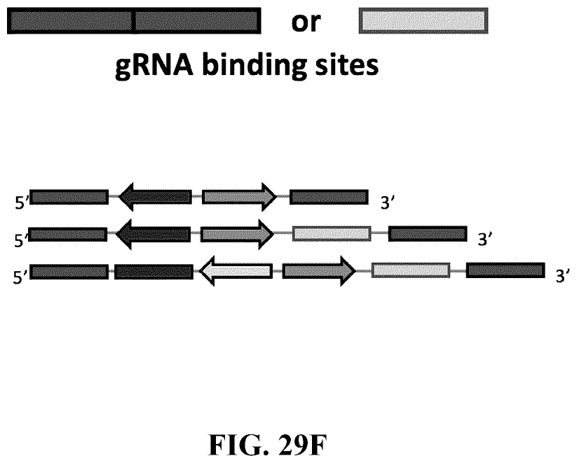

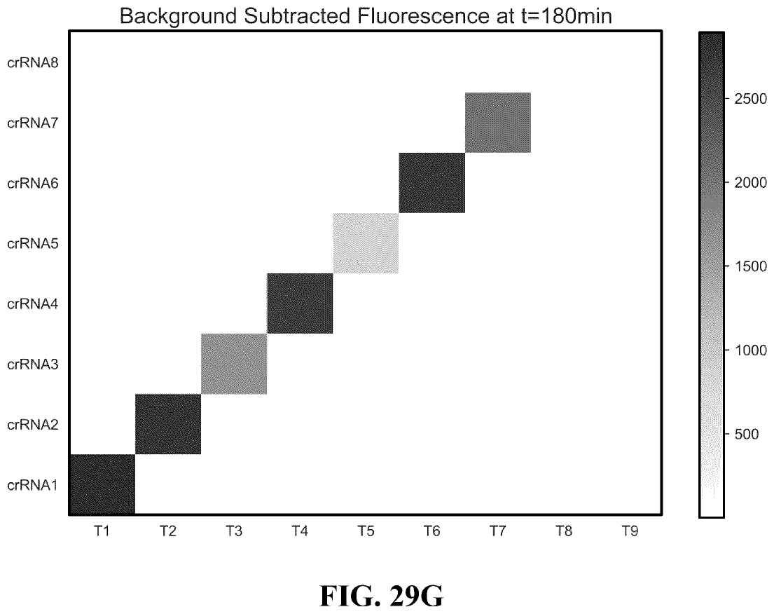

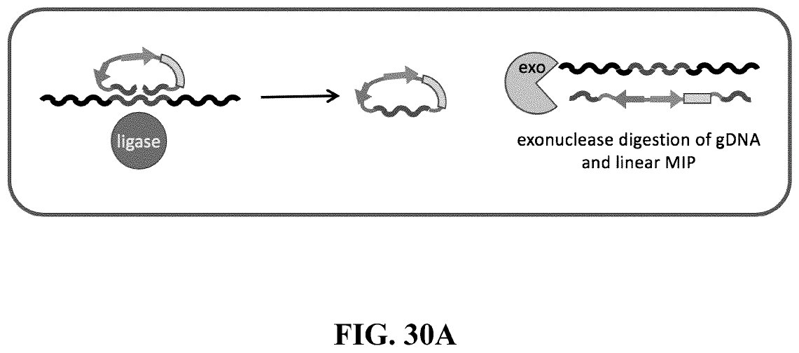

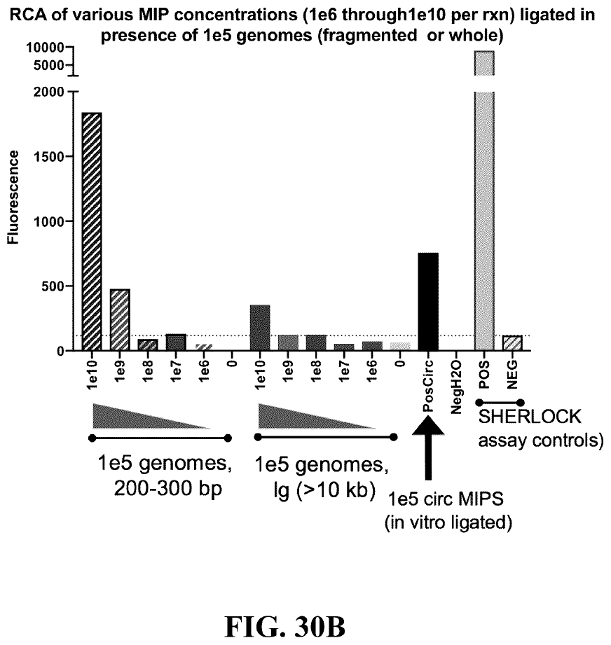

As shown in FIG. 29A, MIP functional elements can include binding arms, primer binding sites, and gRNA binding site. The gRNA binding site can include a 28base randomer that is a defined sequence. The binding arms hybridize to a target sequence. In embodiments, the binding arms can hybridize adjacent to each other or with a gap region at a target sequence. When the binding arms hybridize to a target sequence with a gap, optionally, reverse polymerization can be used in a gap filling step. In embodiments, the detection is about 20 to 200 base pairs.

MIP structures for direct in vitro transcription can be provided, and may comprise T7 promoters. Optionally enhancer elements can be included in the MIP backbone, as well as 1 to N repeats of the gRNA binding sites. The MIP backbone can vary based on detection design, including promoter-dependent transcription approaches and promoter independent transcription. The gRNA binding sites may be designed to be 1 to 50 repeats of the same target, or may be designed for detection of different targets. In certain aspects, 1 to 3 repeats of a gRNA binding site are provided. In an aspect, direct in vitro transcription and detection steps can be sequential or simultaneous.

In certain aspects, MIP structures are amplification competent, wherein the guide sequence recognizes ligated binding arms, sequence from the MIP backbone, or a guide sequence recognition sequence and a promoter on the MIP backbone. See, e.g. FIG. 29C for exemplary designs.

Target selection and binding arm design can comprise identification of N-mers, for example, 48-mers, that are conserved across all sequenced isolates of a given species, for example S. aureus. In an example, this step eliminates ˜50% of S. aureus 48-mers. Identification of N-mers that are not present in a defined outgroup (can include other bacterial species within or outside genus, human, other microbes; ˜70% of S. aureus 48-mers are present in other Staphylococcus species) can be utilized in binding arm design. The N-mers can vary from 20 to 200 mers, and may depend on target, species and sample. Next, the N-mers identified can be split in half, for example for a 48-mer can be split into 24-mers and remove pairs with melting temperature differences greater than 2° C. (eliminates ˜96% of S. aureus pairs). Next, design may comprise filtering to eliminate base pairing between final 5 bases of 3′ binding arm and rest of MIP. An additional step can include applying additional filters based on: Multiplexing (base pairing of 3′ end with other MIPs) and Performance.

When using MIPs in the detection methods disclosed herein, the hybridization and ligation of MIPs can be optimized. Factors one of skill in the art may consider in optimizing this step of the process include: Reagent (MIP) concentration, DNA status (large vs small fragments), duration of ligation, specificity of ligation, cycling to increase number of circularized MIPs, removal of unligated MIPs. Reagent (MIP) concentration can be increased to facilitate ligation. Additionally, DNA status can be adjusted, for example, by sample preparation, where increased ligation are needed, as smaller fragments are better at templating ligation. In an aspect, duration of ligation can be adjusted, with the duration of ligation inversely correlated with MIP concentration. Specificity of ligation can further be optimized, as well as cycling. Cycling can be utilized to increase the number of circularized MIPs, which relates to ligation kinetics, kinetics which are linked to MIP concentration. Cycling requires rapid annealing/ligation, i.e. high MIP concentrations. This MIP concentration can be adjusted using the methods discussed herein. Removal of unligated linear MIPs can be performed by digestion, for example using Exonuclease I/Exonuclease 3.

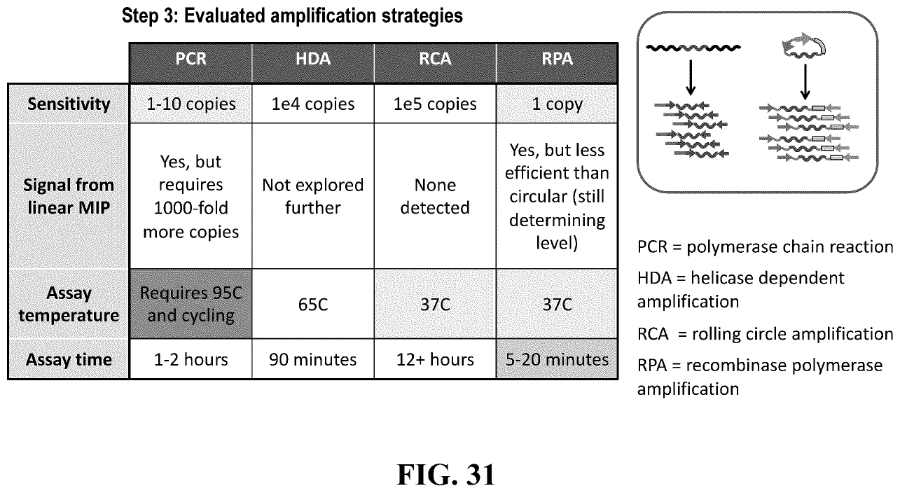

The detection methodologies can include amplification. Evaluation of amplification strategies are shown in FIG. 31. Considerations include sensitivity, the signal from linear MIP, assay temperature and assay time.

Methods disclosed herein include steps of conducting target specific or non-specific preamplification on cell free nucleic acid from a blood or urine sample; generating a first set of droplets, each droplet in the first set of droplets comprising at least one target molecule from the sample and an optical barcode; generating a second set of droplets, each droplet in the second set of droplets comprising one or more detection CRISPR systems comprising an effector protein and one or more guide RNAs tiled to corresponding target sequences unique to the one or more strains or one or more pathogens, an RNA-based masking construct and optionally an optical barcode; combining the first set and second set of droplets into a pool of droplets and flowing the pool of droplets onto a microfluidic device comprising an array of microwells and at least one flow channel beneath the microwells, the microwells sized to capture at least two droplets; detecting the optical barcodes of the droplets captured in each microwell; merging the droplets captured in each microwell to form merged droplets in each microwell, at least a subset of the merged droplets comprising a detection CRISPR system and a target sequence; initiating the detection reaction by incubating at about 37° C.; and measuring a detectable signal of each merged droplet at one or more time periods.

### Droplet Generation and Combining of Droplets

Regarding generation of a first set of droplets, in one aspect generating a first set of droplets, each first droplet containing a detection CRISPR system, the detection CRISPR system can comprise an RNA targeting effector protein and one or more guide RNAs designed to bind to corresponding target molecules, an RNA-based masking construct and an optical barcode as described herein. In particular embodiments the step of generating a second set of droplets each droplet in the second set of droplets comprises at least one target molecule and an optical barcode as provided herein.

Subsequent to generation of a first set of droplets and a second set of droplets, the first set and second set of droplets are combined into a pool of droplets. The combining can be effected by any means to combine the first and second sets. In one exemplary embodiment, the sets of droplets are mixed to combine into a pool of droplets.

Once a pool of droplets is generated, the step of flowing the pool of droplets is performed. The flowing of the pool of droplets is performed by loading the droplets onto a microfluidic device containing a plurality of microwells. The microwells are sized to capture at least two droplets. Optionally, subsequent to loading, surfactant is washed out.

Once the droplets are loaded into the microwell array, a step of detecting the optical barcode of the droplets captured in each microwell is performed. In some instances, the detecting the optical barcode is performed by low magnification fluorescence scan when the optical barcodes are fluorescence barcodes. Regardless of the type of optical barcode, the barcodes for each droplet are unique, and thus the content of each droplet can be identified. The manner of detection will be selected according to the type of optical barcode utilized. The droplets contained in each microwell are then merged. Merging can be performed by applying an electrical field. At least a subset of the merged droplets comprise a detection CRISPR system and a target sequence.

After merging of the droplets, the detection reaction is then initiated. In some embodiments, initiating the detection reaction comprises incubating the merged droplets. Subsequent to the detection reaction, the merged droplets are subjected to an optical assay, which in some instances is a low magnification fluorescence scan to generate an assay score.

In some embodiments, the methods can comprise a step of amplifying target molecules. Amplification of the target molecules can be performed prior to or subsequent to the generation of the first set of droplets.

As described herein, samples containing target molecules to which the guide RNAs are targeted, are loaded into one set of droplets and merged with droplet(s) comprising the guide RNAs and CRISPR system. Reporter systems incorporated in the CRISPR system droplets express an optically detectable marker (e.g. fluorescent protein) in the masking construct. The set of droplets including a CRISPR system comprising an effector protein and one or more guide RNAs designed to bind to corresponding target molecules, and an RNA-based masking construct. After the droplets are merged, the identity of the molecular species in each well can be determined by optically scanning each microwell to read the optical barcode. Optical measurement of the reporter system can occur simultaneously with optical scanning of the barcode. Thus, simultaneous gathering of experimental data and molecular species identification is possible with use of this combinatorial screening system.

In some cases, the microfluidic device is incubated for a period of time prior to imaging and imaged at multiple time points to track changes in the measured amount of reporter over time. Additionally, for some experiments, merged droplets are eluted off of the microfluidic device for off-chip evaluation (see e.g., International Publication No. WO2016/149661, hereby incorporated by reference in its entirety for all purposes, elution is particularly discussed at [0056]-[0059]).

With the disclosed processing strategy, parallel handling of millions of droplets reaches the scale needed for combinatorial screening with the droplets' nanoliter volume reducing compound consumption required for screening. Cell free pathogenic nucleic acids in a host sample can be amplified using the padlock probe approach disclosed herein, with targeted detection utilizing CRISPR-Cas systems in droplet assays. The platform herein leverages the high throughput potential of droplet microfluidic systems, with the sensitivity of CRISPR-Cas detection systems, and tiled genomic probe approach to detect targets of interest in cell free nucleic acid samples. Advantageously, the systems and methods allow for non-invasive, sensitive testing for infections by using samples containing cell free nucleic acids that can be indicative of host infection utilizing the probes as designed herein when combined with SHEROCK droplet technology, that can be massively multiplexed utilizing smaller sample sizes.

### Samples

In preferred embodiments, the sample is blood or urine, in some embodiments, the sample is derived from blood or urine. In certain embodiments, the present invention provides steps of obtaining a sample of biological fluid (e.g., urine, blood plasma or serum, sputum, cerebral spinal fluid), and extracting the nucleic acid. The nucleotide sequence to be detected, may be a fraction of a larger molecule or can be present initially as a discrete molecule.

In certain embodiments, blood samples are collected and plasma immediately separated from the blood cells by centrifugation. Serum may be filtered and stored frozen until nucleic acid extraction.

In certain example embodiments, target nucleic acids are detected directly from a crude or unprocessed sample, such as blood, serum, saliva, cerebrospinal fluid, sputum, or urine. In certain example embodiments, the target nucleic acid is cell free DNA.

The samples contain cfDNA that can be used for detection. Small amounts of nucleic acids float freely in blood and urine, in both healthy and disease states, with the source, abundance and profile of plasma cfDNA believed to vary with physiological condition and disease state. For instance, in pregnant women, a large fraction of cfDNA originates from placental trophoblasts, and a measurable amount of fetal cfDNA is also present. Elevated levels of cfDNA have been observed in disease processes such as cancer and sepsis. More recently, a small fraction of cfDNA has also been shown to be of non-host origin, and comprise of fragments for commensal or pathogenic microbial origin.

### Diagnostic Methods

Detection of pathogens according to the methods herein can be used to monitor or detect host response to infection.

Methods as disclosed herein are also directed to methods of diagnosing a cell or tissue in a subject comprising an infection of a pathogen. In methods of diagnosing, the method comprises the step of detecting one or more targets of interest in a cell free sample. The order of steps provided herein is exemplary, certain steps may be carried out simultaneously or in a different order.

Diagnosis is commonplace and well-understood in medical practice. By means of further explanation and without limitation the term “diagnosis” generally refers to the process or act of recognizing, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition). Identifying a disease state, disease progression, or other abnormal condition, based upon symptoms, signs, and other physiological and anatomical parameters are also encompassed in diagnosis. In certain instances, diagnosis comprises detecting a gene expression profile of a sample, host tissue, cell or cell subpopulation.

The terms “prognosing” or “prognosis” generally refer to an anticipation on the progression of a disease or condition and the prospect (e.g., the probability, duration, and/or extent) of recovery. A good prognosis of the diseases or conditions taught herein may generally encompass anticipation of a satisfactory partial or complete recovery from the diseases or conditions, preferably within an acceptable time period. A good prognosis of such may more commonly encompass anticipation of not further worsening or aggravating of such, preferably within a given time period. A poor prognosis of the diseases or conditions as taught herein may generally encompass anticipation of a substandard recovery and/or unsatisfactorily slow recovery, or to substantially no recovery or even further worsening of such.

A “deviation” of a first value from a second value may generally encompass any direction (e.g., increase: first value >second value; or decrease: first value <second value) and any extent of alteration.

For example, a deviation may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation may refer to a statistically significant observed alteration. For example, a deviation may refer to an observed alteration which falls outside of error margins of reference values in a given population (as expressed, for example, by standard deviation or standard error, or by a predetermined multiple thereof, e.g., ±1×SD or ±2×SD or +3×SD, or ±1×SE or ±2×SE or ±3×SE). Deviation may also refer to a value falling outside of a reference range defined by values in a given population (for example, outside of a range which comprises ≥40%, ≥50%, ≥60%, ≥70%, ≥75% or ≥80% or ≥85% or ≥90% or ≥95% or even ≥100% of values in said population).

In a further embodiment, a deviation may be concluded if an observed alteration is beyond a given threshold or cut-off. Such threshold or cut-off may be selected as generally known in the art to provide for a chosen sensitivity and/or specificity of the prediction methods, e.g., sensitivity and/or specificity of at least 50%, or at least 60%, or at least 70%, or at least 80%, or at least 85%, or at least 90%, or at least 95%.

For example, receiver-operating characteristic (ROC) curve analysis can be used to select an optimal cut-off value of the quantity of a given immune cell population, biomarker or gene or gene product signatures, for clinical use of the present diagnostic tests, based on acceptable sensitivity and specificity, or related performance measures which are well-known per se, such as positive predictive value (PPV), negative predictive value (NPV), positive likelihood ratio (LR+), negative likelihood ratio (LR−), Youden index, or similar.

The term “monitoring” generally refers to the follow-up of a disease or a condition in a subject for any changes which may occur overtime. The term also encompasses prediction of a disease. The terms “predicting” or “prediction” generally refer to an advance declaration, indication or foretelling of a disease or condition in a subject not (yet) having said disease or condition. For example, a prediction of a disease or condition in a subject may indicate a probability, chance or risk that the subject will develop said disease or condition, for example within a certain time period or by a certain age. Said probability, chance or risk may be indicated inter alia as an absolute value, range or statistics, or may be indicated relative to a suitable control subject or subject population (such as, e.g., relative to a general, normal or healthy subject or subject population). Hence, the probability, chance or risk that a subject will develop a disease or condition may be advantageously indicated as increased or decreased, or as fold-increased or fold-decreased relative to a suitable control subject or subject population. As used herein, the term “prediction” of the conditions or diseases as taught herein in a subject may also particularly mean that the subject has a ‘positive’ prediction of such, i.e., that the subject is at risk of having such (e.g., the risk is significantly increased vis-à-vis a control subject or subject population). The term “prediction of no” diseases or conditions as taught herein as described herein in a subject may particularly mean that the subject has a ‘negative’ prediction of such, i.e., that the subject's risk of having such is not significantly increased vis-à-vis a control subject or subject population.

The invention provides a method for monitoring infection in a subject and for determining the severity of a disease or condition by comparing the gene profiles from a healthy subject or reference control with one from a subject suspected of having a disease or condition, or monitoring the progression of the disease.

Method embodiments are also provided for monitoring a subject having no symptoms of disease to determine onset of or diagnose a disease comprising monitoring changes, or velocity of change in the level or presence of one or more target sequences in a cell free DNA sample associated with the disease wherein a change, or alterations in velocity of change in the level or presence of the one or more target sequences in a cell free DNA sample indicates presence of the disease.

In another aspect, a method is provided for monitoring a subject to predict response to treatment for a disease comprising implanting monitoring changes in the level or presence of one or more target sequences associated with a disease wherein a change, or alterations in velocity of change in the level or presence of the one or more target sequences associated with treatment resistance of the disease indicates the presence or absence of resistance of the subject to a disease treatment. Methods may include performing methods of detection at a first time and at one or more subsequent times, detecting the presence of one or more pathogens at the first time and the one or more subsequent times. In particular embodiments, a host is treated with an antibiotic subsequent to the first time and prior to the one or more subsequent times. In this way, antibiotic resistance and identifying genetic markers associated with antibiotic resistance can be detected.

The step of detecting for the purposes of monitoring can, in one embodiment, comprise detecting the presence of target molecules of cfDNA compared to a sample that is not infected. The step of detecting can, in one embodiment, comprise detecting presence, or in some instances quantitation of one or more target molecules of cfDNA in a sample. The step of detecting can also comprise a profile of one or more target molecules.

In one embodiment, the change in the level or presence of the one or more target molecules associated with the infection is compared to normal levels in the subject or a population of healthy or normal subjects where the change, or alterations in velocity of change in the level or presence of the one or more biomolecules indicates the presence of the disease.

In certain example embodiments, the devices, systems, and methods disclosed herein may be used to detect or distinguish pathogens in a cell free nucleic acid sample. In certain example embodiments, a set of guide RNAs may be designed to distinguish each species by a variable region that is unique to each species or strain. Guide RNAs may also be designed to target RNA genes that distinguish microbes at the genus, family, order, class, phylum, kingdom levels, or a combination thereof. In certain example embodiments where amplification is used, a set of amplification primers may be designed to flanking constant regions of the RNA sequence and a guide RNA designed to distinguish each species by a variable internal region. In certain example embodiments, other genes or genomic regions that uniquely variable across species or a subset of species may be used as well. Other suitable phylogenetic markers, and methods for identifying the same, are discussed for example in Wu et al. arXiv:1307.8690 [q-bio.GN].

In certain example embodiments, a method or diagnostic is designed to screen pathogens across multiple phylogenetic and/or phenotypic levels at the same time. For example, the method or diagnostic may comprise the use of multiple CRISPR systems with different guide RNAs. A first set of guide RNAs may distinguish, for example, between strains of a bacterial infection or virus, or between different pathogens. A second set of guide RNA can be designed to distinguish microbes at the species level. The foregoing is for example purposes only. Other means for classifying other types of pathogens are also contemplated and would follow the general structure described above.

In certain example embodiments, the systems, devices, and methods disclosed herein may be used for SNP detection and/or genotyping. The systems, devices and methods disclosed herein may be also used for the detection of any disease state or disorder characterized by aberrant gene expression. Aberrant gene expression includes aberration in the gene expressed, location of expression and level of expression. The embodiments disclosed herein may be used for screening panels of different SNPs associated with a pathogen. As described herein elsewhere, closely related genotypes/alleles or biomarkers (e.g. having only a single nucleotide difference in a given target sequence) may be distinguished by introduction of a synthetic mismatch in the gRNA.

In an aspect, the invention relates to a method for detecting target nucleic acids in cell free nucleic acid samples for detection of one or more pathogens, comprising:
a. distributing a sample or set of samples into one or more individual discrete volumes, the individual discrete volumes comprising a CRISPR system according to the invention as described herein;
b. incubating the sample or set of samples under conditions sufficient to allow binding of the one or more guide RNAs to one or more target molecules;
c. activating the CRISPR effector protein via binding of the one or more guide RNAs to the one or more target molecules, wherein activating the CRISPR effector protein results in modification of the RNA-based masking construct such that a detectable positive signal is generated; and
d. detecting the detectable positive signal, wherein detection of the detectable positive signal indicates a presence of one or more target molecules in the sample.
   Cell-Free DNA (cfDNA)

In certain embodiments, the present invention may be used to detect cell free DNA (cfDNA). Cell free DNA in plasma or serum may be used as a non-invasive diagnostic tool. For example, cell free fetal DNA has been studied and optimized for testing on-compatible RhD factors, sex determination for X-linked genetic disorders, testing for single gene disorders, identification of preeclampsia. For example, sequencing the fetal cell fraction of cfDNA in maternal plasma is a reliable approach for detecting copy number changes associated with fetal chromosome aneuploidy. For another example, cfDNA isolated from cancer patients has been used to detect mutations in key genes relevant for treatment decisions.

In certain example embodiments, the present disclosure provides detecting cfDNA directly from a patient sample. In certain other example embodiment, the present disclosure provides enriching cfDNA using the enrichment embodiments disclosed above and prior to detecting the target cfDNA.

### Exosomes

In one embodiment, exosomes can be assayed with the present invention. Exosomes are small extracellular vesicles that have been shown to contain RNA. Isolation of exosomes by ultracentrifugation, filtration, chemical precipitation, size exclusion chromatography, and microfluidics are known in the art. In one embodiment exosomes are purified using an exosome biomarker. Isolation and purification of exosomes from biological samples may be performed by any known methods (see e.g., WO2016172598A1).

The invention is further described in the following examples, which do not limit the scope of the invention described in the claims.

### EXAMPLES

### Example 1

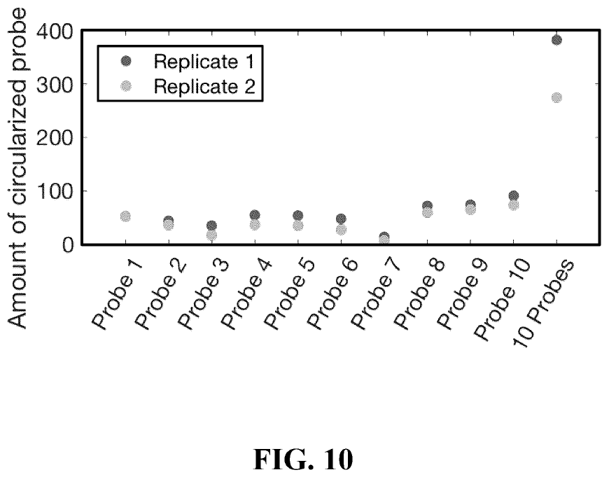

Molecular inversion probes (MIPs) targeting multiple evenly spaced regions of the S. aureus genome were designed. MIPs have been shown to be amenable to extensive multiplexing, and therefore hold promise as a pre-amplification strategy prior to CRISPR-based detection. Based on the list of potential targets generated through our computational workflow, ten evenly spaced targets across the S. aureus genome were selected. MIP-based amplification was then performed using S. aureus gDNA. Single- and multi-probe reactions were performed with the same amount of gDNA and keeping the total probe concentration fixed. Thus, in the ten-probe tiling reaction, each probe had a ten-fold lower concentration than any single-probe reaction. Upon hybridization, ligation and digestion of linear probes, the amount of circularized probes was quantified using qPCR with SYBR green (Bio-Rad Laboratories Inc., CA). Results show a 6-fold increase in signal when using ten tiled probes (FIG. 10).

### Example 2

Development of a detection assay at low volumes using droplet microfluidics is provided herein. Using the droplet system described previously⁵⁶, we sought to determine the compatibility of the SHERLOCK assay with droplet microfluidics. The detection step of SHERLOCK involves combining a RPA pre-amplified target with a CRISPR-Cas13a/reporter mixture. We first attempted to test the stability of nanoliter droplets of these two components using a droplet generator (Bio-Rad QX200). RPA droplets were unstable without dilution, but appeared stable at 10× dilution with nuclease-free water. Detection mix droplets were stable without any dilution.

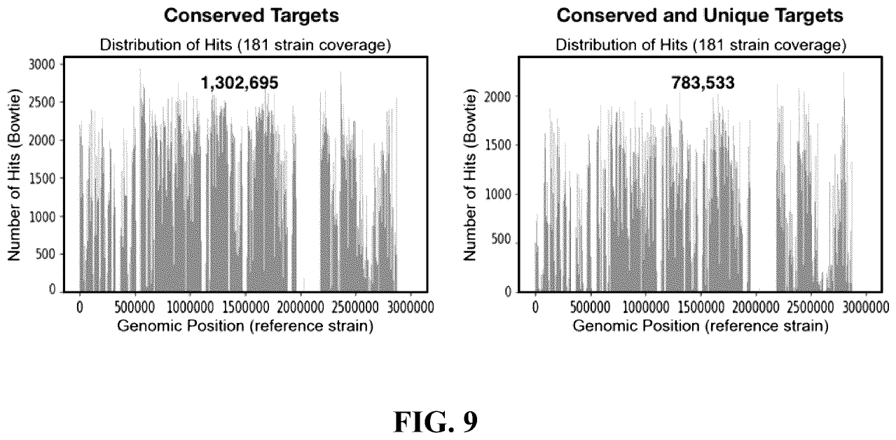

Next, Applicants modified the microscopy setup to detect fluorescence of the reporter used in SHERLOCK, and optimized reporter concentrations for sensitivity. dSHERLOCK experiments were then done on the microfluidic platform to test two things: (1) the performance of the detection assay at nanoliter volumes; and (2) the ability of the droplet platform to run multiple, parallel reactions with different targets and probes. One set of experiments sought to replicate recently published results that demonstrate the ability of SHERLOCK to detect SNPs in Zika viral genomes59. Using eight probe sets and sixteen RPA pre-amplified stocks used in this study, we ran the detection step of SHERLOCK on the droplet microfluidic platform. The results indicate a similar limit of detection (LoD) when running the assay in nanoliter droplets when compared to large volume (20 μl) reactions on plates (FIG. 11A, 111B), although a higher level of background is observed in droplets. Relative signals from probes designed to detect SNPs also look similar between the two setups (FIG. 9 C,D).

In order to assess the impact of this higher background fluorescence on call confidence, we ran bootstrapping analysis to determine the number of replicates as a function of call confidence. This analysis suggests that calls between SNPs can be made with 100% accuracy with less than 10 replicates (FIG. 12) on the droplet. This highlights the potential of using the platform for high-throughput assays with multiple samples against multiple probesets.

### Example 3

It is believed tiled detection of multiple targets can improve sensitivity, without affecting specificity. Accordingly, a platform for genome-wide tiling and detection of nucleic acid targets is described herein.

Computational crRNA probe design: The preliminary analysis of S. aureus described in Example 1 suggested that a sizeable fraction of the genome is likely to be uniquely targetable. For the computational component, a more extensive analysis will be performed on a variety of other bacteria and archaea of clinical relevance, and also expand the “out” group to include other pathogens that are likely to interfere with signal, such as common environmental microbes. Additional tools will also be developed for selecting the best targets/crRNA probes from a candidate list of unique target sequences. This process will include factors that may affect assay performance, such as the melting temperature, sequence orthogonality and genomic distribution. Existing tools designed to address similar questions can also be evaluated.

Experimental evaluation of pooled crRNA probes: Regardless of the complexity of computational design of probes, there are likely to be unaccounted empirical variations. Therefore, experimental strategies for screening probes will also be used. One such approach involves high-throughput screening using the droplet microfluidics system described previously. Different combinations of pooled probes will be made and overall sensitivity in single and multiplexed reactions will be assessed using intensity of fluorescence readout. During this design and evaluation process, we will focus on a single bacterium (S. aureus, Newman strain) to demonstrate our proof-of-concept. S. aureus is easier to work with than say M. tuberculosis, but also represents a major cause of nosocomial infections, making it a good candidate to start with.

Assessment of pre-amplification strategies: Current CRISPR-based diagnostic systems incorporate a pre-amplification step (using RPA). Tiled detection of multiple genomic targets may also benefit from pre-amplification. However, the amplification strategy must amplify all targets of interest, and RPA is limited in the extent to which it can be multiplexed. Since CRISPR-Cas introduces a second layer of specificity, amplification need not be exclusive to the targets of interest, i.e., non-specific amplification of background may be permissible. The choice of amplification strategies range from unbiased amplification of all nucleic acids in the sample, to specific multiplex amplification of just the targets. This spectrum of possible strategies is depicted in FIG. 11B. Each of these approaches will be evaluated for their compatibility and sensitivity gain when coupled with CRISPR-Cas detection.

Simulation of plasma cfDNA landscape: Detection of cfDNA in plasma presents unique challenges. First, although the microbial cfDNA landscape is only beginning to be mapped, there is strong evidence to suggest that these fragments are likely to be much shorter than host cfDNA²⁶. Next, host nuclear cfDNA is likely to be in much higher abundance than microbial cfDNA³⁶. In order to optimize our assay to detect ultra-short microbial fragments in plasma, experiments will be performed using fragmented S. aureus gDNA that mimics the fragmentation profile of host mitochondrial cfDNA. In addition, a background of fragmented human gDNA that mimics the size profile of host nuclear cfDNA (which peaks at ˜150 bp) will also be introduced. Protocols will be developed for fragmentation (using enzymatic or sonication approaches) to simulate physiologically relevant abundances and size profiles of cfDNA.

### Example 4

Tile Assay optimization will continue in clinical samples from healthy subjects as well as those with active infection. Addressing physiologically relevant conditions including the presence of reaction inhibitors in plasma, high patient-to-patient variability in cfDNA abundance, and the presence of contaminating microbial DNA will be addressed in the optimization and benchmarking of the assay. Initial focus will be on three clinically significant pathogens: S. aureus, Aspergillus fumigatus, and M tuberculosis. S. aureus commonly manifests as bloodstream infections, and is therefore likely to result in higher abundance of microbial plasma cfDNA than infections at other body sites, therefore making for a good infection to work with initially. However, the flexibility of the MIPs allow for use with a variety of pathogens, and can further be designed to identify across a genus, species, or other category or class of pathogen. A. fumigatus is an opportunistic pathogen that commonly affect the lung, and can sometimes disseminate to the bloodstream. It is also a common environmental microbe, elevating the risk for contamination. These features will allow us to test sensitivity and robustness of the assay. Finally, we will progress to studying patients with TB, which is the deadliest infectious disease, and therefore requires additional handling precautions, but also holds great potential for the application of novel diagnostics.

### Research Design and Methods

Sample collection: In order to preserve cfDNA, whole blood samples need to be collected in special tubes that stabilize cfDNA fragments (PAXgene blood ccfDNA tubes). In collaboration with clinicians at the Brigham and Women's Hospital, protocols will be developed for recruiting patients with a diagnosis of active S. aureus bloodstream infections, and for collection of blood in cfDNA preservation tubes. Patients are typically treated with antibiotics upon diagnosis of active infection, which may alter the cfDNA landscape. We will therefore attempt to collect samples within 48 hours of infection diagnosis. cfDNA remains stable in PAXgene tubes for up to seven days at room temperature⁶⁴, giving us sufficient time for transport of samples from hospital to lab.

Sample preparation: A key step for any diagnostic assay is sample preparation. Several commercial kits are available for the extraction of cfDNA from blood/plasma⁶⁵, and a novel method for removing SHERLOCK specific inhibitors has been published recently⁵⁹. We will first evaluate the performance of these methods by spiking blood with known amounts of fragmented pathogen gDNA, and quantifying the fraction of fragments extracted. Next, the optimal sample preparation method will be applied to clinical samples.

Independent characterization of microbial cfDNA: To address the paucity of literature on microbial cfDNA, and also have a way of evaluating the performance of our tiled assay, aliquots of extracted cfDNA will be used for independent characterization using two methods: next generation sequencing and ddPCR. Sequencing will allow us to assess cfDNA in terms of fragment size distribution and positional distribution across the genome. ddPCR may be more sensitive than sequencing and allow us to more accurately estimate abundance of pre-defined target sequences.

Benchmarking of tiled assay with current methods: Remaining aliquots of extracted cfDNA from the same patients (including healthy controls) will be used to evaluate the performance of our tiled detection assay. Several benchmarks will be used for comparison: ROC curve analysis between positive and control samples; sensitivity of tiled assay compared to documented patient bacterial load (in CFU/ml), which is a metric used to assess limit of detection of commercial NATs; ddPCR based detection of single (or handful of) pathogen specific targets, which currently has one of the highest sensitivity among NATs reported in the literature.

### Example 5

Genome-wide tiling of detection probes aims to improve sensitivity in detection a single pathogen. Another axis along which reactions can be multiplexed is the number of pathogens. In current clinical practice, patient presentation typically leads clinicians to suspect the presence of certain types of pathogens, rather than any single pathogen in particular. It can be enormously beneficial, therefore, to run a single diagnostic test that detects the presence of multiple pathogens. With CRISPR-based diagnostics, this need has led to the design of multiplexing such as SHERLOCKv2, which detects up to four pathogens in the same reaction. This multiplexing is achieved through the use of Cas proteins with orthogonal cleavage activity. Another possible strategy for multiple pathogen detection is spatial multiplexing, which is enabled by the droplet microfluidic technology described earlier herein. A conceptual use-case for adapting the droplet microfluidic device for CRISPR-diagnostics is illustrated in FIG. 14. The preliminary experiments suggest that such an approach can enable multiplexing at higher orders of magnitude than four. Thus, the focus will be multiplexing the assay along the pathogen axis, and potentially incorporate the assays developed as disclosed herein.

### Research Design and Methods

Optimization of CRISPR-Cas detection in droplets: Preliminary experiments have demonstrated the feasibility of CRISPR-Cas detection in droplets that can be optimized, for example by expanding the space for spatial multiplexing using encoding dyes; three encoding dyes are currently used to generate up to 64 unique droplet barcodes⁵⁶; adding a fourth encoding dye channel could expand this space to 256 barcodes. Next, the setup can be modified to run kinetic, rather than end-point assays, which can allow for greater quantitative readout; initial experiments comprised of imaging droplets at two time points, separated by incubation of droplets at 37° C. in a warm room; adding an incubator to the microscope could allow for imaging to be coupled with incubation, thereby enabling fluorescence kinetics and quantitation.

Extension of tiled detection assay to pathogen panel: Based on the success of Aims 1 and 2, tiled assays can be designed for detecting multiple pathogens, identifying genetic markers of antibiotic resistance, detecting host cfDNA profiles to characterize host response to infection, and various combinations of these. Strategies can also be developed for the identification of co-infections (such as HIV-TB), as well as distinguishing cases of contamination from actual infection. Many of these possibilities will be explored and tested during this phase.

Chip-based sample preparation and pre-amplification: In preliminary experiments, the detection step of SHERLOCK was performed in droplets. Methods will also be explored for performing pre-detection steps in microfluidic systems. One such approach is the use of valve and channel based microfluidic devices, which could lower upstream costs of sample preparation. The coupling of channel-based microfluidics with micro-well systems for target detection will be explored.

Design of portable signal readout: A current limitation of the droplet-based microfluidic system is the use of expensive and bulky microscopy for signal and spatial encoding dye detection, as well as the use of a proprietary droplet generator. More portable setups for droplet generation and dye/reporter sensing will be considered, including digital microfluidics.

The droplet based system modified and developed as disclosed herein can help address some of the potential complications in developing the platform for genome-wide tiling and optimizing the performance of the tile assay in clinical samples. Moreover, given that CRISPR-based detection employs a single probe for target binding and signal amplification (unlike PCR, which uses two primers for each target), the scaling of probe-probe interactions is likely to be different.

### References for the Examples Include

1 Murray C J L, GBD 2015 Mortality and Causes of Death Collaborators. Global, regional, and national age-sex specific all-cause and cause-specific mortality for 240 causes of death, 1990-2013: a systematic analysis for the Global Burden of Disease Study 2013. Lancet 2015; 385:117-71. doi.org/10.1016/S0140-6736(14)61682-2.
2 Hsiao C-J, Cherry D K, Beatty P C, Rechtsteiner E a. National Ambulatory Medical Care Survey: 2007 summary. Natl Health Stat Report 2010:1-32.
3 World Health Organization, UNICEF, UNAIDS. Global Update on HIV Treatment 2013: Results, Impact and Opportunities. 2013.
4 Nathanson N, Kew O M. From emergence to eradication: The epidemiology of poliomyelitis deconstructed. Am J Epidemiol 2010; 172:1213-29. doi.org/10.1093/aje/kwg320.
5 Pontali E, Matteelli A, Migliori G B. Drug-resistant tuberculosis. Curr Opin Pulm Med 2013; 19:266-72. doi.org/10.1097/MCP.0b013e32835flbf3.
6 Schito M, Migliori G B, Fletcher H A, McNerney R, Centis R, D′Ambrosio L, et al. Perspectives on Advances in Tuberculosis Diagnostics, Drugs, and Vaccines. Clin Infect Dis 2015; 61:S102-18. doi.org/10.1093/cid/civ609.
7 Global Tuberculosis Report. World Health Organization; 2017.
8 Osterholm M T, Kelley N S, Sommer A, Belongia E A. Efficacy and effectiveness of influenza vaccines: A systematic review and meta-analysis. Lancet Infect Dis 2012; 12:36-44. doi.org/10.1016/S1473-3099(11)70295-X.
9 Sands P, Mundaca-Shah C, Dzau V J. The Neglected Dimension of Global Security—A Framework for Countering Infectious-Disease Crises. N Engl J Med 2016; 374:1281-7. doi.org/10.1056/NEJMsr1600236.
10 Chin D P, Hanson C L. Finding the Missing Tuberculosis Patients. J Infect Dis 2017; 216:S675-8doi.org/10.1093/infdis/jix368.
11 Subbaraman R, Nathavitharana R R, Satyanarayana S, Pai M, Thomas B E, Chadha V K, et al. The Tuberculosis Cascade of Care in India's Public Sector: A Systematic Review and Meta-analysis. PLOS Med 2016; 13:e1002149. https://doi.org/10.1371/joumal.pmed.1002149.
12 Bloom B R. A Neglected Epidemic. N Engl J Med 2018; 378:291-3. doi.org/10.1056/NEJMe1714609.
13 Hall H I, An Q, Tang T, Song R, Chen M, Green T, et al. Prevalence of Diagnosed and Undiagnosed HIV Infection-United States, 2008-2012. MMWR Morb Mortal Wkly Rep 2015; 64:657-62.
14 Fair R J, Tor Y. Antibiotics and bacterial resistance in the 21st century. Perspect Medicin Chem 2014; 6:25-64. doi.org/10.4137/PMC.S14459.
15 Mancini N, Carletti S, Ghidoli N, Cichero P, Burioni R, Clementi M. The era of molecular and other non-culture-based methods in diagnosis of sepsis. Clin Microbiol Rev 2010; 23:235-51. doi.org/10.1128/CMR.00043-09.
16 Ryu Y J. Diagnosis of pulmonary tuberculosis: recent advances and diagnostic algorithms. Tuberc Respir Dis (Seoul) 2015; 78:64-71. doi.org/10.4046/trd.2015.78.2.64.
17 Lagier J C, Edouard S, Pagnier I, Mediannikov O, Drancourt M, Raoult D. Current and past strategies for bacterial culture in clinical microbiology. Clin Microbiol Rev 2015; 28:208-36. doi.org/10.1128/CMR.00110-14.
18 Tang Y W, Stratton C W. Advanced techniques in diagnostic microbiology. Boston, M A: Springer U S; 2014.
19 Taylor D, Durigon M, Davis H, Archibald C, Konrad B, Coombs D, et al. Probability of a false-negative HIV antibody test result during the window period: a tool for pre- and post-test counselling. Int J STD AIDS 2015; 26:215-24. doi.org/10.1177/0956462414542987.
20 Mothershed E A, Whitney A M. Nucleic acid-based methods for the detection of bacterial pathogens: Present and future considerations for the clinical laboratory. Clin Chim Acta 2006; 363:206-20doi.org/10.1016/J.CCCN.2005.05.050.
21 Anwar A, Wan G, Chua K-B, August J T, Too H-P. Evaluation of pre-analytical variables in the quantification of dengue virus by real-time polymerase chain reaction. J Mol Diagn 2009; 11:537-42. https://doi.org/10.2353/jmoldx.2009.080164.
22 Anker P, Mulcahy H, Chen X Q, Stroun M. Detection of circulating tumour DNA in the blood (plasma/serum) of cancer patients. Cancer Metastasis Rev 1999; 18:65-73. doi.org/10.1023/A:1006260319913.
23 Stroun M, Lyautey J, Lederrey C, Olson-Sand A, Anker P. About the possible origin and mechanism of circulating DNA: Apoptosis and active DNA release. Clin Chim Acta 2001; 313:139-42. doi.org/10.1016/S0009-8981(01)00665-9.
24 Chan A K C, Chiu R W K, Lo Y M D, Clinical Sciences Reviews Committee of the Association of Clinical Biochemists. Cell-free nucleic acids in plasma, serum and urine: anew tool in molecular diagnosis. Ann Clin Biochem 2003; 40:122-30. doi.org/10.1258/000456303763046030.
25 Lui Y Y N, Chik K-W, Chiu R W K, Ho C-Y, Lam C W K, Lo Y M D. Predominant hematopoietic origin of cell-free DNA in plasma and serum after sex-mismatched bone marrow transplantation. Clin Chem 2002; 48:421-7.
26 Lo Y M D, Chan K C A, Sun H, Chen E Z, Jiang P, Lun F M F, et al. Maternal Plasma DNA Sequencing Reveals the Genome-Wide Genetic and Mutational Profile of the Fetus. Sci Transl Med 2010; 2:61ra91-61ra91. doi.org/10.1126/scitranslmed.3001720.
27 Fan H C, Blumenfeld Y J, Chitkara U, Hudgins L, Quake S R. Noninvasive diagnosis of fetal aneuploidy by shotgun sequencing DNA from maternal blood. Proc Natl Acad Sci 2008; 105:16266-71. doi.org/10.1073/pnas.0808319105.
28 Bettegowda C, Sausen M, Leary R J, Kinde I, Wang Y, Agrawal N, et al. Detection of Circulating Tumor DNA in Early- and Late-Stage Human Malignancies. Sci Transl Med 2014; 6:224ra24-224ra24. doi.org/10.1126/scitranslmed.3007094.
29 Dwivedi D J, Toltl L J, Swystun L L, Pogue J, Liaw K-L, Weitz J I, et al. Prognostic utility and characterization of cell-free DNA in patients with severe sepsis. Crit Care 2012; 16:R151. doi.org/10.1186/ccI1466.
30 Kowarsky M, Camunas-Soler J, Kertesz M, De Vlaminck I, Koh W, Pan W, et al. Numerous uncharacterized and highly divergent microbes which colonize humans are revealed by circulating cell-free DNA. Proc Natl Acad Sci USA 2017; 114:9623-8. doi.org/10.1073/pnas.1707009114.
31 Manier S, Park J, Capelletti M, Bustoros M, Freeman S S, Ha G, et al. Whole-exome sequencing of cell-free DNA and circulating tumor cells in multiple myeloma. Nat Commun 2018; 9:1691. doi.org/10.1038/s41467-018-04001-5.
32 Wanda L, Ruffin F, Hill-Rorie J, Hollemon D, Seng H, Hong D, et al. Direct Detection and Quantification of Bacterial Cell-free DNA in Patients with Bloodstream Infection (BSI) Using the Karius Plasma Next Generation Sequencing (NGS) Test. Open Forum Infect Dis 2017; 4:5613-5613. https://doi.org/10.1093/ofid/ofxl63.1613.
33 Click E S, Murithi W, Ouma G S, McCarthy K, Willby M, Musau S, et al. Detection of Apparent Cell-free M. tuberculosis DNA from Plasma. Sci Rep 2018; 8:645. doi.org/10.1038/s41598-017-17683-6.
34 Che N, Yang X, Liu Z, Li K, Chen X. Rapid Detection of Cell-Free Mycobacterium tuberculosis DNA in Tuberculous Pleural Effusion. J Clin Microbiol 2017; 55:1526-32. doi.org/10.1128/JCM.02473-16.
35 Yamamoto M, Ushio R, Watanabe H, Tachibana T, Tanaka M, Yokose T, et al. Detection of Mycobacterium tuberculosis-derived DNA in circulating cell-free DNA from a patient with disseminated infection using digital PCR. Int J Infect Dis 2018; 66:80-2. doi.org/10.1016/J.IJID.2017.11.018.
36 Burnham P, Kim M S, Agbor-Enoh S, Luikart H, Valantine H A, Khush K K, et al. Single-stranded DNA library preparation uncovers the origin and diversity of ultrashort cell-free DNA in plasma. Sci Rep 2016; 6:27859. https://doi.org/10.1038/srep27859.
37 Mullis K, Faloona F, Scharf S, Saiki R, Horn G, Erlich H. Specific enzymatic amplification of DNA in vitro: the polymerase chain reaction. Cold Spring Harb Symp Quant Biol 1986; 51 Pt 1:263-73.
38 Holland P M, Abramson R D, Watson R, Gelfand D H. Detection of specific polymerase chain reaction product by utilizing the 5′-3′ exonuclease activity of Thermus aquaticus DNA polymerase. Proc Natl Acad Sci USA 1991; 88:7276-80.
39 Hindson C M, Chevillet J R, Briggs H A, Gallichotte E N, Ruf I K, Hindson B J, et al. Absolute quantification by droplet digital PCR versus analog real-time PCR. Nat Methods 2013; 10:1003-5. doi.org/10.1038/nmeth.2633.
40 Compton J. Nucleic acid sequence-based amplification. Nature 1991; 350:91-2. doi.org/10.1038/350091a0.
41 Ali M M, Li F, Zhang Z, Zhang K, Kang D-K, Ankrum J A, et al. Rolling circle amplification: a versatile tool for chemical biology, materials science and medicine. Chem Soc Rev 2014; 43:3324. doi.org/10.1039/c3cs60439j.
42 Lutz S, Weber P, Focke M, Faltin B, Hoffmann J, Maller C, et al. Microfluidic lab-on-a-foil for nucleic acid analysis based on isothermal recombinase polymerase amplification (RPA). Lab Chip 2010; 10:887. doi.org/10.1039/b921140c.
43 Piepenburg 0, Williams C H, Stemple D L, Armes N A. DNA Detection Using Recombination Proteins. PLoS Biol 2006; 4:e204. doi.org/10.1371/journal.pbio.0040204.
44 Elnifro E M, Ashshi A M, Cooper R J, Klapper P E. Multiplex PCR: optimization and application in diagnostic virology. Clin Microbiol Rev 2000; 13:559-70.
45 Nilsson M, Malmgren H, Samiotaki M, Kwiatkowski M, Chowdhary B P, Landegren U. Padlock probes: circularizing oligonucleotides for localized DNA detection. Science 1994; 265:2085-8.
46 Hardenbol P, Yu F, Belmont J, Mackenzie J, Bruckner C, Brundage T, et al. Highly multiplexed molecular inversion probe genotyping: Over 10,000 targeted SNPs genotyped in a single tube assay. Genome Res 2005; 15:269-75. doi.org/10.1101/gr.3185605.
47 Hiatt J B, Pritchard C C, Salipante S J, O'Roak B J, Shendure J. Single molecule molecular inversion probes for targeted, high-accuracy detection of low-frequency variation. Genome Res 2013; 23:843-54. doi.org/10.1101/gr.147686.112.
48 Krzywkowski T, Hauling T, Nilsson M. In Situ Single-Molecule RNA Genotyping Using Padlock Probes and Rolling Circle Amplification. Humana Press, New York, N Y; 2017. p. 59-76.
49 Hille F, Richter H, Wong S P, Bratovic M, Ressel S, Charpentier E. The Biology of CRISPR-Cas: Backward and Forward. Cell 2018; 172:1239-59. doi.org/10.1016/J.CELL.2017.11.032.
50 Chertow D S. Next-generation diagnostics with CRISPR. Science 2018; 360:381-2. doi.org/10.1126/science.aat4982.
51 Abudayyeh 00, Gootenberg J S, Konermann S, Joung J, Slaymaker I M, Cox D B T, et al. C2c2 is a single-component programmable RNA-guided RNA-targeting CRISPR effector. Science (80−) 2016; 353:aaf5573. doi.org/10.1126/science.aaf5573.
52 Chen J S, Ma E, Harrington L B, Da Costa M, Tian X, Palefsky J M, et al. CRISPR-Cas12a target binding unleashes indiscriminate single-stranded DNase activity. Science 2018; 360:436-9. doi.org/10.1126/science.aar6245.
53 Gootenberg J S, Abudayyeh 00, Kellner M J, Joung J, Collins J J, Zhang F. Multiplexed and portable nucleic acid detection platform with Cas13, Cas12a, and Csm6. Science (80−) 2018; 360:439-44. doi.org/10.1126/science.aaq0179.
54 Gootenberg J S, Abudayyeh 00, Lee J W, Essletzbichler P, Dy A J, Joung J, et al. Nucleic acid detection with CRISPR-Cas13a/C2c2. Science (80−) 2017; 356:438-42. doi.org/10.1126/science.aam9321.
55 Teh S-Y, Lin R, Hung L-H, Lee A P. Droplet microfluidics. Lab Chip 2008; 8:198. doi.org/10.1039/b715524g.
56 Kulesa A, Kehe J, Hurtado J, Tawde P, Blainey P C. Combinatorial Drug Discovery in Nanoliter Droplets. BioRxiv 2017:210492. doi.org/I0.1101/210492.
57 Langmead B, Trapnell C, Pop M, Salzberg S L. Ultrafast and memory-efficient alignment of short DNA sequences to the human genome. Genome Biol 2009; 10:R25. doi.org/10.1186/gb-2009-10-3-r25.
58 Agarwala R, Barrett T, Beck J, Benson D A, Bollin C, Bolton E, et al. Database Resources of the National Center for Biotechnology Information. Nucleic Acids Res 2017; 45:D12-7. doi.org/10.1093/nar/gkwl071.
59 Myhrvold C, Freije C A, Gootenberg J S, Abudayyeh 00, Metsky H C, Durbin A F, et al. Field-deployable viral diagnostics using CRISPR-Cas13. Science (80−) 2018; 360:444-8. doi.org/10.1126/science.aas8836.
60 Roychowdhury T, Mandal S, Bhattacharya A. Analysis of IS6110 insertion sites provide a glimpse into genome evolution of Mycobacterium tuberculosis. Sci Rep 2015; 5:12567. doi.org/10.1038/srep12567.
61 McEvoy C R E, Falmer A A, van Pittius N C G, Victor T C, van Helden P D, Warren R M. The role of IS6110 in the evolution of Mycobacterium tuberculosis. Tuberculosis 2007; 87:393-404. doi.org/10.1016/j.tube.2007.05.010.
62 Chakravorty S, Simmons A M, Rowneki M, Parmar H, Cao Y, Ryan J, et al. The New Xpert MTB/RIF Ultra: Improving Detection of Mycobacterium tuberculosis and Resistance to Rifampin in an Assay Suitable for Point-of-Care Testing. MBio 2017; 8:e00812-17. doi.org/10.1128/mBio.00812-17.
63 Boyle E A, O'Roak B J, Martin B K, Kumar A, Shendure J. MIPgen: optimized modeling and design of molecular inversion probes for targeted resequencing. Bioinformatics 2014; 30:2670-2. doi.org/10.1093/bioinformatics/btu353.
64 Alidousty C, Brandes D, Heydt C, Wagener S, Wittersheim M, Schafer S C, et al. Comparison of Blood Collection Tubes from Three Different Manufacturers for the Collection of Cell-Free DNA for Liquid Biopsy Mutation Testing. J Mol Diagnostics 2017; 19:801-4. doi.org/10.1016/j.jmoldx.2017.06.004.
65 Sorber L, Zwaenepoel K, Deschoolmeester V, Roeyen G, Lardon F, Rolfo C, et al. A Comparison of Cell-Free DNA Isolation Kits. J Mol Diagnostics 2017; 19:162-8. doi.org/10.1016/j.jmoldx.2016.09.009.

### Example 6—MIP and Target Design Considerations

FIG. 29G is a heatmap of SHERLOCK signal showing strength of interactions between eight de novo designed gRNAs (y-axis) and their cognate and non-cognate in vitro transcribed targets (x-axis). Lack of off-diagonal signal indicates a high level of specificity, and intensity of signal along diagonal indicates strength of signal. To produce the RNA targets, template oligonucleotides were synthesized that contain the T7 promoter sequence, targeting sequence, and T7 terminal promoter binding site. These were amplified by PCR using T7 Promoter and T7 terminal primers to yield a 73 bp double stranded product. These products were then used as templates in the SHERLOCK reaction. gRNAs are generated via in vitro transcription of 89 base oligonucleotides. A T7 promoter oligo was hybridized to the 3′ end of the oligo, which then enabled the production of the guide RNA in vitro using T7 RNA polymerase. Sherlock reactions were performed in microfluidic format. Briefly, droplets were created carrying either a guide RNA or template DNA along with one of a number of selected colored dyes; every gRNA or template was packages in a droplet specifically colored to indicate the presence of that particular reagent. Droplets could be arrayed in random pairs and subsequently fused, with the contents of each droplet indicated by the spectral properties of the 2 fused droplets. In this way, combinations of gRNA and template could be assayed across a large number of replicates.

### Example 7: Hybridization and Ligation of MIPs

Signal generated by ligation in the presence of enzymatically sheared small gDNA fragments is more effective that in comparable concentrations of larger gDNA fragments (>10,000 bp). Strength of signal is also dependent on MIP concentration, with few MIPS required for signal generation in the presence of sheared gDNA. Shown is the SHERLOCK signal (relative fluorescence) from rolling-circle amplified MIP SA31_PM ligated in the presence of 100,000 copies of Staphylococcus aureus genomic DNA.

MIPs were added to the ligation reaction at the indicated copy number (x axis, bars 1-12), and circularization of MIPs by templated interactions with target gDNA was done at 55° C. in the presence of Ampligase for 20 minutes. Ligated material was amplified overnight using rolling circle amplification, and products of this reaction were transferred to SHERLOCK reactions for fluorescence detection. Bars 1-6 indicate strength of signal when enzymatically-sheared genomic DNA fragments of 200-300 bp were used to template the MIP ligation reaction. Bars 7-12 use the same gDNA prior to shearing. Bars 13-14 are controls where 100,000 previously circularized MIP SA31_PM (or water) were put through RCA and SHERLOCK reaction, and bars 15-16 are SHERLOCK assay controls wherein a PCR-amplified DNA template or water was directly introduced into the SHERLOCK assay.

### Example 8: Multiplexing Mips Improves Assay Sensitivity

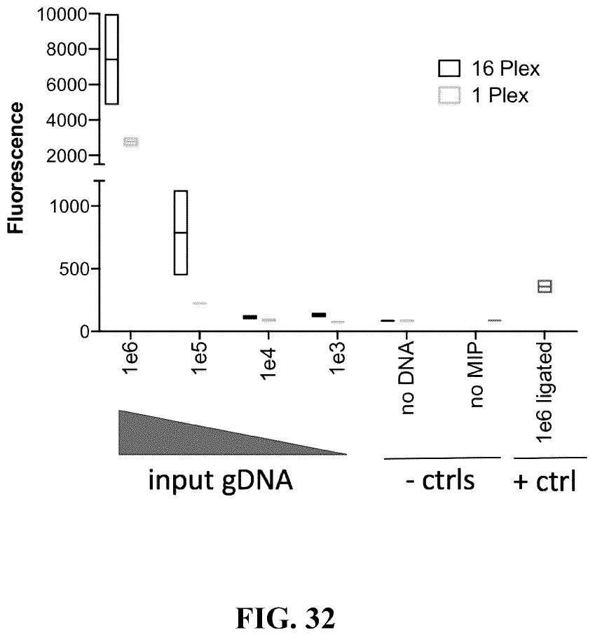

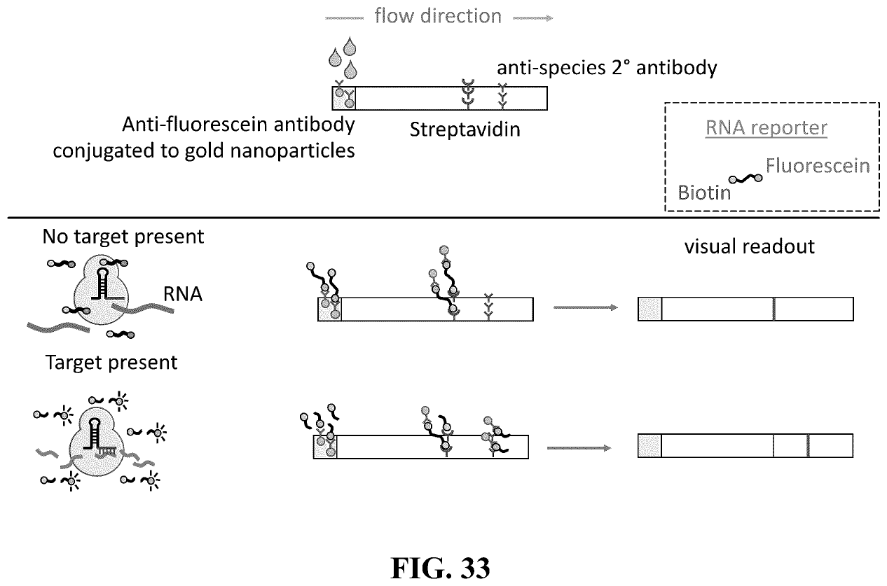

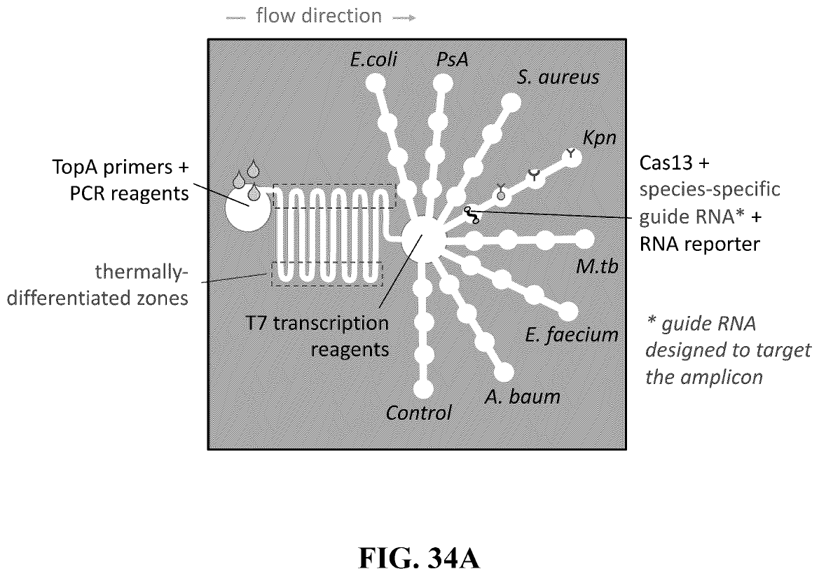

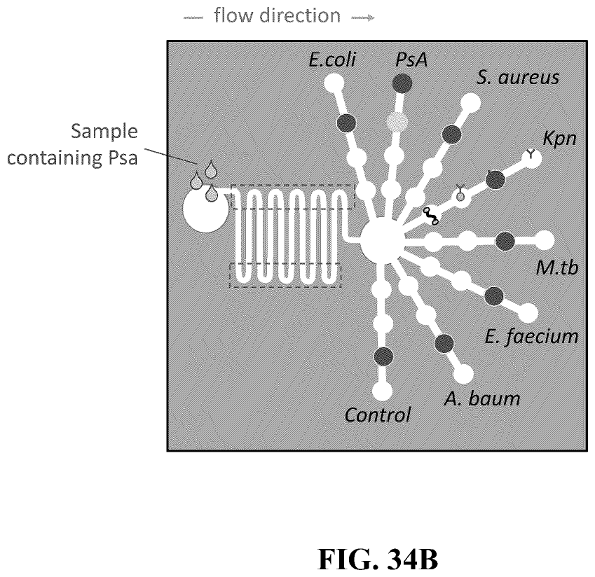

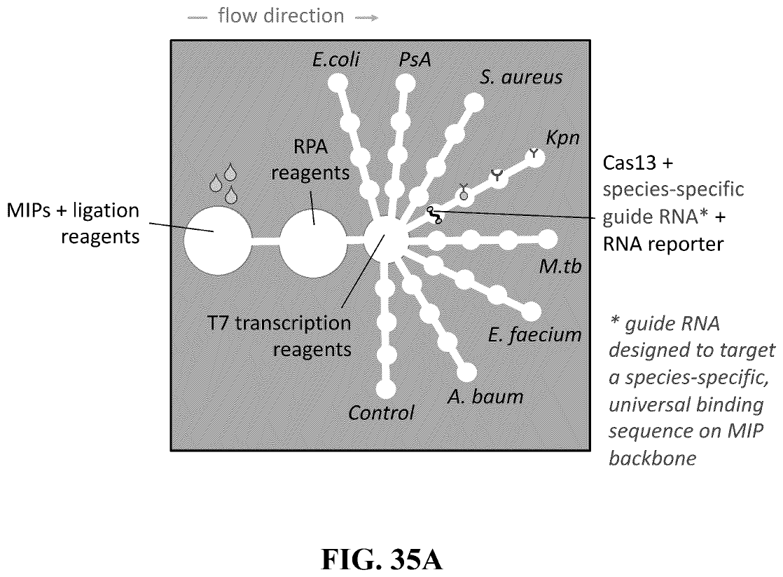

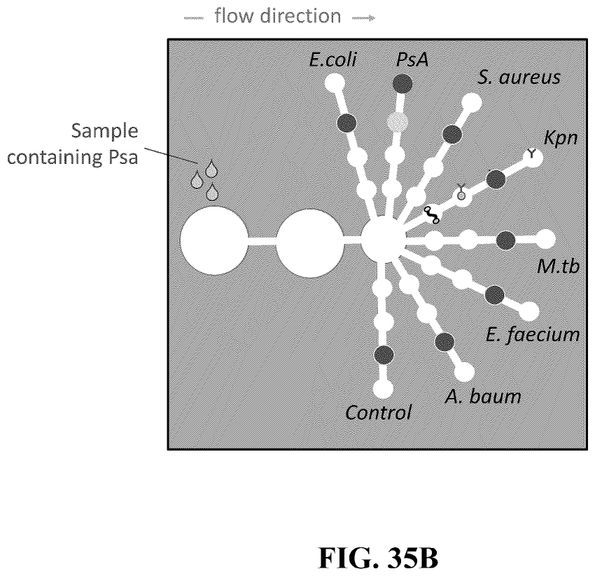

FIG. 32 graphs signal strength and limit of detection was compared when either a single MIP (SA31 MP) or 16 MIPs sharing a common gRNA sequence (SA31 MP along with 15 additional MIPs) were ligated in the presence of various concentration of sheared. S. aureus gDNA. The total number of MIP was constant (le9 copies/ligation) in both assays. Ligation were ExoI/III treated and amplified by RCA.

Circularization of MIPs by templated interactions with target gDNA was done at 55° C. in the presence of Ampligase for 20 minutes. Ligated material was amplified overnight using rolling circle amplification, and products of this reaction were transferred to SHERLOCK reactions for fluorescence detection. Improved signal strength was observed and limit of detection of fragmented S. aureus genomic DNA.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A nucleic acid detection system comprising:
one or more CRISPR-Cas proteins,
an oligonucleotide based detection construct, optionally two or more, comprising a non-target sequence, wherein the CRISPR-Cas protein exhibits collateral activity and cleaves the non-target sequence once activated by the target; and either
one or more sets of proximity dependent probes, each set comprising two or more proximity dependent probes, each proximity dependent probe comprising a guide polynucleotide recognition sequence and a target binding sequence and a guide polynucleotide for each set of ligation dependent probes, each guide polynucleotide comprising a guide sequence capable of hybridizing to the guide polynucleotide recognition sequence of the one or more ligation dependent probe sets, and designed to form a complex with the one or more CRISPR-Cas proteins; or
one or more tiled guide polynucleotide sets, each set comprising a plurality of guide polynucleotides, each guide within a set designed to hybridize to a different portion of a same target sequence and designed to form a complex with the one or more CRISPR-Cas proteins.

2. (canceled)

3. The detection system of claim 1, wherein the proximity dependent probes are further:
optionally linked by ligation, splinted ligation, hybridization, or proximity extension,
optionally comprise one or more of a forward primer binding site, a reverse primer
binding site, and a reverse polymerase binding site;
optionally comprise an origin-specific barcode, a set-specific barcode, and/or a unique
molecular identifier (UMI); or
optionally comprises a gap region that, upon binding to a target sequence and gap filling,
comprises a gap-filled sequence.

4. (canceled)

5. (canceled)

6. The system of claim 1, wherein the proximity dependent probes are molecular inversion probes (MIPs), padlock probes, or split-ligation probes:
optionally wherein the gap-filled sequence comprises the guide polynucleotide recognition sequence;
optionally wherein the gap-filled sequence is at least 1 nucleotide in length; and
optionally wherein the gap-filled sequence comprises modified nucleotides further comprising a capture moiety, optionally further comprising a capture agent that binds the capture moiety of the modified nucleotides; and optionally wherein the modified nucleotides are biotinylated nucleotides and the capture agent is streptavidin or a streptavidin coated surface.

7. (canceled)

8. (canceled)

9. (canceled)

10. (canceled)

11. (canceled)

12. The system of claim 1, wherein the proximity dependent probe is a molecular inversion probe (MIP), and wherein the MIP comprises a first target binding sequence and a second target binding sequence linked by a linking region, the linking region comprising one or more of a forward primer binding sequence, a reverse primer binding sequence, a RNA polymerase binding sequence, a guide polynucleotide binding sequence, and a barcode,
optionally wherein the first target binding sequence and the second target binding sequence hybridize on the target sequence directly adjacent to one another;
optionally wherein the first and second target binding sequence hybridize on the target sequence such that there is at least a single nucleotide gap region between the first and second target binding sequence, preferably wherein filling the gap region between the first and second targeting binding sequence generates the guide polynucleotide recognition sequence;
optionally MIP comprises one or more of forward and reverse primers for amplification optionally including a T7 handle of RNA transcription, an inter-primer element for MIP linearization, and a barcode; and
optionally wherein the 5′ and 3′ ends of the MIP are placed immediately adjacent to each other upon hybridization to the target sequence.

13. (canceled)

14. (canceled)

15. (canceled)

16. The system of claim 1, wherein the tiled guide polynucleotide set comprises 2 to 50 guides per target sequence or wherein the proximity probe set is tiled and comprises 2 to 50 guide per target sequence, or wherein the tiled guide polynucleotide set comprises guide polynucleotides that cover at least 10% of a target sequence, or wherein the proximity probe set is tiled and comprises 2 to 50 guides per target sequence, or wherein the tiled guide polynucleotide set comprises 2 to 200 guides per target sequence.

17. (canceled)

18. The system of claim 1, further comprising amplification reagents for amplifying the proximity dependent probes, or amplification reagents for amplifying the target sequence, optionally wherein the amplification reagents comprise Polymerase Chain Reaction (PCR) reagents, Recombinase Polymerase Amplification (RPA) reagents, Rolling Circle Amplification (RCA) reagents, or Multiple Displacement Amplification (MDA) reagents, optionally wherein the system further comprises DNA methylation enrichment agents and/or size selection reagents to enrich for cfDNA.

19. (canceled)

20. (canceled)

21. (canceled)

22. The system of claim 1, wherein the one or more CRISPR-Cas proteins are a RNA-targeting protein, a DNA-targeting protein or a combination thereof, optionally wherein the CRISPR-Cas protein is a Cas13, a Cas 12 or a combination thereof, optionally wherein the Cas13 is a Cas13a, Cas13b, Cas13c, a Cas13d or a combination thereof, optionally wherein the Cas12 is a Cas12a, Cas12b, or Cas12c, optionally where each CRISPR-Cas has a different polynucleotide cutting preference and wherein the polynucleotide cutting preference is matched to a guide polynucleotide for a particular set of proximity dependent probes or tiled guide polynucleotides.

23. (canceled)

24. (canceled)

25. (canceled)

26. (canceled)

27. (canceled)

28. The detection system of claim 1, wherein the target of interest sequence is an antibiotic resistance gene, a repetitive genetic element, a conserved genomic regions across one or more genus or species, or a species-specific genomic region.

29. (canceled)

30. (canceled)

31. The system of claim 1, comprising two or more CRISPR systems,
wherein the CRISPR systems are RNA-targeting effector proteins, DNA-targeting effector proteins, or a combination thereof, or wherein the CRISPR system is a Cas13 system, as Cas12 system, or a combination thereof, optionally wherein the Cas 12 is Cpf1 or c2c1, the Cas 13 is Cas 13a, Cas 13b, or Cas 13c.

32. (canceled)

33. The system of claim 1, wherein the one or more guide RNAs are about 28 nucleotides in length and have a mismatch of one or less to the corresponding target sequence.

34. The system of claim 1, comprising two or more guide RNAs corresponding to target sequences in two or more pathogens, or two or more strains of a pathogen.

35. (canceled)

36. The system of claim 1, wherein the oligonucleotide based construct is a masking construct that suppresses generation of a detectable positive signal until cleaved by an activated CRISPR effector protein:
optionally wherein the masking construct suppresses generation of a detectable positive signal by masking the detectable positive signal, or generating a detectable negative signal instead;
optionally wherein the masking construct comprises a silencing RNA that suppresses generation of a gene product encoded by a reporting construct, wherein the gene product generates the detectable positive signal when expressed;
optionally wherein the masking construct is a ribozyme that generates the negative detectable signal, and wherein the positive detectable signal is generated when the ribozyme is deactivated,
preferably wherein the ribozyme converts a substrate to a first color and wherein the substrate converts to a second color when the ribozyme is deactivated,
preferably wherein the masking construct comprises a nanoparticle held in aggregate by bridge molecules, wherein at least a portion of the bridge molecules comprises DNA or RNA, and wherein the solution undergoes a color shift when the nanoparticle is disbursed in solution, optionally wherein the nanoparticle is colloidal metal, optionally colloidal gold;
optionally wherein the masking construct is a DNA or RNA aptamer and/or comprises a DNA or RNA-tethered inhibitor:
preferably wherein the aptamer or DNA- or RNA-tethered inhibitor sequesters an enzyme, wherein the enzyme generates a detectable signal upon release from the aptamer or DNA or RNA tethered inhibitor by acting upon a substrate,
preferably wherein the enzyme is thrombin and the substrate is para-nitroanilide covalently linked to a peptide substrate for thrombin, or 7-amino-4 methylcoumarin covalently linked to a peptide substrate for thrombin;
preferably wherein the aptamer is an inhibitor aptamer that inhibits an enzyme and prevents the enzyme from catalyzing generation of a detectable signal from a substance or wherein the DNA- or RNA-tethered inhibitor inhibits an enzyme and prevents the enzyme from catalyzing generation of a detectable signal from a substrate;
preferably wherein the aptamer sequesters a pair of agents that when released from the aptamers combine to generate a detectable signal;
optionally wherein the masking construct comprises a DNA or RNA oligonucleotide to which a detectable ligand and a masking component are attached,
preferably wherein the masking construct comprises DNA or RNA in complex with an intercalating agent, wherein the intercalating agent changes absorbance upon cleavage of the DNA or RNA, optionally wherein the intercalating agent is pyronine-Y or methylene blue, optionally wherein the detectable ligand is a fluorophore and the masking component is a quencher molecule;
optionally wherein the masking construct comprising a quantum dot linked to one or more quencher molecules by a linking molecule, wherein at least a portion of the linking molecule comprises DNA or RNA.

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. The system of claim 1, comprising two or more CRISPR systems, and wherein the masking construct of each CRISPR system is preferentially cut by one of the activated CRISPR proteins.

51. A method for detecting one or more nucleic acids in a sample, the method comprising
contacting one or more samples with the system of claim 1 comprising one or more sets of proximity dependent probes; and
detecting a signal from cleavage of the non-target sequence, thereby detecting the one or more target sequences in the sample optionally wherein:
a. amplifying the nucleic acids in the sample with the proximity dependent probes;
b. generating a first set of droplets, each droplet in the first set of droplets comprising at least one target molecule from the sample and an optical barcode;
c. generating a second set of droplets, each droplet in the second set of droplets comprising one or more detection CRISPR systems comprising the Cas protein and the one or more guide RNAs tiled to corresponding target sequences unique to the one or more strains or one or more pathogens, an RNA-based masking construct and an optical barcode;
d. combining the first set and second set of droplets into a pool of droplets and flowing the pool of droplets onto a microfluidic device comprising an array of microwells and at least one flow channel beneath the microwells, the microwells sized to capture at least two droplets;
e. detecting the optical barcodes of the droplets captured in each microwell;
f. merging the droplets captured in each microwell to form merged droplets in each microwell, at least a subset of the merged droplets comprising a detection CRISPR system and a target sequence;
g. initiating the detection reaction by incubating at about 37° C.; and
h. measuring a detectable signal of each merged droplet at one or more time periods.

52. A method for detecting one or more nucleic acids in a sample, the method comprising
contacting one or more samples with a system of claim 1 comprising one or more tiled guide polynucleotide sets;
detecting a signal from cleavage of the non-target sequence, thereby detecting the one or more target sequences in the sample optionally wherein:
a first optional step of heating the sample;
conducting preamplification on nucleic acid in the sample optionally wherein:
preferably wherein the preamplification is target specific, optionally selected from PCR, RPA, or RCA;
generating a first set of droplets, each droplet in the first set of droplets comprising at least one target molecule from the sample and an optical barcode;
generating a second set of droplets, each droplet in the second set of droplets comprising one or more detection CRISPR systems comprising the Cas protein and the one or more guide RNAs tiled to corresponding target sequences unique to the one or more strains or one or more pathogens, an RNA-based masking construct and an optical barcode;
combining the first set and second set of droplets into a pool of droplets and flowing the pool of droplets onto a microfluidic device comprising an array of microwells and at least one flow channel beneath the microwells, the microwells sized to capture at least two droplets;
detecting the optical barcodes of the droplets captured in each microwell;
merging the droplets captured in each microwell to form merged droplets in each microwell, at least a subset of the merged droplets comprising a detection CRISPR system and a target sequence;
initiating the detection reaction by incubating at about 37° C.; and
measuring a detectable signal of each merged droplet at one or more time periods.

53. The method of claim 51, wherein the proximity dependent probes are further optionally linked by ligation, splinted ligation, hybridization, or proximity extension, optionally comprise one or more of a forward primer binding site, a reverse primer binding site, and a reverse polymerase binding site;
optionally comprise an origin-specific barcode, a set-specific barcode, and/or a unique molecular identifier (UMI); or
optionally comprises a gap region that, upon binding to a target sequence and gap filling, comprises a gap-filled sequence.

54. (canceled)

55. (canceled)

56. The method of claim 51, wherein the proximity dependent probes are molecular inversion probes (MIPs), padlock probes, or split-ligation probes:
optionally wherein the gap-filled sequence comprises the guide polynucleotide recognition sequence;
optionally wherein the gap-filled sequence is at least 1 nucleotide in length; and
optionally wherein the gap-filled sequence comprises modified nucleotides further comprising a capture moiety, optionally further comprising a capture agent that binds the capture moiety of the modified nucleotides; and optionally wherein the modified nucleotides are biotinylated nucleotides and the capture agent is streptavidin or a streptavidin coated surface.

57. (canceled)

58. (canceled)

59. (canceled)

60. (canceled)

61. (canceled)

62. The method of claim 51, wherein the proximity dependent probe is a MIP, and wherein the MIP comprises a first target binding sequence and a second target binding sequence linked by a linking region, the linking region comprising one or more of a forward primer binding sequence, a reverse primer binding sequence, a RNA polymerase binding sequence, a guide polynucleotide binding sequence, and a barcode,
optionally wherein the first target binding sequence and the second target binding sequence hybridize on the target sequence directly adjacent to one another;
optionally wherein the first and second target binding sequence hybridize on the target sequence such that there is at least a single nucleotide gap region between the first and second target binding sequence, preferably wherein filling the gap region between the first and second targeting binding sequence generates the guide polynucleotide recognition sequence;
optionally MIP comprises one or more of forward and reverse primers for amplification optionally including a T7 handle of RNA transcription, an inter-primer element for MIP linearization, and a barcode; and
optionally wherein the 5′ and 3′ ends of the MIP are placed immediately adjacent to each other upon hybridization to the target sequence.

63. (canceled)

64. (canceled)

65. (canceled)

66. (canceled)

67. (canceled)

68. (canceled)

69. (canceled)

70. (canceled)

71. (canceled)

72. The method of claim 52, wherein the preamplification comprises probes developed by:
a. defining ‘in’ group comprising the genomes of interest and an ‘out’ group comprising genomes not of interest;
b. selecting a reference genome in the ‘in’ group and generating a list of all possible genomic targets of a pre-defined size, wherein the pre-defined size is between about 10 nt and 150 nt;
c. identifying matching sequences with all other genomes in the ‘in’ and ‘out’ groups using a sequence alignment tool;
d. generating a candidate list of possible genomic targets comprising sequences that match with all genomes in the ‘in group’ and do not match with any of the genomes in the ‘out group,’ thereby identifying probes for target sequences; and
optionally wherein the ‘out’ group comprises strains with a same genus but of a different species of a bacteria and the ‘in’ group comprises strains of the same bacterial species, optionally Staphylococcus aureus;
optionally wherein the ‘out’ group comprises pathogens not of interest and the ‘in’ group comprises one or more pathogens of interest;
optionally wherein the ‘in’ group comprises two, three or four pathogens of interest;
optionally wherein the one or more pathogens comprise Staphylococcus aureus, Aspergillus fumigatus and Mycobacterium tuberculosis;
optionally wherein the method comprises two or more CRISPR detection systems, wherein the Cas protein of each CRISPR detection system comprise orthogonal base preferences;
optionally wherein the CRISPR/Cas detection system comprises a Cas12a effector protein or a Cas13a effector protein
optionally wherein the preamplification comprises preferential amplification of microbial DNA by exploiting methylation sites and size selecting microbial cfDNA;
optionally wherein the preamplification is non-specific, optionally selected from adapter-ligation, degenerate PCR and MDA; and
optionally wherein the amplification is target-specific and probes comprise molecular inversion probes.

73. (canceled)

74. (canceled)

75. (canceled)

76. (canceled)

77. (canceled)

78. (canceled)

79. (canceled)

80. The method of claim 72, wherein the preamplification is non-specific, optionally selected from adapter-ligation, degenerate PCR and MDA.

81. (canceled)

82. The method of claim 51, wherein the sample is from a subject with active infection optionally further comprising a sample from a healthy subject and is optionally selected from the group consisting of plasma, blood, urine and saliva.

83. The method of claim 51, wherein the nucleic acid is cell free nucleic acid.

84. (canceled)

85. (canceled)

86. The method of claim 52, further comprising extraction of cfDNA from the sample prior to the step of preamplification.

87. The method of claim 52, wherein the guide RNAs are selected by:
defining ‘in’ group comprising the genomes of interest and an ‘out’ group comprising genomes not of interest;
selecting a reference genome in the ‘in’ group and generating a list of all possible genomic targets of 28 nucleotides;
identifying matching sequences with all other genomes in the ‘in’ and ‘out’ groups using a sequence alignment tool;
generating a candidate list of possible genomic targets comprising sequences that match with all genomes in the ‘in group’ and do not match with any of the genomes in the ‘out group,’ thereby identifying probes for target sequences.

88. The method of claim 52, wherein selection of the guide RNAs is further based on one or more of sequence orthogonality, melting temperature and/or genomic distribution.

89. The method of claim 52, wherein the guide RNAs have a mismatch tolerance of one nucleotide.

90. The method of claim 52, wherein the imaging the droplets is performed at intermittent intervals or continuously to measure fluorescence kinetics and/or quantitation.

91. A method of detecting host response to infection comprising:
performing the method of claim 51 on a host sample obtained at a first time and on a host sample obtained at a second time;
detecting the presence of one or more pathogens at the first time and the second time,
optionally wherein the host is treated with an antibiotic subsequent to the first time and prior to the second time;
optionally further comprising detecting antibiotic resistance and identifying genetic markers associated with antibiotic resistance.

92. (canceled)

93. (canceled)

94. The method of claim 52, wherein the preamplification step is performed in the first set of droplets after generating the first set of droplets.

95. A method for detecting target cell free nucleic acids in a sample, comprising:
a) distributing a sample or set of samples into one or more individual discrete volumes, the individual discrete volumes comprising a CRISPR system of claim 1;
b) incubating the sample or set of samples under conditions to allow binding of proximity dependent probes to one or more target molecules and amplifying the one or more target molecules;
c) activating the CRISPR effector protein via binding of the one or more guide RNAs to the one or more target sequences, wherein activating the CRISPR effector protein results in modification of the RNA-based masking construct such that a detectable positive signal is generated; and
d) detecting the one or more detectable positive signal, wherein detection of the one or more detectable positive signal indicates a presence of one or more target molecules in the sample,
optionally wherein the one or more guide RNAs correspond to one or more target molecules defined by evenly spaced regions of a genome of the one or more pathogens;
optionally wherein the one or more guide RNAs wherein the guide RNAs are designed by:
defining ‘in’ group comprising the genomes of interest and an ‘out’ group comprising genomes not of interest;
selecting a reference genome in the ‘in’ group and generating a list of all possible genomic targets of about 10 to about 80 nucleotides, or about 28 nucleotides;
identifying matching sequences with all other genomes in the ‘in’ and ‘out’ groups using a sequence alignment tool;
generating a candidate list of possible genomic targets comprising sequences that match with all genomes in the ‘in group’ and do not match with any of the genomes in the ‘out group,’ thereby identifying probes for target sequences;
optionally wherein selection of the guide RNAs is based on one or more of sequence orthogonality, melting temperature and/or genomic distribution;
optionally wherein the guide RNAs have a mismatch tolerance of one nucleotide.

96. (canceled)

97. (canceled)

98. (canceled)

99. (canceled)

100. A microfluidic device comprising a sample loading region, and one or more flow channels, each channel comprising a detector region comprising a detection construct and one or more nucleic acid detection systems, and at least a first and second capture region, the first capture region comprising a first binding agent and the second capture region comprising a second binding agent,
optionally wherein each region of the microfluidic flow device is a node:
preferably wherein each flow channel is arranged radially from a center node;
preferably wherein the center node comprises transcription reagents;
preferably further comprising one or more thermally differentiated zones disposed between the loading region and the center node;
optionally wherein each flow channel is arranged in parallel;
optionally wherein the detection construct comprises a first molecule on a first end and a second molecule on a second end;
optionally wherein one or more nucleic acid detection systems each comprise a Cas protein, and a species-specific guide RNA;
preferably wherein the guide RNA is designed to target an amplicon of the sample;
optionally further comprising one or more amplification reagents, optionally in the sample loading region;
preferably wherein the one or more amplification reagents are selected from nucleic acid sequence-based amplification (NASBA), recombinase polymerase amplification (RPA), loop-mediated isothermal amplification (LAMP), strand displacement amplification (SDA), helicase-dependent amplification (HDA), nicking enzyme amplification reaction (NEAR), PCR, multiple displacement amplification (MDA), rolling circle amplification (RCA), ligase chain reaction (LCR), or ramification amplification method (RAM);
optionally comprising the molecular inversion probes of claims and ligation reagents in the sample loading region;
preferably further comprising species-specific guide RNA designed to target a species-specific binding sequence on the MIP.

101.-112. (canceled)

113. The method of claim 52, wherein the sample is from a subject with active infection and optionally comprises a sample from a healthy subject and is optionally selected from the group consisting of plasma, blood, urine and saliva, or wherein the sample is cfDNA.

114. A method of detecting host response to infection comprising:
performing the method of claim 52 on a host sample obtained at a first time and on a host sample obtained at a second time; and
detecting the presence of one or more pathogens at the first time and the second time,
optionally wherein the host is treated with an antibiotic subsequent to the first time and prior to the second time;
optionally further comprising detecting antibiotic resistance and identifying genetic markers associated with antibiotic resistance.


SEQUENCE LISTING (23 sequences)

SEQ ID NO: 1 - protein, 6 aa, Artificial Sequence
  Synthetic Peptide
  MISC_FEATURE (2)..(2): Xaa = N, H, or K
  MISC_FEATURE (3)..(3): Xaa = R, S, D, E, Q, N, G, or Y
  MISC_FEATURE (4)..(4): Xaa = I, S, T, V, or L
  MISC_FEATURE (5)..(5): Xaa = L, F, N, Y, V, I, S, D, E, or A
RXXXXH

SEQ ID NO: 2 - DNA, 27 bp, Artificial Sequence
  Synthetic Oligonucleotide
cgattaaagatagaaatacacgatgcg

SEQ ID NO: 3 - DNA, 27 bp, Artificial Sequence
  Synthetic Oligonucleotide
gattaaagatagaaatacacgatgcga

SEQ ID NO: 4 - DNA, 27 bp, Artificial Sequence
  Synthetic Oligonucleotide
attaaagatagaaatacacgatgcgag

SEQ ID NO: 5 - protein, 6 aa, Artificial Sequence
  Synthetic Peptide
  MISC_FEATURE (2)..(2): Xaa = N or H
  MISC_FEATURE (3)..(3): Xaa = R, S, D, E, Q, N, G, Y, or H
  MISC_FEATURE (4)..(4): Xaa = I, S, T, V, or L
  MISC_FEATURE (5)..(5): Xaa = L, F, N, Y, V, I, S, D, E, or A
RXXXXH

SEQ ID NO: 6 - protein, 6 aa, Artificial Sequence
  Synthetic Peptide
  MISC_FEATURE (2)..(2): Xaa = N or K
  MISC_FEATURE (3)..(3): Xaa = R, S, D, E, Q, N, G, Y, or H
  MISC_FEATURE (4)..(4): Xaa = I, S, T, V, or L
  MISC_FEATURE (5)..(5): Xaa = L, F, N, Y, V, I, S, D, E, or A
RXXXXH

SEQ ID NO: 7 - DNA, 12 bp, Artificial Sequence
  Synthetic Oligonucleotide
acttgtttaagt

SEQ ID NO: 8 - DNA, 12 bp, Artificial Sequence
  Synthetic Oligonucleotide
  misc_feature (1)..(4): n = any set of nucleotides that complement nnnn in position 9-12.
  misc_feature (9)..(12): n = any set of nucleotides that complement nnnn in position 1-4.
nnnngtttnnnn

SEQ ID NO: 9 - DNA, 16 bp, Artificial Sequence
  Synthetic Oligonucleotide
ggcaccgagtcggtgc

SEQ ID NO: 10 - DNA, 17 bp, Artificial Sequence
  Synthetic Oligonucleotide
  misc_feature (1)..(7): N = any set of nucleotides that base pairs with nnnnnnn from position 11-17
  misc_feature (11)..(17): N = any set of nucleotides that base pairs with nnnnnnn from position 1-7
nnnnnnnagtnnnnnnn

SEQ ID NO: 11 - RNA, 30 nt, Artificial Sequence
  Synthetic Oligonucleotide
  misc_feature (8)..(8): n is a, c, g, or u
  misc_feature (9)..(12): n = any set of nucleotides that is complementary to n in position 17-20
  misc_feature (13)..(16): n is a, c, g, or u
  misc_feature (17)..(20): n = any set of nucleotides that is complementary to n in position 9-12
  misc_feature (23)..(23): n is a, c, g, or u
gyyyyagnnnnnnnnnnnnnaanuurrrru

SEQ ID NO: 12 - protein, 15 aa, Artificial Sequence
  Synthetic Peptide
GGGGSGGGGSGGGGS

SEQ ID NO: 13 - protein, 30 aa, Artificial Sequence
  Synthetic Peptide
GGGGSGGGGSGGGGSGGGGSGGGGSGGGGS

SEQ ID NO: 14 - protein, 45 aa, Artificial Sequence
  Synthetic Peptide
GGGGSGGGGSGGGGSGGGGSGGGGSGGGGSGGGGSGGGGSGGGGS

SEQ ID NO: 15 - protein, 60 aa, Artificial Sequence
  Synthetic Peptide
GGGGSGGGGSGGGGSGGGGSGGGGSGGGGSGGGGSGGGGSGGGGSGGGGSGGGGSGGGGS

SEQ ID NO: 16 - RNA, 25 nt, Artificial Sequence
  Synthetic Oligonucleotide
gggaacaaagcugaaguacuuaccc

SEQ ID NO: 17 - DNA, 18 bp, Artificial Sequence
  Synthetic Oligonucleotide
gggtagggcgggttggga

SEQ ID NO: 18 - DNA, 25 bp, Artificial Sequence
  Synthetic Oligonucleotide
  misc_feature (25)..(25): 3 prime thiol modification
ttataactattcctaaaaaaaaaaa

SEQ ID NO: 19 - DNA, 26 bp, Artificial Sequence
  Synthetic Oligonucleotide
  misc_feature (1)..(1): 5 prime thiol modification
aaaaaaaaaactcccctaataacaat

SEQ ID NO: 20 - RNA, 45 nt, Artificial Sequence
  Synthetic Oligonucleotide
ggguaggaauaguuauaauuucccuuucccauuguuauuagggag

SEQ ID NO: 21 - RNA, 12 nt, Artificial Sequence
  Synthetic Oligonucleotide
  misc_feature (1)..(1): 5 prime biotin tag
  misc_feature (12)..(12): 3 prime Iowa Black quencher
ucucguacguuc

SEQ ID NO: 22 - RNA, 24 nt, Artificial Sequence
  Synthetic Oligonucleotide
  misc_feature (1)..(1): 5 prime biotin tag
  misc_feature (24)..(24): 3 prime Iowa Black quencher
ucucguacguucucucguacguuc

SEQ ID NO: 23 - DNA, 12 bp, Artificial Sequence
  Synthetic Oligonucleotide
  misc_feature (1)..(12): n is a, c, g, or t
nnnnnnnnnnnn